SEQUENCE LISTING (147 sequences)

SEQ ID NO: 1 - protein, 118 aa, Homo sapiens
QVQVQDSGGELVQPGGSLRVSCKASGFNIKDSYMHWVRQAPGKGLEWVAFIDPETGNTYYADSVKGRFTISRDNSKNTLYLQMNSLRAEDTAVYYCARGSIYWYFDVWGKGTTVTVSS

SEQ ID NO: 2 - protein, 115 aa, Homo sapiens
DIQMTQSPSSLSASVGQRVTITCRASQSLLNSSNQKNSLGWYQQKPGKAPKLLIYFASTRQSGVPSRFSGSGSGTDFTLTISSLQPEDFATYYCQQHYSTPLTFGQGTKVDIKRC

SEQ ID NO: 3 - protein, 15 aa, synthetic construct
  VARIANT 6..10: note = Region may be deleted
  VARIANT 11..15: note = Region may be deleted
GGGGSGGGGSGGGGS

SEQ ID NO: 4 - protein, 13 aa, Human immunodeficiency virus 1
GRKKRRQRRRPPQ

SEQ ID NO: 5 - protein, 16 aa, synthetic construct
RQIKIWFQNRRMKWKK

SEQ ID NO: 6 - protein, 27 aa, synthetic construct
GWTLNSAGYLLGKINLKALAALAKKIL

SEQ ID NO: 7 - protein, 18 aa, synthetic construct
KLALKLALKALKAALKLA

SEQ ID NO: 8 - protein, 21 aa, synthetic construct
KETWWETWWTEWSQPKKKRKV

SEQ ID NO: 9 - protein, 12 aa, synthetic construct
PLILLRLLRGQF

SEQ ID NO: 10 - protein, 12 aa, synthetic construct
PLIYLRLLRGQF

SEQ ID NO: 11 - protein, 15 aa, synthetic construct
KLWMRWYSPTTRRYG

SEQ ID NO: 12 - protein, 27 aa, synthetic construct
GWTLNSAGYLLGKINLKALAALAKKIL

SEQ ID NO: 13 - protein, 23 aa, synthetic construct
MGLGLHLLVLAAALQGAKKKRKV

SEQ ID NO: 14 - protein, 18 aa, synthetic construct
KLALKLALKALKAALKLA

SEQ ID NO: 15 - protein, 26 aa, synthetic construct
KKALLALALHHLAHLALHLALALKKA

SEQ ID NO: 16 - protein, 18 aa, synthetic construct
LLIILRRRIRKQAHAHSK

SEQ ID NO: 17 - protein, 10 aa, synthetic construct
RRIPNRRPRR

SEQ ID NO: 18 - protein, 18 aa, synthetic construct
  MOD_RES 18: note = Amidation of C-terminus
RQWRRWWQRRQWRRWWQR

SEQ ID NO: 19 - protein, 9 aa, synthetic construct
  MOD_RES 9: note = Amidation of C-terminus
RKFRRKFKK

SEQ ID NO: 20 - protein, 16 aa, synthetic construct
RRRRRRRRHHHHLLLL

SEQ ID NO: 21 - protein, 12 aa, synthetic construct
  MOD_RES 1: note = N-terminal PEG12 spacer
  MOD_RES 1..12: note = D-Arginine
RRRRRRRRRRRR

SEQ ID NO: 22 - protein, 12 aa, synthetic construct
  MOD_RES 1: note = N-terminal PEG12 spacer
RRRRRRRRRRRR

SEQ ID NO: 23 - protein, 9 aa, synthetic construct
  MOD_RES 1: note = N-terminal PEG12 spacer
  MOD_RES 1..9: note = D-Arginine
RRRRRRRRR

SEQ ID NO: 24 - protein, 9 aa, synthetic construct
  MOD_RES 1: note = N-terminal PEG12 spacer
RRRRRRRRR

SEQ ID NO: 25 - protein, 12 aa, synthetic construct
RRRRRRRRRRRR

SEQ ID NO: 26 - protein, 19 aa, synthetic construct
RRRRRRRRRRRRRRRRRRC

SEQ ID NO: 27 - protein, 12 aa, synthetic construct
  MOD_RES 1: note = N-terminal PEG24 spacer
RRRRRRRRRRRR

SEQ ID NO: 28 - protein, 9 aa, synthetic construct
  MOD_RES 1: note = N-terminal PEG24 spacer
RRRRRRRRR

SEQ ID NO: 29 - protein, 15 aa, synthetic construct
RRRRRRRRRRRRRRR

SEQ ID NO: 30 - protein, 13 aa, synthetic construct
  MOD_RES 1: note = N-terminal PEG24 spacer
RRRRRRRRRRRRC

SEQ ID NO: 31 - protein, 18 aa, synthetic construct
  MOD_RES 1: note = N-terminal PEG12 spacer
RRRRRRRRRRRRRRRRRR

SEQ ID NO: 32 - protein, 15 aa, synthetic construct
  MOD_RES 1: note = N-terminal PEG12 spacer
RRRRRRRRRRRRRRR

SEQ ID NO: 33 - protein, 13 aa, synthetic construct
  MOD_RES 1: note = N-terminal PEG12 spacer
RRRRRRRRRRRRC

SEQ ID NO: 34 - RNA, 23 nt, synthetic construct
  source 1..23: mol_type = other RNA
  modified_base 1: mod_base = OTHER; note = 2'-fluorouridine
  modified_base 3: mod_base = OTHER; note = 2'-fluorouridine
  modified_base 9: mod_base = OTHER; note = 2'-fluorouridine
  modified_base 18: mod_base = OTHER; note = 2'-fluorouridine
  modified_base 2: mod_base = gm
  modified_base 13: mod_base = gm
  modified_base 15: mod_base = gm
  modified_base 19: mod_base = gm
  modified_base 23: mod_base = gm
  modified_base 4: mod_base = OTHER; note = 2'-O-methyladenosine
  modified_base 6: mod_base = OTHER; note = 2'-O-methyladenosine
  modified_base 8: mod_base = OTHER; note = 2'-O-methyladenosine
  modified_base 21: mod_base = OTHER; note = 2'-O-methyladenosine
  modified_base 5: mod_base = OTHER; note = 2'-fluoroadenosine
  modified_base 16: mod_base = OTHER; note = 2'-fluoroadenosine
  modified_base 22: mod_base = OTHER; note = 2'-fluoroadenosine
  modified_base 7: mod_base = OTHER; note = 2'-fluorocytidine
  modified_base 11: mod_base = OTHER; note = 2'-fluorocytidine
  modified_base 14: mod_base = OTHER; note = 2'-fluorocytidine
  modified_base 10: mod_base = cm
  modified_base 17: mod_base = cm
  modified_base 12: mod_base = um
  modified_base 20: mod_base = OTHER; note = 2'-fluoroguanosine
  misc_feature 21..22: note = phosphorothioate linkage
  misc_feature 22..23: note = phosphorothioate linkage
tgtaaacatcctgcgactggaag

SEQ ID NO: 35 - RNA, 20 nt, synthetic construct
  source 1..20: mol_type = other RNA
  misc_feature 1: note = amino C6 linker
  modified_base 1: mod_base = um
  modified_base 2: mod_base = cm
  modified_base 3: mod_base = cm
  modified_base 18: mod_base = OTHER; note = 2'-O-methyladenosine
  modified_base 20: mod_base = OTHER; note = 2'-O-methyladenosine
  modified_base 19: mod_base = cm
tccagtcgaggatgtttaca

SEQ ID NO: 36 - RNA, 22 nt, synthetic construct
  source 1..22: mol_type = other RNA
tgtaaacatcctcgactggaag

SEQ ID NO: 37 - RNA, 20 nt, synthetic construct
  source 1..20: mol_type = other RNA
ttcagtcggatgtttgcagc

SEQ ID NO: 38 - RNA, 23 nt, synthetic construct
  source 1..23: mol_type = other RNA
  modified_base 11: mod_base = cm
  modified_base 14: mod_base = cm
  modified_base 17: mod_base = cm
  modified_base 12: mod_base = um
  modified_base 18: mod_base = um
  modified_base 13: mod_base = gm
  modified_base 15: mod_base = gm
  modified_base 19: mod_base = gm
  modified_base 23: mod_base = gm
  modified_base 16: mod_base = OTHER; note = 2'-O-methyladenosine
  modified_base 22: mod_base = OTHER; note = 2'-fluoroadenosine
  misc_feature 21..22: note = phosphorothioate linkage
  misc_feature 22..23: note = phosphorothioate linkage
tgtaaacatcctgcgactggaag

SEQ ID NO: 39 - RNA, 20 nt, synthetic construct
  source 1..20: mol_type = other RNA
  misc_feature 1: note = amino C6 linker
  modified_base 1: mod_base = um
  modified_base 2: mod_base = cm
  modified_base 3: mod_base = cm
  modified_base 19: mod_base = cm
  modified_base 18: mod_base = OTHER; note = 2'-O-methyladenosine
  modified_base 20: mod_base = OTHER; note = 2'-O-methyladenosine
tccagtcgaggatgtttaca

SEQ ID NO: 40 - skipped

SEQ ID NO: 41 - RNA, 20 nt, synthetic construct
  source 1..20: mol_type = other RNA
  misc_feature 1: note = amino C6 linker
  misc_feature 20: note = amino C6 linker
  modified_base 1: mod_base = um
  modified_base 2: mod_base = cm
  modified_base 3: mod_base = cm
  modified_base 19: mod_base = cm
  modified_base 18: mod_base = OTHER; note = 2'-O-methyladenosine
  modified_base 20: mod_base = OTHER; note = 2'-O-methyladenosine
tccagtcgaggatgtttaca

SEQ ID NO: 42 - DNA, 16 bp, synthetic construct
  source 1..16: mol_type = other DNA
  modified_base 1: mod_base = OTHER; note = LNA guanine
  modified_base 2: mod_base = OTHER; note = LNA cytosine
  misc_feature 1..16: note = phosphothionate-modified backbone between every residue
  modified_base 15: mod_base = OTHER; note = LNA cytosine
  modified_base 16: mod_base = OTHER; note = LNA cytosine
gccatcgcgggtagcc

SEQ ID NO: 43 - DNA, 16 bp, synthetic construct
  source 1..16: mol_type = other DNA
  modified_base 1: mod_base = OTHER; note = LNA guanosine
  modified_base 2: mod_base = OTHER; note = LNA cytosine
  modified_base 15: mod_base = OTHER; note = LNA cytosine
  modified_base 16: mod_base = OTHER; note = LNA cytosine
  misc_feature 1: note = 5' amino-carbon 6 chain
  misc_feature 1..16: note = phosphothionate-modifined backbone between every residue
gccatcgcgggtagcc

SEQ ID NO: 44 - DNA, 16 bp, synthetic construct
  source 1..16: mol_type = other DNA
  misc_feature 1..16: note = phosphothionate-modified backbone between each residue
  modified_base 1: mod_base = OTHER; note = LNA thymine
  modified_base 2: mod_base = OTHER; note = LNA guanine
  modified_base 15: mod_base = OTHER; note = LNA thymine
  modified_base 16: mod_base = OTHER; note = LNA cytosine
  misc_feature 1: note = 5' amino-carbon 6 chain
tgtcgggagggccatc

SEQ ID NO: 45 - DNA, 16 bp, synthetic construct
  source 1..16: mol_type = other DNA
  misc_feature 1: note = 5- amino-carbon 6 chain
  misc_feature 3..14: note = phosphothionate-modified backbone between every residue
  modified_base 1: mod_base = OTHER; note = LNA thymine
  modified_base 2: mod_base = OTHER; note = LNA guanine
  modified_base 15: mod_base = OTHER; note = LNA thymine
  modified_base 16: mod_base = OTHER; note = LNA cytosine
tgtcgggagggccatc

SEQ ID NO: 46 - DNA, 16 bp, synthetic construct
  source 1..16: mol_type = other DNA
  misc_feature 1: note = 5' amino-carbon 6 chain
  modified_base 1: mod_base = OTHER; note = LNA thymine
  modified_base 2: mod_base = OTHER; note = LNA guanine
  modified_base 15: mod_base = OTHER; note = LNA thymine
  modified_base 16: mod_base = OTHER; note = LNA cytosine
  misc_feature 1..2: note = methylphosphonate-modified backbone
  misc_feature 3..14: note = phosphothionate-modified backbone between every residue
  misc_feature 15..16: note = methylphosphonate-modified backbone
tgtcgggagggccatc

SEQ ID NO: 47 - DNA, 16 bp, synthetic construct
  source 1..16: mol_type = other DNA
  misc_feature 1: note = 5' amino-carbon 6 chain
  modified_base 1: mod_base = OTHER; note = BNA thymine
  modified_base 2: mod_base = OTHER; note = BNA guanine
  modified_base 15: mod_base = OTHER; note = BNA thymine
  modified_base 16: mod_base = OTHER; note = BNA cytosine
  misc_feature 1..16: note = phosphothionate-modified backbone between every residue
tgtcgggagggccatc

SEQ ID NO: 48 - DNA, 16 bp, synthetic construct
  source 1..16: mol_type = other DNA
  misc_feature 1..16: note = phosphothionate-modified backbone between every residue
  modified_base 1: mod_base = OTHER; note = LNA guanine
  modified_base 2: mod_base = OTHER; note = LNA cytosine
  modified_base 15: mod_base = OTHER; note = LNA cytosine
  modified_base 16: mod_base = OTHER; note = LNA cytosine
gccatcgcgggtagcc

SEQ ID NO: 49 - DNA, 16 bp, synthetic construct
  source 1..16: mol_type = other DNA
  modified_base 1: mod_base = OTHER; note = LNA thymine
  modified_base 2: mod_base = OTHER; note = LNA cytosine
  modified_base 15: mod_base = OTHER; note = LNA cytosine
  modified_base 16: mod_base = OTHER; note = LNA guanine
  misc_feature 1..16: note = phosphothionate-modified backbone between every residue
tccaaacgagtctccg

SEQ ID NO: 50 - DNA, 16 bp, synthetic construct
  source 1..16: mol_type = other DNA
  misc_feature 1: note = 5' amino-carbon chain
  misc_feature 1..16: note = phosphothionate-modified backbone between every residue
  modified_base 1: mod_base = OTHER; note = LNA thymine
  modified_base 2: mod_base = OTHER; note = LNA cytosine
  modified_base 15: mod_base = OTHER; note = LNA cytosine
  modified_base 16: mod_base = OTHER; note = LNA guanine
tccaaacgagtctccg

SEQ ID NO: 51 - DNA, 16 bp, synthetic construct
  source 1..16: mol_type = other DNA
  misc_feature 1..16: note = phosphothionate-modified backbone between every residue
  modified_base 1: mod_base = OTHER; note = LNA guanine
  modified_base 2: mod_base = OTHER; note = LNA adenine
  modified_base 15: mod_base = OTHER; note = LNA adenine
  modified_base 16: mod_base = OTHER; note = LNA thymine
gattctgaaaccagat

SEQ ID NO: 52 - DNA, 16 bp, synthetic construct
  source 1..16: mol_type = other DNA
  misc_feature 1: note = 5' amino-carbon 6 chain
  misc_feature 1..16: note = phosphothionate-modified backbone between every residue
  modified_base 1: mod_base = OTHER; note = LNA guanine
  modified_base 2: mod_base = OTHER; note = LNA adenine
  modified_base 15: mod_base = OTHER; note = LNA adenine
  modified_base 16: mod_base = OTHER; note = LNA thymine
gattctgaaaccagat

SEQ ID NO: 53 - DNA, 16 bp, synthetic construct
  source 1..16: mol_type = other DNA
  misc_feature 1..16: note = phosphothionate-modified backbone between every residue
  modified_base 1: mod_base = OTHER; note = LNA guanine
  modified_base 2: mod_base = OTHER; note = LNA cytosine
  modified_base 15: mod_base = OTHER; note = LNA adenine
  modified_base 16: mod_base = OTHER; note = LNA cytosine
gcgggcgccctgccac

SEQ ID NO: 54 - DNA, 16 bp, synthetic construct
  source 1..16: mol_type = other DNA
  misc_feature 1: note = 5' amino-carbon 6 chain
  misc_feature 1..16: note = phosphothionate-modified backbone between every residue
  modified_base 1: mod_base = OTHER; note = LNA guanine
  modified_base 2: mod_base = OTHER; note = LNA cytosine
  modified_base 15: mod_base = OTHER; note = LNA adenine
  modified_base 16: mod_base = OTHER; note = LNA cytosine
gcgggcgccctgccac

SEQ ID NO: 55 - DNA, 16 bp, synthetic construct
  source 1..16: mol_type = other DNA
  misc_feature 1..16: note = phosphothionate-modified backbone between every residue
  modified_base 1: mod_base = OTHER; note = LNA thymine
  modified_base 2: mod_base = OTHER; note = LNA cytosine
  modified_base 15: mod_base = OTHER; note = LNA guanine
  modified_base 16: mod_base = OTHER; note = LNA cytosine
tcatccagcagcaggc

SEQ ID NO: 56 - DNA, 16 bp, synthetic construct
  source 1..16: mol_type = other DNA
  misc_feature 1: note = 5' amino-carbon 6 chain
  misc_feature 1..16: note = phosphothionate-modified backbone between every residue
  modified_base 1: mod_base = OTHER; note = LNA thymine
  modified_base 2: mod_base = OTHER; note = LNA cytosine
  modified_base 15: mod_base = OTHER; note = LNA guanine
  modified_base 16: mod_base = OTHER; note = LNA cytosine
tcatccagcagcaggc

SEQ ID NO: 57 - DNA, 16 bp, synthetic construct
  source 1..16: mol_type = other DNA
  misc_feature 1..16: note = phosphothionate-modified backbone
  modified_base 1: mod_base = OTHER; note = LNA thymine
  modified_base 2: mod_base = OTHER; note = LNA adenine
  modified_base 15..16: mod_base = OTHER; note = LNA thymine
tagccagccaggtgtt

SEQ ID NO: 58 - DNA, 16 bp, synthetic construct
  source 1..16: mol_type = other DNA
  misc_feature 1: note = 5' amino-carbon 6 chain
  misc_feature 1..16: note = phosphothionate-modified backbone between every residue
  modified_base 1: mod_base = OTHER; note = LNA thymine
  modified_base 2: mod_base = OTHER; note = LNA adenine
  modified_base 15..16: mod_base = OTHER; note = LNA thymine
tagccagccaggtgtt

SEQ ID NO: 59 - DNA, 16 bp, synthetic construct
  source 1..16: mol_type = other DNA
  misc_feature 1..16: note = phosphothionate-modified backbone between every residue
  modified_base 1: mod_base = OTHER; note = LNA cytosine
  modified_base 2: mod_base = OTHER; note = LNA adenine
  modified_base 15: mod_base = OTHER; note = LNA guanine
  modified_base 16: mod_base = OTHER; note = LNA guanine
cagcgtcggaaggtgg

SEQ ID NO: 60 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA cytosine
  modified_base 2: mod_base = OTHER; note = LNA adenine
  modified_base 15: mod_base = OTHER; note = LNA guanine
  modified_base 16: mod_base = OTHER; note = LNA guanine
cagcgtcggaaggtgg

SEQ ID NO: 61 - DNA, 16 bp, synthetic construct
  source 1..16: mol_type = other DNA
  misc_feature 1..16: note = phosphothionate-modified backbone between every residue
  modified_base 1: mod_base = OTHER; note = LNA thymine
  modified_base 2: mod_base = OTHER; note = LNA adenine
  modified_base 15: mod_base = OTHER; note = LNA adenine
  modified_base 16: mod_base = OTHER; note = LNA guanine
tagacagcgtcggaag

SEQ ID NO: 62 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA thymine
  modified_base 2: mod_base = OTHER; note = LNA adenine
  modified_base 15: mod_base = OTHER; note = LNA adenine
  modified_base 16: mod_base = OTHER; note = LNA guanine
tagacagcgtcggaag

SEQ ID NO: 63 - DNA, 16 bp, synthetic construct
  source 1..16: mol_type = other DNA
  misc_feature 1..16: note = phosphothionate-modified backbone between every residue
  modified_base 1: mod_base = OTHER; note = LNA adenine
  modified_base 2: mod_base = OTHER; note = LNA thymine
  modified_base 15: mod_base = OTHER; note = LNA guanine
  modified_base 16: mod_base = OTHER; note = LNA adenine
ataggatccacaggga

SEQ ID NO: 64 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA adenine
  modified_base 2: mod_base = OTHER; note = LNA thymine
  modified_base 15: mod_base = OTHER; note = LNA guanine
  modified_base 16: mod_base = OTHER; note = LNA adenine
ataggatccacaggga

SEQ ID NO: 65 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = BNA adenine
  modified_base 2: mod_base = OTHER; note = BNA thymine
  modified_base 15: mod_base = OTHER; note = BNA guanine
  modified_base 16: mod_base = OTHER; note = BNA adenine
ataggatccacaggga

SEQ ID NO: 66 - DNA, 16 bp, synthetic construct
  source 1..16: mol_type = other DNA
  misc_feature 1..16: note = phosphothionate-modified backbone between every residue
  modified_base 1: mod_base = OTHER; note = LNA thymine
  modified_base 2: mod_base = OTHER; note = LNA cytosine
  modified_base 15: mod_base = OTHER; note = LNA adenine
  modified_base 16: mod_base = OTHER; note = LNA guanine
tctataggatccacag

SEQ ID NO: 67 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA thymine
  modified_base 2: mod_base = OTHER; note = LNA cytosine
  modified_base 15: mod_base = OTHER; note = LNA adenine
  modified_base 16: mod_base = OTHER; note = LNA guanine
tctataggatccacag

SEQ ID NO: 68 - DNA, 16 bp, synthetic construct
  source 1..16: mol_type = other DNA
  misc_feature 1..16: note = phosphothionate-modified backbone between every residue
  modified_base 1: mod_base = OTHER; note = LNA guanine
  modified_base 2: mod_base = OTHER; note = LNA cytosine
  modified_base 15: mod_base = OTHER; note = LNA guanine
  modified_base 16: mod_base = OTHER; note = LNA guanine
gcactaatcatccagg

SEQ ID NO: 69 - DNA, 16 bp, synthetic construct
  source 1..16: mol_type = other DNA
  misc_feature 1..16: note = phosphothionate-modified backbone between every residue
  misc_feature 1: note = 5' amino-carbon 6 chain
  modified_base 1: mod_base = OTHER; note = LNA guanine
  modified_base 2: mod_base = OTHER; note = LNA cytosine
  modified_base 15: mod_base = OTHER; note = LNA guanine
  modified_base 16: mod_base = OTHER; note = LNA guanine
gcactaatcatccagg

SEQ ID NO: 70 - DNA, 15 bp, synthetic construct
  source 1..15: mol_type = other DNA
  misc_feature 1..15: note = phosphothionate-modified backbone between every residue
  modified_base 1: mod_base = OTHER; note = LNA cytosine
  modified_base 2: mod_base = OTHER; note = LNA adenine
  modified_base 3: mod_base = OTHER; note = LNA guanine
  modified_base 13: mod_base = OTHER; note = LNA guanine
  modified_base 14: mod_base = OTHER; note = LNA thymine
  modified_base 15: mod_base = OTHER; note = LNA guanine
cagcgtcggaaggtg

SEQ ID NO: 71 - DNA, 15 bp, synthetic construct
  source 1..15: mol_type = other DNA
  misc_feature 1..15: note = phosphothionate-modified backbone between every residue
  misc_feature 1: note = 5' amino-carbon 6 chain
  modified_base 1: mod_base = OTHER; note = LNA cytosine
  modified_base 2: mod_base = OTHER; note = LNA adenine
  modified_base 3: mod_base = OTHER; note = LNA guanine
  modified_base 13: mod_base = OTHER; note = LNA guanine
  modified_base 14: mod_base = OTHER; note = LNA thymine
  modified_base 15: mod_base = OTHER; note = LNA guanine
cagcgtcggaaggtg

SEQ ID NO: 72 - DNA, 17 bp, synthetic construct
  source 1..17: mol_type = other DNA
  misc_feature 1..17: note = phosphothionate-modified backbone between every residue
  modified_base 1: mod_base = OTHER; note = 2'-methyloxyethyl cytosine
  modified_base 2: mod_base = OTHER; note = 2'-methyloxyethyl cytosine
  modified_base 3: mod_base = OTHER; note = 2'-methyloxyethyl adenine
  modified_base 4: mod_base = OTHER; note = 2'-methyloxyethyl guanine
  modified_base 14: mod_base = OTHER; note = 2'-methyloxyethyl guanine
  modified_base 15: mod_base = OTHER; note = 2'-methyloxyethyl thymine
  modified_base 16: mod_base = OTHER; note = 2'-methyloxyethyl guanine
  modified_base 17: mod_base = OTHER; note = 2'-methyloxyethyl thymine
ccagcgtcggaaggtgt

SEQ ID NO: 73 - DNA, 17 bp, synthetic construct
  source 1..17: mol_type = other DNA
  misc_feature 1..17: note = phosphothionate-modified backbone between every residue
  misc_feature 1: note = 5' amino-carbon 6 chain
  modified_base 1: mod_base = OTHER; note = 2'-methyloxyethyl cytosine
  modified_base 2: mod_base = OTHER; note = 2'-methyloxyethyl cytosine
  modified_base 3: mod_base = OTHER; note = 2'-methyloxyethyl adenine
  modified_base 4: mod_base = OTHER; note = 2'-methyloxyethyl guanine
  modified_base 14: mod_base = OTHER; note = 2'-methyloxyethyl guanine
  modified_base 15: mod_base = OTHER; note = 2'-methyloxyethyl thymine
  modified_base 16: mod_base = OTHER; note = 2'-methyloxyethyl guanine
  modified_base 17: mod_base = OTHER; note = 2'-methyloxyethyl thymine
ccagcgtcggaaggtgt

SEQ ID NO: 74 - DNA, 20 bp, synthetic construct
  source 1..20: mol_type = other DNA
  misc_feature 1..20: note = phosphothionate-modified backbone between every residue
  modified_base 1: mod_base = OTHER; note = LNA cytosine
  modified_base 2: mod_base = OTHER; note = LNA cytosine
  modified_base 4: mod_base = OTHER; note = LNA adenine
  modified_base 15: mod_base = OTHER; note = LNA guanine
  modified_base 16: mod_base = OTHER; note = LNA adenine
  modified_base 19: mod_base = OTHER; note = LNA guanine
  modified_base 20: mod_base = OTHER; note = LNA thymine
cctagacagcgtcggaaggt

SEQ ID NO: 75 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA thymine
  modified_base 3: mod_base = OTHER; note = LNA adenine
  modified_base 16: mod_base = OTHER; note = LNA adenine
  modified_base 17: mod_base = OTHER; note = LNA guanine
  modified_base 18: mod_base = OTHER; note = LNA guanine
ataggatccacagggagg

SEQ ID NO: 76 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA guanine
  modified_base 3: mod_base = OTHER; note = LNA guanine
  modified_base 16: mod_base = OTHER; note = LNA cytosine
  modified_base 17: mod_base = OTHER; note = LNA guanine
  modified_base 18: mod_base = OTHER; note = LNA guanine
cggctctgggatccccgg

SEQ ID NO: 77 - DNA, 17 bp, synthetic construct
  source 1..17: mol_type = other DNA
  misc_feature 1..17: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA guanine
  modified_base 15: mod_base = OTHER; note = LNA cytosine
  modified_base 16: mod_base = OTHER; note = LNA cytosine
  modified_base 17: mod_base = OTHER; note = LNA cytosine
ggggcggagacacgccc

SEQ ID NO: 78 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA guanine
  modified_base 3: mod_base = OTHER; note = LNA adenine
  modified_base 16: mod_base = OTHER; note = LNA cytosine
  modified_base 17: mod_base = OTHER; note = LNA adenine
  modified_base 18: mod_base = OTHER; note = LNA guanine
agaaggcaggaatcccag

SEQ ID NO: 79 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA cytosine
  modified_base 3: mod_base = OTHER; note = LNA adenine
  modified_base 16: mod_base = OTHER; note = LNA cytosine
  modified_base 17: mod_base = OTHER; note = LNA guanine
  modified_base 18: mod_base = OTHER; note = LNA guanine
gcaggaatcccaggccgg

SEQ ID NO: 80 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA adenine
  modified_base 16: mod_base = OTHER; note = LNA guanine
  modified_base 17: mod_base = OTHER; note = LNA cytosine
  modified_base 18: mod_base = OTHER; note = LNA cytosine
ggagtctctcaccgggcc

SEQ ID NO: 81 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA guanine
  modified_base 3: mod_base = OTHER; note = LNA adenine
  modified_base 16: mod_base = OTHER; note = LNA cytosine
  modified_base 17: mod_base = OTHER; note = LNA cytosine
  modified_base 18: mod_base = OTHER; note = LNA cytosine
agaggccagcgagctccc

SEQ ID NO: 82 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA cytosine
  modified_base 16: mod_base = OTHER; note = LNA guanine
  modified_base 17: mod_base = OTHER; note = LNA guanine
  modified_base 18: mod_base = OTHER; note = LNA guanine
ggctctgggatccccggg

SEQ ID NO: 83 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA adenine
  modified_base 3: mod_base = OTHER; note = LNA guanine
  modified_base 16: mod_base = OTHER; note = LNA guanine
  modified_base 17: mod_base = OTHER; note = LNA cytosine
  modified_base 18: mod_base = OTHER; note = LNA thymine
cagagaggccagcgagct

SEQ ID NO: 84 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA adenine
  modified_base 3: mod_base = OTHER; note = LNA cytosine
  modified_base 16: mod_base = OTHER; note = LNA thymine
  modified_base 17: mod_base = OTHER; note = LNA guanine
  modified_base 18: mod_base = OTHER; note = LNA guanine
gacagcgtcggaaggtgg

SEQ ID NO: 85 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA thymine
  modified_base 3: mod_base = OTHER; note = LNA adenine
  modified_base 16: mod_base = OTHER; note = LNA guanine
  modified_base 17: mod_base = OTHER; note = LNA thymine
  modified_base 18: mod_base = OTHER; note = LNA thymine
gtaactctaatccaggtt

SEQ ID NO: 86 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA cytosine
  modified_base 3: mod_base = OTHER; note = LNA adenine
  modified_base 16: mod_base = OTHER; note = LNA guanine
  modified_base 17: mod_base = OTHER; note = LNA guanine
  modified_base 18: mod_base = OTHER; note = LNA cytosine
acaagggcacagagaggc

SEQ ID NO: 87 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA adenine
  modified_base 3: mod_base = OTHER; note = LNA guanine
  modified_base 16: mod_base = OTHER; note = LNA thymine
  modified_base 17: mod_base = OTHER; note = LNA cytosine
  modified_base 18: mod_base = OTHER; note = LNA adenine
gagctcccttgcacgtca

SEQ ID NO: 88 - DNA, 17 bp, synthetic construct
  source 1..17: mol_type = other DNA
  misc_feature 1..17: note = phosphothionate-modified backbone between every residue
  modified_base 1: mod_base = OTHER; note = LNA cytosine
  modified_base 2: mod_base = OTHER; note = LNA cytosine
  modified_base 3: mod_base = OTHER; note = LNA guanine
  modified_base 15: mod_base = OTHER; note = LNA guanine
  modified_base 16: mod_base = OTHER; note = LNA thymine
  modified_base 17: mod_base = OTHER; note = LNA cytosine
ccgtccaaccccgcgtc

SEQ ID NO: 89 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA cytosine
  modified_base 3: mod_base = OTHER; note = LNA thymine
  modified_base 16: mod_base = OTHER; note = LNA guanine
  modified_base 17: mod_base = OTHER; note = LNA cytosine
  modified_base 18: mod_base = OTHER; note = LNA adenine
cctaaagctcctccagca

SEQ ID NO: 90 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA cytosine
  modified_base 3: mod_base = OTHER; note = LNA guanine
  modified_base 16: mod_base = OTHER; note = LNA cytosine
  modified_base 17: mod_base = OTHER; note = LNA cytosine
  modified_base 18: mod_base = OTHER; note = LNA guanine
gcgaggcggcctcttccg

SEQ ID NO: 91 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA cytosine
  modified_base 3: mod_base = OTHER; note = LNA cytosine
  modified_base 16: mod_base = OTHER; note = LNA guanine
  modified_base 17: mod_base = OTHER; note = LNA guanine
  modified_base 18: mod_base = OTHER; note = LNA guanine
gcctccagctcccccggg

SEQ ID NO: 92 - DNA, 17 bp, synthetic construct
  source 1..17: mol_type = other DNA
  misc_feature 1..17: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA thymine
  modified_base 15: mod_base = OTHER; note = LNA cytosine
  modified_base 16: mod_base = OTHER; note = LNA adenine
  modified_base 17: mod_base = OTHER; note = LNA thymine
ggtgtcgggagggccat

SEQ ID NO: 93 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA thymine
  modified_base 3: mod_base = OTHER; note = LNA guanine
  modified_base 16: mod_base = OTHER; note = LNA thymine
  modified_base 17: mod_base = OTHER; note = LNA cytosine
  modified_base 18: mod_base = OTHER; note = LNA thymine
ctgggccagccgttctct

SEQ ID NO: 94 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA guanine
  modified_base 3: mod_base = OTHER; note = LNA guanine
  modified_base 16: mod_base = OTHER; note = LNA thymine
  modified_base 17: mod_base = OTHER; note = LNA guanine
  modified_base 18: mod_base = OTHER; note = LNA guanine
gggccagccgttctctgg

SEQ ID NO: 95 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA adenine
  modified_base 15: mod_base = OTHER; note = LNA thymine
  modified_base 17: mod_base = OTHER; note = LNA cytosine
  modified_base 18: mod_base = OTHER; note = LNA thymine
caatttcaggctttttct

SEQ ID NO: 96 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA guanine
  modified_base 3: mod_base = OTHER; note = LNA cytosine
  modified_base 16: mod_base = OTHER; note = LNA thymine
  modified_base 17: mod_base = OTHER; note = LNA thymine
  modified_base 18: mod_base = OTHER; note = LNA guanine
tgcctacagaaggctttg

SEQ ID NO: 97 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA thymine
  modified_base 15: mod_base = OTHER; note = LNA cytosine
  modified_base 17: mod_base = OTHER; note = LNA cytosine
  modified_base 18: mod_base = OTHER; note = LNA adenine
atctctgcactcatcaca

SEQ ID NO: 98 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA thymine
  modified_base 15: mod_base = OTHER; note = LNA thymine
  modified_base 17: mod_base = OTHER; note = LNA thymine
  modified_base 18: mod_base = OTHER; note = LNA guanine
ctgatcaccgaagttctg

SEQ ID NO: 99 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA thymine
  modified_base 15: mod_base = OTHER; note = LNA guanine
  modified_base 17: mod_base = OTHER; note = LNA guanine
  modified_base 18: mod_base = OTHER; note = LNA guanine
ataggatccacagggagg

SEQ ID NO: 100 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA cytosine
  modified_base 15: mod_base = OTHER; note = LNA thymine
  modified_base 17: mod_base = OTHER; note = LNA thymine
  modified_base 18: mod_base = OTHER; note = LNA adenine
ccaggagatgtaactcta

SEQ ID NO: 101 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA adenine
  modified_base 3: mod_base = OTHER; note = LNA adenine
  modified_base 16: mod_base = OTHER; note = LNA guanine
  modified_base 17: mod_base = OTHER; note = LNA cytosine
  modified_base 18: mod_base = OTHER; note = LNA cytosine
gaaagagaggccaccgcc

SEQ ID NO: 102 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA thymine
  modified_base 3: mod_base = OTHER; note = LNA adenine
  modified_base 16: mod_base = OTHER; note = LNA thymine
  modified_base 17: mod_base = OTHER; note = LNA thymine
  modified_base 18: mod_base = OTHER; note = LNA cytosine
gtagccagccaggtgttc

SEQ ID NO: 103 - DNA, 18 bp, synthetic construct
  source 1..18: mol_type = other DNA
  misc_feature 1..18: note = phosphothionate-modified backbone between every residue
  modified_base 1: mod_base = OTHER; note = LNA guanine
  modified_base 2: mod_base = OTHER; note = LNA cytosine
  modified_base 3: mod_base = OTHER; note = LNA cytosine
  modified_base 16: mod_base = OTHER; note = LNA guanine
  modified_base 17: mod_base = OTHER; note = LNA cytosine
  modified_base 18: mod_base = OTHER; note = LNA cytosine
gcccctccgtagccagcc

SEQ ID NO: 104 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA guanine
  modified_base 3: mod_base = OTHER; note = LNA cytosine
  modified_base 16: mod_base = OTHER; note = LNA thymine
  modified_base 17: mod_base = OTHER; note = LNA cytosine
  modified_base 18: mod_base = OTHER; note = LNA guanine
tgctgtccgagggtgtcg

SEQ ID NO: 105 - DNA, 18 bp, synthetic construct
  source 1..18: mol_type = other DNA
  misc_feature 1..18: note = phosphothionate-modified backbone between every residue
  modified_base 1: mod_base = OTHER; note = LNA adenine
  modified_base 2: mod_base = OTHER; note = LNA guanine
  modified_base 3: mod_base = OTHER; note = LNA guanine
  modified_base 16: mod_base = OTHER; note = LNA guanine
  modified_base 17: mod_base = OTHER; note = LNA adenine
  modified_base 18: mod_base = OTHER; note = LNA guanine
aggggtgcttccagcgag

SEQ ID NO: 106 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA thymine
  modified_base 3: mod_base = OTHER; note = LNA cytosine
  modified_base 16: mod_base = OTHER; note = LNA guanine
  modified_base 17: mod_base = OTHER; note = LNA guanine
  modified_base 18: mod_base = OTHER; note = LNA guanine
ttcttcctcgctgagggg

SEQ ID NO: 107 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA guanine
  modified_base 3: mod_base = OTHER; note = LNA guanine
  modified_base 16: mod_base = OTHER; note = LNA cytosine
  modified_base 17: mod_base = OTHER; note = LNA thymine
  modified_base 18: mod_base = OTHER; note = LNA guanine
cggtattcttcctcgctg

SEQ ID NO: 108 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA cytosine
  modified_base 3: mod_base = OTHER; note = LNA cytosine
  modified_base 16: mod_base = OTHER; note = LNA cytosine
  modified_base 17: mod_base = OTHER; note = LNA guanine
  modified_base 18: mod_base = OTHER; note = LNA guanine
tcctccagcagagcccgg

SEQ ID NO: 109 - DNA, 18 bp, synthetic construct
  source 1..18: mol_type = other DNA
  misc_feature 1..18: note = phosphothionate-modified backbone between every residue
  modified_base 1: mod_base = OTHER; note = LNA cytosine
  modified_base 2: mod_base = OTHER; note = LNA cytosine
  modified_base 3: mod_base = OTHER; note = LNA thymine
  modified_base 16: mod_base = OTHER; note = LNA guanine
  modified_base 17: mod_base = OTHER; note = LNA guanine
  modified_base 18: mod_base = OTHER; note = LNA adenine
cctgggccggctctggga

SEQ ID NO: 110 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA guanine
  modified_base 3: mod_base = OTHER; note = LNA cytosine
  modified_base 16: mod_base = OTHER; note = LNA cytosine
  modified_base 17: mod_base = OTHER; note = LNA guanine
  modified_base 18: mod_base = OTHER; note = LNA guanine
tgctggtacctgggccgg

SEQ ID NO: 111 - DNA, 18 bp, synthetic construct
  source 1..18: mol_type = other DNA
  misc_feature 1..18: note = phosphothionate-modified backbone between every residue
  modified_base 1: mod_base = OTHER; note = LNA thymine
  modified_base 2: mod_base = OTHER; note = LNA cytosine
  modified_base 3: mod_base = OTHER; note = LNA thymine
  modified_base 16: mod_base = OTHER; note = LNA guanine
  modified_base 17: mod_base = OTHER; note = LNA guanine
  modified_base 18: mod_base = OTHER; note = LNA guanine
tctataggatccacaggg

SEQ ID NO: 112 - protein, 4 aa, synthetic construct
GGFG

SEQ ID NO: 113 - protein, 4 aa, synthetic construct
ALAL

SEQ ID NO: 114 - protein, 4 aa, synthetic construct
GFLG

SEQ ID NO: 115 - protein, 12 aa, synthetic construct
  MOD_RES 1: note = N-terminal PEG12 spacer
  MOD_RES 12: note = C-terminal PEG12 spacer
RRRRRRRRRRRR

SEQ ID NO: 116 - protein, 18 aa, synthetic construct
RGRRGRRGRRGRRGRRGR

SEQ ID NO: 117 - protein, 18 aa, synthetic construct
RQRRQRRQRRQRRQRRQR

SEQ ID NO: 118 - protein, 18 aa, synthetic construct
RKRRKRRKRRKRRKRRKR

SEQ ID NO: 119 - protein, 18 aa, synthetic construct
RSRRSRRSRRSRRSRRSR

SEQ ID NO: 120 - protein, 18 aa, synthetic construct
RTRRTRRTRRTRRTRRTR

SEQ ID NO: 121 - protein, 18 aa, synthetic construct
RWRRWRRWRRWRRWRRWR

SEQ ID NO: 122 - protein, 15 aa, synthetic construct
SSSRRRRRRRRRRRR

SEQ ID NO: 123 - protein, 18 aa, synthetic construct
SSSSSSRRRRRRRRRRRR

SEQ ID NO: 124 - protein, 21 aa, synthetic construct
SSSSSSSSSRRRRRRRRRRRR

SEQ ID NO: 125 - protein, 18 aa, synthetic construct
SSSRRRRRRRRRRRRSSS

SEQ ID NO: 126 - protein, 18 aa, synthetic construct
TTTTTTRRRRRRRRRRRR

SEQ ID NO: 127 - protein, 6 aa, synthetic construct
RRRRRR

SEQ ID NO: 128 - protein, 6 aa, synthetic construct
  MOD_RES 1: note = N-terminal PEG12 spacer
RRRRRR

SEQ ID NO: 129 - protein, 7 aa, synthetic construct
  MOD_RES 1: note = N-terminal PEG12 spacer
RRRRRRC

SEQ ID NO: 130 - protein, 13 aa, synthetic construct
RRRRRRRRRRRRC

SEQ ID NO: 131 - protein, 11 aa, synthetic construct
RRRRRRRRRRR

SEQ ID NO: 132 - protein, 13 aa, synthetic construct
  MOD_RES 13: note = C-terminal maleimide-linked 2kDa PEG spacer
RRRRRRRRRRRRC

SEQ ID NO: 133 - protein, 13 aa, synthetic construct
  MOD_RES 13: note = C-terminal maleimide-linked 5kDa PEG spacer
RRRRRRRRRRRRC

SEQ ID NO: 134 - protein, 13 aa, synthetic construct
  MOD_RES 13: note = C-terminal 5kDa QuadPEG spacer
RRRRRRRRRRRRC

SEQ ID NO: 135 - protein, 13 aa, synthetic construct
  MOD_RES 13: note = C-terminal maleimide-linked 5kDa Dextran spacer
RRRRRRRRRRRRC

SEQ ID NO: 136 - protein, 9 aa, synthetic construct
RRRRRRRRR

SEQ ID NO: 137 - protein, 10 aa, synthetic construct
RRRRRRRRRC

SEQ ID NO: 138 - protein, 12 aa, synthetic construct
  MOD_RES 1: note = N-terminal 1kDa PEG spacer
RRRRRRRRRRRR

SEQ ID NO: 139 - protein, 12 aa, synthetic construct
  MOD_RES 1: note = N-terminal 2kDa PEG spacer
RRRRRRRRRRRR

SEQ ID NO: 140 - protein, 12 aa, synthetic construct
  MOD_RES 1: note = N-terminal 5kDa PEG spacer
RRRRRRRRRRRR

SEQ ID NO: 141 - protein, 14 aa, synthetic construct
CRRRRRRRRRRRRC

SEQ ID NO: 142 - protein, 13 aa, synthetic construct
  MOD_RES 13: note = C-terminal maleimide-linked 1.5kDa branched PEG spacer
RRRRRRRRRRRRC

SEQ ID NO: 143 - protein, 13 aa, synthetic construct
  MOD_RES 13: note = C-terminal maleimide-linked 3.9kDa branched PEG spacer
RRRRRRRRRRRRC

SEQ ID NO: 144 - protein, 13 aa, synthetic construct
  MOD_RES 13: note = C-terminal maleimide-linked 16kDa branched PEG spacer
RRRRRRRRRRRRC

SEQ ID NO: 145 - protein, 14 aa, synthetic construct
  MOD_RES 1: note = N-terminal maleimide-linked 1.5kDa branched PEG spacer
  MOD_RES 14: note = C-terminal maleimide-linked 1.5kDa branched PEG spacer
CRRRRRRRRRRRRC

SEQ ID NO: 146 - protein, 13 aa, synthetic construct
  MOD_RES 13: note = C-terminal maleimide-linked 2kDa branched PEG spacer
RRRRRRRRRRRRC

SEQ ID NO: 147 - protein, 12 aa, synthetic construct
  MOD_RES 12: note = C-terminal NHS Ester-linked 2kDa branched PEG spacer
RRRRRRRRRRRR

(19) United States
(12) Patent Application Publication
(10) Pub. No.: US 2025/0127912 A1
(43) Pub. Date: Apr. 24, 2025

(54) COMPOSITIONS FOR DELIVERY OF POLYNUCLEOTIDES

(71) Applicant: miRecule, Inc., Gaithersburg, MD (US)

(72) Inventors: Anthony Saleh, Gaithersburg, MD (US); Charles Marusak, Gaithersburg, MD (US); Tishan Williams, Gaithersburg, MD (US); Fu-An Kang, Collegeville, PA (US)

(21) Appl. No.: 18/749,736

(22) Filed: Jun. 21, 2024

Related U.S. Application Data
(63) Continuation of application No. PCT/US22/53998, filed on Dec. 23, 2022.
(60) Provisional application No. 63/293,614, filed on Dec. 23, 2021.

(51) Int. Cl. A61K 47/64; A61K 47/60

(52) U.S. Cl. CPC A61K 47/6455; A61K 47/60

(57) ABSTRACT

The present disclosure provides compositions for delivering polynucleotides and methods of use thereof for treating genetic diseases. The present disclosure also provides polynucleotide conjugates. The present disclosure further provides anti-transferrin receptor antibodies.

### CROSS-REFERENCE TO RELATED APPLICATIONS

This application is a Continuation Application of International Application No. PCT/US2022/053988, filed Dec. 23, 2022, which claims priority to and benefit of U.S. Provisional Application No. 63/293,614, filed Dec. 23, 2021, the disclosures of which are hereby incorporated by reference in their entireties.

### REFERENCE TO AN ELECTRONIC SEQUENCE LISTING

The contents of the electronic sequence listing (MRC-0003US_Update.xml; Size: 387,714 bytes; and Date of Creation: Nov. 12, 2024) is herein incorporated by reference in its entirety.

### BACKGROUND

Polynucleotides have become useful entities for the treatment of human disease. Targeted delivery of polynucleotides to tissues (including solid tumors, muscle, or immune cells) other than liver has been an on-going challenge in oligonucleotide therapeutics. Due to their chemical compositions including charged phosphate backbones and macromolecular sizes ranging between 6,000˜18,000 daltons, the delivery of polynucleotides to cells has typically relied on formulation technologies using nanoparticle carrier systems, such as lipid nanoparticles, liposomes, dendriplexes, gold particles, viral-like particles, and the like. While these strategies have proven effective, clinical development has typically been restricted to the liver as a consequence of the innate accumulation of most delivery carrier systems in hepatic tissue. In addition, these nanocarrier formulations often contain multiple molecular components that have their own subsets of toxicities, which affect the dosing limits of most polynucleotide therapeutics.

Conjugation of oligonucleotides to targeting moieties such as peptides, proteins, or antibodies has been demonstrated to be effective in recent years. Antibodies (mAbs) are a highly attractive platform for generating targeted oligonucleotide therapeutics. Antibody-Drug conjugates (ADCs) are a clinically proven versatile means to specifically target highly cytotoxic payloads to numerous cancer cell types, thereby improving the therapeutic index of promising anticancer toxic agents. Oligonucleotides bearing tris-galNAc moieties that target the asialoglycoprotein receptor (ASGPR) on liver hepatocytes have shown significant successes in multiple clinical trials. These encouraging results show the path forward for the targeted delivery of polynucleotides to other non-hepatic tissues.

Some progress has been made through bioconjugation of oligonucleotide molecules to peptide or protein targeting moieties displaying promise in preclinical models. However, there are a number of challenges that have limited the application of this approach. These challenges include: 1) achieving even cellular distribution and penetration in dense tissues, such as tumors and muscles; 2) protecting the chemically labile oligonucleotides from degradation caused by extracellular nucleases, until they have been taken up by cells; 3) having efficient endosomal escape of conjugates to the cytosol following uptake; 4) maintaining desirable solubility of protein components following conjugation of hydrophobic linkers that can assist in endosomal escape; 5) designing chemistry and processes that allow precise ratios of oligo to protein and specific conjugation points to create therapeutic effects with appropriate consistency and quality to pass CMC regulatory standards for human use; 6) stabilizing the antibody oligo conjugates through proper formulations to increase long term stability, and avoid aggregations when higher DAR is achieved.

Therefore, there remains a need for new compositions for delivering therapeutic polynucleotides.

### SUMMARY OF THE DISCLOSURE

A first aspect of the present disclosure provides compositions for delivery of polynucleotides. In some embodiments, the composition comprises a hybrid polymer and a polynucleotide. In some embodiments, the hybrid polymer comprises a cationic portion and a neutral portion. In some embodiments, the cationic portion of the hybrid polymer interacts non-covalently with the polynucleotide, e.g., via an ionic interaction.

In some embodiments, the cationic portion of the hybrid polymer is a cationic polypeptide. In some embodiments, the cationic polypeptide is a poly-arginine polypeptide. In some embodiments, the cationic polypeptide is a poly-lysine polypeptide. In some embodiments, the cationic polypeptide comprises arginine and lysine residues. In some embodiments, the cationic polypeptide comprises protamine. In some embodiments, the cationic polypeptide comprises L-amino acid residues. In some embodiments, the cationic polypeptide comprises D-amino acid residues. In some embodiments, the cationic polypeptide comprises L-amino acid residues and D-amino acid residues. In some embodiments, the cationic polypeptide comprises between 9 and 18 amino acid residues. In some embodiments, the cationic polypeptide comprises 12 amino acid residues. In some embodiments, the cationic polypeptide is selected from the group of cationic polypeptides disclosed in Table 1.

In some embodiments, the cationic portion of the hybrid polymer comprises a cationic polymer between about 600 and about 2500 Daltons in size. In some embodiments, the cationic polymer is a linear polymer. In some embodiments, the cationic polymer is a branched polymer. In some embodiments, the cationic polymer is selected from the group consisting of gelatin, glucosamine, N-acetylglucosamine, chitosan, cationic dextran, cationic cyclodextrin, cationic cellulose, polyethylenimine (PEI), polyamidoamine (PAA), poly(amino-co-ester)s (PAEs), poly[2-(N,N-dimethylamino)ethyl methacrylate](PDMAEMA), or cationic lipids, such as DOTAP (N-(1-(2,3-dioleoyloxy) propyl)-N,N,N trimethylammonium) chloride, poly[N,N-Diethylaminoethyl Methacrylate](PDEAEMA), a cationic mucic acid polymer (cMAP) and DOPE (dioleoyl phosphatidylethanolamine). In some embodiments, the cationic polymer is linear PEI. In some embodiments, the cationic polymer is branched PEI (BPEI).

In some embodiments, the hybrid polymer is any of the polymers disclosed in Tables 5, 7, 8 and 9, infra. Each hybrid polymer recited in Tables 5, 7, 8 and 9 is considered a separate embodiment. In some embodiments, the hybrid polymer is selected from the group consisting of PEG12PolyArg12{d}, PEG12PolyArg6, PEG12PolyArg6C, PEG24PolyArg12C, PEG24PolyArg12, PEG24PolyArg9, PolyArg12C-PEG2000 Da, PolyArg12C-PEG5000 Da, PolyArg12C-Dextran5000 Da, PEG12PolyArg12, PEG12PolyArg9d, PEG1000DaPolyArg12, PEG2000DaPolyArg12, PEG5000DaPolyArg12, PolyArg12Cbp1.5 kDa, PolyArg12Cbp3.9 kDa, PolyArg12Cbp16 kDa, CPolyArg12Cbp1.5 kDa, PolyArg12Cbp2 kDa, PolyArg12bp2 kDa, Amide Dextran, Lysine Dextran, PEG PEI 15kda, BPEI-G-PEG 550, and BPEI-G-PEG 5000.

In some embodiments, the neutral portion of the hybrid polymer comprises a polymer between about 100 and about 100,000 Daltons in size. In some embodiments, the neutral portion of the hybrid polymer comprises poly(ethylene glycol)(PEG). In some embodiments, the neutral portion of the hybrid polymer comprises a linear poly(ethylene glycol)(PEG). In some embodiments, the neutral portion of the hybrid polymer comprises a branched poly(ethylene glycol)(PEG). In some embodiments, the hybrid polymer is a PEGylated cationic polypeptide. In some embodiments, the hybrid polymer comprises a PEG9 to PEG1000 polymer. In some embodiments, the hybrid polymer comprises a PEG12 to PEG24 polymer.

In some embodiments, the hybrid polymer and the polynucleotide do not form aggregates or nanoparticles.

In some embodiments, the charge ratio of the cationic polypeptide to the polynucleotide is between 0.25:1 and 5:1. In some embodiments, the charge ratio of the cationic polypeptide to the polynucleotide is between 0.5:1 and 5:1. In some embodiments, the charge ratio of the cationic polypeptide to the polynucleotide is between 1:1 and 4:1. In some embodiments, the charge ratio of the cationic polypeptide to the polynucleotide is between 1:1 and 2:1. In some embodiments, the charge ratio of the cationic polypeptide to the polynucleotide is 1:1 or 2:1.

In some embodiments, the polynucleotide is conjugated to a targeting molecule. In some embodiments, the targeting molecule is an antibody or an antigen-binding fragment thereof, or a binding protein. In some embodiments, the antibody or antigen-binding fragment thereof is selected from the group consisting of a monoclonal antibody, a bispecific antibody, a Fab, a Fab-Fc, a Fv, a single chain Fv (scFv), a diabody, a minibody, and an immunoglobulin single variable domain (ISV) such as an Nanobody® molecule. In some embodiments, the bispecific antibody is a bispecific T-cell engager (BiTE) or a dual-affinity retargeting antibody (DART). In some embodiments, the Nanobody® is a Nanobody-HSA®.

In some embodiments, the antibody or antigen-binding fragment thereof is an IgG molecule or is derived from an IgG molecule. In some embodiments, the IgG molecule is an IgG1 or an IgG4 molecule.

In some embodiments, the binding protein is a soluble receptor or a soluble ligand.

In some embodiments, the soluble receptor comprises the extracellular domain of a receptor.

In some embodiments, the soluble receptor is a Fc fusion protein.

In some embodiments, the targeting molecule is a therapeutically active molecule or a biologically active molecule.

In some embodiments, the polynucleotide is selected from the group consisting of a siRNA, an ncRNA mimic, a short-harpin RNA (shRNA), a dicer-dependent siRNA (di-siRNA), an antisense oligonucleotide (ASO), a gapmer, a mixmer, a double-stranded RNA (dsRNA), a single stranded RNAi, (ssRNAi), a DNA-directed RNA interference (ddRNAi), an RNA activating oligonucleotide (RNAa), an aptamer, an exon skipping oligonucleotide, a miRNA, a miRNA mimic, an mRNA, and a guide RNA. In some embodiments, the polynucleotide is a miRNA mimic. In some embodiments, the miRNA mimic mimics miR-30. In some embodiments, the polynucleotide is miRNA mimic is selected from the group consisting of M30 ml, M30m2, M30m3, and M30m4. In some embodiments, the polynucleotide is M30m3.

In some embodiments, the polynucleotide is an ASO. In some embodiments, the ASO is a DUX4-targeted ASO. In some embodiments, the DUX4-targeted ASO is selected from the group consisting of the DUX4-targeted ASOs disclosed in Table 4. In some embodiments, the DUX4-targeted ASO is selected from the group consisting of ASDX2, ASDX4, ASDX23, ASDX26 and ASDX32.

In some embodiments, the targeting molecule and the polynucleotide result in a synergistic therapeutic or biological effect.

In some embodiments, the polynucleotide is conjugated directly to the targeting molecule. In some embodiments, the polynucleotide is conjugated to the targeting molecule via a linker. In some embodiments, the linker is a hydrophobic linker. In some embodiments, the linker is a peptide linker. In some embodiments, the linker is a chemical linker. In some embodiments, the chemical linker is a polymeric linker. In some embodiments, the chemical linker is linear. In some embodiments, the chemical linker is cyclic. In some embodiments, the polymeric linker comprises PEG, a sugar, a fatty acid, a phosphate, a pyrophosphate or a polysarcosine. In some embodiments, the linker is a high molecular weight PEG linker. In some embodiments, the linker is a low molecular weight PEG linker.

In some embodiments, the linker is non-cleavable. In some embodiments, the linker is cleavable. In some embodiments, the linker is cleavable in vivo. In some embodiments, the cleavable linker is selected from the group consisting of a disulfide linker, a self-immolative peptide polymer hybrid, and a sulfatase-promoted arylsulfate linker. In some embodiments, the self-immolative peptide polymer hybrid comprises glucuronic acid, para-amino-benzoyloxy (PAB), 7-amino-3-hydroxyethyl-coumarin (7-AHC), or Fe(II)-reactive 1,2,4-trioxolane scaffold (TRX). In some embodiments, the cleavable linker may be cleaved through reduction, hydrolysis, proteolysis, photo cleavage, chemical cleavage, enzymatic cleavage, and bio-orthogonal-cleavage. In some embodiments, the chemical cleavage is by Fe II mediated R elimination of TRX. In some embodiments, the enzymatic cleavage is by non-proteolytic sulfatase, β-galactosidase/glucuronidase or pyrophosphatase. In some embodiments, the bio-orthogonal cleavage is by Cu I-BTTAA or free copper ion mediated cleavage.

In some embodiments, the linker is conjugated to a lysine residue, a cysteine residue, histidine residue, or a non-natural amino acid residue in the targeting molecule. In some embodiments, the linker is conjugated to the targeting molecule by a chemical conjugation or an enzymatic conjugation. In some embodiments, the chemical conjugation comprises acylation and click chemistry. In some embodiments, the enzymatic conjugation is via a sortase or a transferase enzyme.

In some embodiments, each targeting molecule is conjugated to between one and eight polynucleotide molecules (DAR of between 1 and 8). In some embodiments, each targeting molecule is conjugated to one polynucleotide molecule (DAR 1). In some embodiments, each targeting molecule is conjugated to two polynucleotide molecules (DAR 2). In some embodiments, each targeting molecule is conjugated to three polynucleotide molecules (DAR 3). In some embodiments, each targeting molecule is conjugated to four polynucleotide molecules (DAR 4). In some embodiments, each targeting molecule is conjugated to five polynucleotide molecules (DAR 5). In some embodiments, each targeting molecule is conjugated to six polynucleotide molecules (DAR 6). In some embodiments, each targeting molecule is conjugated to seven polynucleotide molecules (DAR 7). In some embodiments, each targeting molecule is conjugated to eight polynucleotide molecules (DAR 8).

In some embodiments, the polynucleotide-conjugated targeting molecule has a molecular weight greater than 30 kDa. In some embodiments, the polynucleotide-conjugated targeting molecule has a molecular weight greater than 40 kDa. In some embodiments, the polynucleotide-conjugated targeting molecule has a molecular weight greater than 50 kDa.

In some embodiments, the polynucleotide-conjugated targeting molecule has a molecular weight greater than 60 kDa. In some embodiments, the polynucleotide-conjugated targeting molecule has a molecular weight greater than 500 Da. In some embodiments, the polynucleotide-conjugated targeting molecule has a molecular weight no greater than 7,500 kDa.

In some embodiments, the polynucleotide conjugate is selected from the group consisting of Cetuximab-DBCO-C9-M30m3 (DAR3); Cetuximab-DBCO-C4/P5-M30m3 (DAR3); Cetuximab-DBCO-PEG9-M30m3 (DAR3); Cetuximab-DBCO-PEG9-M30m3 (DAR2); Cetuximab-DBCO-PEG9-M30m3 (DAR4); Cetuximab-DBCO-PEG9-M30m3 (DAR6); Cetuximab-Linear-PEG13-M30m3 (DAR4); Cetuximab-PEG4-azide-DBCO-PEG5-M30m3 (DAR1), Cetuximab-PEG4-azide-DBCO-PEG5-M30m3 (DAR2), Cetuximab-PEG4-azide-DBCO-PEG5-M30m3 (DAR4), Cetuximab-PEG4-azide-DBCO-PEG5-M30m3 (DAR2.5), Cetuximab-PEG4-azide-DBCO-PEG5-M30m3 (DAR4.5), Cetuximab-PEG4-azide-DBCO-PEG5-M30m3 (DAR6.5), Cetuximab-SMCC-M30m3 (DAR4) (SMCC), Cetuximab-MCVCPABcPNP-M30m3 (DAR4) (MCVCPABcPNP), Cetuximab-MCPEG4VCPABcPNP-M30m3 (DAR4) (MCPEG4VCPABcPNP), Cetuximab-C4-Azide-DBCO-C5-M30m3, Cetuximab-PEG4-azide-DBCO-PEG4-m30m3 (DAR4), Cetuximab-MC-PEG4-ValCit-PABc-M30m3 (DAR1), Cetuximab-MC-PEG4-ValCit-PABc-M30m3 (DAR2), Cetuximab-MC-PEG4-ValCit-PABc-M30m3 (DAR3), Cetuximab-MC-PEG4-ValCit-PABc-M30m3 (DAR4), 3tf12-DBCO-PEG8-NCD5 (DAR1); 3tf12-DBCO-PEG8-M30m3 (DAR1); Fv55-SMCC-M30m3 (DAR1); Fv55-PEG8-DBCO-M30m3 (DAR1), Fv55-PEG8-DBCO-M30m3 (DAR2), Fv55-linker-M30m3 (DAR2), Fv55-DBCO-PEG8-M30 ml(DAR1), Fv55-DBCO-PEG8-M30 ml(DAR2), and ASO-carbon4-DBCO-Carbon5-3tf12 (DAR1). In some embodiments, the polynucleotide conjugate is selected from the group consisting for the antibody-polynucleotide conjugates disclosed in Table 5 or Table 6. Each of the APCs disclosed in Table 5 or Table 6 is considered a separate embodiment.

In some embodiments, the composition comprises: (a) Cetuximab-DBCO-C9-M30m3 (DAR3) and PEG12-Poly-(D-Arg)12; (b) Cetuximab-DBCO-C4/P5-M30m3 (DAR3) and PEG12-Poly-(D-Arg)12; (c) Cetuximab-DBCO-PEG9-M30m3 (DAR3) and PEG12-Poly-(D-Arg)12; (d) Cetuximab-DBCO-PEG9-M30m3 (DAR2) and PEG12-Poly-(D-Arg)12; (e) Cetuximab-DBCO-PEG9-M30m3 (DAR4) and PEG12-Poly-(D-Arg)12; (f) Cetuximab-DBCO-PEG9-M30m3 (DAR6) and PEG12-Poly-(D-Arg)12; (g) Cetuximab-Linear-PEG13-M30m3 (DAR4) and PEG12-Poly-(D-Arg)12; (h) 3tf12-DBCO-PEG8-NCD5 and Poly(L-Arg)9; (i) 3tf12-DBCO-PEG8-M30m3 and Poly(L-Arg)9; (j) Fv55-SMCC-M30m3 and PEG12-Poly(L-Arg)12; (k) Fv55-PEG30-M30m3 and PEG12-Poly(L-Arg)12; (1) Cetuximab-PEG4-azide-DBCO-PEG5-M30m3 (DAR2) and PEG12PolyArg12{d}; (m) Cetuximab-PEG4-azide-DBCO-PEG5-M30m3 (DAR2) and PolyArg12Cbp3.9 kDa; (n) Cetuximab-PEG4-azide-DBCO-PEG5-M30m3 (DAR4) and PEG12PolyArg12{d}; (o) Cetuximab-PEG4-azide-DBCO-PEG5-M30m3 (DAR4) and PolyArg12Cbp3.9 kDa; (p) Cetuximab-PEG4-azide-DBCO-PEG5-M30m3 (DAR4) and PolyArg12C-PEG2000 Da; (q) Cetuximab-PEG4-azide-DBCO-PEG5-M30m3 (DAR4) and PolyArg12C-PEG5000 Da; (r) Cetuximab-PEG4-azide-DBCO-PEG5-M30m3 (DAR4) and PolyArg12C-Dextran5000 Da; (s) Cetuximab-SMCC-M30m3 (DAR4) and PEG12PolyArg12{d}; (t) Cetuximab-MCVCPABcPNP-M30m3 (DAR4) and PEG12PolyArg12{d}; (u) Cetuximab-MCPEG4VCPABcPNP-M30m3 (DAR4) and PEG12PolyArg12{d}; (v) Cetuximab-PEG4-azide-DBCO-PEG5-M30m3 (DAR2.5) and PEG12PolyArg12{d}; (w) Cetuximab-PEG4-azide-DBCO-PEG5-M30m3 (DAR4.5) and PEG12PolyArg12{d}; (x) Cetuximab-PEG4-azide-DBCO-PEG5-M30m3 (DAR6.5) and PEG12PolyArg12{d}; (y) Cetuximab-C4(Azide-DBCO)C5-M30m3 and PEG12PolyArg12; or (z) any of the antibody-polynucleotide conjugate and hybrid polymer combinations disclosed in Table 5. Each of the of the antibody-polynucleotide conjugates and the associated hybrid polymers disclosed in Table 5 is considered a separate embodiment.

In a second aspect, the present disclosure provides a polynucleotide conjugate comprising a polynucleotide conjugated to a targeting molecule.

In some embodiments, the targeting molecule is an antibody or an antigen-binding fragment thereof, or a binding protein. In some embodiments, the antibody or antigen-binding fragment thereof is selected from the group consisting of a monoclonal antibody, a bispecific antibody, a Fab, a Fab-Fc, a Fv, a single chain Fv (scFv), a diabody, a minibody, a vNAR, a Centyrin, and an immunoglobulin single variable domain (ISV) such as an Nanobody® molecule. In some embodiments, the bispecific antibody is a bispecific T-cell engager (BiTE) or a dual-affinity retargeting antibody (DART). In some embodiments, the Nanobody® is a Nanobody-HSA®. In some embodiments, the antibody or antigen-binding fragment thereof is an IgG molecule or is derived from an IgG molecule. In some embodiments, the IgG molecule is an IgG1 or an IgG4 molecule.

In some embodiments, the binding protein is a soluble receptor or a soluble ligand. In some embodiments, the soluble receptor comprises the extracellular domain of a receptor. In some embodiments, the soluble receptor is a Fc fusion protein.

In some embodiments, the targeting molecule is a therapeutically active molecule or a biologically active molecule.

In some embodiments, the polynucleotide is selected from the group consisting of an oligonucleotide, a siRNA, an ncRNA mimic, a short-harpin RNA (shRNA), a dicer-dependent siRNA (di-siRNA), an antisense oligonucleotide (ASO), a gapmer, a mixmer, a double-stranded RNA (dsRNA), a single stranded RNAi, (ssRNAi), a DNA-directed RNA interference (ddRNAi), an RNA activating oligonucleotide (RNAa), an aptamer, an exon skipping oligonucleotide, a miRNA, a miRNA mimic, an mRNA, and a guide RNA. In some embodiments, the polynucleotide is a miRNA mimic. In some embodiments, the miRNA mimic mimics miR-30. In some embodiments, the polynucleotide is miRNA mimic is selected from the group consisting of M30 ml, M30m2, M30m3, and M30m4. In some embodiments, the polynucleotide is M30m3.

In some embodiments, the polynucleotide is an ASO. In some embodiments, the ASO is a DUX4-targeted ASO. In some embodiments, the DUX4-targeted ASO is selected from the group consisting of the DUX4-targeted ASOs disclosed in Table 4. In some embodiments, the DUX4-targeted ASO is selected from the group consisting of ASDX2, ASDX4, ASDX23, ASDX26 and ASDX32.

In some embodiments, the targeting molecule and the polynucleotide result in a synergistic therapeutic or biological effect.

In some embodiments, the polynucleotide is conjugated directly to the targeting molecule. In some embodiments, the polynucleotide is conjugated to the targeting molecule via a linker. In some embodiments, the linker is a hydrophobic linker. In some embodiments, the linker is a peptide linker. In some embodiments, the linker is a chemical linker. In some embodiments, the chemical linker is a polymeric linker. In some embodiments, the chemical linker is linear. In some embodiments, the chemical linker is cyclic. In some embodiments, the polymeric linker comprises PEG, a sugar, a fatty acid, a phosphate, a pyrophosphate or a polysarcosine. In some embodiments, the linker is a high molecular weight PEG linker. In some embodiments, the linker is a low molecular weight PEG linker.

In some embodiments, the linker is non-cleavable. In some embodiments, the linker is cleavable. In some embodiments, the linker is cleavable in vivo. In some embodiments, the cleavable linker is selected from the group consisting of a disulfide linker, a self-immolative peptide polymer hybrid, and a sulfatase-promoted arylsulfate linker. In some embodiments, the self-immolative peptide polymer hybrid comprises glucuronic acid, para-amino-benzoyloxy (PAB), 7-amino-3-hydroxyethyl-coumarin (7-AHC), or Fe(II)-reactive 1,2,4-trioxolane scaffold (TRX). In some embodiments, the cleavable linker may be cleaved through reduction, hydrolysis, proteolysis, photo cleavage, chemical cleavage, enzymatic cleavage, and bio-orthogonal-cleavage. In some embodiments, the chemical cleavage is by Fe II mediated β elimination of TRX. In some embodiments, the enzymatic cleavage is by non-proteolytic sulfatase, β-galactosidase/glucuronidase or pyrophosphatase. In some embodiments, the bio-orthogonal cleavage is by Cu I-BTTAA or free copper ion mediated cleavage.

In some embodiments, the linker is conjugated to a lysine residue, a cysteine residue, histidine residue, or a non-natural amino acid residue in the targeting molecule. In some embodiments, the linker is conjugated to the targeting molecule by a chemical conjugation or an enzymatic conjugation. In some embodiments, the chemical conjugation comprises acylation and click chemistry. In some embodiments, the enzymatic conjugation is via a sortase or a transferase enzyme.

In some embodiments, each targeting molecule is conjugated to between one and eight polynucleotide molecules (DAR 1-8). In some embodiments, each targeting molecule is conjugated to one polynucleotide molecule (DAR 1). In some embodiments, each targeting molecule is conjugated to two polynucleotide molecules (DAR 2). In some embodiments, each targeting molecule is conjugated to three polynucleotide molecules (DAR 3). In some embodiments, each targeting molecule is conjugated to four polynucleotide molecules (DAR 4). In some embodiments, each targeting molecule is conjugated to five polynucleotide molecules (DAR 5). In some embodiments, each targeting molecule is conjugated to six polynucleotide molecules (DAR 6). In some embodiments, each targeting molecule is conjugated to seven polynucleotide molecules (DAR 7). In some embodiments, each targeting molecule is conjugated to eight polynucleotide molecules (DAR 8).

In some embodiments, the polynucleotide-conjugated targeting molecule has a molecular weight greater than 30 kDa. In some embodiments, the polynucleotide-conjugated targeting molecule has a molecular weight greater than 40 kDa. In some embodiments, the polynucleotide-conjugated targeting molecule has a molecular weight greater than 50 kDa.

In some embodiments, the polynucleotide-conjugated targeting molecule has a molecular weight greater than 60 kDa. In some embodiments, the polynucleotide-conjugated targeting molecule has a molecular weight no greater than 7,500 kDa.

In some embodiments, the polynucleotide conjugate is selected from the group consisting of Cetuximab-DBCO-C9-M30m3 (DAR3); Cetuximab-DBCO-C4/P5-M30m3 (DAR3); Cetuximab-DBCO-PEG9-M30m3 (DAR3); Cetuximab-DBCO-PEG9-M30m3 (DAR2); Cetuximab-DBCO-PEG9-M30m3 (DAR4); Cetuximab-DBCO-PEG9-M30m3 (DAR6); Cetuximab-Linear-PEG13-M30m3 (DAR4); Cetuximab-PEG4-azide-DBCO-PEG5-M30m3 (DAR1), Cetuximab-PEG4-azide-DBCO-PEG5-M30m3 (DAR2), Cetuximab-PEG4-azide-DBCO-PEG5-M30m3 (DAR4), Cetuximab-PEG4-azide-DBCO-PEG5-M30m3 (DAR2.5), Cetuximab-PEG4-azide-DBCO-PEG5-M30m3 (DAR4.5), Cetuximab-PEG4-azide-DBCO-PEG5-M30m3 (DAR6.5), Cetuximab-SMCC-M30m3 (DAR4) (SMCC), Cetuximab-MCVCPABcPNP-M30m3 (DAR4) (MCVCPABcPNP), Cetuximab-MCPEG4VCPABcPNP-M30m3 (DAR4) (MCPEG4VCPABcPNP), Cetuximab-C4-Azide-DBCO-C5-M30m3, Cetuximab-PEG4-azide-DBCO-PEG4-m30m3 (DAR4), Cetuximab-MC-PEG4-ValCit-PABc-M30m3 (DAR1), Cetuximab-MC-PEG4-ValCit-PABc-M30m3 (DAR2), Cetuximab-MC-PEG4-ValCit-PABc-M30m3 (DAR3), Cetuximab-MC-PEG4-ValCit-PABc-M30m3 (DAR4), 3tf12-DBCO-PEG8-NCD5 (DAR1); 3tf12-DBCO-PEG8-M30m3 (DAR1); Fv55-SMCC-M30m3 (DAR1); Fv55-PEG8-DBCO-M30m3 (DAR1), Fv55-PEG8-DBCO-M30m3 (DAR2), Fv55-linker-M30m3 (DAR2), Fv55-DBCO-PEG8-M30 ml(DAR1), Fv55-DBCO-PEG8-M30 ml(DAR2), and ASO-carbon4-DBCO-Carbon5-3tf12 (DAR1). In some embodiments, the polynucleotide conjugate is selected from the group consisting for the antibody-polynucleotide conjugates disclosed in Table 5 or Table 6. Each of the APCs disclosed in Table 5 or Table 6 is considered a separate embodiment.

A third aspect of the present disclosure provides a method of treating a genetic disease in a subject in need thereof. In some embodiments, the method comprises administering to the subject a therapeutically effective amount of any of the compositions disclosed herein or any of the polynucleotide conjugates disclosed herein.

In some embodiments, the genetic disease is a viral infection. In some embodiments, the viral infection is by a virus selected from the group consisting of an adenovirus, an anellovirus, an arenavirus, an astrovirus, a bunyavirus, a calicivirus, a coronavirus, a filovirus, a flavivirus, a hepadnavirus, a herpesvirus, an orthomyxovirus, a papillomavirus, a paramyxovirus, a parvovirus, a picornavirus, a pneumovirus, a polyomavirus, a poxvirus, a reovirus, a retrovirus, a rhabdovirus, and a togavirus. In some embodiments, the virus is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus. In some embodiments, the polynucleotide comprises a siRNA, a miRNA, a miRNA mimic, an ASO, or a guide RNA that targets a viral gene. In some embodiments, the polynucleotide is conjugated to a targeting molecule that specifically binds to a viral protein or a protein on the surface of a host cell for the virus. In some embodiments, the polynucleotide and the targeting molecule synergize in the treatment of the viral infection.

In some embodiments, the genetic disease is cancer. In some embodiments, the cancer is characterized by overexpression of an oncogene. In some embodiments, the polynucleotide comprises a siRNA, a miRNA, a miRNA mimic, an ASO, or a guide RNA that targets the oncogene. In some embodiments, the cancer is characterized by reduced expression of a tumor suppressor gene. In some embodiments, the polynucleotide comprises a mRNA molecule encoding the tumor suppressor gene. In some embodiments, the polynucleotide comprises a guide RNA that that restores expression of the tumor suppressor gene.

In some embodiments, the polynucleotide is conjugated to a targeting molecule that specifically binds a tumor cell of the cancer. In some embodiments, the targeting molecule specifically binds epidermal growth factor receptor; and wherein the polynucleotide is a miR-30 miRNA or a mimic thereof. In some embodiments, the targeting molecule specifically binds TFR. In some embodiments, the targeting molecule is selected from the group consisting of FV55 scFv, Fv55 diabody, and 3TF12. In some embodiments, the targeting molecule specifically binds ACVR1, and the polynucleotide is a miR-30 miRNA or a mimic thereof. In some embodiments, the targeting molecule specifically binds ACVR1, and the polynucleotide is a miR-26 miRNA or a mimic thereof. In some embodiments, the polynucleotide and the targeting molecule synergize in the treatment of the cancer.

In some embodiments, the genetic disease is a neuromuscular disorder. In some embodiments, the neuromuscular disorder is a muscular dystrophy. In some embodiments, the muscular dystrophy is facioscapulohumeral muscular dystrophy (FSHD). In some embodiments, the polynucleotide comprises a siRNA, a miRNA, a miRNA mimic, an ASO, or a guide RNA that targets DUX4, DMPK or CAPN3. In some embodiments, the polynucleotide is an ASO that targets DUX4. In some embodiments, the DUX4-targeted ASO is selected from the group consisting of the DUX4-targeted ASOs disclosed in Table 4. In some embodiments, the DUX4-targeted ASO is selected from the group consisting of ASDX2, ASDX4, ASDX23, ASDX26 and ASDX32. In some embodiments, the muscular dystrophy is Duchenne muscular dystrophy. In some embodiments, the polynucleotide is a mRNA, a cDNA, or a vector encoding dystrophin or utrophin. In some embodiments, the polynucleotide is a guide RNA that restores the expression of dystrophin or utrophin.

In some embodiments, the polynucleotide is conjugated to a targeting molecule that specifically binds a marker on the surface of a skeletal muscle cell of the subject. In some embodiments, the targeting molecule specifically binds ACVR1. In some embodiments, the targeting molecule specifically binds ACVR1, and the polynucleotide is a DUX4-targeted ASO. In some embodiments, the polynucleotide and the targeting molecule synergize in the treatment of the muscular dystrophy.

A fourth aspect of the present disclosure provides an antibody or an antigen-binding fragment thereof that specifically binds human transferrin receptor (TfR1). In some embodiments, the antibody or antigen-binding fragment thereof comprises a heavy chain variable region (VH) comprising the amino acid sequence QVQVQDSGGELVQPGGSLRVSCKASGFNIKDSYMHWVRQAPGKGLEWVAFIDPET GNTYYADSVKGRFTISRDNSKNTLYLQMNSLRAEDTAVYYCARGSIYWYFDVWGK GTTVTVSS (SEQ ID NO: 1) and a light chain variable region (VL) comprising the amino acid sequence DIQMTQSPSSLSASVGQRVTITCRASQSLLNSSNQKNSLGWYQQKPGKAPKLLIYFAS TRQSGVPSRFSGSGSGTDFTLTISSLQPEDFATYYCQQHYSTPLTFGQGTKVDIKRC (SEQ ID NO: 2).

In some embodiments, the antibody or antigen-binding fragment thereof is selected from the group consisting of a full-length antibody, a Fab, a Fab-Fc, a Fv, a single chain Fv (scFv), a diabody, a minibody, and a Nanobody®. In some embodiments, the antibody or antigen-binding fragment thereof is a scFv. In some embodiments, the antibody or antigen-binding fragment thereof is a diabody.

In some embodiments, the VH and VL are connected a linker. In some embodiments, the linker comprises the amino acid sequence GGGGS (SEQ ID NO: 3). In some embodiments, the linker comprises the amino acid sequence (GGGGS)_{N} (SEQ ID NO: 3), wherein N is 1-3. In some embodiments, if the antibody or antigen-binding fragment thereof is a scFv, then the linker comprises the amino acid sequence (GGGGS)₃ (SEQ ID NO: 3). In some embodiments, if the antibody or antigen-binding fragment thereof is a diabody, then the linker comprises the amino acid sequence (GGGGS)_{N} (SEQ ID NO: 3), wherein N is 1 or 2.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, 1D, 1E, 1F, 1G, 1H, 1I, 1J, and 1K depicts representative hybrid polymers and conjugates comprising them. FIGS. 1A-D depict structures of representative hybrid polymers, FIG. E depicts a representative hybrid polymer coated antibody-oligonucleotide conjugate, FIG. F depicts a representative hybrid polymer coated diabody-siRNA conjugate, FIG. G depicts a representative hybrid polymer coated Nanobody-ASO conjugate, FIG. H depicts a representative hybrid polymer coated antibody-mRNA conjugate, FIG. 1 depicts a representative hybrid polymer coated cytokine-gRNA conjugate, FIG. J depicts a representative hybrid polymer coated antibody-DNA plasmid conjugate, and FIG. K depicts a representative hybrid polymer coated antibody-cDNA conjugate.

FIG. 2A depicts a representative synthetic scheme of antibody oligonucleotide conjugate via click chemistry.

FIG. 2B depicts a representative synthetic scheme of antibody oligonucleotide conjugate via acylation chemistry.

FIG. 2C depicts a representative synthetic scheme of antibody oligonucleotide conjugate via site-specific cysteine conjugation with a cleavable linker.

FIGS. 2D.I, 2D.II, and 2D.III depict a representative manufacturing process of hybrid polymer coated antibody oligonucleotide conjugate.

FIGS. 3A and 3B depict a representative purification process of diabody-oligonucleotide conjugates using SEC FPLC.

FIGS. 4A and 4B depict a representative purification process of an antibody-oligonucleotide conjugates using SEC FPLC.

FIG. 5 depicts a representative purification process of antibody-miRNA conjugates having different DARs using SEC FPLC.

FIGS. 6A and 6B depict precipitation and aggregation statuses of Fv55-DBCO-PEG8-miRNA-AF488 conjugates with various Arg12 peptides.

FIG. 7 depicts ELISA antigen affinity of various diabody-miRNA conjugates.

FIG. 8A depicts structure of a representative hybrid polymer coated antibody-miRNA conjugate.

FIG. 8B depicts ELISA antigen affinity of hybrid polymer coated mAb-miRNA conjugate.

FIGS. 9A, 9B, 9C, and 9D depict the stability of an miRNA mimic, alone or with a cationic peptide or a hybrid polymer in human serum.

FIG. 10 depicts the stability of Cetuximab-miRNA Conjugate, alone or with a cationic peptide or a hybrid polymer in human serum.

FIG. 11 depicts the stability of diabody ScFv-miRNA Conjugate, alone or with a hybrid polymer in human serum.

FIG. 12 depicts the stability of ScFv-Duplex Conjugate, with different linkers, in human serum.

FIG. 13A depicts in vitro delivery efficacies of Cetuximab-miRNA Conjugates in human cancer cells in the presence (left bar) and absence (right bar) of the hybrid polymer Peg12PolyArg12{d} at three different DARs. Mean fluorescence intensity was averaged based on ASD647 signal in each cell. * denotes p<0.005 by students t test as compared to no delivery. Error bars represent standard deviation.

FIG. 13B provides representative images for the transfection experiments summarized in FIG. 13A.

FIG. 13C provides the results of a reporter assay using a luciferase reporter containing miR-30 target sites within its 3′ untranslated region (UTR) of the luciferase transcript. Decreased luminescence demonstrates knock down of the reporter construct by the test miR-30 microRNA mimics. Data are reported as average+/−SEM for 3 replicates.

FIG. 14A depicts the structure of Peg12PolyArg12{d}.

FIG. 14B depicts the structure of PolyArg12Cbp (3.9 kDa).

FIGS. 14C, 14D, 14E, and 14G provide the results of reporter assays using a luciferase reporter containing miR-30 target sites within its 3′ untranslated region (UTR) of the luciferase transcript using various antibody polynucleotide conjugates (APCs) and various hybrid polymers. Data are reported as average+/−SEM for 3 replicates.

FIG. 14F provides qPCR results for miR30 downstream genes following treatment with various APCs in the presence or absence of the hybrid polymer Peg12PolyArg12{d}.

FIG. 15 depicts in vitro cellular activity of 3tf12-M30m3 XTT and Lum.

FIG. 16 depicts in vitro activity of fv55-SMCC-M30m3 with hybrid polymer peptide.

FIGS. 17A, 17B, and 17C depict in vitro efficacy of various Cetuximab-miRNA Conjugates in the presence and absence of a hybrid polymer. Data are reported as average+/−SEM for 3 replicates.

FIGS. 18A and 18B depict in vitro efficacy of ScFv-ASO Conjugates.

FIGS. 19A.I, 19A.II, 19B.I, 19B.II, 19C.I, 19C.II, and 19D show that Cetuximab-miRNA Conjugate with peptide hybrid coating improves delivery to tumor. FIG. 19A.II, FIG. 19B.II, and FIG. 19C.II provide photographic images demonstrating tumor delivery of the Cetuxamab-miRNA conjugate, and FIG. 19A.I, FIG. 19B.I, and FIG. 19C.I, respectively, comprise graphic images of those photographic images.

FIGS. 20A.I, 20A.II, 20B.I, 20B.II, and 20C show IR750-FV55 diabody-miRNA Conjugate with peptide hybrid coating improves delivery to tumor. FIG. 20A.II and FIG. 20B.II provide photographic images demonstrating tumor delivery of the IR2750-FV55 diabody-miRNA conjugate, and FIG. 20A.I and FIG. 20B.I, respectively, comprise graphic images of those photographic images.

FIGS. 21A.I, 21A.II, and 21B show that complexing Cetuximab-C9-M30m3 Conjugate with the hybrid polymer PEG12PolyArg12{d}improves anti-tumor efficacy. FIG. 21A.II provides photographic images demonstrating tumor delivery of the Cetuximab-C9-M30m3 Conjugate. FIG. 21A.I comprises graphic images of those photographic images.

FIG. 21C depicts the growth of UMSCC109 tumors following treatment with Cetuximab-MCVCPABcPNP-M30m3 DAR4, Cetuximab-SMCC-M30m3 DAR4, a Cetuximab control, and phosphate buffered saline (PBS).

FIG. 21D depicts the growth of various tumors following treatment with Cetuximab-PEG4-azide-DBCO-PEG5-M30m3 DAR2 (APC1), a Cetuximab control, and PBS.

FIG. 21E depicts growth of UMSCC109 tumors following treatment with PBS and Cetuximab-PEG4-azide-DBCO-PEG5-M30m3 at DAR 2.5, 4.5 and 6.5 combined with Peg12PolyArg12{d} at a charge:charge ratio of 1:1.

FIG. 21F depicts ELISA results illustrating delivery of M30m3 in various mouse tissues after treatment with PBS control, unconjugated control mixture (Cetuximab, M30m3, and Peg12PolyArg12{d}), APC2 (Cetuximab-PEG4-azide-DBCO-PEG5-M30m3 at DAR2) with and without either Peg12PolyArg12{d} or R12Cbp3.9kda at a charge:charge ratio of 0.5:1.

### DETAILED DESCRIPTION

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P.M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the invention, unless explicitly disclaimed or improper. Combination of embodiments are not limited to those specific combinations claimed via the multiple dependent claims.

All of the above, and any other publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components.

Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including” is used to mean “including but not limited to.” “Including” and “including but not limited to” are used interchangeably.

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones, 2′-deoxy-, 2′-O-methyl-, 2′-deoxy-2′-fluoro-modified nucleotides, and a terminal cap molecule at the 3′-end, the 5′-end, or both of the 3′- and 5′-ends). In general, an analogue of a particular nucleotide has the same base-pairing specificity; i.e., an analogue of A will base-pair with T. The terms also encompass polymers comprising one or more chemically modified nucleotides. Non-limiting examples of polynucleotides include small interfering RNAs (siRNAs), microRNAs (miRNAs), miRNA mimics, short hairpin RNA (shRNA), double-stranded RNA (dsRNA), transfer RNA (tRNA), ribosomal RNA (rRNA), heterogeneous nuclear RNA (hnRNA), antisense oligonucleotides (ASOs, including exon-skipping ASOs), messenger RNAs (mRNAs), complementary DNAs (cDNAs), plasmids and vectors, and guide RNAs (gRNAs).

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “residue,” as used herein, refers to a position in a protein and its associated amino acid identity.

As used herein, the terms “Fc,” “Fc region” or “Fc domain” are used interchangeably herein and refer to the polypeptide comprising the constant region of an antibody excluding, in some instances, the first constant region immunoglobulin domain (e.g., CH1) or a portion thereof, and in some cases, part of the hinge. Thus, an Fc can refer to the last two constant region immunoglobulin domains (e.g., CH2 and CH3) of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains C72 and C73 (C72 and C73) and the lower hinge region between Cγ1 (Cγ1) and Cγ2 (Cγ2). In some embodiments, an Fc refers to a truncated CH1 domain, and CH2 and CH3 of an immunoglobulin. Although the boundaries of the Fc region may vary, the human IgG heavy chain Fc region is usually defined to include residues E216 or C226 or P230 to its carboxyl-terminus, wherein the numbering is according to the EU numbering. In some embodiments, the Fc domain is derived from a human IgG1 heavy chain Fc domain. In some embodiments, the Fc domain is derived from a human IgG2 heavy chain Fc domain. The “EU format as set forth in Edelman” or “EU numbering” or “EU index” refers to the residue numbering of the human Fc domain as described in Edelman G M et al. (Proc. Natl. Acad. USA (1969), 63, 78-85, hereby entirely incorporated by reference).

As used herein, the terms “Fc fusion protein” refers to a protein comprising an Fc region, generally linked (optionally through a linker moiety) to a different protein.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, human antibodies, engineered antibodies (including humanized antibodies, fully human antibodies, chimeric antibodies, single-chain antibodies, artificially selected antibodies, CDR-granted antibodies, etc.) that specifically bind to a given antigen. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds. There are two types of light chain: λ and κ. In humans, λ and κ light chains are similar, but only one type is present in each antibody. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety).

An “antigen-binding fragment” of an antibody refers to a fragment of a full-length antibody that retains the ability to specifically bind to an antigen (preferably with substantially the same binding affinity). Examples of an antigen-binding fragment includes (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR), disulfide-linked Fvs (dsFv), and anti-idiotypic (anti-Id) antibody and intrabody. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al. Science 242:423-426 (1988) and Huston et al., Proc. Natl. Acad. Sci. USA 85:5879-5883 (1988)). Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. Proc. Natl. Acad. Sci. USA 90:6444-6448 (1993); Poljak et al., 1994, Structure 2:1121-1123).

The terms “microRNA,” “miRNA” and “miR” are used interchangeably herein and refer to a single stranded non-coding RNA that functions in RNA silencing and post-transcriptional regulation of gene expression. miRNAs function by complementary base pairing with mRNA molecules, that silences the mRNA, by, inter alia, one or more of the following: (a) cleavage of the mRNA strand into two pieces, (b) destabilization of the mRNA through shortening of its poly(A) tail, and (c) less efficient translation of the mRNA into proteins by ribosomes.

The terms “subject,” “patient” and “individual” are used interchangeably and refer to mammals including, but not limited to, human patients and non-human primates, as well as experimental animals such as rabbits, dogs, cats, rats, mice, and other animals. Accordingly, the term “subject” or “patient” as used herein means any mammalian patient or subject to which the compositions of the disclosure can be administered. Subjects of the present invention include those with a disorder.

The term “genetic disease” as used herein refers to a disease or disorder that is treatable with a polynucleotide therapeutic. Examples of genetic diseases include, but are not limited to, any disease or disorder caused by a genetic mutation, cancers, and viral infections, diseases or disorders caused by a mutation that may be corrected using gene editing (e.g., CRISPR/Cas9, or Zinc Finger Nucleases), diseases or disorders caused by overexpression of a gene, and diseases or disorders caused by decreased or lack of expression of a gene.

The terms “charge ratio,” “N/P,” “N/O” and “N⁺/O⁻” ratio are used interchangeably and refer to the ratio of positively charged amine (N or nitrogen) groups in the cationic polymer to the negatively charged phosphate groups (P or O) in the polynucleotide. “N” refers to the nitrogen (N) in the cationic polymer. “P” and “O” are used interchangeably and refer to the phosphate (P) group or oxygen (O) in the phosphate group in the polynucleotide.

The term “targeting molecule” as used herein refers to a molecule that binds or localizes at a particular target or location. The molecule may be for example, be an antibody or an antigen-binding fragment thereof, or a binding protein.

The term “hybrid polymer” as used herein refers to a polymer that comprises at least two portions that differ from each other in composition. In some embodiments, at least one portion of the hybrid polymer is a cationic polymer and at least one portion of the hybrid polymer is a neutral polymer.

As used herein, the term “aggregate” refers to non-covalent association of molecules to form a solid or mesophase complex structure. The onset of aggregation and the onset of two-molecule attraction are not the same. The aggregate phase involves interactions between multiple molecules, whereas molecules in the dilute phase interact very weakly with each other. Aggregation starts when the free energy per molecule is equal in the dilute and aggregate phases. In the context of a targeting molecule conjugated polynucleotide, an aggregate requires at least ten non-covalently linked conjugates. In the context of unconjugated polynucleotides, aggregation depends on the length of the polynucleotide. For example, for shorter polynucleotides (e.g., an oligonucleotide) an aggregate requires at least ten non-covalently linked polynucleotides, whereas a gene-length polynucleotide (e.g., 10kb to 15kb on average for a human gene) requires fewer polynucleotides for aggregation.

The terms “pharmaceutically effective amount,” “therapeutically effective amount,” or “therapeutically effective dose” refer to an amount effective to treat a disease in a patient, e.g., effecting a beneficial and/or desirable alteration in the general health of a patient suffering from a disease (e.g., a genetic disease as described herein), treatment, healing, inhibition or amelioration of a physiological response or condition, etc. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of disease, the therapeutics or combination of therapeutics selected for administration, and the mode of administration. The skilled worker can readily determine the effective amount for a given situation by routine experimentation. The skilled worker will recognize that, for example, treating cancer includes, but is not limited to, killing cancer cells, preventing the growth of new cancer cells, causing tumor regression (a decrease in tumor size), causing a decrease in metastasis, improving vital functions of a patient, improving the well-being of the patient, decreasing pain, improving appetite, improving the patient's weight, and any combination thereof. The terms “pharmaceutically effective amount,” “therapeutically effective amount,” or (therapeutically effective dose” also refer to the amount required to improve the clinical symptoms of a patient. The therapeutic methods or methods of treating described herein are not to be interpreted or otherwise limited to “curing” the disease.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation, amelioration, or slowing the progression, of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein.

“Administering” or “administration of” a composition as disclosed herein to a subject can be carried out using one of a variety of methods known to those skilled in the art. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one pharmaceutical composition or treatment modality, the disclosure contemplates that the pharmaceutical compositions may be administered at the same or differing times and via the same or differing routes of administration.

### Compositions for Delivering Polynucleotides

A first aspect of the present disclosure provides compositions for delivering polynucleotides. In some embodiments, the composition comprises a hybrid polymer and a polynucleotide. In some embodiments, the hybrid polymer comprises a cationic portion and a neutral portion. In some embodiments, the cationic portion of the hybrid polymer interacts with the polynucleotide non-covalently. In some embodiments, the cationic portion of the hybrid polymer interacts via an ionic interaction with the polynucleotide. In some embodiments, the cationic portion of the hybrid polymer and the polynucleotide form an ionic complex. In some embodiments, the hybrid polymer protects the polynucleotide from biological degradation in vivo. Cationic polymers (including cationic polypeptides) are known in the art. See, e.g., WO 2017/173408, incorporated herein by reference in its entirety. Without being bound by theory, the cationic portion (e.g. the cationic polypeptide) of the hybrid polymer binds the polynucleotide and closely associates it with a cell membrane for uptake by the cell.

In some embodiments, the cationic portion of the hybrid polymer is a cationic polypeptide. In some embodiments, the cationic polypeptide comprises positively charged amino acid residues. In some embodiments, the cationic polypeptide comprises positively charged amino acid residues and non-positively charged amino acid residues. In some embodiments, the non-positively charged amino acid residues are neutral amino acid residues. In some embodiments, the non-positively charged amino acid residues are negatively charged amino acid residues. In some embodiments, the non-positively charged amino acid residues are neutral amino acid residues and negatively charged residues. In some embodiments, the cationic peptide does not insert into and disrupt a cell membrane. In some embodiments, no more than 40% of the amino acid residues in the cationic peptide are hydrophobic residues. In some embodiments, no more than 35% of the amino acid residues in the cationic peptide are hydrophobic residues. In some embodiments, no more than 30% of the amino acid residues in the cationic peptide are hydrophobic residues. In some embodiments, no more than 25% of the amino acid residues in the cationic peptide are hydrophobic residues. In some embodiments, no more than 20% of the amino acid residues in the cationic peptide are hydrophobic residues. In some embodiments, no more than 15% of the amino acid residues in the cationic peptide are hydrophobic residues. In some embodiments, no more than 10% of the amino acid residues in the cationic peptide are hydrophobic residues. In some embodiments, no more than 5% of the amino acid residues in the cationic peptide are hydrophobic residues. In some embodiments, the cationic peptide does not contain any hydrophobic residues.

In some embodiments, at least 25% of the amino acid residues in the cationic polypeptide are positively charged amino acid residues. In some embodiments, at least 30% of the amino acid residues in the cationic polypeptide are positively charged amino acid residues. In some embodiments, at least 35% of the amino acid residues in the cationic polypeptide are positively charged amino acid residues. In some embodiments, at least 40% of the amino acid residues in the cationic polypeptide are positively charged amino acid residues. In some embodiments, at least 45% of the amino acid residues in the cationic polypeptide are positively charged amino acid residues. In some embodiments, at least 50% of the amino acid residues in the cationic polypeptide are positively charged amino acid residues. In some embodiments, at least 55% of the amino acid residues in the cationic polypeptide are positively charged amino acid residues. In some embodiments, at least 60% of the amino acid residues in the cationic polypeptide are positively charged amino acid residues. In some embodiments, at least 65% of the amino acid residues in the cationic polypeptide are positively charged amino acid residues. In some embodiments, at least 70% of the amino acid residues in the cationic polypeptide are positively charged amino acid residues. In some embodiments, at least 75% of the amino acid residues in the cationic polypeptide are positively charged amino acid residues. In some embodiments, at least 80% of the amino acid residues in the cationic polypeptide are positively charged amino acid residues. In some embodiments, at least 85% of the amino acid residues in the cationic polypeptide are positively charged amino acid residues. In some embodiments, at least 90% of the amino acid residues in the cationic polypeptide are positively charged amino acid residues. In some embodiments, at least 95% of the amino acid residues in the cationic polypeptide are positively charged amino acid residues. In some embodiments, all of the amino acid residues in the cationic polypeptide are positively charged amino acid residues.

In some embodiments, the cationic polypeptide comprises at least 6 positively charged amino acid residues. In some embodiments, the cationic polypeptide comprises at least 7 positively charged amino acid residues. In some embodiments, the cationic polypeptide comprises at least 8 positively charged amino acid residues. In some embodiments, the cationic polypeptide comprises between 6 and 20 positively charged amino acid residues. In some embodiments, the cationic polypeptide comprises between 7 and 18 positively charged amino acid residues. In some embodiments, the cationic polypeptide comprises between 8 and 18 positively charged amino acid residues. In some embodiments, the cationic polypeptide has a net positive charge of at least 6. In some embodiments, the cationic polypeptide has a net positive charge of at least 7. In some embodiments, the cationic polypeptide has a net positive charge of at least 8. In some embodiments, the cationic polypeptide has a net positive charge of between 6 and 20. In some embodiments, the cationic polypeptide has a net positive charge of between 7 and 18. In some embodiments, the cationic polypeptide has a net positive charge of between 8 and 18. The net charge of a peptide is determined by subtracting the number negatively charged amino acid residues from the number of positively charged amino acid residues. By way of example, a cationic polypeptide comprising 10 positively charged amino acid residues and 3 negatively charged amino acid residues has a net positive charge of 7. Similarly, a cationic polypeptide comprising 7 positively charged amino acid residues and 10 neutral amino acid residues has a net positive charge of 7.

Positively charged amino acid residues include arginine (“Arg” or R), lysine (“Lys” or K), and histidine (“His” or H). In some embodiments, the cationic polypeptide comprises arginine, lysine and/or histidine residues. In some embodiments, the cationic polypeptide comprises arginine residues. In some embodiments, the cationic polypeptide comprises lysine residues. In some embodiments, the cationic polypeptide comprises histidine residues. In some embodiments, the cationic polypeptide comprises arginine and lysine residues. In some embodiments, the cationic polypeptide comprises arginine and histidine residues. In some embodiments, the cationic polypeptide comprises histidine and lysine residues. In some embodiments, the cationic polypeptide comprises arginine, lysine, and histidine residues. The cationic polypeptide may be a poly-arginine polypeptide. In some embodiments, the cationic polypeptide is a poly-lysine polypeptide. Optionally, the cationic polypeptide is a poly-histidine polypeptide. Poly-arginine and poly-lysine have been shown to be less immunogenic that other cationic polypeptides, including cationic polypeptides comprising neutral and/or negatively charged amino acid residues. Indeed, the FDA has approved poly-arginine for use as a coagulant and as a vaccine adjuvant. The skilled artisan would recognize that arginine residues have a stronger affinity for a polynucleotide than lysine residues or histidine residues. Further, lysine residues have a stronger affinity for a polynucleotide than histidine residues. Accordingly, poly-lysine polypeptides used in the compositions and methods of the disclosure will typically be longer than poly-arginine polypeptides used in the compositions and methods of the disclosure. Similarly, poly-histidine polypeptides used in the compositions and methods of the disclosure will typically be longer than poly-arginine polypeptides or poly-lysine polypeptides used in the compositions and methods of the disclosure. In some embodiments, the cationic polypeptide comprises a cross-linking amino acid residue. In some embodiments, the cross-linking amino acid residue is a cysteine. In some embodiments, the cationic polypeptide comprises one or more hydrophobic amino acid residues. In some embodiments, the one or more hydrophobic amino acid residues are selected from the group consisting of phenylalanine, tryptophan, leucine, alanine, isoleucine and a combination thereof. It is within the skill of the art to determine the correct length of a cationic polypeptide based on its composition (i.e. the number and type of its amino acid residues).

In some embodiments, the cationic polypeptide comprises L-amino acid residues. The cationic polypeptide may comprise D-amino acid residues. In some embodiments the cationic polypeptide comprises L-amino acid residues and D-amino acid residues. The cationic polypeptide may comprise between 9 and 18 amino acid residues. In some embodiments, the cationic polypeptide comprises 12 amino acid residues. For the formation of nanoparticles, cationic polypeptides longer than 18 residues and comprising only cationic amino acid residues are preferred. Accordingly, to avoid the formation of nanoparticles, in some embodiments, when the cationic polypeptide comprises only cationic amino acid residues, it comprises 18 or fewer amino acid residues.

The cationic polypeptide may comprise protamine. In some embodiments, the cationic peptide is an arginine-glycine-aspartic acid (RGD) polypeptide. In some embodiments, the cationic polypeptide is a cell penetrating peptide (CPP) with a cationic charge. Non-limiting examples of suitable CPPs are provided in Table 1, infra. Each CPP in Table 1 is considered a separate embodiment.

TABLE 1
Exemplary CPPs

| | | SEQ ID |
|---|---|---|
| Type of CPP | Sequences | NO: |
| Cationic | Tat: GRKKRRQRRRPPQ | 4 |
| | Penetratin: RQIKIWFQNRRMKWKK | 5 |
| Amphipathic | Transporant: GWTLNSAGYLLGKINLKALAALAKKIL | 6 |
| | MAP: KLALKLALKALKAALKLA | 7 |
| | Pep-1: KETWWETWWTEWSQPKKKRKV | 8 |
| Hydrophobic | Pept 1: PLILLRLLRGQF | 9 |
| | Pept 2: PLIYLRLLRGQF | 10 |
| | IVV-14: KLWMRWYSPTTRRYG | 11 |
| Chimeric | Transportan: GWTLNSAGYLLGKINLKALAALAKKIL | 12 |
| | Ig(v): MGLGLHLLVLAAALQGAKKKRKV | 13 |
| Synthetic | Amphiphilic model peptide: KLALKLALKALKAALKLA | 14 |
| | LAH4: KKALLALALHHLAHLALHLALALKKA | 15 |
| Protein- | pVEC: LLIILRRRIRKQAHAHSK | 16 |
| derived | HRSV: RRIPNRRPRR | 17 |
| Synthetic | RQWRRWWQRRQWRRWWQR-NH2 and | 18 |
| | RKFRRKFKK-NH2 | 19 |
| Synthetic | R8H4L4 | 20 |

In some embodiments, the cationic polypeptide is selected from the group of cationic polypeptides disclosed in Table 2, infra. In some embodiments, the hybrid polymer is selected from the group of hybrid polymer disclosed in Table 2, infra. Each cationic peptide and hybrid polymer in Table 2 is considered a separate embodiment.

TABLE 2
Exemplary Cationic Polypeptides/Hybrid Polymers Comprising Cationic Peptides

| | | | SEQ |
|---|---|---|---|
| Sequence¹ | Name | Description | ID NO: |
| {Peg12}R{D}R{D}R | Peg12-Poly | polyarginine containing peptide with | 21 |
| {D}R{D}R{D}R{D}R | (D-Arg)₁₂ | {D} unnatural amino acids and a PEG12 | |
| {D}R{D}R{D}R{D}R | | spacer inserted into a glycine residue. | |
| {D}R{D} | | | |
| {Peg12}RRRRRRRRR | Peg12-Poly | polyarginine containing peptide with | 22 |
| RRR | (L-Arg)₁₂ | natural amino acids and a PEG12 spacer | |
| | | inserted into a glycine residue. | |
| {Peg12}R{D}R{D}R | Peg12- Poly | polyarginine containing peptide with | 23 |
| {D}R{D}R{D}R{D}R | (D-Arg)₉ | {D} unnatural amino acids and a PEG12 | |
| {D}R{D}R{D} | | spacer inserted into a glycine residue. | |
| {Peg12}RRRRRRRRR | Peg12-Poly | polyarginine containing peptide with | 24 |
| | (L-Arg)₉ | natural amino acids. | |
| RRRRRRRRRRRR | Poly(L-Arg)₁₂ | polyarginine containing peptide with | 25 |
| | | natural amino acids. | |
| RRRRRRRRRRRRRR | PolyR18C | polyarginine containing peptide with | 26 |
| RRRRC | | natural amino acids and a terminal | |
| | | cysteine residue. | |
| {Peg12}{Peg12}RRRR | Peg24- Poly | polyarginine containing peptide with | 27 |
| RRRRRRRR | (L-Arg)₁₂ | natural amino acids and a PEG12 spacer | |
| | | inserted into two terminal glycine | |
| | | residues. | |
| {Peg12}{Peg12}RRRR | Peg24- Poly | polyarginine containing peptide with | 28 |
| RRRRR | (L-Arg)9 | natural amino acids and a PEG12 spacer | |
| | | inserted into two terminal glycine | |
| | | residues. | |
| RRRRRRRRRRRRRR | Poly(L-Arg)₁₅ | polyarginine containing peptide with | 29 |
| R | | natural amino acids. | |
| [Peg12}{Peg12}RRRR | Peg24- Poly | polyarginine containing peptide with | 30 |
| RRRRRRRRC | (L-Arg)₁₂C | natural amino acids and a PEG12 spacer | |
| | | inserted into two terminal glycine | |
| | | residues with a final terminal cysteine. | |
| [Peg12}RRRRRRRRR | Peg12- Poly | polyarginine containing peptide with | 31 |
| RRRRRRRRR | (L-Arg)₁₈ | natural amino acids and a PEG12 spacer | |
| | | inserted into a glycine residue. | |
| [Peg12}RRRRRRRRR | Peg12- Poly | polyarginine containing peptide with | 32 |
| RRRRRR | (L-Arg)₁₅ | natural amino acids and a PEG12 spacer | |
| | | inserted into a glycine residue. | |
| {Peg12}RRRRRRRRR | Peg12- Poly | polyarginine containing peptide with | 33 |
| RRRC | (L-Arg)₁₂C | natural amino acids and a PEG12 spacer | |
| | | inserted into a glycine residue and | |
| | | terminal cysteine. | |

¹R = L-Arg = L-Arginine; R{D} = D-Arg = D-Arginine; C = Cys = Cysteine

In some embodiments, the cationic polymer is a linear polymer. In some embodiments, the cationic polymer is a branched polymer. For the formation of nanoparticles, high molecular weight cationic polymers are preferred. Accordingly, in some embodiments, the cationic portion of the hybrid polymer comprises a low molecular weight polymer. In some embodiments, the low molecular weight polymer has a molecular weight between about 600 and about 2,000 Daltons. In some embodiments, the low molecular weight polymer has a molecular weight between about 700 and about 1,200 Daltons. In some embodiments, the low molecular weight polymer has a molecular weight between about 800 and about 1,000 Daltons. In some embodiments, the low molecular weight polymer has a molecular weight of about 600 Daltons. In some embodiments, the low molecular weight polymer has a molecular weight of about 700 Daltons. In some embodiments, the low molecular weight polymer has a molecular weight of about 800 Daltons. In some embodiments, the low molecular weight polymer has a molecular weight of about 900 Daltons. In some embodiments, the low molecular weight polymer has a molecular weight of about 1000 Daltons. In some embodiments, the low molecular weight polymer has a molecular weight of about 1100 Daltons. In some embodiments, the low molecular weight polymer has a molecular weight of about 1200 Daltons. In some embodiments, the low molecular weight polymer has a molecular weight of about 1300 Daltons. In some embodiments, the low molecular weight polymer has a molecular weight of about 1400 Daltons. In some embodiments, the low molecular weight polymer has a molecular weight of about 1500 Daltons. In some embodiments, the low molecular weight polymer has a molecular weight of about 1600 Daltons. In some embodiments, the low molecular weight polymer has a molecular weight of about 1700 Daltons. In some embodiments, the low molecular weight polymer has a molecular weight of about 1800 Daltons. In some embodiments, the low molecular weight polymer has a molecular weight of about 1900 Daltons. In some embodiments, the low molecular weight polymer has a molecular weight of about 2000 Daltons. In some embodiments, the low molecular weight polymer has a molecular weight between 600 and 2,000 Daltons. In some embodiments, the low molecular weight polymer has a molecular weight between 700 and 1,200 Daltons. In some embodiments, the low molecular weight polymer has a molecular weight between 800 and 1,000 Daltons. In some embodiments, the low molecular weight polymer has a molecular weight of 600 Daltons. In some embodiments, the low molecular weight polymer has a molecular weight of 700 Daltons. In some embodiments, the low molecular weight polymer has a molecular weight of 800 Daltons. In some embodiments, the low molecular weight polymer has a molecular weight of 900 Daltons. In some embodiments, the low molecular weight polymer has a molecular weight of 1000 Daltons. In some embodiments, the low molecular weight polymer has a molecular weight of 1100 Daltons. In some embodiments, the low molecular weight polymer has a molecular weight of 1200 Daltons. In some embodiments, the low molecular weight polymer has a molecular weight of 1300 Daltons. In some embodiments, the low molecular weight polymer has a molecular weight of 1400 Daltons. In some embodiments, the low molecular weight polymer has a molecular weight of 1500 Daltons. In some embodiments, the low molecular weight polymer has a molecular weight of 1600 Daltons. In some embodiments, the low molecular weight polymer has a molecular weight of 1700 Daltons. In some embodiments, the low molecular weight polymer has a molecular weight of 1800 Daltons. In some embodiments, the low molecular weight polymer has a molecular weight of 1900 Daltons. In some embodiments, the low molecular weight polymer has a molecular weight of 2000 Daltons. In some embodiments, the low molecular weight polymer is selected from the group consisting of gelatin, glucosamine, N-acetylglucosamine, chitosan, cationic dextran, cationic cyclodextrin, cationic cellulose, polyethylenimine (PEI), polyamidoamine (PAA), poly(amino-co-ester)s (PAEs), poly[2-(N,N-dimethylamino)ethyl methacrylate](PDMAEMA), or cationic lipids, such as DOTAP (N-(1-(2,3-dioleoyloxy) propyl)-N,N,N trimethylammonium) chloride, a cationic mucic acid polymer (cMAP) and DOPE (dioleoyl phosphatidylethanolamine). In some embodiments, the cationic polymer is linear PEI. In some embodiments, the cationic polymer is branched PEI (BPEI).

In some embodiments, the hybrid polymer is any of the polymers disclosed in Tables 5, 7, 8 and 9, infra. Each hybrid polymer recited in Tables 5, 7, 8 and 9 is considered a separate embodiment. In some embodiments, the hybrid polymer is selected from the group consisting of PEG12PolyArg12{d}, PEG12PolyArg6, PEG12PolyArg6C, PEG24PolyArg12C, PEG24PolyArg12, PEG24PolyArg9, PolyArg12C-PEG2000 Da, PolyArg12C-PEG5000 Da, PolyArg12C-Dextran5000 Da, PEG12PolyArg12, PEG12PolyArg9d, PEG1000DaPolyArg12, PEG2000DaPolyArg12, PEG5000DaPolyArg12, PolyArg12Cbp1.5 kDa, PolyArg12Cbp3.9 kDa, PolyArg12Cbp16 kDa, CPolyArg12Cbp1.5 kDa, PolyArg12Cbp2 kDa, PolyArg12bp2 kDa, Amide Dextran, Lysine Dextran, PEG PEI 15kda, BPEI-G-PEG 550, and BPEI-G-PEG 5000. In some embodiments, the hybrid polymer is PEG12PolyArg12{d}. In some embodiments, the hybrid polymer is PEG12PolyArg6. In some embodiments, the hybrid polymer is PEG12PolyArg6C. In some embodiments, the hybrid polymer is PEG24PolyArg12C. In some embodiments, the hybrid polymer is PEG24PolyArg12. In some embodiments, the hybrid polymer is PEG24PolyArg9. In some embodiments, the hybrid polymer is PolyArg12C-PEG2000 Da. In some embodiments, the hybrid polymer is PolyArg12C-PEG5000 Da. In some embodiments, the hybrid polymer is PolyArg12C-Dextran5000 Da. In some embodiments, the hybrid polymer is PEG12PolyArg12. In some embodiments, the hybrid polymer is PEG12PolyArg9d. In some embodiments, the hybrid polymer is PEG1000DaPolyArg12. In some embodiments, the hybrid polymer is PEG2000DaPolyArg12. In some embodiments, the hybrid polymer is PEG5000DaPolyArg12. In some embodiments, the hybrid polymer is PolyArg12Cbp1.5 kDa. In some embodiments, the hybrid polymer is PolyArg12Cbp3.9 kDa.

In some embodiments, the hybrid polymer is PolyArg12Cbp16 kDa. In some embodiments, the hybrid polymer is CPolyArg12Cbp1.5 kDa. In some embodiments, the hybrid polymer is PolyArg12Cbp2 kDa. In some embodiments, the hybrid polymer is PolyArg12bp2 kDa. In some embodiments, the hybrid polymer is Amide Dextran. In some embodiments, the hybrid polymer is Lysine Dextran. In some embodiments, the hybrid polymer is PEG PEI 15kda. In some embodiments, the hybrid polymer is BPEI-G-PEG 550. In some embodiments, the hybrid polymer is BPEI-G-PEG 5000.

In some embodiments, the neutral portion of the hybrid polymer has a molecular weight of about 100 to about 10,000 Daltons. In some embodiments, the neutral portion of the hybrid polymer has a molecular weight of about 100 Daltons. In some embodiments, the neutral portion of the hybrid polymer has a molecular weight of about 200 Daltons. In some embodiments, the neutral portion of the hybrid polymer has a molecular weight of about 300 Daltons. In some embodiments, the neutral portion of the hybrid polymer has a molecular weight of about 400 Daltons. In some embodiments, the neutral portion of the hybrid polymer has a molecular weight of about 500 Daltons. In some embodiments, the neutral portion of the hybrid polymer has a molecular weight of about 600 Daltons. In some embodiments, the neutral portion of the hybrid polymer has a molecular weight of about 700 Daltons. In some embodiments, the neutral portion of the hybrid polymer has a molecular weight of about 800 Daltons. In some embodiments, the neutral portion of the hybrid polymer has a molecular weight of about 900 Daltons. In some embodiments, the neutral portion of the hybrid polymer has a molecular weight of about 1000 Daltons. In some embodiments, the neutral portion of the hybrid polymer has a molecular weight of about 2000 Daltons. In some embodiments, the neutral portion of the hybrid polymer has a molecular weight of about 3000 Daltons. In some embodiments, the neutral portion of the hybrid polymer has a molecular weight of about 4000 Daltons. In some embodiments, the neutral portion of the hybrid polymer has a molecular weight of about 5000 Daltons. In some embodiments, the neutral portion of the hybrid polymer has a molecular weight of about 6000 Daltons. In some embodiments, the neutral portion of the hybrid polymer has a molecular weight of about 7000 Daltons. In some embodiments, the neutral portion of the hybrid polymer has a molecular weight of about 8000 Daltons. In some embodiments, the neutral portion of the hybrid polymer has a molecular weight of about 9000 Daltons. In some embodiments, the neutral portion of the hybrid polymer has a molecular weight of about 10000 Daltons.

In some embodiments, the neutral portion of the hybrid polymer has a molecular weight of 100 to 10,000 Daltons. In some embodiments, the neutral portion of the hybrid polymer has a molecular weight of 100 Daltons. In some embodiments, the neutral portion of the hybrid polymer has a molecular weight of 200 Daltons. In some embodiments, the neutral portion of the hybrid polymer has a molecular weight of 300 Daltons. In some embodiments, the neutral portion of the hybrid polymer has a molecular weight of 400 Daltons. In some embodiments, the neutral portion of the hybrid polymer has a molecular weight of 500 Daltons. In some embodiments, the neutral portion of the hybrid polymer has a molecular weight of 600 Daltons. In some embodiments, the neutral portion of the hybrid polymer has a molecular weight of 700 Daltons. In some embodiments, the neutral portion of the hybrid polymer has a molecular weight of 800 Daltons. In some embodiments, the neutral portion of the hybrid polymer has a molecular weight of 900 Daltons. In some embodiments, the neutral portion of the hybrid polymer has a molecular weight of 1000 Daltons. In some embodiments, the neutral portion of the hybrid polymer has a molecular weight of 2000 Daltons. In some embodiments, the neutral portion of the hybrid polymer has a molecular weight of 3000 Daltons. In some embodiments, the neutral portion of the hybrid polymer has a molecular weight of 4000 Daltons. In some embodiments, the neutral portion of the hybrid polymer has a molecular weight of 5000 Daltons. In some embodiments, the neutral portion of the hybrid polymer has a molecular weight of 6000 Daltons. In some embodiments, the neutral portion of the hybrid polymer has a molecular weight of 7000 Daltons. In some embodiments, the neutral portion of the hybrid polymer has a molecular weight of 8000 Daltons. In some embodiments, the neutral portion of the hybrid polymer has a molecular weight of 9000 Daltons. In some embodiments, the neutral portion of the hybrid polymer has a molecular weight of 10000 Daltons.

In some embodiments, the neutral portion of the hybrid polymer is a natural or synthetic polymer, consisting of long chains of branched or unbranched monomers, and/or cross-linked network of monomers in two or three dimensions. In some embodiments, the neutral portion of the hybrid polymer comprises a polysaccharide, lignin, rubber, or polyalkylene oxide (e.g., polyethylene glycol). Examples of the neutral portion of the hybrid polymer include, but are not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalate (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some embodiments, block copolymers are polymers wherein at least one section of a polymer is built up from monomers of another polymer. In some embodiments, the neutral portion of the hybrid polymer comprises polyalkylene oxide. In some embodiments, the neutral portion of the hybrid polymer comprises poly(ethylene glycol)(PEG). In some embodiments, the neutral portion of the hybrid polymer comprises a branched poly(ethylene glycol)(PEG). In some embodiments, the neutral portion of the hybrid polymer comprises a linear poly(ethylene glycol)(PEG). In some embodiments, the hybrid polymer is a PEGylated cationic polypeptide. The hybrid polymer may comprise a PEG12 to PEG24 polymer. Other suitable polymers for the neutral portion of the hybrid polymer are known in the art. See, e.g., Thi TTH, et al., Polymers, 2020 vol. 12:298; doi:10.3390/polym12020298, incorporated herein by reference in its entirety.

In some embodiments, the hybrid polymer and the polynucleotide do not form aggregates or nanoparticles. In some embodiments, the charge ratio of the cationic polypeptide to the polynucleotide is between about 0.25:1 and about 5:1. In some embodiments, the charge ratio of the cationic peptide to the polynucleotide is between about 0.5:1 and about 5:1. In some embodiments, the charge ratio of the cationic peptide to the polynucleotide is between about 1:1 and about 4:1. The charge ratio of the cationic polypeptide to the polynucleotide may be between about 1:1 and about 2:1. In some embodiments, the cationic polypeptide to the polynucleotide is about 1:1 or about 2:1.

In some embodiments, the hybrid polymer and the polynucleotide do not form aggregates or nanoparticles. In some embodiments, the charge ratio of the cationic polymer to the polynucleotide is between 0.25:1 and 5:1. In some embodiments, the charge ratio of the cationic polymer to the polynucleotide is between 0.5:1 and 5:1. In some embodiments, the charge ratio of the cationic polymer to the polynucleotide is between 1:1 and 4:1. The charge ratio of the cationic polymer to the polynucleotide may be between 1:1 and 2:1. In some embodiments, the cationic polymer to the polynucleotide is 1:1 or 2:1.

In some embodiments, at least 50% of the polynucleotide in the composition is present as a monomer species. In some embodiments, at least 55% of the polynucleotide in the composition is present as a monomer species. In some embodiments, at least 60% of the polynucleotide in the composition is present as a monomer species. In some embodiments, at least 65% of the polynucleotide in the composition is present as a monomer species. In some embodiments, at least 70% of the polynucleotide in the composition is present as a monomer species. In some embodiments, at least 75% of the polynucleotide in the composition is present as a monomer species. In some embodiments, at least 80% of the polynucleotide in the composition is present as a monomer species. In some embodiments, at least 85% of the polynucleotide in the composition is present as a monomer species. In some embodiments, at least 90% of the polynucleotide in the composition is present as a monomer species. In some embodiments, at least 95% of the polynucleotide in the composition is present as a monomer species. In some embodiments, at least 97% of the polynucleotide in the composition is present as a monomer species. In some embodiments, at least 99% of the polynucleotide in the composition is present as a monomer species. In some embodiments, all of the polynucleotide in the composition is present as a monomer species. In the context of an unconjugated polynucleotide of the composition, the term “monomer species” refers to complex comprising a single polynucleotide molecule and one or more hybrid polymers of the disclosure. For example, for an unconjugated polymer, a monomer species comprises a single polynucleotide and the one or more hybrid polymers to which it is ionically bound. In the context of polynucleotides conjugated to a targeting molecule, because a targeting molecule can be conjugated to multiple polynucleotides (e.g. DAR greater than 1), the term “monomer species” refers to a complex comprising only polynucleotides conjugated to the same targeting molecule. For example, for polynucleotides conjugated to a targeting molecule, a monomer species comprises a single targeting molecule, the polynucleotides conjugated to the targeting molecule (and only those polynucleotides), and the one or more hybrid polymers to which the polynucleotides are ionically bound.

In some embodiments, the polynucleotide is designed to hybridize to another polynucleotide or to dimerize with another polynucleotide. In some embodiments, the targeting molecule conjugated to one or more polynucleotides is designed to bind to or dimerize with another targeting molecule. In some embodiments, at least 50% of the polynucleotide in the composition is present as a dimer species. In some embodiments, at least 55% of the polynucleotide in the composition is present as a dimer species. In some embodiments, at least 60% of the polynucleotide in the composition is present as a dimer species. In some embodiments, at least 65% of the polynucleotide in the composition is present as a dimer species. In some embodiments, at least 70% of the polynucleotide in the composition is present as a dimer species. In some embodiments, at least 75% of the polynucleotide in the composition is present as a dimer species. In some embodiments, at least 80% of the polynucleotide in the composition is present as a dimer species. In some embodiments, at least 85% of the polynucleotide in the composition is present as a dimer species. In some embodiments, at least 90% of the polynucleotide in the composition is present as a dimer species. In some embodiments, at least 95% of the polynucleotide in the composition is present as a dimer species. In some embodiments, at least 97% of the polynucleotide in the composition is present as a dimer species. In some embodiments, at least 99% of the polynucleotide in the composition is present as a dimer species. In some embodiments, all of the polynucleotide in the composition is present as a dimer species. In the context of an unconjugated polynucleotide of the composition, the term “dimer species” refers to complex comprising two polynucleotide molecules and one or more hybrid polymers of the disclosure. For example, for an unconjugated polymer, a dimer species comprises two polynucleotides and the one or more hybrid polymers to which they are ionically bound. In the context of polynucleotides conjugated to a targeting molecule, because a targeting molecule can be conjugated to multiple polynucleotides (e.g., DAR greater than 1), the term “dimer species” refers to a complex comprising only polynucleotides conjugated to the two targeting molecules. For example, for polynucleotides conjugated to a targeting molecule, a dimer species comprises two targeting molecules, the polynucleotides conjugated to the two targeting molecules (and only those polynucleotides), and the one or more hybrid polymers to which the polynucleotides are ionically bound.

In some embodiments, the polynucleotide is designed to hybridize to two other polynucleotides or to trimerize with two other polynucleotides. In some embodiments, the targeting molecule conjugated to one or more polynucleotides is designed to bind to or trimerize with two targeting molecules. In some embodiments, at least 50% of the polynucleotide in the composition is present as a trimer species. In some embodiments, at least 55% of the polynucleotide in the composition is present as a trimer species. In some embodiments, at least 60% of the polynucleotide in the composition is present as a trimer species. In some embodiments, at least 65% of the polynucleotide in the composition is present as a trimer species. In some embodiments, at least 70% of the polynucleotide in the composition is present as a trimer species. In some embodiments, at least 75% of the polynucleotide in the composition is present as a trimer species. In some embodiments, at least 80% of the polynucleotide in the composition is present as a trimer species. In some embodiments, at least 85% of the polynucleotide in the composition is present as a trimer species. In some embodiments, at least 90% of the polynucleotide in the composition is present as a trimer species. In some embodiments, at least 95% of the polynucleotide in the composition is present as a trimer species. In some embodiments, at least 97% of the polynucleotide in the composition is present as a trimer species. In some embodiments, at least 99% of the polynucleotide in the composition is present as a trimer species. In some embodiments, all of the polynucleotide in the composition is present as a trimer species. In the context of an unconjugated polynucleotide of the composition, the term “trimer species” refers to complex comprising three polynucleotide molecules and one or more hybrid polymers of the disclosure. For example, for an unconjugated polymer, a trimer species comprises three polynucleotides and the one or more hybrid polymers to which they are ionically bound. In the context of polynucleotides conjugated to a targeting molecule, because a targeting molecule can be conjugated to multiple polynucleotides (e.g., DAR greater than 1), the term “trimer species” refers to a complex comprising only polynucleotides conjugated to three targeting molecules. For example, for polynucleotides conjugated to a targeting molecule, a trimer species comprises three targeting molecules, the polynucleotides conjugated to the two targeting molecules (and only those polynucleotides), and the one or more hybrid polymers to which the polynucleotides are ionically bound.

In some embodiments, the polynucleotide is conjugated to a targeting molecule. In some embodiments, the targeting moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of targeting moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some embodiments, the targeting moiety is an antibody or binding fragment thereof.

The targeting molecule may be an antibody or an antigen-binding fragment thereof, or a binding protein. In some embodiments, the targeting molecule is an antibody or an antigen binding fragment thereof (e.g. a polynucleotide-antibody conjugate). In some embodiments, the antibody or binding fragment thereof is a human antibody or an antigen-binding fragment thereof, a humanized antibody or an antigen-binding fragment thereof, a murine antibody or an antigen-binding fragment thereof, a chimeric antibody or an antigen-binding fragment thereof, a monoclonal antibody or an antigen-binding fragment thereof, a monovalent Fab′, a divalent Fab2, a F(ab)′3 fragment, a single-chain variable fragment (scFv), a bis-scFv, a (scFv)₂, a diabody, a minibody, an immunoglobulin single variable domain (ISV) such as an Nanobody® molecule, a triabody, a tetrabody, a disulfide stabilized Fv protein (dsFv), a single-domain antibody (sdAb), an Ig NAR, a vNAR, a Centyrin, a camelid antibody or an antigen-binding fragment thereof, a bispecific antibody or an antigen-biding fragment thereof, or a chemically modified derivative thereof. In some embodiments, the antibody or antigen-binding fragment thereof is selected from the group consisting of a monoclonal antibody, a bispecific antibody, a Fab, a Fab-Fc, a Fv, a single chain Fv (scFv), a diabody, a minibody, and a Nanobody®. In some embodiments, the antibody or antigen-binding fragment thereof is a monoclonal antibody. In some embodiments, the antibody or antigen-binding fragment thereof is a bispecific antibody. Non-limiting examples of bispecific antibodies in bispecific T-cell engagers (BiTEs) and a dual-affinity retargeting antibodies (DARTs). In some embodiments, the bispecific antibody is a trifunctional antibody or a bispecific mini-antibody. In some embodiments, the bispecific antibody is a trifunctional antibody. In some embodiments, the trifunctional antibody is a full-length monoclonal antibody comprising binding sites for two different antigens. In some embodiments, the bispecific antibody is a bispecific mini-antibody. In some embodiments, the bispecific mini-antibody comprises divalent Fab2, F(ab)′3 fragments, bis-scFv, (scFv)2, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens.

In some embodiments, the antibody or antigen-binding fragment thereof is a Fab. In some embodiments, the antibody or antigen-binding fragment thereof is a Fab-Fc. In some embodiments, the antibody or antigen-binding fragment thereof is a Fv. In some embodiments, the antibody or antigen-binding fragment thereof is a single chain Fv (scFv). In some embodiments, when the antibody or antigen-binding portion is a scFv, the polynucleotide does not comprise a cross-linking residue. In some embodiments, when the antibody or antigen-binding portion is a scFv, the polynucleotide does not comprise a cysteine. In some embodiments, the antibody or antigen-binding fragment thereof is a diabody. In some embodiments, the antibody or antigen-binding fragment thereof is a minibody. In some embodiments, the antibody or antigen-binding fragment thereof is an immunoglobulin single variable domain (ISV) such as an Nanobody® molecule. The Nanobody® may be a Nanobody-HSA®.

In some embodiments, the antibody or antigen-binding fragment thereof is an IgG molecule or is derived from an IgG molecule. The IgG molecule may be an IgG1 or an IgG4 molecule. The antibody or antigen-binding fragment thereof may be an IgG1 molecule or derived therefrom. The antibody or antigen-binding fragment thereof may be an IgG2 molecule or derived therefrom. The antibody or antigen-binding fragment thereof may be an IgG3 molecule or derived therefrom. The antibody or antigen-binding fragment thereof may be an IgG4 molecule or derived therefrom.

Non-limiting examples of antibodies and antigen-binding fragments that may be used as targeting molecules in the present disclosure include FV55scFv, Fv55 diabody, 3TF12, and cetuximab. The FV55 scFv is a monospecific scFv that binds to human transferrin receptor (TfR1). The CDRs of the FV55 scFv are identical to HB21, and the FV55 scFv is oriented V_{H}-V_{L} connected by (G4S)*3 (SEQ ID NO: 3) and has a c-terminal cysteine for conjugation. The molecular weight of the FV55 scFv is ˜26.5 kDa. See, e.g., Haynes B F, Hemler M, Cotner T, Mann D L, Eisenbarth G S, Strominger J L, Fauci A S. Characterization of a monoclonal antibody (5E9) that defines a human cell surface antigen of cell activation. J Immunol. 1981; 127:347-351. [PubMed: 6787129]. The Fv55 diabody comprises two copies of the FV55 scFv, except the linker is (G4S)*N (SEQ ID NO: 3), where N is 1 or 2. The molecular weight of the Fv55 diabody is ˜53 kDa. 3TF12 is a monospecific scFv that binds to human transferrin receptor (TfR1). 3TF12 is oriented V_{H}-V_{L} connected by (G4S)*N; when N is 3 (SEQ ID NO: 3), 3tf12 is a monomeric scFv; when N is 1 (SEQ ID NO: 3), 3tf12 dimerizes to form a diabody. See, e.g., Crepin R. et al. Development of Human Single-Chain Antibodies to the Transferrin Receptor that Effectively Antagonize the Growth of Leukemias and Lymphomas. Cancer Research, 2010, 70(13):5497-506. Cetuximab is a chimeric (mouse/human) monoclonal antibody and an epidermal growth factor receptor (EGFR) inhibitor medication used for the treatment of metastatic colorectal cancer and head and neck cancer. Cetuximab has a molecular weight of 145,781.92 g/mol.

In some embodiments, the targeting molecule is a binding protein. The binding protein may be a soluble receptor or a soluble ligand. In some embodiments, the soluble receptor comprises the extracellular domain of a receptor. In some embodiments, the soluble receptor is a Fc fusion protein.

In some embodiments, the targeting molecule is a plasma protein. In some embodiments, the plasma protein comprises albumin. In some embodiments, the albumin is conjugated by one or more of the conjugation chemistries disclosed herein to a polynucleotide. In some instances, the albumin is conjugated by native ligation chemistry to a polynucleotide. In some instances, albumin is conjugated by lysine conjugation to a polynucleotide.

In some instances, the targeting molecule is a steroid. Non-limiting exemplary steroids include cholesterol, phospholipids, di- and triacylglycerols, fatty acids, hydrocarbons that are saturated, unsaturated, comprise substitutions, or combinations thereof. In some embodiments, the steroid is cholesterol or a cholesterol derivative. In some embodiments, the targeting molecule is cholesterol. In some embodiments, the steroid is conjugated by one or more of the conjugation chemistries disclosed herein to a polynucleotide. In some embodiments, the steroid is conjugated by native ligation chemistry to a polynucleotide.

In some embodiments, the targeting molecule is a polymer, including but not limited to polynucleotide aptamers that bind to specific surface markers on cells. In some embodiments, the targeting molecule is a polynucleotide that does not hybridize to a target gene or mRNA, but instead is capable of selectively binding to a cell surface marker similarly to an antibody binding to its specific epitope of a cell surface marker.

In some embodiments, the targeting molecule is a polypeptide. In some embodiments, the polypeptide has a size between about 1 and about 3 kDa. In some embodiments, the polypeptide has a size between about 1.2 and about 2.8 kDa, between about 1.5 and about 2.5 kDa, or between about 1.5 and about 2 kDa. In some embodiments, the targeting molecule is a polypeptide. In some embodiments, the polypeptide has a size between 1 and 3 kDa. In some embodiments, the polypeptide has a size between 1.2 and 2.8 kDa, between 1.5 and 2.5 kDa, or between 1.5 and 2 kDa. In some embodiments, the polypeptide is a bicyclic polypeptide. In some embodiments, the bicyclic polypeptide is a constrained bicyclic polypeptide. In some embodiments, the targeting molecule is a bicyclic polypeptide (e.g., bicycles from Bicycle Therapeutics).

In additional embodiments, the targeting molecule is a small molecule. In some embodiments, the small molecule is an antibody-recruiting small molecule. In some embodiments, the antibody-recruiting small molecule comprises a target-binding terminus and an antibody-binding terminus, in which the target-binding terminus is capable of recognizing and interacting with a cell surface receptor.

In some embodiments, the targeting molecule is a therapeutically active molecule or a biologically active molecule.

In some embodiments, the polynucleotide is from about 5 to about 100 nucleotides in length. In some embodiments, the polynucleotide is from about 5 to about 50 nucleotides in length. In some embodiments, the polynucleotide is from about 10 to about 30, from about 15 to about 30, from about 18 to about 25, from about 18 to about 24, from about 19 to about 23, or from about 20 to about 22 nucleotides in length. In some embodiments, the polynucleotide is about 50 nucleotides in length. In some embodiments, the polynucleotide is about 49 nucleotides in length. In some embodiments, the polynucleotide is about 48 nucleotides in length. In some embodiments, the polynucleotide is about 47 nucleotides in length. In some embodiments, the polynucleotide is about 46 nucleotides in length. In some embodiments, the polynucleotide is about 45 nucleotides in length. In some embodiments, the polynucleotide is about 44 nucleotides in length. In some embodiments, the polynucleotide is about 43 nucleotides in length. In some embodiments, the polynucleotide is about 42 nucleotides in length. In some embodiments, the polynucleotide is about 41 nucleotides in length. In some embodiments, the polynucleotide is about 40 nucleotides in length. In some embodiments, the polynucleotide is about 39 nucleotides in length. In some embodiments, the polynucleotide is about 38 nucleotides in length. In some embodiments, the polynucleotide is about 37 nucleotides in length. In some embodiments, the polynucleotide is about 36 nucleotides in length. In some embodiments, the polynucleotide is about 35 nucleotides in length. In some embodiments, the polynucleotide is about 34 nucleotides in length. In some embodiments, the polynucleotide is about 33 nucleotides in length. In some embodiments, the polynucleotide is about 32 nucleotides in length. In some embodiments, the polynucleotide is about 31 nucleotides in length. In some embodiments, the polynucleotide is about 30 nucleotides in length. In some embodiments, the polynucleotide is about 29 nucleotides in length. In some embodiments, the polynucleotide is about 28 nucleotides in length. In some embodiments, the polynucleotide is about 27 nucleotides in length. In some embodiments, the polynucleotide is about 26 nucleotides in length. In some embodiments, the polynucleotide is about 25 nucleotides in length. In some embodiments, the polynucleotide is about 24 nucleotides in length. In some embodiments, the polynucleotide is about 23 nucleotides in length. In some embodiments, the polynucleotide is about 22 nucleotides in length. In some embodiments, the polynucleotide is about 21 nucleotides in length. In some embodiments, the polynucleotide is about 20 nucleotides in length. In some embodiments, the polynucleotide is about 19 nucleotides in length. In some embodiments, the polynucleotide is about 18 nucleotides in length. In some embodiments, the polynucleotide is about 17 nucleotides in length. In some embodiments, the polynucleotide is about 16 nucleotides in length. In some embodiments, the polynucleotide is about 15 nucleotides in length. In some embodiments, the polynucleotide is about 14 nucleotides in length. In some embodiments, the polynucleotide is about 13 nucleotides in length. In some embodiments, the polynucleotide is about 12 nucleotides in length. In some embodiments, the polynucleotide is about 11 nucleotides in length. In some embodiments, the polynucleotide is about 10 nucleotides in length. In some embodiments, the polynucleotide is about 9 nucleotides in length. In some embodiments, the polynucleotide is about 8 nucleotides in length. In some embodiments, the polynucleotide is about 7 nucleotides in length. In some embodiments, the polynucleotide is about 6 nucleotides in length. In some embodiments, the polynucleotide is about 5 nucleotides in length. In some embodiments, the polynucleotide is from about 10 to about 50 nucleotides in length. In some embodiments, the polynucleotide is from about 10 to about 45 nucleotides in length. In some embodiments, the polynucleotide is from about 10 to about 40 nucleotides in length. In some embodiments, the polynucleotide is from about 10 to about 35 nucleotides in length. In some embodiments, the polynucleotide is from about 10 to about 30 nucleotides in length. In some embodiments, the polynucleotide is from about 10 to about 25 nucleotides in length. In some embodiments, the polynucleotide is from about 10 to about 20 nucleotides in length. In some embodiments, the polynucleotide is from about 15 to about 25 nucleotides in length. In some embodiments, the polynucleotide is from about 15 to about 30 nucleotides in length. In some embodiments, the polynucleotide is from about 12 to about 30 nucleotides in length.

In some embodiments, the polynucleotide is from 5 to 100 nucleotides in length. In some embodiments, the polynucleotide is from 5 to 50 nucleotides in length. In some embodiments, the polynucleotide is from 10 to 30, from 15 to 30, from 18 to 25, from 18 to 24, from 19 to 23, or from 20 to 22 nucleotides in length. In some embodiments, the polynucleotide is 50 nucleotides in length. In some embodiments, the polynucleotide is 49 nucleotides in length. In some embodiments, the polynucleotide is 48 nucleotides in length.

In some embodiments, the polynucleotide is 47 nucleotides in length. In some embodiments, the polynucleotide 46 nucleotides in length. In some embodiments, the polynucleotide is 45 nucleotides in length. In some embodiments, the polynucleotide is 44 nucleotides in length.

In some embodiments, the polynucleotide is 43 nucleotides in length. In some embodiments, the polynucleotide is 42 nucleotides in length. In some embodiments, the polynucleotide is 41 nucleotides in length. In some embodiments, the polynucleotide is 40 nucleotides in length. In some embodiments, the polynucleotide is 39 nucleotides in length. In some embodiments, the polynucleotide is 38 nucleotides in length. In some embodiments, the polynucleotide is 37 nucleotides in length. In some embodiments, the polynucleotide is 36 nucleotides in length. In some embodiments, the polynucleotide is 35 nucleotides in length.

In some embodiments, the polynucleotide is 34 nucleotides in length. In some embodiments, the polynucleotide is 33 nucleotides in length. In some embodiments, the polynucleotide is 32 nucleotides in length. In some embodiments, the polynucleotide is 31 nucleotides in length.

In some embodiments, the polynucleotide is 30 nucleotides in length. In some embodiments, the polynucleotide is 29 nucleotides in length. In some embodiments, the polynucleotide is 28 nucleotides in length. In some embodiments, the polynucleotide is 27 nucleotides in length. In some embodiments, the polynucleotide is 26 nucleotides in length. In some embodiments, the polynucleotide is 25 nucleotides in length. In some embodiments, the polynucleotide is 24 nucleotides in length. In some embodiments, the polynucleotide is 23 nucleotides in length. In some embodiments, the polynucleotide is 22 nucleotides in length.

In some embodiments, the polynucleotide is 21 nucleotides in length. In some embodiments, the polynucleotide is 20 nucleotides in length. In some embodiments, the polynucleotide is 19 nucleotides in length. In some embodiments, the polynucleotide is 18 nucleotides in length.

In some embodiments, the polynucleotide is 17 nucleotides in length. In some embodiments, the polynucleotide is 16 nucleotides in length. In some embodiments, the polynucleotide is 15 nucleotides in length. In some embodiments, the polynucleotide is 14 nucleotides in length. In some embodiments, the polynucleotide is 13 nucleotides in length. In some embodiments, the polynucleotide is 12 nucleotides in length. In some embodiments, the polynucleotide is 11 nucleotides in length. In some embodiments, the polynucleotide is 10 nucleotides in length. In some embodiments, the polynucleotide is 9 nucleotides in length. In some embodiments, the polynucleotide is 8 nucleotides in length. In some embodiments, the polynucleotide is 7 nucleotides in length. In some embodiments, the polynucleotide is 6 nucleotides in length. In some embodiments, the polynucleotide is 5 nucleotides in length. In some embodiments, the polynucleotide is from 10 to 50 nucleotides in length. In some embodiments, the polynucleotide is from 10 to 45 nucleotides in length. In some embodiments, the polynucleotide is from 10 to 40 nucleotides in length. In some embodiments, the polynucleotide is from 10 to 35 nucleotides in length. In some embodiments, the polynucleotide is from 10 to 30 nucleotides in length. In some embodiments, the polynucleotide is from 10 to 25 nucleotides in length. In some embodiments, the polynucleotide is from 10 to 20 nucleotides in length. In some embodiments, the polynucleotide is from 15 to 25 nucleotides in length. In some embodiments, the polynucleotide is from 15 to 30 nucleotides in length. In some embodiments, the polynucleotide is from 12 to 30 nucleotides in length.

In some embodiments, the polynucleotide comprises RNA, DNA or a combination thereof. In some cases, the polynucleotide comprises RNA. In some cases, the polynucleotide comprises DNA. In some cases, the polynucleotide comprises RNA and DNA. In some embodiments, the polynucleotide comprises combinations of DNA, RNA and/or artificial nucleotide analogues. In some embodiments, the polynucleotide is a regulatory non-coding RNA (ncRNA). In some embodiments, the ncRNA comprises short non-coding RNA sequences expressed in a genome that regulates expression or function of other biomolecules in mammalian cells. An ncRNA is generally <200 nucleotides in length and can be single stranded or double stranded and may form non-linear secondary or tertiary structures. An ncRNA can comprise exogenously derived small interfering RNA (siRNA), MicroRNA (miRNA), small nuclear RNA (U-RNA), Small nucleolar RNA (snoRNA), Piwi-interacting RNA (piRNA), repeat associated small interfering RNA (rasiRNA), small rDNA-derived RNA (srRNA), transfer RNA derived small RNA (tsRNA), ribosomal RNA derived small RNA (rsRNA), large non-coding RNA derived small RNA (lncsRNA), or a messenger RNA derived small RNA (msRNA). In some embodiments, the polynucleotide is an engineered polynucleotide. The engineered polynucleotide may comprise DNA or RNA. In some embodiments, the engineered polynucleotide comprises a plurality of nucleotides. In some embodiments, the engineered polynucleotide comprises an artificial nucleotide analogue. In some embodiments, the engineered polynucleotide comprises DNA. In some embodiments, the DNA is genomic DNA, cell-free DNA, cDNA, fetal DNA, viral DNA, or maternal DNA. In some embodiments, the engineered polynucleotide comprises RNA. In some embodiments, the RNA is an siRNA, an ncRNA mimic, a short-harpin RNA (shRNA), a dicer-dependent siRNA (di-siRNA), an antisense oligonucleotide (ASO), a gapmer, a mixmer, double-stranded RNAs (dsRNA), single stranded RNAi, (ssRNAi), DNA-directed RNA interference (ddRNAi), an RNA activating oligonucleotide (RNAa), a transfer RNA (tRNA), a ribosomal RNA (rRNA), a heterogeneous nuclear RNA (hnRNA), promoter-associated RNAs (pRNAs), non-coding RNA element which regulates ribosomal RNA transcription by interacting with TIP5 (NoRC RNA), a ribozyme, anti-microRNA (antimiR), an aptamer, or an exon skipping oligonucleotide. In some embodiments, the engineered polynucleotide comprises a completely synthetic miRNA. A completely synthetic miRNA is one that is not derived or based upon an ncRNA. Instead, a completely synthetic miRNA may be based upon an analysis of multiple potential target sequences or may be based upon isolated natural non-coding sequences that are not ncRNAs. In some embodiments, the polynucleotide is selected from the group consisting of a siRNA, a miRNA, a miRNA mimic, an antisense oligonucleotide (ASO), an mRNA, and a guide RNA. The polynucleotide may be a siRNA. In some embodiments, the polynucleotide is a miRNA. In some embodiments, the polynucleotide is a miRNA mimic. The polynucleotide may be a miR-30 or a mimic of miR-30. Non-limiting examples of mimics of miR-30 are provided in Table 3, infra. Each miR-30 mimic in Table 3 is considered a separate embodiment.

TABLE 3
Sequences of exemplary mimics of miR-30

| Duplex | Guide Strand | Passenger Strand |
|---|---|---|
| Name | Sequence | Sequence |
| M30ml | fUmGfUmAfAmAfCmAf | Amino C6-mUmCmC |
| | UmCfCmUmGfCmGfA | AGUCGAGGAUGUUU |
| | mCfUmGfGmAsfAsmG | mAmCmA |
| | (SEQ ID NO: 34) | (SEQ ID NO: 35) |
| M30m2 | UGUAAACAUCCUCGAC | UUCAGUCGGAU |
| | UGGAAG (SEQ ID | GUUUGCAGC |
| | NO: 36) | (SEQ ID NO: 37) |
| M30m3 | UGUAAACAUCmCmUm | Amino C6- |
| | GmCmGmAmCmUmG | mUmCmCAGUC |
| | GAsfAsmG | GAGGAUGUUU |
| | (SEQ ID NO: 38) | mAmCmA |
| | | (SEQ ID NO: 39) |
| M30m4 | fUmGfUmAfAmAfCmAf | Amino C6-mUmCmCA |
| | UmCfCmUmGfCmGfA | GUCGAGGAUGUUU |
| | mCfUmGfGmAsfAsmG | mAmCmA-C6 Amino |
| | (SEQ ID NO: 34) | (SEQ ID NO: 41) |

In Table 3, capital N depicts any RNA nucleotide AUTGC, mN depicts a 2′O-Methyl modified nucleotide, fN depicts a 2′ Fluoro modified nucleotide, Amino C6- depicts a terminal amine linked to a terminal phosphate of the oligonucleotide by a C6 hydrocarbon linker, and s depicts a phosphorothioate linkage between the adjacent residues.

In some embodiments, the miR-30 mimic is selected from the group consisting of M30 ml, M30m2, M30m3, and M30m4. In some embodiments, the miR-30 mimic is M30 ml. In some embodiments, the miR-30 mimic is M30m2. In some embodiments, the miR-30 mimic is M30m3. In some embodiments, the miR-30 mimic is M30m4.

In some embodiments, is an ASO. In some embodiments, the ASO is an DMPK ASO. In some embodiments, the ASO is a CAPN3 ASO. The ASO may be a Dystrophy targeted exon skipping ASO. The ASO may be a DUX4-targeted ASO. DUX4-targeted ASOs are known in the art. See, e.g., WO 2021/203043 and U.S. Provisional Patent Application No. 63/221,568, each of which is incorporated herein by reference in its entirety. Additional non-limiting examples of DUX4-targeted ASOs are provided in Table 4, infra. Each DUX4-targeted ASO in Table 4 is considered a separate embodiment. In some embodiments, the DUX4-targeted ASO is selected from the group consisting of ASDX2, ASDX4, ASDX23, ASDX26, and ASDX32. In some embodiments, the DUX4-targeted ASO is ASDX2. In some embodiments, the DUX4-targeted ASO is ASDX4. In some embodiments, the DUX4-targeted ASO is ASDX23. In some embodiments, the DUX4-targeted ASO is ASDX26. In some embodiments, the DUX4-targeted ASO is ASDX32.

TABLE 4
Sequences of DUX4-targeted Antisense oligonucleotides

| | | SEQ ID |
|---|---|---|
| Oligo | Sequence (5′-3′)² | NO: |
| ASDX1 | {G}*{C}*c*a*t*c*g*c*g*g*g*t*a*g*{C}*{C} | 42 |
| ASDX2 | Amino C6-{G}*{C}*c*a*t*c*g*c*g*g*g*t*a*g*{C}*{C} | 43 |
| ASDX3 | Amino C6-{T}*{G}*t*c*g*g*g*a*g*g*g*c*c*a*{T}*{C} | 44 |
| ASDX4 | Amino C6-{T}{G}t*c*g*g*g*a*g*g*g*c*c*a{T}{C} | 45 |
| ASDX5 | Amino C6-{T}mp{G}t*c*g*g*g*a*g*g*g*c*c*a{T}mp{C} | 46 |
| ASDX6 | Amino C6-[T]*[G]*t*c*g*g*g*a*g*g*g*c*c*a*[T]*[C] | 47 |
| ASDX7 | {G}*{C}*c*a*t*c*g*c*g*g*g*t*a*g*{C}*{C} | 48 |
| ASDX8 | {T}*{C}*c*a*a*a*c*g*a*g*t*c*t*c*{C}*{G} | 49 |
| ASDX9 | Amino C6-{T}*{C}*c*a*a*a*c*g*a*g*t*c*t*c*{C}*{G} | 50 |
| ASDX10 | {G}*{A}*t*t*c*t*g*a*a*a*c*c*a*g*{A}*{T} | 51 |
| ASDX11 | Amino C6-{G}*{A}*t*t*c*t*g*a*a*a*c*c*a*g*{A}*{T} | 52 |
| ASDX12 | {G}*{C}*g*g*g*c*g*c*c*c*t*g*c*c*{A}*{C} | 53 |
| ASDX13 | Amino C6-{G}*{C}*g*g*g*c*g*c*c*c*t*g*c*c*{A}*{C} | 54 |
| ASDX14 | {T}*{C}*a*t*c*c*a*g*c*a*g*c*a*g*{G}*{C} | 55 |
| ASDX15 | Amino C6-{T}*{C}*a*t*c*c*a*g*c*a*g*c*a*g*{G}*{C} | 56 |
| ASDX16 | [T]*{A}*g*c*c*a*g*c*c*a*g*g*t*g*{T}*{T} | 57 |
| ASDX17 | Amino C6-{T}*{A}*g*c*c*a*g*c*c*a*g*g*t*g*{T}*{T} | 58 |
| ASDX18 | {C}*{A}*g*c*g*t*c*g*g*a*a*g*g*t*{G}*{G} | 59 |
| ASDX19 | Amino C6-{C}*{A}*g*c*g*t*c*g*g*a*a*g*g*t*{G}*{G} | 60 |
| ASDX20 | {T}*{A}*g*a*c*a*g*c*g*t*c*g*g*a*{A}*{G} | 61 |
| ASDX21 | Amino C6-{T}*{A}*g*a*c*a*g*c*g*t*c*g*g*a*{A}*{G} | 62 |
| ASDX22 | {A}*{T}*a*g*g*a*t*c*c*a*c*a*g*g*{G}*{A} | 63 |
| ASDX23 | Amino C6-{A}*{T}*a*g*g*a*t*c*c*a*c*a*g*g*{G}*{A} | 64 |
| ASDX24 | Amino C6-[A]*[T]*a*g*g*a*t*c*c*a*c*a*g*g*[G]*[A] | 65 |
| ASDX25 | {T}*{C}*t*a*t*a*g*g*a*t*c*c*a*c*{A}*{G} | 66 |
| ASDX26 | Amino C6-{T}*{C}*t*a*t*a*g*g*a*t*c*c*a*c*{A}*{G} | 67 |
| ASDX27 | {G}*{C}*a*c*t*a*a*t*c*a*t*c*c*a*{G}*{G} | 68 |
| ASDX28 | Amino C6-{G}*{C}*a*c*t*a*a*t*c*a*t*c*c*a*{G}*{G} | 69 |
| ASDX29 | {C}*{A}*{G}*c*g*t*c*g*g*a*a*g*{G}*{T}*{G} | 70 |
| ASDX30 | Amino C6-{C}*{A}*{G}*c*g*t*c*g*g*a*a*g*{G}*[T]*{G} | 71 |
| ASDX31 | (C)*(C)*(A)*(G)*c*g*t*c*g*g*a*a*g*(G)*(T)*(G)*(T) | 72 |
| ASDX32 | Amino C6-(C)*(C)*(A)*(G)*c*g*t*c*g*g*a*a*g*(G)*(T)*(G)*(T) | 73 |
| ASDX33 | {C}*{C}*t*{A}*g*a*c*a*g*c*g*t*c*g*{G}*{A}*a*g*{G}*{T} | 74 |
| ASDX34 | {A}*{T}*{A}*g*g*a*t*c*c*a*c*a*g*g*g*{A}*{G}*{G} | 75 |
| ASDX35 | {C}*{G}*{G}*c*t*c*t*g*g*g*a*t*c*c*c*{C}*{G}*{G} | 76 |
| ASDX36 | {G}*{G}*{G}*g*c*g*g*a*g*a*c*a*c*g*{C}*{C}*{C} | 77 |
| ASDX37 | {A}*{G}*{A}*a*g*g*c*a*g*g*a*a*t*c*c*{C}*{A}*{G} | 78 |
| ASDX38 | {G}*{C}*{A}*g*g*a*a*t*c*c*c*a*g*g*c*{C}*{G}*{G} | 79 |
| ASDX39 | {G}*{G}*{A}*g*t*c*t*c*t*c*a*c*c*g*g*{G}*{C}*{C} | 80 |
| ASDX40 | {A}*{G}*{A}*g*g*c*c*a*g*c*g*a*g*c*t*{C}*{C]*{C} | 81 |
| ASDX41 | {G}*{G}*{C}*t*c*t*g*g*g*a*t*c*c*c*c*{G}*{G}*{G} | 82 |
| ASDX42 | {C}*{A}*{G}*a*g*a*g*g*c*c*a*g*c*g*a*{G}*{C}*{T} | 83 |
| ASDX43 | {G}*{A}*{C}*a*g*c*g*t*c*g*g*a*a*g*g*{T}*{G}*{G} | 84 |
| ASDX44 | {G}*{T}*{A}*a*c*t*c*t*a*a*t*c*c*a*g*{G}*{T}*{T} | 85 |
| ASDX45 | {A}*{C}*{A}*a*g*g*g*c*a*c*a*g*a*g*a*{G}*{G}*{C} | 86 |
| ASDX46 | {G}*{A}*{G}*c*t*c*c*c*t***g*c*a*c*g*{T}*{C}*{A} | 87 |
| ASDX47 | {C}*{C}*{G}*t*c*c*a*a*c*c*c*c*g*c*{G}*{T}*{C} | 88 |
| ASDX48 | {C}*{C}*{T}*a*a*a*g*c*t*c*c*t*c*c*a*{G}*{C}*{A} | 89 |
| ASDX49 | {G}*{C}*{G}*a*g*g*c*g*g*c*c*t*c*t*t*{C}*{C}*{G} | 90 |
| ASDX50 | {G}*{C}*{C}*t*c*c*a*g*c*t*c*c*c*c*c*{G}*{G}*{G} | 91 |
| ASDX51 | {G}*{G}*{T}*g*t*c*g*g*g*a*g*g*g*c*{C}*{A}*{T} | 92 |
| ASDX52 | {C}*{T}*{G}*g*g*c*c*a*g*c*c*g*t*t*c*{T}*{C}*{T} | 93 |
| ASDX53 | {G}*{G}*{G}*c*c*a*g*c*c*g*t*t*c*t*c*{T}*{G}*{G} | 94 |
| ASDX54 | {C}*{A}*a*t*t*t*c*a*g*g*c*t*t*t*{T}*t*{C}*{T} | 95 |
| ASDX55 | {T}*{G}*{C}*c*t*a*c*a*g*a*a*g*g*c*t*{T}*{T}*{G} | 96 |
| ASDX56 | {A}*{T}*c*t*c*t*g*c*a*c*t*c*a*t*{C}*a*{C}*{A} | 97 |
| ASDX57 | {C}*{T}*g*a*t*c*a*c*c*g*a*a*g*t*{T}*c*{T}*{G} | 98 |
| ASDX58 | {A}*{T}*a*g*g*a*t*c*c*a*c*a*g*g*{G}*a*{G}*{G} | 99 |
| ASDX59 | {C}*{C}*a*g*g*a*g*a*t*g*t*a*a*c*{T}*c*{T}*{A} | 100 |
| ASDX60 | {G}*{A}*{A}*a*g*a*g*a*g*g*c*c*a*c*c*{G}*{C}*{C} | 101 |
| ASDX61 | [G]*{T}*{A}*g*c*c*a*g*c*c*a*g*g*t*g*{T}*{T}*{C} | 102 |
| ASDX62 | [G}*{C}*{C}*c*c*t*c*c*g*t*a*g*c*c*a*{G}*{C}*{C} | 103 |
| ASDX63 | {T}*{G}*{C}*t*g*t*c*c*g*a*g*g*g*t*g*{T}*{C}*{G} | 104 |
| ASDX64 | {A}*{G}*{G}*g*g*t*g*c*t*t*c*c*a*g*c*{G}*{A}*{G} | 105 |
| ASDX65 | {T}*{T}*{C}*t*t*c*c*t*c*g*c*t*g*a*g*{G}*{G}*{G} | 106 |
| ASDX66 | {C}*{G}*{G}*t*a*t*t*c*t*t*c*c*t*c*g*{C}*{T}*{G} | 107 |
| ASDX67 | {T}*{C}*{C}*t*c*c*a*g*c*a*g*a*g*c*c*{C}*{G}*{G} | 108 |
| ASDX68 | {C}*{C}*{T}*g*g*g*c*c*g*g*c*t*c*t*g*{G}*{G}*{A} | 109 |
| ASDX69 | {T}*{G}*{C}*t*g*g*t*a*c*c*t*g*g*g*c*{C}*{G}*{G} | 110 |
| ASDX70 | {T}*{C}*{T}*a*t*a*g*g*a*t*c*c*a*c*a*{G}*{G}*{G} | 111 |

²lowercase n depicts any DNA nucleotide a,t,c, or g; {N} is a locked nucleic acid (LNA) modified nucleotide; (N) is a 2′ methoxy ethyl modified nucleotide; [N] is a branched nucleic acid (BNA);*is a phosphorothioate -modified backbone.
Note:
For simplicity, for any sequence listed in Table 4 any phosphorothioate modification is optional, any deoxy cytosine (c) base could be a 5-Methylcytosine modified base, and any sequences could be further modified with an Amino C6- to form a conjugate.

In some embodiments, the polynucleotide comprises a siRNA, a miRNA, a miRNA mimic, an ASO, or a guide RNA that targets DUX4, DMPK or CAPN3. In some embodiments, the polynucleotide comprises a siRNA that targets DUX4. In some embodiments, the polynucleotide comprises a miRNA that targets DUX4. In some embodiments, the polynucleotide comprises a miRNA mimic that targets DUX4. In some embodiments, the polynucleotide comprises an ASO that targets DUX4. In some embodiments, the polynucleotide comprises a guide RNA that targets DUX4. In some embodiments, the polynucleotide comprises a siRNA that targets DMPK. In some embodiments, the polynucleotide comprises a miRNA that targets DMPK. In some embodiments, the polynucleotide comprises a miRNA mimic that targets DMPK. In some embodiments, the polynucleotide comprises an ASO that targets DMPK. In some embodiments, the polynucleotide comprises a siRNA that targets CAPN3. In some embodiments, the polynucleotide comprises a miRNA that targets CAPN3. In some embodiments, the polynucleotide comprises a miRNA mimic that targets CAPN3. In some embodiments, the polynucleotide comprises an ASO that targets CAPN3.

In some embodiments, the polynucleotide is a coding RNA. In some embodiments, the polynucleotide is a mRNA. In some embodiments, the polynucleotide is a non-coding RNA. In some embodiments, the polynucleotide is a long non-coding RNA. In some embodiments, the polynucleotide is a guide RNA.

In some embodiments, the polynucleotide comprises one or more artificial nucleotide analogues. In some instances, the artificial nucleotide analogues comprise modifications at one or more of ribose moiety, phosphate moiety, nucleoside moiety, or a combination thereof. In some embodiments, one or more of the artificial nucleotide analogues are resistant toward nucleases such as for example ribonuclease such as RNase, deoxyribunuclease such as DNase, or exonuclease such as 5′-3′ exonuclease and 3′-5′ exonuclease when compared to natural polynucleotides. In some embodiments, artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, BNA, 2′-O-Ethyl (cEt), morpholino, methylphosphonate nucleotides, thiolphosphonate nucleotides, 2′-fluoro N3-P5′-phosphoramidites, or combinations thereof are resistant toward nucleases such as for example ribonuclease such as RNase, deoxyribunuclease such as DNase, or exonuclease such as 5′-3′ exonuclease and 3′-5′ exonuclease. In some embodiments, 2′-O-methyl modified polynucleotide is nuclease resistant (e.g., RNase, DNase, 5′-3′ exonuclease or 3′-5′ exonuclease resistant). In some embodiments, 2′O-methoxyethyl (2′-O-MOE) modified polynucleotide is nuclease resistant (e.g., RNase, DNase, 5′-3′ exonuclease or 3′-5′ exonuclease resistant). In some embodiments, 2′-O-aminopropyl modified polynucleotide is nuclease resistant (e.g., RNase, DNase, 5′-3′ exonuclease or 3′-5′ exonuclease resistant). In some embodiments, 2′-deoxy modified polynucleotide is nuclease resistant (e.g., RNase, DNase, 5′-3′ exonuclease or 3′-5′ exonuclease resistant). In some embodiments, T-deoxy-2′-fluoro modified polynucleotide is nuclease resistant (e.g., RNase, DNase, 5′-3′ exonuclease or 3′-5′ exonuclease resistant). In some embodiments, 2′-O-aminopropyl (2′-O-AP) modified polynucleotide is nuclease resistant (e.g., RNase, DNase, 5′-3′ exonuclease or 3′-5′ exonuclease resistant). In some embodiments, 2′-O-dimethylaminoethyl (2′-O-DMAOE) modified polynucleotide is nuclease resistant (e.g., RNase, DNase, 5′-3′ exonuclease or 3′-5′ exonuclease resistant). In some embodiments, 2′-O-dimethylaminopropyl (2′-O-DMAP) modified polynucleotide is nuclease resistant (e.g., RNase, DNase, 5′-3′ exonuclease or 3′-5′ exonuclease resistant). In some embodiments, T-O-dimethylaminoethyloxyethyl (2′-O-DMAEOE) modified polynucleotide is nuclease resistant (e.g., RNase, DNase, 5′-3′ exonuclease or 3′-5′ exonuclease resistant). In some embodiments, 2′-O—N-methylacetamido (2′-O-NMA) modified polynucleotide is nuclease resistant (e.g., RNase, DNase, 5′-3′ exonuclease or 3′-5′ exonuclease resistant). In some embodiments, LNA-modified polynucleotide is nuclease resistant (e.g., RNase, DNase, 5′-3′ exonuclease or 3′-5′ exonuclease resistant). In some embodiments, ENA-modified polynucleotide is nuclease resistant (e.g., RNase, DNase, 5′-3′ exonuclease or 3′-5′ exonuclease resistant). In some embodiments, HNA-modified polynucleotide is nuclease resistant (e.g., RNase, DNase, 5′-3′ exonuclease or 3′-5′ exonuclease resistant). Morpholinos may be nuclease resistant (e.g., RNase, DNase, 5′-3′ exonuclease or 3′-5′ exonuclease resistant). In some embodiments, PNA-modified polynucleotide is resistant to nucleases (e.g., RNase, DNase, 5′-3′ exonuclease or 3′-5′ exonuclease resistant). In some embodiments, methylphosphonate nucleotide-modified polynucleotide is nuclease resistant (e.g., RNase, DNase, 5′-3′ exonuclease or 3′-5′ exonuclease resistant). In some embodiments, thiolphosphonate nucleotide-modified polynucleotide is nuclease resistant (e.g., RNase, DNase, 5′-3′ exonuclease or 3′-5′ exonuclease resistant). In some embodiments, polynucleotide comprising 2′-fluoro N3-P5′-phosphoramidites is nuclease resistant (e.g., RNase, DNase, 5′-3′ exonuclease or 3′-5′ exonuclease resistant). In some embodiments, the 5′ conjugates described herein inhibit 5′-3′ exonucleolytic cleavage. In some embodiments, the 3′ conjugates described herein inhibit 3′-5′ exonucleolytic cleavage.

In some embodiments, one or more of the artificial nucleotide analogues described herein have increased binding affinity toward their mRNA target relative to an equivalent natural polynucleotide. In some embodiments, the artificial nucleotide analogue comprises a nucleic acid with a modification at a 2′ hydroxyl group of the ribose moiety. In some embodiments, the modification includes an H, OR, R, halo, SH, SR, NH2, NHR, NR2, or CN, wherein R is an alkyl moiety. Exemplary alkyl moieties include, but are not limited to, halogens, sulfurs, thiols, thioethers, thioesters, amines (primary, secondary, or tertiary), amides, ethers, esters, alcohols and oxygen. In some embodiments, the alkyl moiety further comprises a modification. In some embodiments, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some embodiments, the alkyl moiety further comprises a hetero substitution. In some embodiments, the carbon of the heterocyclic group is substituted by a nitrogen, oxygen or sulfur. In some embodiments, the heterocyclic substitution includes but is not limited to, morpholino, imidazole, and pyrrolidino. The one or more of the artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites can have increased binding affinity toward their mRNA target relative to an equivalent natural polynucleotide. In some embodiments, 2′-O-methyl modified polynucleotide has increased binding affinity toward their mRNA target relative to an equivalent natural polynucleotide. In some embodiments, 2′-O-methoxyethyl (2′-O-MOE) modified polynucleotide has increased binding affinity toward their mRNA target relative to an equivalent natural polynucleotide. In some embodiments, 2′-O-aminopropyl modified polynucleotide has increased binding affinity toward their mRNA target relative to an equivalent natural polynucleotide. In some embodiments, 2′-deoxy modified polynucleotide has increased binding affinity toward their mRNA target relative to an equivalent natural polynucleotide. In some embodiments, T-deoxy-2′-fluoro modified polynucleotide has increased binding affinity toward their mRNA target relative to an equivalent natural polynucleotide. In some embodiments, 2′-O-aminopropyl (2′-O-AP) modified polynucleotide has increased binding affinity toward their mRNA target relative to an equivalent natural polynucleotide. In some embodiments, 2′-O-dimethylaminoethyl (2′-O-DMAOE) modified polynucleotide has increased binding affinity toward their mRNA target relative to an equivalent natural polynucleotide. In some embodiments, 2′-O-dimethylaminopropyl (2′-O-DMAP) modified polynucleotide has increased binding affinity toward their mRNA target relative to an equivalent natural polynucleotide. In some embodiments, T-O-dimethylaminoethyloxyethyl (2′-O-DMAEOE) modified polynucleotide has increased binding affinity toward their mRNA target relative to an equivalent natural polynucleotide. In some embodiments, 2′-O—N-methylacetamido (2′-O-NMA) modified polynucleotide has increased binding affinity toward their mRNA target relative to an equivalent natural polynucleotide. In some embodiments, LNA-modified polynucleotide has increased binding affinity toward their mRNA target relative to an equivalent natural polynucleotide. In some embodiments, ENA-modified polynucleotide has increased binding affinity toward their mRNA target relative to an equivalent natural polynucleotide. In some embodiments, PNA-modified polynucleotide has increased binding affinity toward their mRNA target relative to an equivalent natural polynucleotide. In some embodiments, HNA-modified polynucleotide has increased binding affinity toward their mRNA target relative to an equivalent natural polynucleotide. In some embodiments, morpholino-modified polynucleotide has increased binding affinity toward their mRNA target relative to an equivalent natural polynucleotide. In some embodiments, methylphosphonate nucleotide-modified polynucleotide has increased binding affinity toward their mRNA target relative to an equivalent natural polynucleotide. In some embodiments, thiolphosphonate nucleotide-modified polynucleotide has increased binding affinity toward their mRNA target relative to an equivalent natural polynucleotide. In some embodiments, polynucleotide comprising 2′-fluoro N3-P5′-phosphoramidites has increased binding affinity toward their mRNA target relative to an equivalent natural polynucleotide. In some embodiments, the increased affinity is illustrated with a lower Kd, a higher melt temperature (Tm), or a combination thereof.

In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, 2′-O-Ethyl (cEt), morpholino, methylphosphonate nucleotides, thiolphosphonate nucleotides, phosphorodithioate nucleotides, 2′-fluoro N3-P5′-phosphoramidites, or a combination thereof.

In some embodiments, the artificial nucleotide analogue comprises a modified base such as, but not limited to, 5-propynyluridine, 5-propynylcytidine, 6-methyladenine, 6-methylguanine, N,N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, In some embodiments are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, the polynucleotide comprises one or more phosphorothioate internucleotide linkages. In some embodiments, the polynucleotide comprises 2′-5′ internucleotide linkages. In some embodiments, the 2′-5′ internucleotide linkage(s) is at the 3′-end, the 5′-end, or both of the 3′- and 5′-ends of one or both sequence strands. In some embodiments, the 2′-5′ internucleotide linkage(s) is present at various other positions within one or both sequence strands. In some embodiments, the polynucleotide comprises a terminal cap molecule at the 3′-end, the 5′-end, or both of the 3′- and 5′-ends.

In some embodiments, the targeting molecule and the polynucleotide combined to provide a synergistic therapeutic or biological effect.

In some embodiments, the polynucleotide is conjugated directly to the targeting molecule. The polynucleotide may be conjugated to the targeting molecule via a linker.

Suitable linkers for conjugating polynucleotides to targeting molecules are known in the art.

See, e.g., WO 2017/173408, incorporated herein by reference in its entirety. In some embodiments, the linker is a hydrophobic linker. The linker may be a peptide linker. In some embodiments, the linker is a chemical linker. The chemical linker may be a polymeric linker. In some embodiments, the chemical linker is linear. In some embodiments, the chemical linker is cyclic.

In some embodiments, the polymeric linker comprises PEG, a sugar, a fatty acid, a phosphate, a pyrophosphate or a polysarcosine. In some embodiments, the polymeric linker comprises PEG. In some embodiments, the polymeric linker comprises a sugar. In some embodiments, the polymeric linker comprises a fatty acid. In some embodiments, the polymeric linker comprises a phosphate. In some embodiments, the polymeric linker comprises a pyrophosphate. In some embodiments, the polymeric linker comprises a polysarcosine. The linker may be a high molecular weight PEG linker. In some embodiments, the high molecular weight PEG linker comprises between 1,000 and 5,000 PEG monomers (i.e. is between PEG1k and PEG5k). In some embodiments, the high molecular weight PEG linker is PEG1k. In some embodiments, the high molecular weight PEG linker is PEG1.5k. In some embodiments, the high molecular weight PEG linker is PEG2k. In some embodiments, the high molecular weight PEG linker is PEG3k. In some embodiments, the high molecular weight PEG linker is PEG4k. In some embodiments, the high molecular weight PEG linker is PEG5k.

In some embodiments, the linker is a low molecular weight PEG linker. In some embodiments, the low molecular weight PEG linker comprises between 4 and 100 PEG monomers (i.e. is between PEG4 and PEG100). In some embodiments, the low molecular PEG linker is between PEG12 and PEG48. In some embodiments, the low molecular PEG linker is between PEG12 and PEG24. In some embodiments, the low molecular PEG linker is between PEG12 and PEG18. In some embodiments, the low molecular PEG linker is between PEG6 and PEG18. In some embodiments, the low molecular weight PEG linker is PEG4. In some embodiments, the low molecular weight PEG linker is PEG5. In some embodiments, the low molecular weight PEG linker is PEG6. In some embodiments, the low molecular weight PEG linker is PEG7. In some embodiments, the low molecular weight PEG linker is PEG8. In some embodiments, the low molecular weight PEG linker is PEG9.

In some embodiments, the low molecular weight PEG linker is PEG10. In some embodiments, the low molecular weight PEG linker is PEG11. In some embodiments, the low molecular weight PEG linker is PEG12. In some embodiments, the low molecular weight PEG linker is PEG13. In some embodiments, the low molecular weight PEG linker is PEG14. In some embodiments, the low molecular weight PEG linker is PEG15. In some embodiments, the low molecular weight PEG linker is PEG16. In some embodiments, the low molecular weight PEG linker is PEG17. In some embodiments, the low molecular weight PEG linker is PEG18. In some embodiments, the low molecular weight PEG linker is PEG19. In some embodiments, the low molecular weight PEG linker is PEG20. In some embodiments, the low molecular weight PEG linker is PEG21. In some embodiments, the low molecular weight PEG linker is PEG22. In some embodiments, the low molecular weight PEG linker is PEG23. In some embodiments, the low molecular weight PEG linker is PEG24. In some embodiments, the low molecular weight PEG linker is PEG25. In some embodiments, the low molecular weight PEG linker is PEG26. In some embodiments, the low molecular weight PEG linker is PEG27. In some embodiments, the low molecular weight PEG linker is PEG28. In some embodiments, the low molecular weight PEG linker is PEG29. In some embodiments, the low molecular weight PEG linker is PEG30. In some embodiments, the low molecular weight PEG linker is PEG31. In some embodiments, the low molecular weight PEG linker is PEG32. In some embodiments, the low molecular weight PEG linker is PEG33. In some embodiments, the low molecular weight PEG linker is PEG34. In some embodiments, the low molecular weight PEG linker is PEG35. In some embodiments, the low molecular weight PEG linker is PEG36. In some embodiments, the low molecular weight PEG linker is PEG37. In some embodiments, the low molecular weight PEG linker is PEG38. In some embodiments, the low molecular weight PEG linker is PEG39. In some embodiments, the low molecular weight PEG linker is PEG40. In some embodiments, the low molecular weight PEG linker is PEG41. In some embodiments, the low molecular weight PEG linker is PEG42. In some embodiments, the low molecular weight PEG linker is PEG43. In some embodiments, the low molecular weight PEG linker is PEG44. In some embodiments, the low molecular weight PEG linker is PEG45. In some embodiments, the low molecular weight PEG linker is PEG46. In some embodiments, the low molecular weight PEG linker is PEG47. In some embodiments, the low molecular weight PEG linker is PEG48. In some embodiments, the low molecular weight PEG linker is PEG49. In some embodiments, the low molecular weight PEG linker is PEG50. In some embodiments, the low molecular weight PEG linker is PEG51. In some embodiments, the low molecular weight PEG linker is PEG52. In some embodiments, the low molecular weight PEG linker is PEG53. In some embodiments, the low molecular weight PEG linker is PEG54. In some embodiments, the low molecular weight PEG linker is PEG55. In some embodiments, the low molecular weight PEG linker is PEG56. In some embodiments, the low molecular weight PEG linker is PEG57. In some embodiments, the low molecular weight PEG linker is PEG58. In some embodiments, the low molecular weight PEG linker is PEG59. In some embodiments, the low molecular weight PEG linker is PEG60. In some embodiments, the low molecular weight PEG linker is PEG61. In some embodiments, the low molecular weight PEG linker is PEG62. In some embodiments, the low molecular weight PEG linker is PEG63. In some embodiments, the low molecular weight PEG linker is PEG64. In some embodiments, the low molecular weight PEG linker is PEG65. In some embodiments, the low molecular weight PEG linker is PEG66. In some embodiments, the low molecular weight PEG linker is PEG67. In some embodiments, the low molecular weight PEG linker is PEG68. In some embodiments, the low molecular weight PEG linker is PEG69. In some embodiments, the low molecular weight PEG linker is PEG70. In some embodiments, the low molecular weight PEG linker is PEG71. In some embodiments, the low molecular weight PEG linker is PEG72. In some embodiments, the low molecular weight PEG linker is PEG73. In some embodiments, the low molecular weight PEG linker is PEG74. In some embodiments, the low molecular weight PEG linker is PEG75. In some embodiments, the low molecular weight PEG linker is PEG76. In some embodiments, the low molecular weight PEG linker is PEG77. In some embodiments, the low molecular weight PEG linker is PEG78. In some embodiments, the low molecular weight PEG linker is PEG79. In some embodiments, the low molecular weight PEG linker is PEG80. In some embodiments, the low molecular weight PEG linker is PEG81. In some embodiments, the low molecular weight PEG linker is PEG82. In some embodiments, the low molecular weight PEG linker is PEG83. In some embodiments, the low molecular weight PEG linker is PEG84. In some embodiments, the low molecular weight PEG linker is PEG85. In some embodiments, the low molecular weight PEG linker is PEG86. In some embodiments, the low molecular weight PEG linker is PEG87. In some embodiments, the low molecular weight PEG linker is PEG88. In some embodiments, the low molecular weight PEG linker is PEG89. In some embodiments, the low molecular weight PEG linker is PEG90. In some embodiments, the low molecular weight PEG linker is PEG91. In some embodiments, the low molecular weight PEG linker is PEG92. In some embodiments, the low molecular weight PEG linker is PEG93. In some embodiments, the low molecular weight PEG linker is PEG94. In some embodiments, the low molecular weight PEG linker is PEG95. In some embodiments, the low molecular weight PEG linker is PEG96. In some embodiments, the low molecular weight PEG linker is PEG97. In some embodiments, the low molecular weight PEG linker is PEG98. In some embodiments, the low molecular weight PEG linker is PEG99. In some embodiments, the low molecular weight PEG linker is PEG100.

In some embodiments, the linker is non-cleavable. In some embodiments, the linker is cleavable. The linker may be cleavable in vivo. In some embodiments, the cleavable linker is selected from the group consisting of a disulfide linker, a self-immolative peptide polymer hybrid, and a sulfatase-promoted arylsulfate linker. In some embodiments, the cleavable linker is a disulfide linker. The cleavable linker may be a self-immolative peptide polymer hybrid. In some embodiments, the cleavable linker is a sulfatase-promoted arylsulfate linker. In some embodiments, the self-immolative peptide polymer hybrid comprises glucuronic acid, para-amino-benzoyloxy (PAB), 7-amino-3-hydroxyethyl-coumarin (7-AHC), or Fe(II)-reactive 1,2,4-trioxolane scaffold (TRX). In some embodiments, the self-immolative peptide polymer hybrid comprises glucuronic acid. In some embodiments, the self-immolative peptide polymer hybrid comprises para-amino-benzoyloxy (PAB). In some embodiments, the self-immolative peptide polymer hybrid comprises 7-amino-3-hydroxyethyl-coumarin (7-AHC). In some embodiments, the self-immolative peptide polymer hybrid comprises Fe(II)-reactive 1,2,4-trioxolane scaffold (TRX).

In some embodiments, the cleavable linker is cleaved through reduction, hydrolysis, proteolysis, photo cleavage, chemical cleavage, enzymatic cleavage, or bio-orthogonal-cleavage. In some embodiments, the cleavable linker is cleaved through reduction. In some embodiments, the cleavable linker is cleaved through hydrolysis. In some embodiments, the cleavable linker is cleaved through proteolysis. In some embodiments, the cleavable linker is cleaved through photo cleavage. In some embodiments, the cleavable linker is cleaved through chemical cleavage. The chemical cleavage may be by Fe II mediated R elimination of TRX. In some embodiments, the cleavable linker is cleaved through enzymatic cleavage.

The enzymatic cleavage may be by non-proteolytic sulfatase, β-galactosidase/glucuronidase or pyrophosphatase. In some embodiments, the enzymatic cleavage is by non-proteolytic sulfatase. In some embodiments, the enzymatic cleavage is by β-galactosidase/glucuronidase. In some embodiments, the enzymatic cleavage is by pyrophosphatase. In some embodiments, the cleavable linker is cleaved through bio-orthogonal-cleavage. The bio-orthogonal cleavage may be by Cu I-BTTAA or free copper ion mediated cleavage. In some embodiments, the linker is an acid cleavable linker.

In some embodiments, the linker includes a C1-C6 alkyl group (e.g., a C5, C4, C3, C2, or C1 alkyl group). In some embodiments, the linker includes homobifunctional cross linkers, heterobifunctional cross linkers, and the like. In some embodiments, the liker is a traceless linker (or a zero-length linker). In some embodiments, the linker is a non-polymeric linker. In some embodiments, the linker is a non-peptide linker or a linker that does not contain an amino acid residue.

In some embodiments, the linker comprises a homobifuctional linker. Exemplary homobifuctional linkers include, but are not limited to, Lomant's reagent dithiobis (succinimidylpropionate) DSP, 3,3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[2-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-ρ-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(y-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl) 1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as 1-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some embodiments, the linker comprises a reactive functional group. In some embodiments, the reactive functional group comprises a nucleophilic group that is reactive to an electrophilic group present on a binding moiety. Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (mc). In some embodiments, the linker comprises maleimidocaproyl (mc). In some embodiments, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some embodiments, the self-stabilizing maleimide is a maleimide group described in Lyon, et al, “Self-hydrolyzing maleimides improve the stability and pharmacological properties of antibody-drug conjugates,” Nat. Biotechnol. 32(10): 1059-1062 (2014). In some embodiments, the linker comprises a self-stabilizing maleimide. In some embodiments, the linker is a self-stabilizing maleimide.

In some embodiments, the linker comprises a peptide moiety. In some embodiments, the peptide moiety comprises at least 2, 3, 4, 5, 6, 7, 8, or more amino acid residues. In some embodiments, the peptide moiety is a cleavable peptide moiety (e.g., either enzymatically or chemically). In some embodiments, the peptide moiety is a non-cleavable peptide moiety. In some embodiments, the peptide moiety comprises Val-Cit (valine-citrulline), Gly-Gly-Phe-Gly (SEQ ID NO: 112), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 113), or Gly-Phe-Leu-Gly (SEQ ID NO: 114). In some embodiments, the linker comprises a peptide moiety such as: Val-Cit (valine-citrulline), Gly-Gly-Phe-Gly (SEQ ID NO: 112), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 113), or Gly-Phe-Leu-Gly (SEQ ID NO: 114). In some embodiments, the linker comprises Val-Cit. In some embodiments, the linker is Val-Cit.

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (mc). In some embodiments, the peptide group is val-cit. In some embodiments, the benzoic acid group is PABA. In some embodiments, the linker comprises a mc-val-cit group. In some embodiments, the linker comprises a val-cit-PABA group. In additional cases, the linker comprises a mc-val-cit-PABA group.

In some embodiments, the linker is a self-immolative linker or a self-elimination linker. In some embodiments, the linker is a self-immolative linker. In other cases, the linker is a self-elimination linker (e.g., a cyclization self-elimination linker). In some embodiments, the linker comprises a linker described in U.S. Pat. No. 9,089,614 or PCT Publication No. WO2015038426, each of which is incorporated herein by reference in its entirety.

In some embodiments, the linker is a dendritic type linker. In some embodiments, the dendritic type linker comprises a branching, multifunctional linker moiety. In some embodiments, the dendritic type linker is used to increase the molar ratio of polynucleotide B to the binding moiety A. In some embodiments, the dendritic type linker comprises PAMAM dendrimers.

In some embodiments, the linker is a traceless linker or a linker in which after cleavage does not leave behind a linker moiety (e.g., an atom or a linker group) to a polynucleotide or a targeting molecule. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some embodiments, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., “Traceless solid-phase organic synthesis,” Chem. Rev. 102: 2607-2024 (2002). In some embodiments, a linker is a traceless linker as described in U.S. Pat. No. 6,821,783, incorporated herein by reference in its entirety.

In some embodiments, the linker comprises a functional group that exerts steric hinderance at the site of bonding between the linker and a conjugating moiety (e.g., a polynucleotide or a targeting molecule disclosed herein). In some embodiments, the steric hinderance is a steric hindrance around a disulfide bond. Exemplary linkers that exhibit steric hinderance comprises a heterobifunctional linker, such as a heterobifunctional linker described above. In some embodiments, a linker that exhibits steric hinderance comprises SMCC and SPDB.

In some embodiments, the linker is an acid cleavable linker. In some embodiments, the acid cleavable linker comprises a hydrazone linkage, which is susceptible to hydrolytic cleavage. In some embodiments, the acid cleavable linker comprises a thiomaleamic acid linker. In some embodiments, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some embodiments, the linker is a linker described in U.S. Pat. Nos. 6,884,869; 7,498,298; 8,288,352; 8,609, 105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042, each of which is incorporated herein by reference in its entirety.

In some embodiments, the linker is conjugated to a lysine residue, a cysteine residue, a histidine residue, or a non-natural amino acid residue in the targeting molecule. In some embodiments, the linker is conjugated to a lysine residue in the targeting molecule. In some embodiments, the linker is conjugated to a cysteine residue in the targeting molecule. In some embodiments, the linker is conjugated to a histidine residue in the targeting molecule. In some embodiments, the linker is conjugated to a non-natural amino acid residue in the targeting molecule.

In some embodiments, the linker is conjugated to the targeting molecule by a chemical conjugation or an enzymatic conjugation. In some embodiments, the linker is conjugated to the targeting molecule by a chemical conjugation. The chemical conjugation may comprise acylation and click chemistry. In some embodiments, the linker is conjugated to the targeting molecule by an enzymatic conjugation. The enzymatic conjugation may be via a sortase or a transferase enzyme.

In some embodiments, the polynucleotide is conjugated to the targeting molecule by a chemical ligation process. In some embodiments, the polynucleotide is conjugated to the targeting molecule by a native ligation. In some embodiments, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some embodiments, the conjugation is as described in U.S. Pat. No. 8,936,910. In some embodiments, the polynucleotide is conjugated to the targeting molecule either site-specifically or non-specifically via native ligation chemistry.

In some embodiments, the polynucleotide is conjugated to the targeting molecule by a site-directed method utilizing a “traceless” coupling technology (Philochem). In some embodiments, the “traceless” coupling technology utilizes an N-terminal 1,2-aminothiol group on the targeting molecule which is then conjugate with a polynucleotide containing an aldehyde group. (see Casi et al., “Site-specific traceless coupling of potent cytotoxic drugs to recombinant antibodies for pharmacodelivery,” JACS 134(13): 5887-5892 (2012)).

In some embodiments, the polynucleotide is conjugated to the targeting molecule by a site-directed method utilizing an unnatural amino acid incorporated into the targeting molecule. In some embodiments, the unnatural amino acid comprises p-acetylphenylalanine (pAcPhe). In some embodiments, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some embodiments, the polynucleotide is conjugated to the targeting molecule by a site-directed method utilizing an enzyme-catalyzed process. In some embodiments, the site-directed method utilizes SMARTag™ technology (Redwood). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleotide via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some embodiments, the enzyme-catalyzed process comprises microbial transglutaminase (mTG). In some embodiments, the polynucleotide is conjugated to the targeting molecule utilizing a microbial transglutaminze catalyzed process. In some embodiments, mTG catalyzes the formation of a covalent bond between the amide side chain of a glutamine within the recognition sequence and a primary amine of a functionalized polynucleotide. In some embodiments, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some embodiments, the polynucleotide is conjugated to the targeting molecule by a method as described in PCT Publication No. WO2014/140317 (incorporated herein by reference in its entirety), which utilizes a sequence-specific transpeptidase. In some embodiments, the polynucleotide is conjugated to the targeting molecule by a method as described in U.S. Patent Publication Nos. 2015/0105539 and 2015/0105540, each of which is incorporated herein by reference in its entirety.

In some embodiments, each targeting molecule is conjugated to between one and eight polynucleotide molecules (i.e. a Drug:Antibody Ratio (DAR) between 1 and 8). In some embodiments, each targeting molecule is conjugated to one polynucleotide molecule (DAR of 1). In some embodiments, each targeting molecule is conjugated to two polynucleotide molecules (DAR of 2). In some embodiments, each targeting molecule is conjugated to three polynucleotide molecules (DAR of 3). In some embodiments, each targeting molecule is conjugated to four polynucleotide molecules (DAR of 4). In some embodiments, each targeting molecule is conjugated to five polynucleotide molecules (DAR of 5). In some embodiments, each targeting molecule is conjugated to six polynucleotide molecules (DAR of 6). In some embodiments, each targeting molecule is conjugated to seven polynucleotide molecules (DAR of 7). In some embodiments, each targeting molecule is conjugated to eight polynucleotide molecules (DAR of 8).

In some embodiments, the polynucleotide-conjugated targeting molecule has a molecular weight greater than about 30 kDa. In some embodiments, the polynucleotide-conjugated targeting molecule has a molecular weight greater than about 40 kDa. The polynucleotide-conjugated targeting molecule may have a molecular weight greater than about 50 kDa. In some embodiments, the polynucleotide-conjugated targeting molecule has a molecular weight greater than about 60 kDa. In some embodiments, the polynucleotide-conjugated targeting molecule has a molecular weight no greater than about 7,500 kDa.

In some embodiments, the polynucleotide-conjugated targeting molecule has a molecular weight greater than 30 kDa. In some embodiments, the polynucleotide-conjugated targeting molecule has a molecular weight greater than 40 kDa. The polynucleotide-conjugated targeting molecule may have a molecular weight greater than 50 kDa. In some embodiments, the polynucleotide-conjugated targeting molecule has a molecular weight greater than 60 kDa. In some embodiments, the polynucleotide-conjugated targeting molecule has a molecular weight no greater than 7,500 kDa.

In some embodiments, the polynucleotide conjugate is selected from the group consisting of Cetuximab-DBCO-C9-M30m3 (DAR of 3); Cetuximab-DBCO-C4/P5-M30m3 (DAR of 3); Cetuximab-DBCO-PEG9-M30m3 (DAR of 3); Cetuximab-DBCO-PEG9-M30m3 (DAR of 2); Cetuximab-DBCO-PEG9-M30m3 (DAR of 4); Cetuximab-DBCO-PEG9-M30m3 (DAR of 6); Cetuximab-Linear-PEG13-M30m3 (DAR of 4); 3tf12-DBCO-PEG8-NCD5 (DAR of 1); 3tf12-DBCO-PEG8-M30m3 (DAR of 1); Fv55-SMCC-M30m3 (DAR of 1); Fv55-PEG8-DBCO-M30m3(DAR of 1) and Fv55-PEG8-DBCO-M30m3(DAR of 2). In some embodiments, the polynucleotide conjugate is Cetuximab-DBCO-C9-M30m3 (DAR of 3). In some embodiments, the polynucleotide conjugate is Cetuximab-DBCO-C4/P5-M30m3 (DAR of 3). In some embodiments, the polynucleotide conjugate is Cetuximab-DBCO-PEG9-M30m3 (DAR of 3). In some embodiments, the polynucleotide conjugate is Cetuximab-DBCO-PEG9-M30m3 (DAR of 2). In some embodiments, the polynucleotide conjugate is Cetuximab-DBCO-PEG9-M30m3 (DAR of 4). In some embodiments, the polynucleotide conjugate is Cetuximab-DBCO-PEG9-M30m3 (DAR of 6). In some embodiments, the polynucleotide conjugate is Cetuximab-Linear-PEG13-M30m3 (DAR of 4). In some embodiments, the polynucleotide conjugate is 3tf12-DBCO-PEG8-NCD5 (DAR of 1). In some embodiments, the polynucleotide conjugate is 3tf12-DBCO-PEG8-M30m3 (DAR of 1). In some embodiments, the polynucleotide conjugate is Fv55-SMCC-M30m3 (DAR of 1). In some embodiments, the polynucleotide conjugate is Fv55-PEG8-DBCO-M30m3(DAR of 1). In some embodiments, the polynucleotide conjugate is Fv55-PEG8-DBCO-M30m3(DAR of 2). In some embodiments, the polynucleotide conjugate is selected from the antibody-polynucleotide conjugates listed in Table 5, infra. Each antibody-polynucleotide conjugate in Table 5 is considered a separate embodiment. In some embodiments, the polynucleotide conjugate is selected from the antibody-polynucleotide conjugates listed in Table 6, infra. Each antibody-polynucleotide conjugate in Table 6 is considered a separate embodiment.

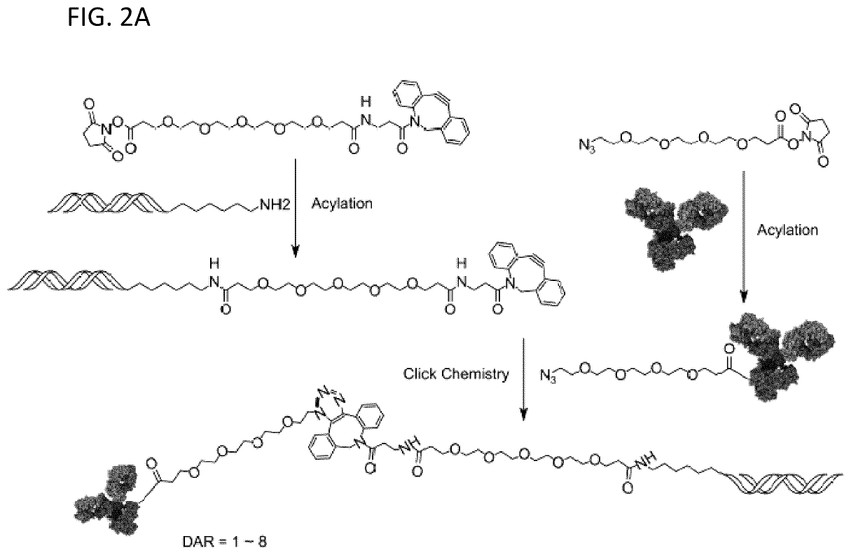

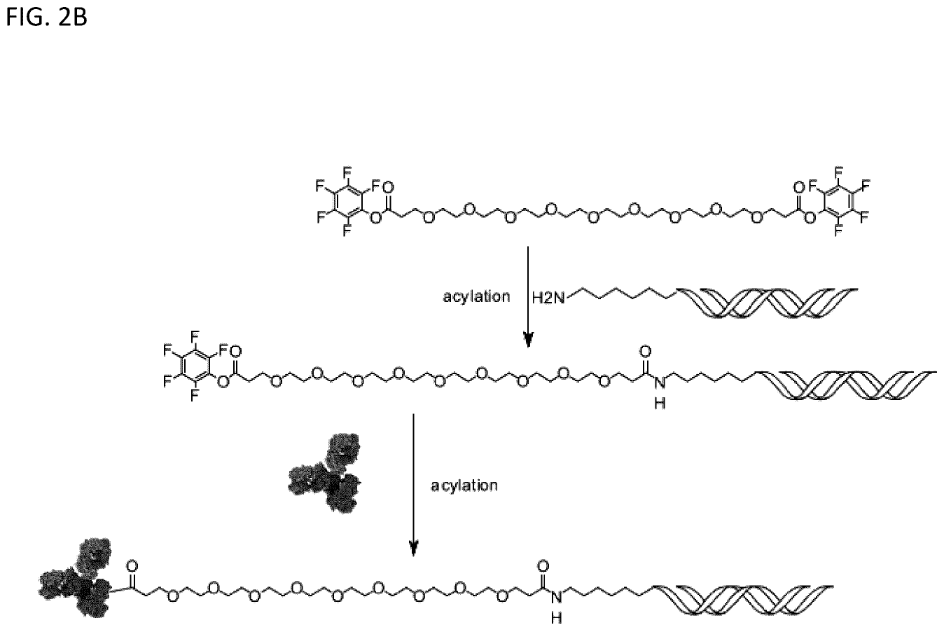

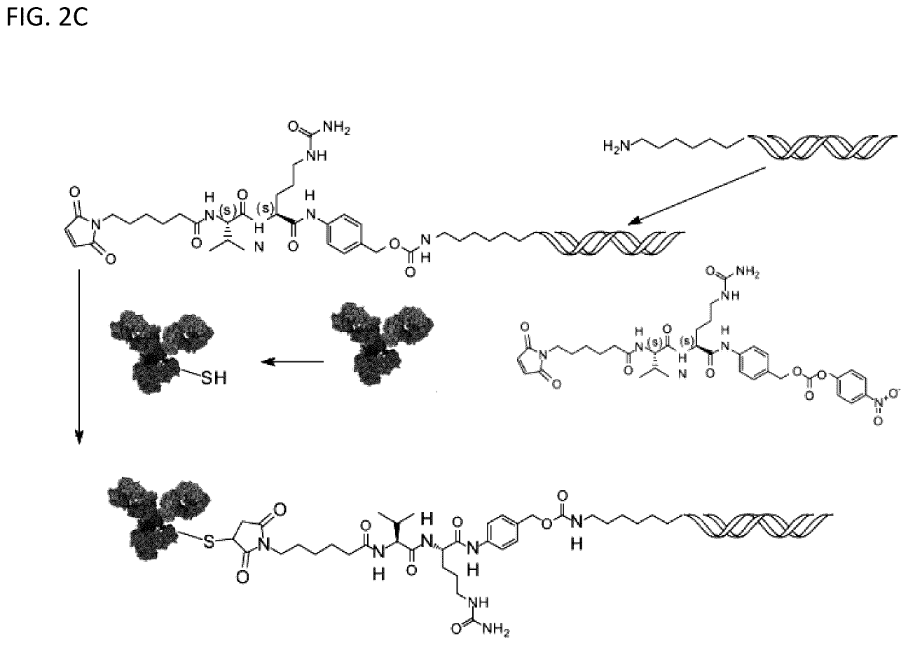

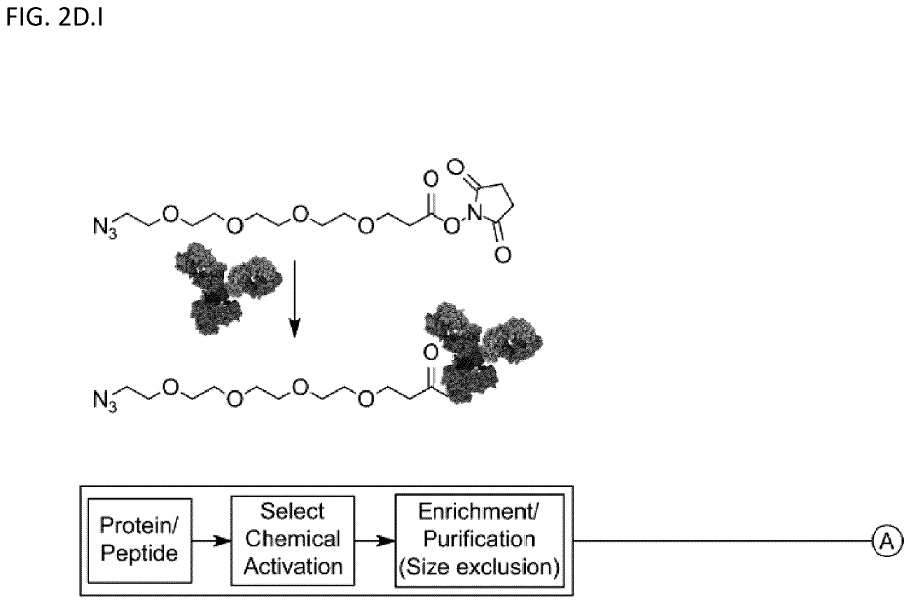

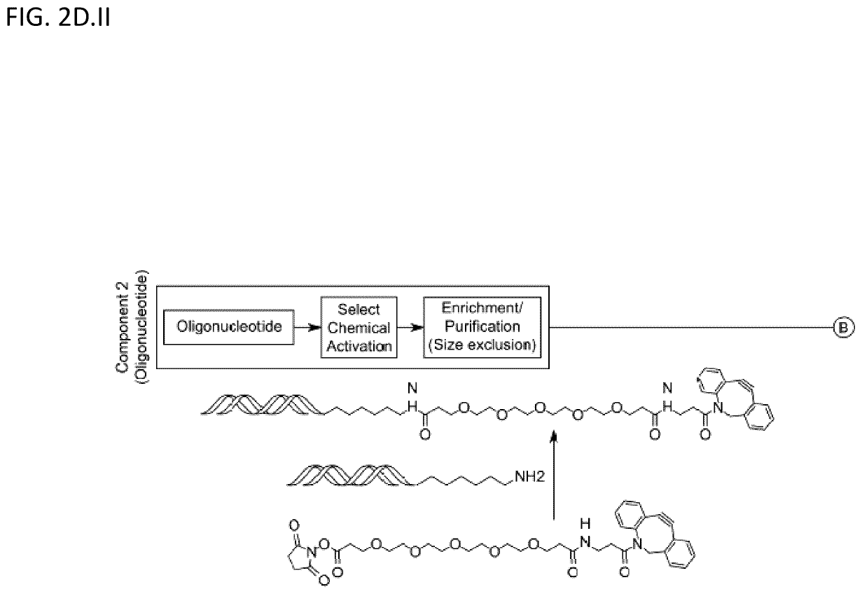

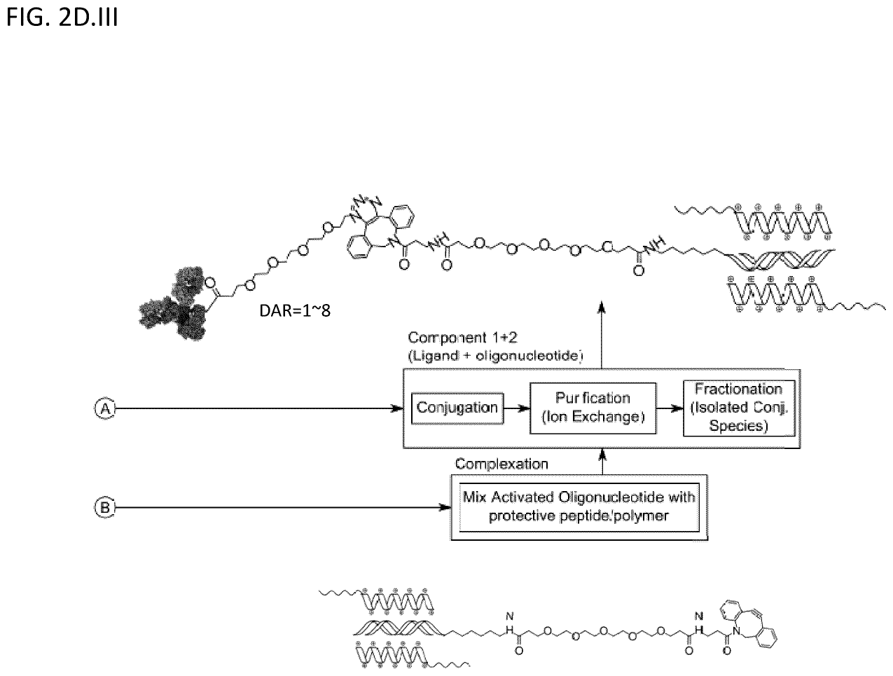

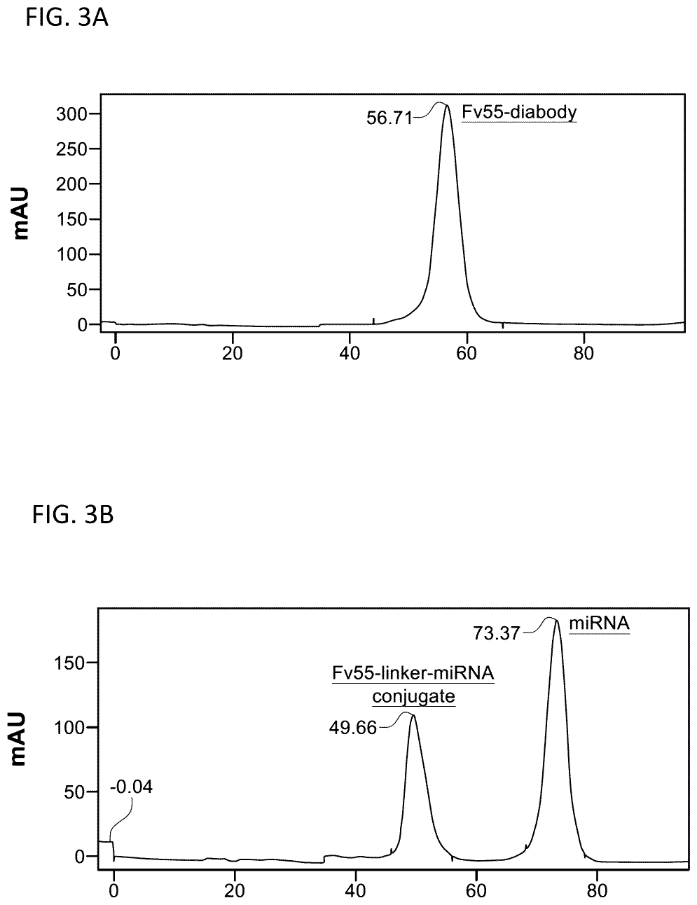

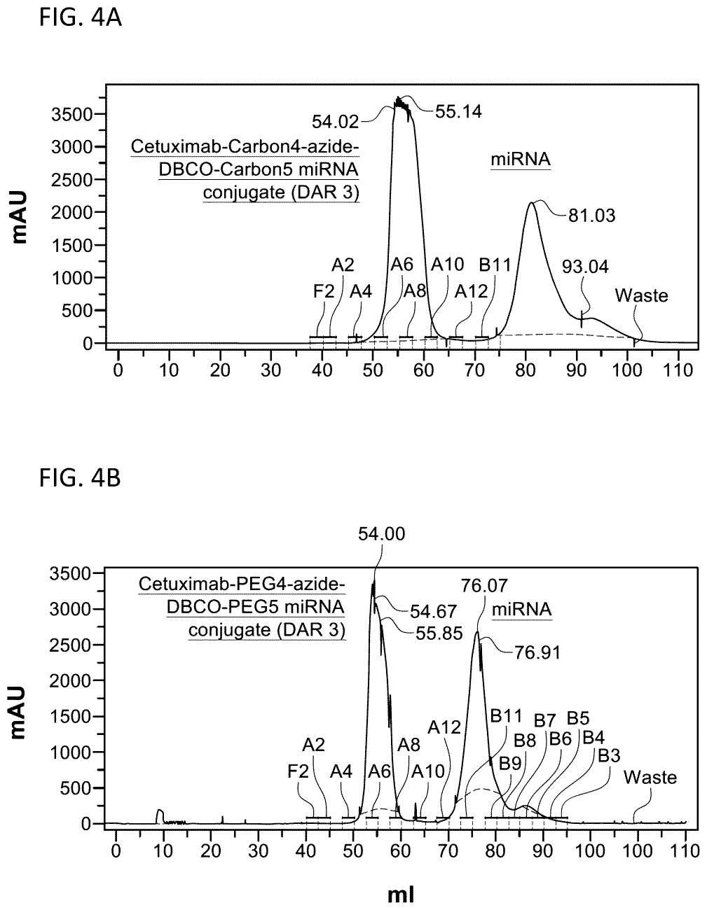

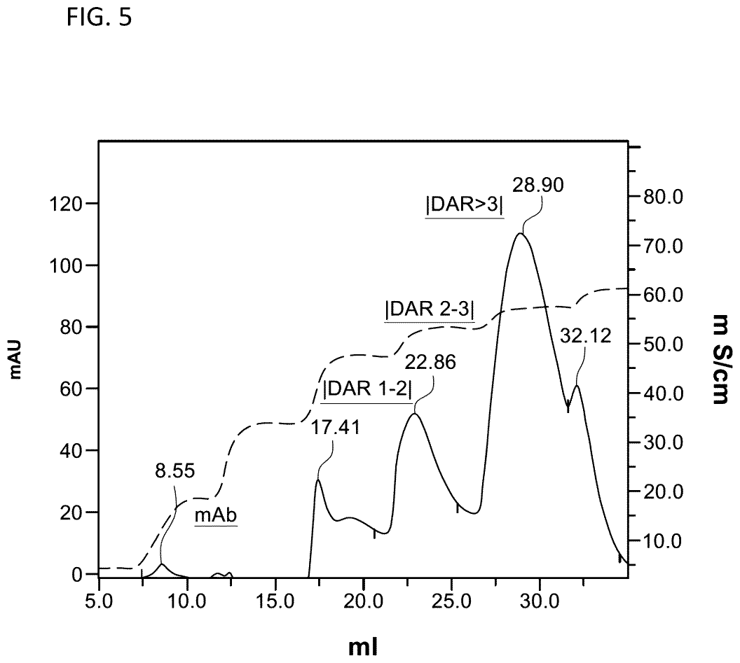

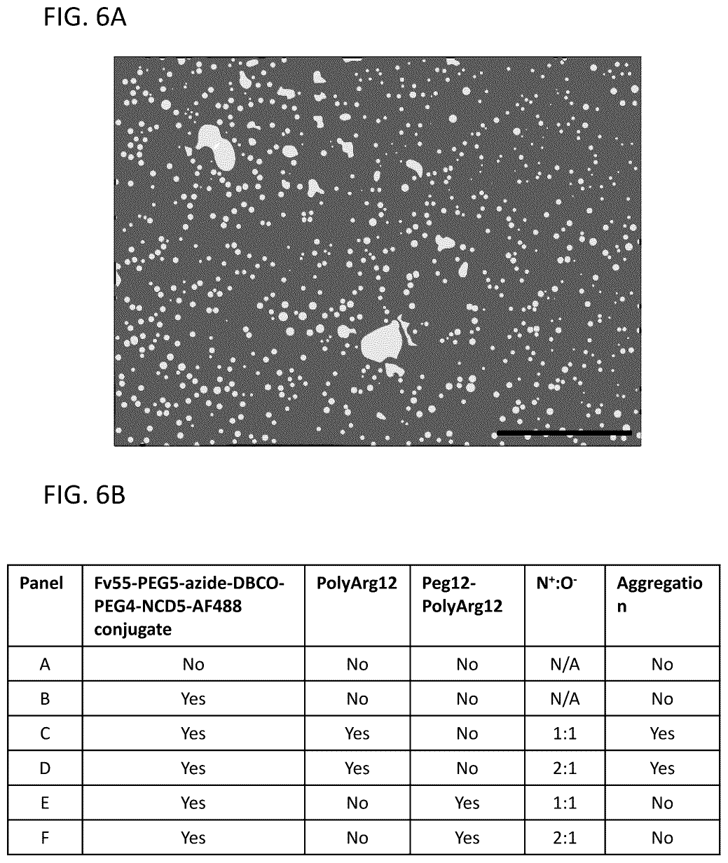

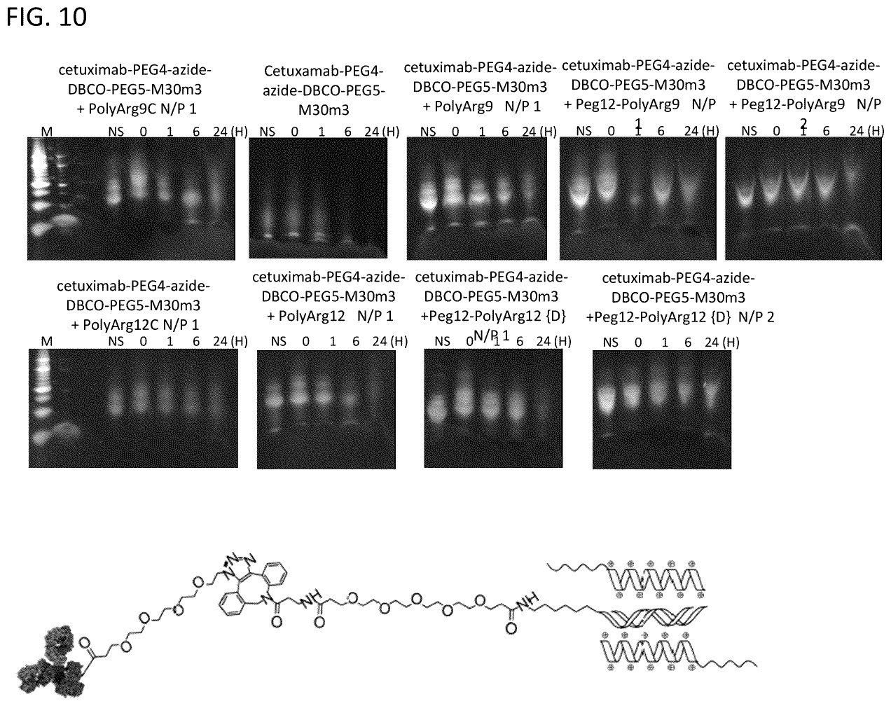

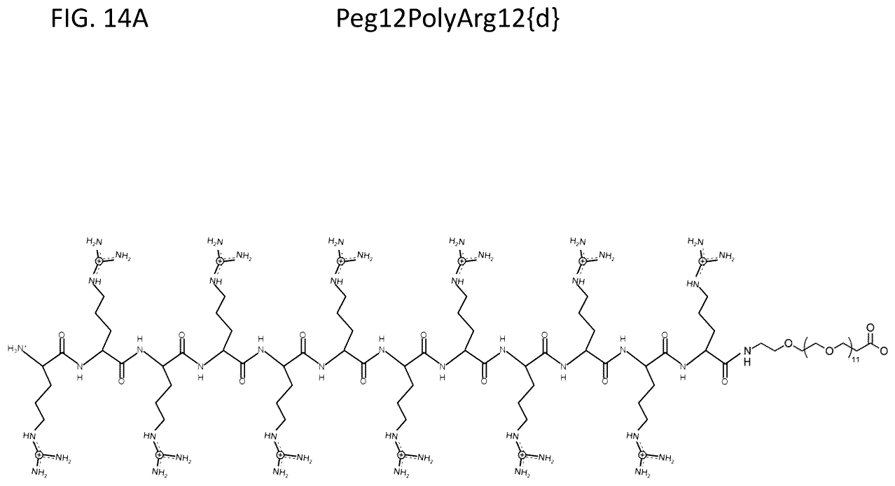

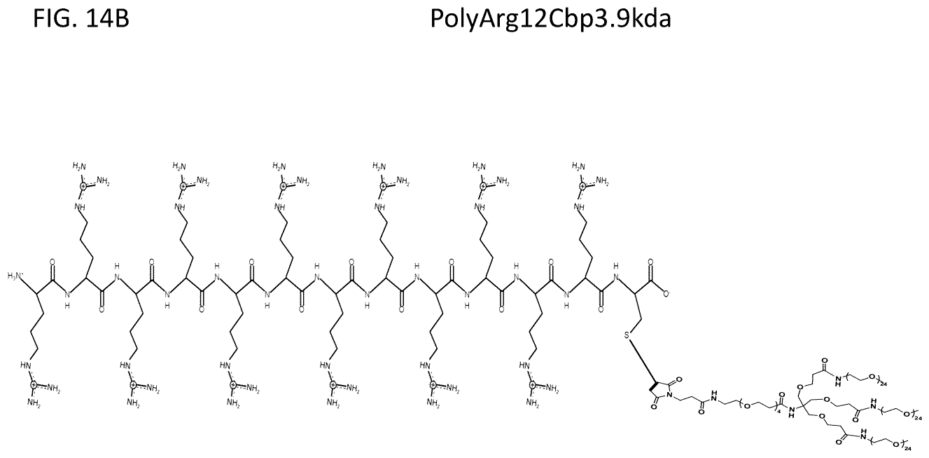

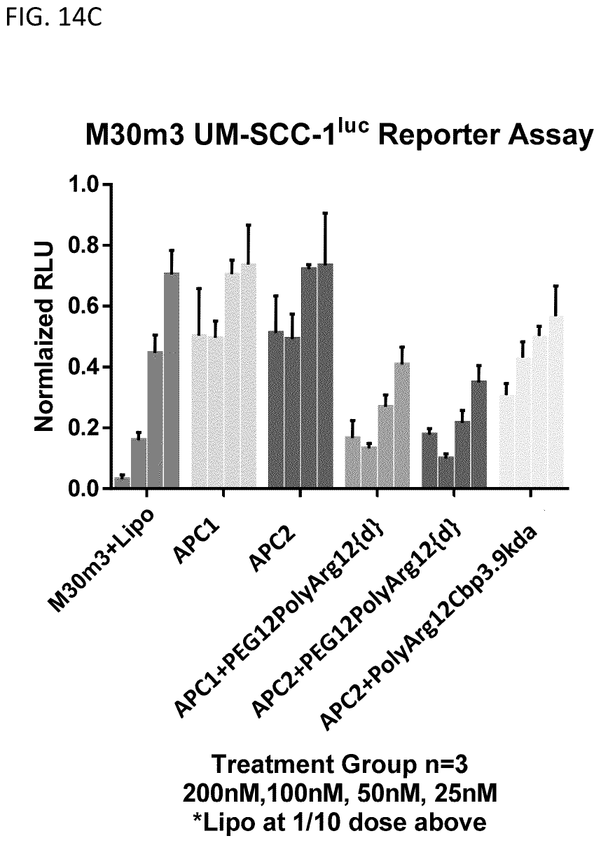

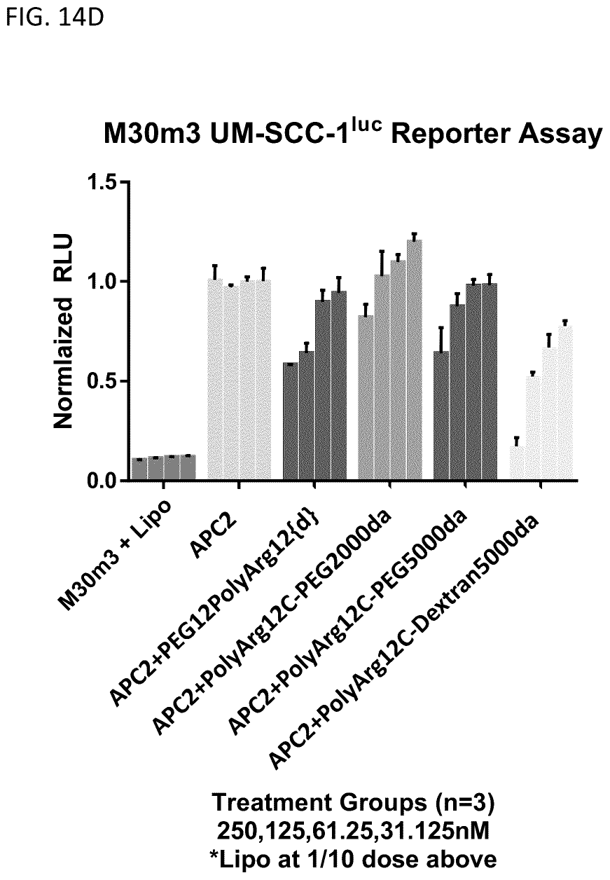

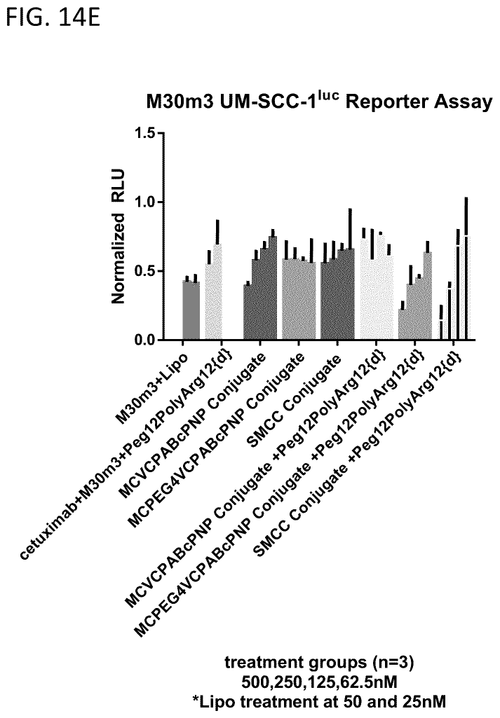

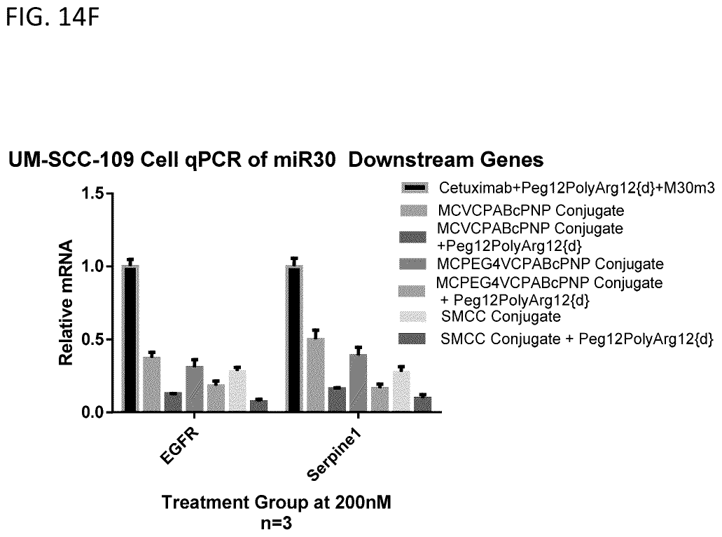

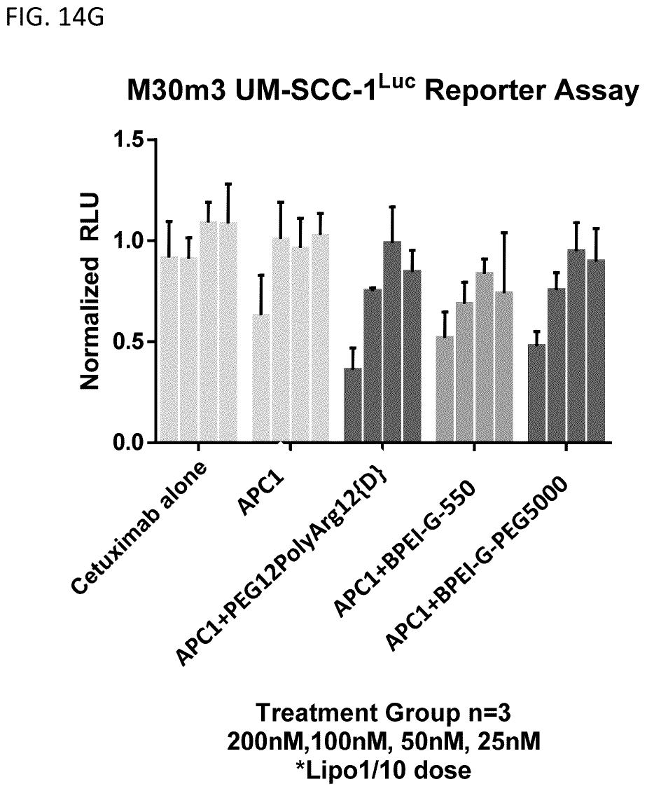

TABLE 5
Antibody-Polynucleotide Conjugates and Associated Hybrid Polymers

| Antibody-Polynucleotide | | |
|---|---|---|
| Conjugate (APC) | APC \| Hybrid Polymer Complex | Note |
| Cetuximab-Carbon4-Azide- | Cetuximab-Carbon4-Azide-DBCO- | FIG. 5B |
| DBCO-Carbon5-M30m3 | Carbon5-M30m3 DAR 1 \| PEG12- | |
| DAR 1 | PolyArg12 {d} | |
| Cetuximab-Carbon4-Azide- | Cetuximab-Carbon4-Azide-DBCO- | FIG. 5B |
| DBCO-Carbon5-M30m3 | Carbon5-M30m3 DAR 2 \| PEG12- | |
| DAR 2 | PolyArg12 {d} | |
| Cetuximab-Carbon4-Azide- | Cetuximab-Carbon4-Azide-DBCO- | FIG. 5B |
| DBCO-Carbon5-M30m3 | Carbon5-M30m3 DAR 3 \| PEG12- | |
| DAR 3 | PolyArg12 {d} | |
| Cetuximab-Carbon4-Azide- | Cetuximab-Carbon4-Azide-DBCO- | FIG. 5B |
| DBCO-Carbon5-M30m3 | Carbon5-M30m3 DAR 4 \| PEG12- | |
| DAR 4 | PolyArg12 {d} | |
| Cetuximab-Carbon4-Azide- | Cetuximab-Carbon4-Azide-DBCO- | FIG. 5B |
| DBCO-Carbon5-M30m3 | Carbon5-M30m3 DAR 5 \| PEG12- | |
| DAR 5 | PolyArg12 {d} | |
| Cetuximab-Carbon4-Azide- | Cetuximab-Carbon4-Azide-DBCO- | FIG. 5B |
| DBCO-Carbon5-M30m3 | Carbon5-M30m3 DAR 6 \| PEG12- | |
| DAR 6 | PolyArg12 {d} | |
| Cetuximab-PEG4-Azide- | Cetuximab-PEG4-Azide-DBCO-Carbon5- | FIG. 10A |
| DBCO-Carbon5-M30m3 | M30m3 DAR 1 \| PEG12-PolyArg12 {d} | |
| DAR 1 | | |
| Cetuximab-PEG4-Azide- | Cetuximab-PEG4-Azide-DBCO-Carbon5- | FIG. 10A |
| DBCO-Carbon5-M30m3 | M30m3 DAR 2 \| PEG12-PolyArg12 {d} | |
| DAR 2 | | |
| Cetuximab-PEG4-Azide- | Cetuximab-PEG4-Azide-DBCO-Carbon5- | FIG. 10A |
| DBCO-Carbon5-M30m3 | M30m3 DAR 3 \| PEG12-PolyArg12 {d} | |
| DAR 3 | | |
| Cetuximab-PEG4-Azide- | Cetuximab-PEG4-Azide-DBCO-Carbon5- | FIG. 10A |
| DBCO-Carbon5-M30m3 | M30m3 DAR 4 \| PEG12-PolyArg12 {d} | |
| DAR 4 | | |
| Cetuximab-PEG4-Azide- | Cetuximab-PEG4-Azide-DBCO-Carbon5- | FIG. 10A |
| DBCO-Carbon5-M30m3 | M30m3 DAR 5 \| PEG12-PolyArg12 {d} | |
| DAR 5 | | |
| Cetuximab-PEG4-Azide- | Cetuximab-PEG4-Azide-DBCO-Carbon5- | FIG. 10A |
| DBCO-Carbon5-M30m3 | M30m3 DAR 6 \| PEG12-PolyArg12 {d} | |
| DAR 6 | | |
| Cetuximab-PEG4-Azide- | Cetuximab-PEG4-Azide-DBCO-Carbon5- | |
| DBCO-Carbon5-M30m1 | M30m1 DAR 1 \| PEG12-PolyArg12 {d} | |
| DAR 1 | | |
| Cetuximab-PEG4-Azide- | Cetuximab-PEG4-Azide-DBCO-Carbon5- | |
| DBCO-Carbon5-M30m1 | M30m1 DAR 2 \| PEG12-PolyArg12 {d} | |
| DAR 2 | | |
| Cetuximab-PEG4-Azide- | Cetuximab-PEG4-Azide-DBCO-Carbon5- | FIG. 3C |
| DBCO-Carbon5-M30m1 | M30m1 DAR 3 \| PEG12-PolyArg12 {d} | |
| DAR 3 | | |
| Cetuximab-PEG4-Azide- | Cetuximab-PEG4-Azide-DBCO-Carbon5- | |
| DBCO-Carbon5-M30m1 | M30m1 DAR 4 \| PEG12-PolyArg12 {d} | |
| DAR 4 | | |
| Cetuximab-PEG4-Azide- | Cetuximab-PEG4-Azide-DBCO-Carbon5- | |
| DBCO-Carbon5-M30m1 | M30m1 DAR 5 \| PEG12-PolyArg12 {d} | |
| DAR 5 | | |
| Cetuximab-PEG4-Azide- | Cetuximab-PEG4-Azide-DBCO-Carbon5- | |
| DBCO-Carbon5-M30m1 | M30m1 DAR 6 \| PEG12-PolyArg12 {d} | |
| DAR 6 | | |
| Cetuximab-PEG4-Azide- | Cetuximab-PEG4-Azide-DBCO-PEG5- | FIG. 2A, 2D, |
| DBCO-PEG5-M30m3 DAR 1 | M30m3 DAR 1 \| PEG12-PolyArg12 {d} | 4C, 5B, 6B, 7F, |
| | | 7G |
| Cetuximab-PEG4-Azide- | Cetuximab-PEG4-Azide-DBCO-PEG5- | FIG. 2A, 2D, |
| DBCO-PEG5-M30m3 DAR 2 | M30m3 DAR 2 \| PEG12-PolyArg12 {d} | 5B, 6B, 7F, 7G |
| Cetuximab-PEG4-Azide- | Cetuximab-PEG4-Azide-DBCO-PEG5- | FIG. 2A, 2D, |
| DBCO-PEG5-M30m3 DAR 3 | M30m3 DAR 3 \| PEG12-PolyArg12 {d} | 3B, 5B, 6B, 10B, |
| | | 12 |
| Cetuximab-PEG4-Azide- | Cetuximab-PEG4-Azide-DBCO-PEG5- | FIG. 2A, 2D, |
| DBCO-PEG5-M30m3 DAR 4 | M30m3 DAR 4 \| PEG12-PolyArg12 {d} | 5B, 6B |
| Cetuximab-PEG4-Azide- | Cetuximab-PEG4-Azide-DBCO-PEG5- | FIG. 2A, 2D, |
| DBCO-PEG5-M30m3 DAR 5 | M30m3 DAR 5 \| PEG12-PolyArg12 {d} | 5B, 6B |
| Cetuximab-PEG4-Azide- | Cetuximab-PEG4-Azide-DBCO-PEG5- | FIG. 2A, 2D, |
| DBCO-PEG5-M30m3 DAR 6 | M30m3 DAR 6 \| PEG12-PolyArg12 {d} | 5B, 6B |
| Cetuximab-PEG(n)-M30m3 | Cetuximab-PEG(n)-M30m3 (n = 1~100) | FIG. 2B |
| (n = 1~100) DAR 1 | DAR 1 \| PEG12-PolyArg12 {d} | |
| Cetuximab-PEG(n)-M30m3 | Cetuximab-PEG(n)-M30m3 (n = 1~100) | FIG. 2B |
| (n = 1~100) DAR 2 | DAR 2 \| PEG12-PolyArg12 {d} | |
| Cetuximab-PEG(n)-M30m3 | Cetuximab-PEG(n)-M30m3 (n = 1~100) | FIG. 2B |
| (n = 1~100) DAR 3 | DAR 3 \| PEG12-PolyArg12 {d} | |
| Cetuximab-PEG(n)-M30m3 | Cetuximab-PEG(n)-M30m3 (n = 1~100) | FIG. 2B |
| (n = 1~100) DAR 4 | DAR 4 \| PEG12-PolyArg12 {d} | |
| Cetuximab-PEG(n)-M30m3 | Cetuximab-PEG(n)-M30m3 (n = 1~100) | FIG. 2B |
| (n = 1~100) DAR 5 | DAR 5 \| PEG12-PolyArg12 {d} | |
| Cetuximab-PEG(n)-M30m3 | Cetuximab-PEG(n)-M30m3 (n = 1~100) | FIG. 2B |
| (n = 1~100) DAR 6 | DAR 6 \| PEG12-PolyArg12 {d} | |
| 3tf12-DBCO-PEG8-NCD5 | 3tf12-DBCO-PEG8-NCD5 \| PolyArg9 | |
| 3tf12-DBCO-PEG8-M30m3 | 3tf12-DBCO-PEG8-M30m3 \| PolyArg9 | Table 7 |
| Fv55-SMCC-M30m3 | Fv55-SMCC-M30m3 \| PEG12-PolyArg12 | Table 7 |
| Fv55-PEG8-DBCO-M30m3 | Fv55-PEG8-M30m3 \| PEG12-PolyArg12 | FIG. 6C |
| Fv55-PEG8-DBCO-M30m1 | Fv55-PEG8-M30m1 \| PEG12-PolyArg12 | FIG. 3A, 5A |
| Fv55-PEG8-DBCO-NCD5 | Fv55-PEG8-NCD5 \| PEG12-PolyArg12 | FIG. 4A |
| Cetuximab-MC-ValCit- | Cetuximab-MC-ValCit-PABc-M30m3 | FIG. 14E, 14F, |
| PABc-M30m3 DAR 4 | DAR 4\| PEG12-PolyArg12 | 21C |
| Cetuximab-MC-PEG4- | Cetuximab-MC-PEG4-ValCit-PABc- | FIG. 14E, 14F, |
| ValCit-PABc-M30m3 DAR 4 | M30m3 DAR 4\| PEG12-PolyArg12 | 21C |
| Cetuximab-SMCC-M30m3 | Cetuximab-SMCC-M30m3 DAR 4 \| | FIG. 14E, 14F, |
| DAR 4 | PEG12-PolyArg12 | 21C |
| Cetuximab-PEG4-azide- | Cetuximab-PEG4-azide-DBCO-PEG4- | FIG. 14C |
| DBCO-PEG4-m30m3 DAR4 | m30m3 DAR4 | |
| | \|PolyArg12Cbp3.9kda | |
| Cetuximab-PEG4-azide- | Cetuximab-PEG4-azide-DBCO-PEG4- | FIG. 14D |
| DBCO-PEG4-m30m3 DAR4 | m30m3 DAR4 | |
| | \|PolyArg12C-PEG2000da | |
| Cetuximab-PEG4-azide- | Cetuximab-PEG4-azide-DBCO-PEG4- | FIG. 14D |
| DBCO-PEG4-m30m3 DAR4 | m30m3 DAR4 | |
| | \|PolyArg12C-PEG5000da | |
| Cetuximab-PEG4-azide- | Cetuximab-PEG4-azide-DBCO-PEG4- | FIG. 14D |
| DBCO-PEG4-m30m3 DAR4 | m30m3 DAR4 | |
| | \|PolyArg12C-Dextran5000da | |

TABLE 6
Antibody-Polynucleotide Conjugates.
Antibody Polynucleotide Conjugate

3TF12 Diabody-Bis-Mal-Lysine-PEG4-TFP ester-M30m1 DAR 1
3TF12 Diabody-Bis-Mal-Lysine-PEG4-TFP ester-M30m1 DAR 2
3TF12 Diabody-Bis-Mal-Lysine-PEG4-TFP ester-M30m3 DAR 1
3TF12 Diabody-Bis-Mal-Lysine-PEG4-TFP ester-M30m3 DAR 2
3TF12 Diabody-Carbon4-Azide-DBCO-Carbon5-ASDX2 DAR 1
3TF12 Diabody-Carbon4-Azide-DBCO-Carbon5-ASDX2 DAR 2
3TF12 Diabody-Carbon4-Azide-DBCO-Carbon5-ASDX23 DAR 1
3TF12 Diabody-Carbon4-Azide-DBCO-Carbon5-ASDX23 DAR 2
3TF12 Diabody-Carbon4-Azide-DBCO-Carbon5-ASDX26 DAR 1
3TF12 Diabody-Carbon4-Azide-DBCO-Carbon5-ASDX26 DAR 2
3TF12 Diabody-Carbon4-Azide-DBCO-Carbon5-ASDX32 DAR 1
3TF12 Diabody-Carbon4-Azide-DBCO-Carbon5-ASDX32 DAR 2
3TF12 Diabody-Carbon4-Azide-DBCO-Carbon5-ASDX4 DAR 1
3TF12 Diabody-Carbon4-Azide-DBCO-Carbon5-ASDX4 DAR 2
3TF12 Diabody-Carbon4-Azide-DBCO-Carbon5-M30m1 DAR 1
3TF12 Diabody-Carbon4-Azide-DBCO-Carbon5-M30m1 DAR 2
3TF12 Diabody-Carbon4-Azide-DBCO-Carbon5-M30m3 DAR 1
3TF12 Diabody-Carbon4-Azide-DBCO-Carbon5-M30m3 DAR 2
3TF12 Diabody-Carbon4-Azide-DBCO-Carbon5-NCD5-647 DAR 1
3TF12 Diabody-Carbon4-Azide-DBCO-Carbon5-NCD5-647 DAR 2
3TF12 Diabody-KMUS-M30m1 DAR 1
3TF12 Diabody-KMUS-M30m1 DAR 2
3TF12 Diabody-KMUS-M30m3 DAR 1
3TF12 Diabody-KMUS-M30m3 DAR 2
3TF12 Diabody-KMUS-NCD5-647 DAR 1
3TF12 Diabody-KMUS-NCD5-647 DAR 2
3TF12 Diabody-PEG4-Azide-DBCO-Carbon5-ASDX2 DAR 1
3TF12 Diabody-PEG4-Azide-DBCO-Carbon5-ASDX2 DAR 2
3TF12 Diabody-PEG4-Azide-DBCO-Carbon5-ASDX23 DAR 1
3TF12 Diabody-PEG4-Azide-DBCO-Carbon5-ASDX23 DAR 2
3TF12 Diabody-PEG4-Azide-DBCO-Carbon5-ASDX26 DAR 1
3TF12 Diabody-PEG4-Azide-DBCO-Carbon5-ASDX26 DAR 2
3TF12 Diabody-PEG4-Azide-DBCO-Carbon5-ASDX32 DAR 1
3TF12 Diabody-PEG4-Azide-DBCO-Carbon5-ASDX32 DAR 2
3TF12 Diabody-PEG4-Azide-DBCO-Carbon5-ASDX4 DAR 1
3TF12 Diabody-PEG4-Azide-DBCO-Carbon5-ASDX4 DAR 2
3TF12 Diabody-PEG4-Azide-DBCO-Carbon5-M30m1 DAR 1
3TF12 Diabody-PEG4-Azide-DBCO-Carbon5-M30m1 DAR 2
3TF12 Diabody-PEG4-Azide-DBCO-Carbon5-M30m3 DAR 1
3TF12 Diabody-PEG4-Azide-DBCO-Carbon5-M30m3 DAR 2
3TF12 Diabody-PEG4-Azide-DBCO-Carbon5-NCD5-647 DAR 1
3TF12 Diabody-PEG4-Azide-DBCO-Carbon5-NCD5-647 DAR 2
3TF12 Diabody-PEG4-Azide-DBCO-PEG5-ASDX2 DAR 1
3TF12 Diabody-PEG4-Azide-DBCO-PEG5-ASDX2 DAR 2
3TF12 Diabody-PEG4-Azide-DBCO-PEG5-ASDX23 DAR 1
3TF12 Diabody-PEG4-Azide-DBCO-PEG5-ASDX23 DAR 2
3TF12 Diabody-PEG4-Azide-DBCO-PEG5-ASDX26 DAR 1
3TF12 Diabody-PEG4-Azide-DBCO-PEG5-ASDX26 DAR 2
3TF12 Diabody-PEG4-Azide-DBCO-PEG5-ASDX32 DAR 1
3TF12 Diabody-PEG4-Azide-DBCO-PEG5-ASDX32 DAR 2
3TF12 Diabody-PEG4-Azide-DBCO-PEG5-ASDX4 DAR 1
3TF12 Diabody-PEG4-Azide-DBCO-PEG5-ASDX4 DAR 2
3TF12 Diabody-PEG4-Azide-DBCO-PEG5-M30m1 DAR 1
3TF12 Diabody-PEG4-Azide-DBCO-PEG5-M30m1 DAR 2
3TF12 Diabody-PEG4-Azide-DBCO-PEG5-M30m3 DAR 1
3TF12 Diabody-PEG4-Azide-DBCO-PEG5-M30m3 DAR 2
3TF12 Diabody-PEG4-Azide-DBCO-PEG5-NCD5-647 DAR 1
3TF12 Diabody-PEG4-Azide-DBCO-PEG5-NCD5-647 DAR 2
3TF12-Bis-Mal-Lysine-PEG4-TFP ester-M30m1 DAR 1
3TF12-Bis-Mal-Lysine-PEG4-TFP ester-M30m3 DAR 1
3TF12-Carbon4-Azide-DBCO-Carbon5-ASDX2 DAR 1
3TF12-Carbon4-Azide-DBCO-Carbon5-ASDX23 DAR 1
3TF12-Carbon4-Azide-DBCO-Carbon5-ASDX26 DAR 1
3TF12-Carbon4-Azide-DBCO-Carbon5-ASDX32 DAR 1
3TF12-Carbon4-Azide-DBCO-Carbon5-ASDX4 DAR 1
3TF12-Carbon4-Azide-DBCO-Carbon5-M30m1 DAR 1
3TF12-Carbon4-Azide-DBCO-Carbon5-M30m4 DAR 0.5
3TF12-Carbon4-Azide-DBCO-Carbon5-M30m3 DAR 1
3TF12-Carbon4-Azide-DBCO-Carbon5-NCD5-647 DAR 1
3TF12-KMUS-M30m1 DAR 1
3TF12-KMUS-M30m3 DAR 1
3TF12-KMUS-NCD5-647 DAR 1
3TF12-PEG4-Azide-DBCO-Carbon5-ASDX2 DAR 1
3TF12-PEG4-Azide-DBCO-Carbon5-ASDX23 DAR 1
3TF12-PEG4-Azide-DBCO-Carbon5-ASDX26 DAR 1
3TF12-PEG4-Azide-DBCO-Carbon5-ASDX32 DAR 1
3TF12-PEG4-Azide-DBCO-Carbon5-ASDX4 DAR 1
3TF12-PEG4-Azide-DBCO-Carbon5-M30m1 DAR 1
3TF12-PEG4-Azide-DBCO-Carbon5-M30m4 DAR 0.5
3TF12-PEG4-Azide-DBCO-Carbon5-M30m3 DAR 1
3TF12-PEG4-Azide-DBCO-Carbon5-NCD5-647 DAR 1
3TF12-PEG4-Azide-DBCO-PEG5-ASDX2 DAR 1
3TF12-PEG4-Azide-DBCO-PEG5-ASDX23 DAR 1
3TF12-PEG4-Azide-DBCO-PEG5-ASDX26 DAR 1
3TF12-PEG4-Azide-DBCO-PEG5-ASDX32 DAR 1
3TF12-PEG4-Azide-DBCO-PEG5-ASDX4 DAR 1
3TF12-PEG4-Azide-DBCO-PEG5-M30m1 DAR 1
3TF12-PEG4-Azide-DBCO-PEG5-M30m4 DAR 0.5
3TF12-PEG4-Azide-DBCO-PEG5-M30m3 DAR 1
3TF12-PEG4-Azide-DBCO-PEG5-NCD5-647 DAR 1
Cetuximab-Bis-Mal-Lysine-PEG4-TFP ester-M30m1 DAR 1
Cetuximab-Bis-Mal-Lysine-PEG4-TFP ester-M30m1 DAR 2
Cetuximab-Bis-Mal-Lysine-PEG4-TFP ester-M30m1 DAR 3
Cetuximab-Bis-Mal-Lysine-PEG4-TFP ester-M30m1 DAR 4
Cetuximab-Bis-PFP Ester Peg 13-M30m3 DAR 1
Cetuximab-Bis-PFP Ester Peg 13-M30m3 DAR 2
Cetuximab-Bis-PFP Ester Peg 13-M30m3 DAR 3
Cetuximab-Bis-PFP Ester Peg 13-M30m3 DAR 4
Cetuximab-Bis-TFP Ester Peg 13-M30m3 DAR 1
Cetuximab-Bis-TFP Ester Peg 13-M30m3 DAR 2
Cetuximab-Bis-TFP Ester Peg 13-M30m3 DAR 3
Cetuximab-Bis-TFP Ester Peg 13-M30m3 DAR 4
Cetuximab-Carbon4-Azide-DBCO-Carbon5-M30m1 DAR 1
Cetuximab-Carbon4-Azide-DBCO-Carbon5-M30m1 DAR 2
Cetuximab-Carbon4-Azide-DBCO-Carbon5-M30m1 DAR 3
Cetuximab-Carbon4-Azide-DBCO-Carbon5-M30m1 DAR 4
Cetuximab-Carbon4-Azide-DBCO-Carbon5-M30m1 DAR 5
Cetuximab-Carbon4-Azide-DBCO-Carbon5-M30m1 DAR 6
Cetuximab-Carbon4-Azide-DBCO-Carbon5-M30m3 DAR 1
Cetuximab-Carbon4-Azide-DBCO-Carbon5-M30m3 DAR 2
Cetuximab-Carbon4-Azide-DBCO-Carbon5-M30m3 DAR 3
Cetuximab-Carbon4-Azide-DBCO-Carbon5-M30m3 DAR 4
Cetuximab-Carbon4-Azide-DBCO-Carbon5-M30m3 DAR 5
Cetuximab-Carbon4-Azide-DBCO-Carbon5-M30m3 DAR 6
Cetuximab-Carbon4-Azide-DBCO-Carbon5-NCD5-647 DAR 1
Cetuximab-Carbon4-Azide-DBCO-Carbon5-NCD5-647 DAR 2
Cetuximab-Carbon4-Azide-DBCO-Carbon5-NCD5-647 DAR 3
Cetuximab-Carbon4-Azide-DBCO-Carbon5-NCD5-647 DAR 4
Cetuximab-Carbon4-Azide-DBCO-Carbon5-NCD5-647 DAR 5
Cetuximab-Carbon4-Azide-DBCO-Carbon5-NCD5-647 DAR 6
Cetuximab-KMUS-M30m1 DAR 1
Cetuximab-KMUS-M30m1 DAR 2
Cetuximab-KMUS-M30m1 DAR 3
Cetuximab-KMUS-M30m1 DAR 4
Cetuximab-KMUS-M30m3 DAR 1
Cetuximab-KMUS-M30m3 DAR 2
Cetuximab-KMUS-M30m3 DAR 3
Cetuximab-KMUS-M30m3 DAR 4
Cetuximab-KMUS-M30m3 DAR 5
Cetuximab-KMUS-M30m3 DAR 6
Cetuximab-KMUS-NCD5-647 DAR 1
Cetuximab-KMUS-NCD5-647 DAR 2
Cetuximab-KMUS-NCD5-647 DAR 3
Cetuximab-KMUS-NCD5-647 DAR 4
Cetuximab-MC-ValCit-PABc-M30m3 DAR 1
Cetuximab-MC-ValCit-PABc-M30m3 DAR 2
Cetuximab-MC-ValCit-PABc-M30m3 DAR 3
Cetuximab-MC-ValCit-PABc-M30m3 DAR 4
Cetuximab-Azide-DBCO-PEG5-M30m3 DAR 1
Cetuximab-Azide-DBCO-PEG5-M30m3 DAR 2
Cetuximab-Azide-DBCO-PEG5-M30m3 DAR 3
Cetuximab-Azide-DBCO-PEG5-M30m3 DAR 4
Cetuximab-Azide-DBCO-PEG5-M30m3 DAR 5
Cetuximab-Azide-DBCO-PEG5-M30m3 DAR 6
Cetuximab-PEG0.5k-Azide-DBCO-PEG1k-M30m3 DAR 1
Cetuximab-PEG0.5k-Azide-DBCO-PEG1k-M30m3 DAR 2
Cetuximab-PEG1.5k-Azide-DBCO-PEG1.5k-M30m3 DAR 1
Cetuximab-PEG1.5k-Azide-DBCO-PEG1.5k-M30m3 DAR 2
Cetuximab-PEG1k-Azide-DBCO-PEG1k-M30m3 DAR 1
Cetuximab-PEG1k-Azide-DBCO-PEG1k-M30m3 DAR 2
Cetuximab-PEG2k-Azide-DBCO-PEG2k-M30m3 DAR 1
Cetuximab-PEG2k-Azide-DBCO-PEG2k-M30m3 DAR 1
Cetuximab-PEG2k-Azide-DBCO-PEG2k-M30m3 DAR 2
Cetuximab-PEG2k-Azide-DBCO-PEG2k-M30m3 DAR 2
Cetuximab-PEG3k-Azide-DBCO-PEG3k-M30m3 DAR 1
Cetuximab-PEG3k-Azide-DBCO-PEG3k-M30m3 DAR 2
Cetuximab-PEG4-Azide-DBCO-Carbon5-M30m1 DAR 1
Cetuximab-PEG4-Azide-DBCO-Carbon5-M30m1 DAR 2
Cetuximab-PEG4-Azide-DBCO-Carbon5-M30m1 DAR 3
Cetuximab-PEG4-Azide-DBCO-Carbon5-M30m1 DAR 4
Cetuximab-PEG4-Azide-DBCO-Carbon5-M30m1 DAR 5
Cetuximab-PEG4-Azide-DBCO-Carbon5-M30m1 DAR 6
Cetuximab-PEG4-Azide-DBCO-Carbon5-M30m3 DAR 1
Cetuximab-PEG4-Azide-DBCO-Carbon5-M30m3 DAR 2
Cetuximab-PEG4-Azide-DBCO-Carbon5-M30m3 DAR 3
Cetuximab-PEG4-Azide-DBCO-Carbon5-M30m3 DAR 4
Cetuximab-PEG4-Azide-DBCO-Carbon5-M30m3 DAR 5
Cetuximab-PEG4-Azide-DBCO-Carbon5-M30m3 DAR 6
Cetuximab-PEG4-Azide-DBCO-Carbon5-NCD5-647 DAR 1
Cetuximab-PEG4-Azide-DBCO-Carbon5-NCD5-647 DAR 2
Cetuximab-PEG4-Azide-DBCO-Carbon5-NCD5-647 DAR 3
Cetuximab-PEG4-Azide-DBCO-Carbon5-NCD5-647 DAR 4
Cetuximab-PEG4-Azide-DBCO-Carbon5-NCD5-647 DAR 5
Cetuximab-PEG4-Azide-DBCO-Carbon5-NCD5-647 DAR 6
Cetuximab-PEG4-Azide-DBCO-PEG5-ASDX32DAR 1
Cetuximab-PEG4-Azide-DBCO-PEG5-ASDX32DAR 2
Cetuximab-PEG4-Azide-DBCO-PEG5-ASDX32DAR 3
Cetuximab-PEG4-Azide-DBCO-PEG5-ASDX32DAR 4
Cetuximab-PEG4-Azide-DBCO-PEG5-ASDX32 DAR 5
Cetuximab-PEG4-Azide-DBCO-PEG5-ASDX32 DAR 6
Cetuximab-PEG4-Azide-DBCO-PEG5-ASDX32 DAR 7
Cetuximab-PEG4-Azide-DBCO-PEG5-ASDX32 DAR 8
Cetuximab-PEG4-Azide-DBCO-PEG5-M30m1 DAR 1
Cetuximab-PEG4-Azide-DBCO-PEG5-M30m1 DAR 2
Cetuximab-PEG4-Azide-DBCO-PEG5-M30m1 DAR 3
Cetuximab-PEG4-Azide-DBCO-PEG5-M30m1 DAR 4
Cetuximab-PEG4-Azide-DBCO-PEG5-M30m1 DAR 5
Cetuximab-PEG4-Azide-DBCO-PEG5-M30m1 DAR 6
Cetuximab-PEG4-Azide-DBCO-PEG5-M30m3 DAR 1
Cetuximab-PEG4-Azide-DBCO-PEG5-M30m3 DAR 2
Cetuximab-PEG4-Azide-DBCO-PEG5-M30m3 DAR 3
Cetuximab-PEG4-Azide-DBCO-PEG5-M30m3 DAR 4
Cetuximab-PEG4-Azide-DBCO-PEG5-M30m3 DAR 5
Cetuximab-PEG4-Azide-DBCO-PEG5-M30m3 DAR 6
Cetuximab-PEG4-Azide-DBCO-PEG5-NCD5-647 DAR 1
Cetuximab-PEG4-Azide-DBCO-PEG5-NCD5-647 DAR 2
Cetuximab-PEG4-Azide-DBCO-PEG5-NCD5-647 DAR 3
Cetuximab-PEG4-Azide-DBCO-PEG5-NCD5-647 DAR 4
Cetuximab-PEG4-Azide-DBCO-PEG5-NCD5-647 DAR 5
Cetuximab-PEG4-Azide-DBCO-PEG5-NCD5-647 DAR 6
Cetuximab-PEG5k-Azide-DBCO-PEG5k-M30m3 DAR 1
Cetuximab-PEG5k-Azide-DBCO-PEG5k-M30m3 DAR 1
Cetuximab-PEG5k-Azide-DBCO-PEG5k-M30m3 DAR 2
Cetuximab-PEG5k-Azide-DBCO-PEG5k-M30m3 DAR 2
Cetuximab-SMCC-M30m3 DAR 1
Cetuximab-SMCC-M30m3 DAR 2
Cetuximab-SMCC-M30m3 DAR 3
Cetuximab-SMCC-M30m3 DAR 4
Cetuximab-Sulfo-SMCC-M30m3 DAR 1
Cetuximab-Sulfo-SMCC-M30m3 DAR 2
Cetuximab-Sulfo-SMCC-M30m3 DAR 3
Cetuximab-Sulfo-SMCC-M30m3 DAR 4
FV55 Diabody-Bis-Mal-Lysine-PEG4-TFP ester-M30m1 DAR 1
FV55 Diabody-Bis-Mal-Lysine-PEG4-TFP ester-M30m1 DAR 2
FV55 Diabody-Bis-Mal-Lysine-PEG4-TFP ester-M30m3 DAR 1
FV55 Diabody-Bis-Mal-Lysine-PEG4-TFP ester-M30m3 DAR 2
FV55 Diabody-Carbon4-Azide-DBCO-Carbon5-ASDX2 DAR 1
FV55 Diabody-Carbon4-Azide-DBCO-Carbon5-ASDX2 DAR 2
FV55 Diabody-Carbon4-Azide-DBCO-Carbon5-ASDX23 DAR 1
FV55 Diabody-Carbon4-Azide-DBCO-Carbon5-ASDX23 DAR 2
FV55 Diabody-Carbon4-Azide-DBCO-Carbon5-ASDX26 DAR 1
FV55 Diabody-Carbon4-Azide-DBCO-Carbon5-ASDX26 DAR 2
FV55 Diabody-Carbon4-Azide-DBCO-Carbon5-ASDX32 DAR 1
FV55 Diabody-Carbon4-Azide-DBCO-Carbon5-ASDX32 DAR 2
FV55 Diabody-Carbon4-Azide-DBCO-Carbon5-ASDX4 DAR 1
FV55 Diabody-Carbon4-Azide-DBCO-Carbon5-ASDX4 DAR 2
FV55 Diabody-Carbon4-Azide-DBCO-Carbon5-M30m1 DAR 1
FV55 Diabody-Carbon4-Azide-DBCO-Carbon5-M30m1 DAR 2
FV55 Diabody-Carbon4-Azide-DBCO-Carbon5-M30m3 DAR 1
FV55 Diabody-Carbon4-Azide-DBCO-Carbon5-M30m3 DAR 2
FV55 Diabody-Carbon4-Azide-DBCO-Carbon5-NCD5-647 DAR 1
FV55 Diabody-Carbon4-Azide-DBCO-Carbon5-NCD5-647 DAR 2
FV55 Diabody-KMUS-M30m1 DAR 1
FV55 Diabody-KMUS-M30m1 DAR 2
FV55 Diabody-KMUS-M30m3 DAR 1
FV55 Diabody-KMUS-M30m3 DAR 2
FV55 Diabody-KMUS-NCD5-647 DAR 1
FV55 Diabody-KMUS-NCD5-647 DAR 2
FV55 Diabody-PEG4-Azide-DBCO-Carbon5-ASDX2 DAR 1
FV55 Diabody-PEG4-Azide-DBCO-Carbon5-ASDX2 DAR 2
FV55 Diabody-PEG4-Azide-DBCO-Carbon5-ASDX23 DAR 1
FV55 Diabody-PEG4-Azide-DBCO-Carbon5-ASDX23 DAR 2
FV55 Diabody-PEG4-Azide-DBCO-Carbon5-ASDX26 DAR 1
FV55 Diabody-PEG4-Azide-DBCO-Carbon5-ASDX26 DAR 2
FV55 Diabody-PEG4-Azide-DBCO-Carbon5-ASDX32 DAR 1
FV55 Diabody-PEG4-Azide-DBCO-Carbon5-ASDX32 DAR 2
FV55 Diabody-PEG4-Azide-DBCO-Carbon5-ASDX4 DAR 1
FV55 Diabody-PEG4-Azide-DBCO-Carbon5-ASDX4 DAR 2
FV55 Diabody-PEG4-Azide-DBCO-Carbon5-M30m1 DAR 1
FV55 Diabody-PEG4-Azide-DBCO-Carbon5-M30m1 DAR 2
FV55 Diabody-PEG4-Azide-DBCO-Carbon5-M30m3 DAR 1
FV55 Diabody-PEG4-Azide-DBCO-Carbon5-M30m3 DAR 2
FV55 Diabody-PEG4-Azide-DBCO-Carbon5-NCD5-647 DAR 1
FV55 Diabody-PEG4-Azide-DBCO-Carbon5-NCD5-647 DAR 2
FV55 Diabody-PEG4-Azide-DBCO-PEG5-ASDX2 DAR 1
FV55 Diabody-PEG4-Azide-DBCO-PEG5-ASDX2 DAR 2
FV55 Diabody-PEG4-Azide-DBCO-PEG5-ASDX23 DAR 1
FV55 Diabody-PEG4-Azide-DBCO-PEG5-ASDX23 DAR 2
FV55 Diabody-PEG4-Azide-DBCO-PEG5-ASDX26 DAR 1
FV55 Diabody-PEG4-Azide-DBCO-PEG5-ASDX26 DAR 2
FV55 Diabody-PEG4-Azide-DBCO-PEG5-ASDX32 DAR 1
FV55 Diabody-PEG4-Azide-DBCO-PEG5-ASDX32 DAR 2
FV55 Diabody-PEG4-Azide-DBCO-PEG5-ASDX4 DAR 1
FV55 Diabody-PEG4-Azide-DBCO-PEG5-ASDX4 DAR 2
FV55 Diabody-PEG4-Azide-DBCO-PEG5-M30m1 DAR 1
FV55 Diabody-PEG4-Azide-DBCO-PEG5-M30m1 DAR 2
FV55 Diabody-PEG4-Azide-DBCO-PEG5-M30m3 DAR 1
FV55 Diabody-PEG4-Azide-DBCO-PEG5-M30m3 DAR 2
FV55 Diabody-PEG4-Azide-DBCO-PEG5-NCD5-647 DAR 1
FV55 Diabody-PEG4-Azide-DBCO-PEG5-NCD5-647 DAR 2
FV55-Bis-Mal-Lysine-PEG4-TFP ester-M30m1 DAR 1
FV55-Bis-Mal-Lysine-PEG4-TFP ester-M30m3 DAR 1
FV55-Carbon4-Azide-DBCO-Carbon5-ASDX2 DAR 1
FV55-Carbon4-Azide-DBCO-Carbon5-ASDX23 DAR 1
FV55-Carbon4-Azide-DBCO-Carbon5-ASDX26 DAR 1
FV55-Carbon4-Azide-DBCO-Carbon5-ASDX32 DAR 1
FV55-Carbon4-Azide-DBCO-Carbon5-ASDX4 DAR 1
FV55-Carbon4-Azide-DBCO-Carbon5-M30m1 DAR 1
FV55-Carbon4-Azide-DBCO-Carbon5-M30m3 DAR 1
FV55-Carbon4-Azide-DBCO-Carbon5-NCD5-647 DAR 1
FV55-KMUS-M30m1 DAR 1
FV55-KMUS-M30m3 DAR 1
FV55-KMUS-NCD5-647 DAR 1
FV55-PEG4-Azide-DBCO-Carbon5-ASDX2 DAR 1
FV55-PEG4-Azide-DBCO-Carbon5-ASDX23 DAR 1
FV55-PEG4-Azide-DBCO-Carbon5-ASDX26 DAR 1
FV55-PEG4-Azide-DBCO-Carbon5-ASDX32 DAR 1
FV55-PEG4-Azide-DBCO-Carbon5-ASDX4 DAR 1
FV55-PEG4-Azide-DBCO-Carbon5-M30m1 DAR 1
FV55-PEG4-Azide-DBCO-Carbon5-M30m3 DAR 1
FV55-PEG4-Azide-DBCO-Carbon5-NCD5-647 DAR 1
FV55-PEG4-Azide-DBCO-PEG5-ASDX2 DAR 1
FV55-PEG4-Azide-DBCO-PEG5-ASDX23 DAR 1
FV55-PEG4-Azide-DBCO-PEG5-ASDX26 DAR 1
FV55-PEG4-Azide-DBCO-PEG5-ASDX32 DAR 1
FV55-PEG4-Azide-DBCO-PEG5-ASDX4 DAR 1
FV55-PEG4-Azide-DBCO-PEG5-M30m1 DAR 1
FV55-PEG4-Azide-DBCO-PEG5-M30m3 DAR 1
FV55-PEG4-Azide-DBCO-PEG5-NCD5-647 DAR 1
Cetuximab-PEG(n)-M30m3 (n = 1~100) DAR 1
Cetuximab-PEG(n)-M30m3 (n = 1~100) DAR 2
Cetuximab-PEG(n)-M30m3 (n = 1~100) DAR 3
Cetuximab-PEG(n)-M30m3 (n = 1~100) DAR 4
Cetuximab-PEG(n)-M30m3 (n = 1~100) DAR 5
Cetuximab-PEG(n)-M30m3 (n = 1~100) DAR 6
Cetuximab-MC-PEG4-ValCit-PABc-M30m3 DAR 1
Cetuximab-MC-PEG4-ValCit-PABc-M30m3 DAR 2
Cetuximab-MC-PEG4-ValCit-PABc-M30m3 DAR 3
Cetuximab-MC-PEG4-ValCit-PABc-M30m3 DAR 4

In some embodiments, the composition comprises: (a) Cetuximab-DBCO-C9-M30m3 (DAR3) and PEG 12-Poly-(D-Arg) 12; (b) Cetuximab-DBCO-C4/P5-M30m3 (DAR3) and PEG 12-Poly-(D-Arg) 12; (c) Cetuximab-DBCO-PEG9-M30m3 (DAR3) and PEG 12-Poly-(D-Arg) 12; (d) Cetuximab-DBCO-PEG9-M30m3 (DAR2) and PEG12-Poly-(D-Arg) 12; (e) Cetuximab-DBCO-PEG9-M30m3 (DAR4) and PEG 12-Poly-(1D-Arg) 12; (f) Cetuximab-DBCO-PEG9-M30m3 (1DAR6) and PEG 12-Poly-(D-Arg) 12; (g) Cetuximab-Linear-PEG13-M30m3 (DAR4) and PEG 12-Poly-(D-Arg) 12; (h) 3tf12-DBCO-PEG8-NCD5 and Poly(L-Arg)9; (i) 3tf12-DBCO-PEG8-M30m3 and Poly(L-Arg)9; (j) Fv55-SMCC-M30m3 and PEG12-Poly(L-Arg)12; (k) Fv55-PEG30-M30m3 and PEG12-Poly(L-Arg)12; (1) Cetuximab-PEG4-azide-DBCO-PEG5-M30m3 (DAR2) and PEG12PolyArg12{d}; (m) Cetuximab-PEG4-azide-DBCO-PEG5-M30m3 (DAR2) and PolyArg12Cbp3.9 kDa; (n) Cetuximab-PEG4-azide-DBCO-PEG5-M30m3 (DAR4) and PEG12PolyArg12{d}; (o) Cetuximab-PEG4-azide-DBCO-PEG5-M30m3 (DAR4) and PolyArg12Cbp3.9 kDa; (p) Cetuximab-PEG4-azide-DBCO-PEG5-M30m3 (DAR4) and PolyArg12C-PEG2000 Da; (q) Cetuximab-PEG4-azide-DBCO-PEG5-M30m3 (DAR4) and PolyArg12C-PEG5000 Da; (r) Cetuximab-PEG4-azide-DBCO-PEG5-M30m3 (DAR4) and PolyArg12C-Dextran5000 Da; (s) Cetuximab-SMCC-M30m3 (DAR4) and PEG12PolyArg12{d}; (t) Cetuximab-MCVCPABcPNP-M30m3 (DAR4) and PEG12PolyArg12{d}; (u) Cetuximab-MCPEG4VCPABcPNP-M30m3 (DAR4) and PEG12PolyArg12{d}; (v) Cetuximab-PEG4-azide-DBCO-PEG5-M30m3 (DAR2.5) and PEG12PolyArg12{d}; (w) Cetuximab-PEG4-azide-DBCO-PEG5-M30m3 (DAR4.5) and PEG12PolyArg12{d}; (x) Cetuximab-PEG4-azide-DBCO-PEG5-M30m3 (DAR6.5) and PEG12PolyArg12{d}; or (y) Cetuximab-C4(Azide-DBCO)C5-M30m3 and PEG12PolyArg12. In some embodiments, the composition comprises Cetuximab-DBCO-C9-M30m3 (DAR3) and PEG12-Poly-(D-Arg)₁₂. In some embodiments, the composition comprises Cetuximab-DBCO-C4/P5-M30m3 (DAR3) and PEG12-Poly-(D-Arg)₁₂. In some embodiments, the composition comprises Cetuximab-DBCO-PEG9-M30m3 (DAR3) and PEG12-Poly-(D-Arg)₁₂. In some embodiments, the composition comprises Cetuximab-DBCO-PEG9-M30m3 (DAR2) and PEG12-Poly-(D-Arg)₁₂. In some embodiments, the composition comprises Cetuximab-DBCO-PEG9-M30m3 (DAR4) and PEG12-Poly-(D-Arg)₁₂. In some embodiments, the composition comprises Cetuximab-DBCO-PEG9-M30m3 (DAR6) and PEG12-Poly-(D-Arg)₁₂. In some embodiments, the composition comprises Cetuximab-Linear-PEG13-M30m3 (DAR4) and PEG12-Poly-(D-Arg)₁₂. In some embodiments, the composition comprises 3tf12-DBCO-PEG8-NCD5 and Poly(L-Arg)₉. In some embodiments, the composition comprises 3tf12-DBCO-PEG8-M30m3 and Poly(L-Arg)₉. In some embodiments, the composition comprises Fv55-SMCC-M30m3 and PEG12-Poly(L-Arg)₁₂. In some embodiments, the composition comprises Fv55-PEG30-M30m3 and PEG12-Poly(L-Arg)₁₂. In some embodiments, the composition comprises Cetuximab-PEG4-azide-DBCO-PEG5-M30m3 (DAR2) and PEG12PolyArg12{d}. In some embodiments, the composition comprises Cetuximab-PEG4-azide-DBCO-PEG5-M30m3 (DAR2) and PolyArg12Cbp3.9 kDa. In some embodiments, the composition comprises Cetuximab-PEG4-azide-DBCO-PEG5-M30m3 (DAR4) and PEG12PolyArg12{d}. In some embodiments, the composition comprises Cetuximab-PEG4-azide-DBCO-PEG5-M30m3 (DAR4) and PolyArg12Cbp3.9 kDa. In some embodiments, the composition comprises Cetuximab-PEG4-azide-DBCO-PEG5-M30m3 (DAR4) and PolyArg12C-PEG2000 Da. In some embodiments, the composition comprises Cetuximab-PEG4-azide-DBCO-PEG5-M30m3 (DAR4) and PolyArg12C-PEG5000 Da. In some embodiments, the composition comprises Cetuximab-PEG4-azide-DBCO-PEG5-M30m3 (DAR4) and PolyArg12C-Dextran5000 Da. In some embodiments, the composition comprises Cetuximab-SMCC-M30m3 (DAR4) and PEG12PolyArg12{d}. In some embodiments, the composition comprises Cetuximab-MCVCPABcPNP-M30m3 (DAR4) and PEG12PolyArg12{d}. In some embodiments, the composition comprises Cetuximab-MCPEG4VCPABcPNP-M30m3 (DAR4) and PEG12PolyArg12{d}. In some embodiments, the composition comprises Cetuximab-PEG4-azide-DBCO-PEG5-M30m3 (DAR2.5) and PEG12PolyArg12{d}. In some embodiments, the composition comprises Cetuximab-PEG4-azide-DBCO-PEG5-M30m3 (DAR4.5) and PEG12PolyArg12{d}. In some embodiments, the composition comprises Cetuximab-PEG4-azide-DBCO-PEG5-M30m3 (DAR6.5) and PEG12PolyArg12{d}. In some embodiments, the composition comprises Cetuximab-C4(Azide-DBCO)C5-M30m3 and PEG12PolyArg12. In some embodiments, the composition comprises any of the antibody-polynucleotide conjugates and the associated hybrid polymers disclosed in Table 5, supra. Each of the of the antibody-polynucleotide conjugates and the associated hybrid polymers disclosed in Table 5 is considered a separate embodiment.

### Polynucleotide Conjugates

A second aspect of this disclosure provides polynucleotide conjugates. In some embodiments, the polynucleotide conjugate comprise a polynucleotide conjugated to a targeting molecule.

In some embodiments, the targeting moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of targeting moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some embodiments, the targeting moiety is an antibody or binding fragment thereof.

The targeting molecule may be an antibody or an antigen-binding fragment thereof, or a binding protein. In some embodiments, the targeting molecule is an antibody or an antigen binding fragment thereof (e.g. a polynucleotide-antibody conjugate). In some embodiments, the antibody or binding fragment thereof is a human antibody or an antigen-binding fragment thereof, a humanized antibody or an antigen-binding fragment thereof, a murine antibody or an antigen-binding fragment thereof, a chimeric antibody or an antigen-binding fragment thereof, a monoclonal antibody or an antigen-binding fragment thereof, a monovalent Fab′, a divalent Fab2, a F(ab)′3 fragment, a single-chain variable fragment (scFv), a bis-scFv, a (scFv)₂, a diabody, a minibody, an immunoglobulin single variable domain (ISV) such as an Nanobody® molecule, a triabody, a tetrabody, a disulfide stabilized Fv protein (dsFv), a single-domain antibody (sdAb), an Ig NAR, a vNAR, a Centyrin, a camelid antibody or an antigen-binding fragment thereof, a bispecific antibody or an antigen-biding fragment thereof, or a chemically modified derivative thereof. In some embodiments, the antibody or antigen-binding fragment thereof is selected from the group consisting of a monoclonal antibody, a bispecific antibody, a Fab, a Fab-Fc, a Fv, a single chain Fv (scFv), a diabody, a minibody, and an immunoglobulin single variable domain (ISV) such as an Nanobody® molecule. In some embodiments, the antibody or antigen-binding fragment thereof is a monoclonal antibody. In some embodiments, the antibody or antigen-binding fragment thereof is a bispecific antibody. Non-limiting examples of bispecific antibodies in bispecific T-cell engagers (BiTEs) and a dual-affinity retargeting antibodies (DARTs). In some embodiments, the bispecific antibody is a trifunctional antibody or a bispecific mini-antibody. In some embodiments, the bispecific antibody is a trifunctional antibody. In some embodiments, the trifunctional antibody is a full-length monoclonal antibody comprising binding sites for two different antigens. In some embodiments, the bispecific antibody is a bispecific mini-antibody. In some embodiments, the bispecific mini-antibody comprises divalent Fab2, F(ab)′3 fragments, bis-scFv, (scFv)2, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens.

In some embodiments, the antibody or antigen-binding fragment thereof is a Fab. In some embodiments, the antibody or antigen-binding fragment thereof is a Fab-Fc. In some embodiments, the antibody or antigen-binding fragment thereof is a Fv. In some embodiments, the antibody or antigen-binding fragment thereof is a single chain Fv (scFv). In some embodiments, when the antibody or antigen-binding portion is a scFv, the polynucleotide does not comprise a cross-linking residue. In some embodiments, when the antibody or antigen-binding portion is a scFv, the polynucleotide does not comprise a cysteine. In some embodiments, the antibody or antigen-binding fragment thereof is a diabody. In some embodiments, the antibody or antigen-binding fragment thereof is a minibody. In some embodiments, the antibody or antigen-binding fragment thereof is an immunoglobulin single variable domain (ISV) such as an Nanobody® molecule. The Nanobody® may be a Nanobody-HSA®.

In some embodiments, the antibody or antigen-binding fragment thereof is an IgG molecule or is derived from an IgG molecule. The IgG molecule may be an IgG1 or an IgG4 molecule. The antibody or antigen-binding fragment thereof may be an IgG1 molecule or derived therefrom. The antibody or antigen-binding fragment thereof may be an IgG2 molecule or derived therefrom. The antibody or antigen-binding fragment thereof may be an IgG3 molecule or derived therefrom. The antibody or antigen-binding fragment thereof may be an IgG4 molecule or derived therefrom.

Non-limiting examples of antibodies and antigen-binding fragments that may be used as targeting molecules in the present disclosure include FV55scFv, Fv55 diabody, 3TF12, and cetuximab. The FV55 scFv is a monospecific scFv that binds to human transferrin receptor (TfR1). The CDRs of the FV55 scFv are identical to HB21, and the FV55 scFv is oriented V_{H}-V_{L} connected by (G4S)*3 (SEQ ID NO: 3) and has a c-terminal cysteine for conjugation. The molecular weight of the FV55 scFv is ˜26.5 kDa. See, e.g., Haynes B F, Hemler M, Cotner T, Mann D L, Eisenbarth G S, Strominger J L, Fauci A S. Characterization of a monoclonal antibody (5E9) that defines a human cell surface antigen of cell activation. J Immunol. 1981; 127:347-351. [PubMed: 6787129]. The Fv55 diabody comprises two copies of the FV55 scFv, except the linker is (G4S)*N (SEQ ID NO: 3), where N is 1 or 2. The molecular weight of the Fv55 diabody is ˜53 kDa. 3TF12 is a monospecific scFv that binds to human transferrin receptor (TfR1). 3TF12 is oriented V_{H}-V_{L} connected by (G4S)*N; when N is 3 (SEQ ID NO: 3), 3tf12 is a monomeric scFv; when N is 1 (SEQ ID NO: 3), 3tf12 dimerizes to form a diabody. See, e.g., Crepin R. et al. Development of Human Single-Chain Antibodies to the Transferrin Receptor that Effectively Antagonize the Growth of Leukemias and Lymphomas. Cancer Research, 2010, 70(13):5497-506. Cetuximab is a chimeric (mouse/human) monoclonal antibody and an epidermal growth factor receptor (EGFR) inhibitor medication used for the treatment of metastatic colorectal cancer and head and neck cancer. Cetuximab has a molecular weight of 145,781.92 g/mol.

In some embodiments, the targeting molecule is a binding protein. The binding protein may be a soluble receptor or a soluble ligand. In some embodiments, the soluble receptor comprises the extracellular domain of a receptor. In some embodiments, the soluble receptor is a Fc fusion protein.

In some embodiments, the targeting molecule is a plasma protein. In some embodiments, the plasma protein comprises albumin. In some embodiments, the albumin is conjugated by one or more of the conjugation chemistries disclosed herein to a polynucleotide. In some instances, the albumin is conjugated by native ligation chemistry to a polynucleotide. In some instances, albumin is conjugated by lysine conjugation to a polynucleotide.

In some instances, the targeting molecule is a steroid. Non-limiting exemplary steroids include cholesterol, phospholipids, di- and triacylglycerols, fatty acids, hydrocarbons that are saturated, unsaturated, comprise substitutions, or combinations thereof. In some embodiments, the steroid is cholesterol or a cholesterol derivative. In some embodiments, the targeting molecule is cholesterol. In some embodiments, the steroid is conjugated by one or more of the conjugation chemistries disclosed herein to a polynucleotide. In some embodiments, the steroid is conjugated by native ligation chemistry to a polynucleotide.

In some embodiments, the targeting molecule is a polymer, including but not limited to polynucleotide aptamers that bind to specific surface markers on cells. In some embodiments, the targeting molecule is a polynucleotide that does not hybridize to a target gene or mRNA, but instead is capable of selectively binding to a cell surface marker similarly to an antibody binding to its specific epitope of a cell surface marker.

In some embodiments, the targeting molecule is a polypeptide. In some embodiments, the polypeptide has a size between about 1 and about 3 kDa. In some embodiments, the polypeptide has a size between about 1.2 and about 2.8 kDa, about 1.5 and about 2.5 kDa, or about 1.5 and about 2 kDa. In some embodiments, the polypeptide is a bicyclic polypeptide. In some embodiments, the bicyclic polypeptide is a constrained bicyclic polypeptide. In some embodiments, the targeting molecule is a bicyclic polypeptide (e.g., bicycles from Bicycle Therapeutics).

In additional embodiments, the targeting molecule is a small molecule. In some embodiments, the small molecule is an antibody-recruiting small molecule. In some embodiments, the antibody-recruiting small molecule comprises a target-binding terminus and an antibody-binding terminus, in which the target-binding terminus is capable of recognizing and interacting with a cell surface receptor.

In some embodiments, the targeting molecule is a therapeutically active molecule or a biologically active molecule.

In some embodiments, the polynucleotide comprises RNA, DNA or a combination thereof. In some cases, the polynucleotide comprises RNA. In some cases, the polynucleotide comprises DNA. In some cases, the polynucleotide comprises RNA and DNA. In some embodiments, the polynucleotide comprises combinations of DNA, RNA and/or artificial nucleotide analogues. In some embodiments, the polynucleotide is a regulatory non-coding RNA (ncRNA). In some embodiments, the ncRNA comprises short non-coding RNA sequences expressed in a genome that regulates expression or function of other biomolecules in mammalian cells. An ncRNA is generally <200 nucleotides in length and can be single stranded or double stranded and may form non-linear secondary or tertiary structures. An ncRNA can comprise exogenously derived small interfering RNA (siRNA), MicroRNA (miRNA), small nuclear RNA (U-RNA), Small nucleolar RNA (snoRNA), Piwi-interacting RNA (piRNA), repeat associated small interfering RNA (rasiRNA), small rDNA-derived RNA (srRNA), transfer RNA derived small RNA (tsRNA), ribosomal RNA derived small RNA (rsRNA), large non-coding RNA derived small RNA (lncsRNA), or a messenger RNA derived small RNA (msRNA). In some embodiments, the polynucleotide is an engineered polynucleotide. The engineered polynucleotide may comprise DNA or RNA. In some embodiments, the engineered polynucleotide comprises a plurality of nucleotides. In some embodiments, the engineered polynucleotide comprises an artificial nucleotide analogue. In some embodiments, the engineered polynucleotide comprises DNA. In some embodiments, the DNA is genomic DNA, cell-free DNA, cDNA, fetal DNA, viral DNA, or maternal DNA. In some embodiments, the engineered polynucleotide comprises RNA. In some embodiments, the RNA is an siRNA, an ncRNA mimic, a short-harpin RNA (shRNA), a dicer-dependent siRNA (di-siRNA), an antisense oligonucleotide (ASO), a gapmer, a mixmer, double-stranded RNAs (dsRNA), single stranded RNAi, (ssRNAi), DNA-directed RNA interference (ddRNAi), an RNA activating oligonucleotide (RNAa), an aptamer, or an exon skipping oligonucleotide. In some embodiments, the engineered polynucleotide comprises a completely synthetic miRNA. A completely synthetic miRNA is one that is not derived or based upon an ncRNA. Instead, a completely synthetic miRNA may be based upon an analysis of multiple potential target sequences or may be based upon isolated natural non-coding sequences that are not ncRNAs. In some embodiments, the polynucleotide is selected from the group consisting of a siRNA, a miRNA, a miRNA mimic, an antisense oligonucleotide (ASO), an mRNA, and a guide RNA. The polynucleotide may be a siRNA. In some embodiments, the polynucleotide is a miRNA. In some embodiments, the polynucleotide is a miRNA mimic. The polynucleotide may be a miR-30 or a mimic of miR-30. Non-limiting examples of mimics of miR-30 are provided in Table 3, supra.

In some embodiments, the miR-30 mimic is selected from the group consisting of M30 ml, M30m2, M30m3, and M30m4. In some embodiments, the miR-30 mimic is M30 ml. In some embodiments, the miR-30 mimic is M30m2. In some embodiments, the miR-30 mimic is M30m3. In some embodiments, the miR-30 mimic is M30m4.

In some embodiments, is an ASO. In some embodiment the ASO can target and repress multiple genes related to a disorder. In some embodiments, the ASO targets an autosomal dominant mutant gene that causes a genetic disorder. In some embodiments, the ASO targets DMPK. In some embodiments, the ASO targets CAPN3. The ASO may target DUX4. DUX4-targeted ASOs are known in the art. See, e.g., WO 2021/203043 and U.S. Provisional Patent Application No. 63/221,568, each of which is incorporated herein by reference in its entirety. Additional non-limiting examples of DUX4-targeted ASOs are provided in Table 4, supra. In some embodiments, the DUX4-targeted ASO is selected from the group consisting of ASDX2, ASDX4, ASDX23, ASDX26, and ASDX32. In some embodiments, the DUX4-targeted ASO is ASDX2. In some embodiments, the DUX4-targeted ASO is ASDX4. In some embodiments, the DUX4-targeted ASO is ASDX23. In some embodiments, the DUX4-targeted ASO is ASDX26. In some embodiments, the DUX4-targeted ASO is ASDX32.

In some embodiments, the polynucleotide comprises a siRNA, a miRNA, a miRNA mimic, an ASO, or a guide RNA that targets DUX4, DMPK or CAPN3. In some embodiments, the polynucleotide comprises a siRNA that targets DUX4. In some embodiments, the polynucleotide comprises a miRNA that targets DUX4. In some embodiments, the polynucleotide comprises a miRNA mimic that targets DUX4. In some embodiments, the polynucleotide comprises an ASO that targets DUX4. In some embodiments, the polynucleotide comprises a guide RNA that targets DUX4. In some embodiments, the polynucleotide comprises a siRNA that targets DMPK. In some embodiments, the polynucleotide comprises a miRNA that targets DMPK. In some embodiments, the polynucleotide comprises a miRNA mimic that targets DMPK. In some embodiments, the polynucleotide comprises an ASO that targets DMPK. In some embodiments, the polynucleotide comprises a siRNA that targets CAPN3. In some embodiments, the polynucleotide comprises a miRNA that targets CAPN3. In some embodiments, the polynucleotide comprises a miRNA mimic that targets CAPN3. In some embodiments, the polynucleotide comprises an ASO that targets CAPN3.

In some embodiments, the polynucleotide is a coding RNA. In some embodiments, the polynucleotide is a mRNA. In some embodiments, the polynucleotide is a non-coding RNA. In some embodiments, the polynucleotide is a long non-coding RNA. In some embodiments, the polynucleotide is a guide RNA.

In some embodiments, the polynucleotide comprises one or more artificial nucleotide analogues. In some embodiments, one or more of the artificial nucleotide analogues described herein are resistant toward nucleases such as for example ribonuclease such as RNase, deoxyribunuclease such as DNase, or exonuclease such as 5′-3′ exonuclease and 3′-5′ exonuclease when compared to natural polynucleotides. In some embodiments, artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase, deoxyribunuclease such as DNase, or exonuclease such as 5′-3′ exonuclease and 3′-5′ exonuclease. In some embodiments, 2′-O-methyl modified polynucleotide is nuclease resistant (e.g., RNase, DNase, 5′-3′ exonuclease or 3′-5′ exonuclease resistant). In some embodiments, 2′O-methoxyethyl (2′-O-MOE) modified polynucleotide is nuclease resistant (e.g., RNase, DNase, 5′-3′ exonuclease or 3′-5′ exonuclease resistant). In some embodiments, 2′-O-aminopropyl modified polynucleotide is nuclease resistant (e.g., RNase, DNase, 5′-3′ exonuclease or 3′-5′ exonuclease resistant). In some embodiments, 2′-deoxy modified polynucleotide is nuclease resistant (e.g., RNase, DNase, 5′-3′ exonuclease or 3′-5′ exonuclease resistant). In some embodiments, T-deoxy-2′-fluoro modified polynucleotide is nuclease resistant (e.g., RNase, DNase, 5′-3′ exonuclease or 3′-5′ exonuclease resistant). In some embodiments, 2′-O-aminopropyl (2′-O-AP) modified polynucleotide is nuclease resistant (e.g., RNase, DNase, 5′-3′ exonuclease or 3′-5′ exonuclease resistant). In some embodiments, 2′-O-dimethylaminoethyl (2′-O-DMAOE) modified polynucleotide is nuclease resistant (e.g., RNase, DNase, 5′-3′ exonuclease or 3′-5′ exonuclease resistant). In some embodiments, 2′-O-dimethylaminopropyl (2′-O-DMAP) modified polynucleotide is nuclease resistant (e.g., RNase, DNase, 5′-3′ exonuclease or 3′-5′ exonuclease resistant). In some embodiments, T-O-dimethylaminoethyloxyethyl (2′-O-DMAEOE) modified polynucleotide is nuclease resistant (e.g., RNase, DNase, 5′-3′ exonuclease or 3′-5′ exonuclease resistant). In some embodiments, 2′-O—N-methylacetamido (2′-O-NMA) modified polynucleotide is nuclease resistant (e.g., RNase, DNase, 5′-3′ exonuclease or 3′-5′ exonuclease resistant). In some embodiments, LNA-modified polynucleotide is nuclease resistant (e.g., RNase, DNase, 5′-3′ exonuclease or 3′-5′ exonuclease resistant). In some embodiments, ENA-modified polynucleotide is nuclease resistant (e.g., RNase, DNase, 5′-3′ exonuclease or 3′-5′ exonuclease resistant). In some embodiments, HNA-modified polynucleotide is nuclease resistant (e.g., RNase, DNase, 5′-3′ exonuclease or 3′-5′ exonuclease resistant). Morpholinos may be nuclease resistant (e.g., RNase, DNase, 5′-3′ exonuclease or 3′-5′ exonuclease resistant). In some embodiments, PNA-modified polynucleotide is resistant to nucleases (e.g., RNase, DNase, 5′-3′ exonuclease or 3′-5′ exonuclease resistant). In some embodiments, methylphosphonate nucleotide-modified polynucleotide is nuclease resistant (e.g., RNase, DNase, 5′-3′ exonuclease or 3′-5′ exonuclease resistant). In some embodiments, thiolphosphonate nucleotide-modified polynucleotide is nuclease resistant (e.g., RNase, DNase, 5′-3′ exonuclease or 3′-5′ exonuclease resistant). In some embodiments, polynucleotide comprising 2′-fluoro N3-P5′-phosphoramidites is nuclease resistant (e.g., RNase, DNase, 5′-3′ exonuclease or 3′-5′ exonuclease resistant). In some embodiments, the 5′ conjugates described herein inhibit 5′-3′ exonucleolytic cleavage. In some embodiments, the 3′ conjugates described herein inhibit 3′-5′ exonucleolytic cleavage.

In some embodiments, one or more of the artificial nucleotide analogues described herein have increased binding affinity toward their mRNA target relative to an equivalent natural polynucleotide. In some embodiments, the artificial nucleotide analogue comprises a nucleic acid with a modification at a 2′ hydroxyl group of the ribose moiety. In some embodiments, the modification includes an H, OR, R, halo, SH, SR, NH2, NHR, NR2, or CN, wherein R is an alkyl moiety. Exemplary alkyl moieties include, but are not limited to, halogens, sulfurs, thiols, thioethers, thioesters, amines (primary, secondary, or tertiary), amides, ethers, esters, alcohols and oxygen. In some embodiments, the alkyl moiety further comprises a modification. In some embodiments, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some embodiments, the alkyl moiety further comprises a hetero substitution. In some embodiments, the carbon of the heterocyclic group is substituted by a nitrogen, oxygen or sulfur. In some embodiments, the heterocyclic substitution includes but is not limited to, morpholino, imidazole, and pyrrolidino. The one or more of the artificial nucleotide analogues comprising 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites can have increased binding affinity toward their mRNA target relative to an equivalent natural polynucleotide. In some embodiments, 2′-O-methyl modified polynucleotide has increased binding affinity toward their mRNA target relative to an equivalent natural polynucleotide. In some embodiments, 2′-O-methoxyethyl (2′-O-MOE) modified polynucleotide has increased binding affinity toward their mRNA target relative to an equivalent natural polynucleotide. In some embodiments, 2′-O-aminopropyl modified polynucleotide has increased binding affinity toward their mRNA target relative to an equivalent natural polynucleotide. In some embodiments, 2′-deoxy modified polynucleotide has increased binding affinity toward their mRNA target relative to an equivalent natural polynucleotide. In some embodiments, T-deoxy-2′-fluoro modified polynucleotide has increased binding affinity toward their mRNA target relative to an equivalent natural polynucleotide. In some embodiments, 2′-O-aminopropyl (2′-O-AP) modified polynucleotide has increased binding affinity toward their mRNA target relative to an equivalent natural polynucleotide. In some embodiments, 2′-O-dimethylaminoethyl (2′-O-DMAOE) modified polynucleotide has increased binding affinity toward their mRNA target relative to an equivalent natural polynucleotide. In some embodiments, 2′-O-dimethylaminopropyl (2′-O-DMAP) modified polynucleotide has increased binding affinity toward their mRNA target relative to an equivalent natural polynucleotide. In some embodiments, T-O-dimethylaminoethyloxyethyl (2′-O-DMAEOE) modified polynucleotide has increased binding affinity toward their mRNA target relative to an equivalent natural polynucleotide. In some embodiments, 2′-O—N-methylacetamido (2′-O-NMA) modified polynucleotide has increased binding affinity toward their mRNA target relative to an equivalent natural polynucleotide. In some embodiments, LNA-modified polynucleotide has increased binding affinity toward their mRNA target relative to an equivalent natural polynucleotide. In some embodiments, ENA-modified polynucleotide has increased binding affinity toward their mRNA target relative to an equivalent natural polynucleotide. In some embodiments, PNA-modified polynucleotide has increased binding affinity toward their mRNA target relative to an equivalent natural polynucleotide. In some embodiments, HNA-modified polynucleotide has increased binding affinity toward their mRNA target relative to an equivalent natural polynucleotide. In some embodiments, morpholino-modified polynucleotide has increased binding affinity toward their mRNA target relative to an equivalent natural polynucleotide. In some embodiments, methylphosphonate nucleotide-modified polynucleotide has increased binding affinity toward their mRNA target relative to an equivalent natural polynucleotide. In some embodiments, thiolphosphonate nucleotide-modified polynucleotide has increased binding affinity toward their mRNA target relative to an equivalent natural polynucleotide. In some embodiments, polynucleotide comprising 2′-fluoro N3-P5′-phosphoramidites has increased binding affinity toward their mRNA target relative to an equivalent natural polynucleotide. In some embodiments, the increased affinity is illustrated with a lower Kd, a higher melt temperature (Tm), or a combination thereof.

In some embodiments, the artificial nucleotide analogues include 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some embodiments, the artificial nucleotide analogue comprises a modified base such as, but not limited to, 5-propynyluridine, 5-propynylcytidine, 6-methyladenine, 6-methylguanine, N,N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, In some embodiments are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some embodiments, the polynucleotide comprises one or more phosphorothioate internucleotide linkages. In some embodiments, the polynucleotide comprises 2′-5′ internucleotide linkages. In some embodiments, the 2′-5′ internucleotide linkage(s) is at the 3′-end, the 5′-end, or both of the 3′- and 5′-ends of one or both sequence strands. In some embodiments, the 2′-5′ internucleotide linkage(s) is present at various other positions within one or both sequence strands. In some embodiments, the polynucleotide comprises a terminal cap molecule at the 3′-end, the 5′-end, or both of the 3′- and 5′-ends.

In some embodiments, the targeting molecule and the polynucleotide combined to provide a synergistic therapeutic or biological effect.

In some embodiments, the polynucleotide is conjugated directly to the targeting molecule. The polynucleotide may be conjugated to the targeting molecule via a linker. Suitable linkers for conjugating polynucleotides to targeting molecules are known in the art. See, e.g., WO 2017/173408, incorporated herein by reference in its entirety. In some embodiments, the linker is a hydrophobic linker. The linker may be a peptide linker. In some embodiments, the linker is a chemical linker. The chemical linker may be a polymeric linker. In some embodiments, the chemical linker is linear. In some embodiments, the chemical linker is cyclic.

In some embodiments, the polymeric linker comprises PEG, a sugar, a fatty acid, a phosphate, a pyrophosphate or a polysarcosine. In some embodiments, the polymeric linker comprises PEG. In some embodiments, the polymeric linker comprises a sugar. In some embodiments, the polymeric linker comprises a fatty acid. In some embodiments, the polymeric linker comprises a phosphate. In some embodiments, the polymeric linker comprises a pyrophosphate. In some embodiments, the polymeric linker comprises a polysarcosine. The linker may be a high molecular weight PEG linker. In some embodiments, the high molecular weight PEG linker comprises between 1,000 and 5,000 PEG monomers (i.e. is between PEG1k and PEG5k). In some embodiments, the high molecular weight PEG linker is PEG1k. In some embodiments, the high molecular weight PEG linker is PEG1.5k. In some embodiments, the high molecular weight PEG linker is PEG2k. In some embodiments, the high molecular weight PEG linker is PEG3k. In some embodiments, the high molecular weight PEG linker is PEG4k. In some embodiments, the high molecular weight PEG linker is PEG5k.

In some embodiments, the linker is a low molecular weight PEG linker. In some embodiments, the low molecular weight PEG linker comprises between 4 and 100 PEG monomers (i.e. is between PEG4 and PEG100). In some embodiments, the low molecular PEG linker is between PEG12 and PEG48. In some embodiments, the low molecular PEG linker is between PEG12 and PEG24. In some embodiments, the low molecular PEG linker is between PEG12 and PEG18. In some embodiments, the low molecular PEG linker is between PEG6 and PEG18. In some embodiments, the low molecular weight PEG linker is PEG4. In some embodiments, the low molecular weight PEG linker is PEG5. In some embodiments, the low molecular weight PEG linker is PEG6. In some embodiments, the low molecular weight PEG linker is PEG7. In some embodiments, the low molecular weight PEG linker is PEG8. In some embodiments, the low molecular weight PEG linker is PEG9. In some embodiments, the low molecular weight PEG linker is PEG10. In some embodiments, the low molecular weight PEG linker is PEG11. In some embodiments, the low molecular weight PEG linker is PEG12. In some embodiments, the low molecular weight PEG linker is PEG13. In some embodiments, the low molecular weight PEG linker is PEG14. In some embodiments, the low molecular weight PEG linker is PEG15. In some embodiments, the low molecular weight PEG linker is PEG16. In some embodiments, the low molecular weight PEG linker is PEG17. In some embodiments, the low molecular weight PEG linker is PEG18. In some embodiments, the low molecular weight PEG linker is PEG19. In some embodiments, the low molecular weight PEG linker is PEG20. In some embodiments, the low molecular weight PEG linker is PEG21. In some embodiments, the low molecular weight PEG linker is PEG22. In some embodiments, the low molecular weight PEG linker is PEG23. In some embodiments, the low molecular weight PEG linker is PEG24. In some embodiments, the low molecular weight PEG linker is PEG25. In some embodiments, the low molecular weight PEG linker is PEG26. In some embodiments, the low molecular weight PEG linker is PEG27. In some embodiments, the low molecular weight PEG linker is PEG28. In some embodiments, the low molecular weight PEG linker is PEG29. In some embodiments, the low molecular weight PEG linker is PEG30. In some embodiments, the low molecular weight PEG linker is PEG31. In some embodiments, the low molecular weight PEG linker is PEG32. In some embodiments, the low molecular weight PEG linker is PEG33. In some embodiments, the low molecular weight PEG linker is PEG34. In some embodiments, the low molecular weight PEG linker is PEG35. In some embodiments, the low molecular weight PEG linker is PEG36. In some embodiments, the low molecular weight PEG linker is PEG37. In some embodiments, the low molecular weight PEG linker is PEG38. In some embodiments, the low molecular weight PEG linker is PEG39. In some embodiments, the low molecular weight PEG linker is PEG40. In some embodiments, the low molecular weight PEG linker is PEG41. In some embodiments, the low molecular weight PEG linker is PEG42. In some embodiments, the low molecular weight PEG linker is PEG43. In some embodiments, the low molecular weight PEG linker is PEG44. In some embodiments, the low molecular weight PEG linker is PEG45. In some embodiments, the low molecular weight PEG linker is PEG46. In some embodiments, the low molecular weight PEG linker is PEG47. In some embodiments, the low molecular weight PEG linker is PEG48. In some embodiments, the low molecular weight PEG linker is PEG49. In some embodiments, the low molecular weight PEG linker is PEG50. In some embodiments, the low molecular weight PEG linker is PEG51. In some embodiments, the low molecular weight PEG linker is PEG52. In some embodiments, the low molecular weight PEG linker is PEG53. In some embodiments, the low molecular weight PEG linker is PEG54. In some embodiments, the low molecular weight PEG linker is PEG55. In some embodiments, the low molecular weight PEG linker is PEG56. In some embodiments, the low molecular weight PEG linker is PEG57. In some embodiments, the low molecular weight PEG linker is PEG58. In some embodiments, the low molecular weight PEG linker is PEG59. In some embodiments, the low molecular weight PEG linker is PEG60. In some embodiments, the low molecular weight PEG linker is PEG61. In some embodiments, the low molecular weight PEG linker is PEG62. In some embodiments, the low molecular weight PEG linker is PEG63. In some embodiments, the low molecular weight PEG linker is PEG64. In some embodiments, the low molecular weight PEG linker is PEG65. In some embodiments, the low molecular weight PEG linker is PEG66. In some embodiments, the low molecular weight PEG linker is PEG67. In some embodiments, the low molecular weight PEG linker is PEG68. In some embodiments, the low molecular weight PEG linker is PEG69. In some embodiments, the low molecular weight PEG linker is PEG70. In some embodiments, the low molecular weight PEG linker is PEG71. In some embodiments, the low molecular weight PEG linker is PEG72. In some embodiments, the low molecular weight PEG linker is PEG73. In some embodiments, the low molecular weight PEG linker is PEG74. In some embodiments, the low molecular weight PEG linker is PEG75. In some embodiments, the low molecular weight PEG linker is PEG76. In some embodiments, the low molecular weight PEG linker is PEG77. In some embodiments, the low molecular weight PEG linker is PEG78. In some embodiments, the low molecular weight PEG linker is PEG79. In some embodiments, the low molecular weight PEG linker is PEG80. In some embodiments, the low molecular weight PEG linker is PEG81. In some embodiments, the low molecular weight PEG linker is PEG82. In some embodiments, the low molecular weight PEG linker is PEG83. In some embodiments, the low molecular weight PEG linker is PEG84. In some embodiments, the low molecular weight PEG linker is PEG85. In some embodiments, the low molecular weight PEG linker is PEG86. In some embodiments, the low molecular weight PEG linker is PEG87. In some embodiments, the low molecular weight PEG linker is PEG88. In some embodiments, the low molecular weight PEG linker is PEG89. In some embodiments, the low molecular weight PEG linker is PEG90. In some embodiments, the low molecular weight PEG linker is PEG91. In some embodiments, the low molecular weight PEG linker is PEG92. In some embodiments, the low molecular weight PEG linker is PEG93. In some embodiments, the low molecular weight PEG linker is PEG94. In some embodiments, the low molecular weight PEG linker is PEG95. In some embodiments, the low molecular weight PEG linker is PEG96. In some embodiments, the low molecular weight PEG linker is PEG97. In some embodiments, the low molecular weight PEG linker is PEG98. In some embodiments, the low molecular weight PEG linker is PEG99. In some embodiments, the low molecular weight PEG linker is PEG100.

In some embodiments, the linker is non-cleavable. In some embodiments, the linker is cleavable. The linker may be cleavable in vivo. In some embodiments, the cleavable linker is selected from the group consisting of a disulfide linker, a self-immolative peptide polymer hybrid, and a sulfatase-promoted arylsulfate linker. In some embodiments, the cleavable linker is a disulfide linker. The cleavable linker may be a self-immolative peptide polymer hybrid. In some embodiments, the cleavable linker is a sulfatase-promoted arylsulfate linker. In some embodiments, the self-immolative peptide polymer hybrid comprises glucuronic acid, para-amino-benzoyloxy (PAB), 7-amino-3-hydroxyethyl-coumarin (7-AHC), or Fe(II)-reactive 1,2,4-trioxolane scaffold (TRX). In some embodiments, the self-immolative peptide polymer hybrid comprises glucuronic acid. In some embodiments, the self-immolative peptide polymer hybrid comprises para-amino-benzoyloxy (PAB). In some embodiments, the self-immolative peptide polymer hybrid comprises 7-amino-3-hydroxyethyl-coumarin (7-AHC). In some embodiments, the self-immolative peptide polymer hybrid comprises Fe(II)-reactive 1,2,4-trioxolane scaffold (TRX).

In some embodiments, the cleavable linker is cleaved through reduction, hydrolysis, proteolysis, photo cleavage, chemical cleavage, enzymatic cleavage, or bio-orthogonal-cleavage. In some embodiments, the cleavable linker is cleaved through reduction. In some embodiments, the cleavable linker is cleaved through hydrolysis. In some embodiments, the cleavable linker is cleaved through proteolysis. In some embodiments, the cleavable linker is cleaved through photo cleavage. In some embodiments, the cleavable linker is cleaved through chemical cleavage. The chemical cleavage may be by Fe II mediated β elimination of TRX. In some embodiments, the cleavable linker is cleaved through enzymatic cleavage. The enzymatic cleavage may be by non-proteolytic sulfatase, β-galactosidase/glucuronidase or pyrophosphatase. In some embodiments, the enzymatic cleavage is by non-proteolytic sulfatase. In some embodiments, the enzymatic cleavage is by β-galactosidase/glucuronidase. In some embodiments, the enzymatic cleavage is by pyrophosphatase. In some embodiments, the cleavable linker is cleaved through bio-orthogonal-cleavage. The bio-orthogonal cleavage may be by Cu I-BTTAA or free copper ion mediated cleavage. In some embodiments, the linker is an acid cleavable linker.

In some embodiments, the linker includes a C1-C6 alkyl group (e.g., a C5, C4, C3, C2, or C1 alkyl group). In some embodiments, the linker includes homobifunctional cross linkers, heterobifunctional cross linkers, and the like. In some embodiments, the liker is a traceless linker (or a zero-length linker). In some embodiments, the linker is a non-polymeric linker. In some embodiments, the linker is a non-peptide linker or a linker that does not contain an amino acid residue.

In some embodiments, the linker comprises a homobifuctional linker. Exemplary homobifuctional linkers include, but are not limited to, Lomant's reagent dithiobis (succinimidylpropionate) DSP, 3,3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[2-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-ρ-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(y-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl) 1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such as 1-(p-Azidosalicylamido)-4-(iodoacetamido)butane (AsIB), N-[4-(p-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(p-azidosalicylamido)butylamine (AsBA), and arginine-reactive and photoreactive cross-linkers such as p-azidophenyl glyoxal (APG).

In some embodiments, the linker comprises a reactive functional group. In some embodiments, the reactive functional group comprises a nucleophilic group that is reactive to an electrophilic group present on a binding moiety. Exemplary electrophilic groups include carbonyl groups—such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (mc). In some embodiments, the linker comprises maleimidocaproyl (mc). In some embodiments, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some embodiments, the self-stabilizing maleimide is a maleimide group described in Lyon, et al, “Self-hydrolyzing maleimides improve the stability and pharmacological properties of antibody-drug conjugates,” Nat. Biotechnol. 32(10): 1059-1062 (2014). In some embodiments, the linker comprises a self-stabilizing maleimide. In some embodiments, the linker is a self-stabilizing maleimide.

In some embodiments, the linker comprises a peptide moiety. In some embodiments, the peptide moiety comprises at least 2, 3, 4, 5, 6, 7, 8, or more amino acid residues. In some embodiments, the peptide moiety is a cleavable peptide moiety (e.g., either enzymatically or chemically). In some embodiments, the peptide moiety is a non-cleavable peptide moiety. In some embodiments, the peptide moiety comprises Val-Cit (valine-citrulline), Gly-Gly-Phe-Gly (SEQ ID NO: 112), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 113), or Gly-Phe-Leu-Gly (SEQ ID NO: 114). In some embodiments, the linker comprises a peptide moiety such as: Val-Cit (valine-citrulline), Gly-Gly-Phe-Gly (SEQ ID NO: 112), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 113), or Gly-Phe-Leu-Gly (SEQ ID NO: 114). In some embodiments, the linker comprises Val-Cit. In some embodiments, the linker is Val-Cit.

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (mc). In some embodiments, the peptide group is val-cit. In some embodiments, the benzoic acid group is PABA. In some embodiments, the linker comprises a mc-val-cit group. In some embodiments, the linker comprises a val-cit-PABA group. In additional cases, the linker comprises a mc-val-cit-PABA group.

In some embodiments, the linker is a self-immolative linker or a self-elimination linker. In some embodiments, the linker is a self-immolative linker. In other cases, the linker is a self-elimination linker (e.g., a cyclization self-elimination linker). In some embodiments, the linker comprises a linker described in U.S. Pat. No. 9,089,614 or PCT Publication No. WO2015038426, each of which is incorporated herein by reference in its entirety.

In some embodiments, the linker is a dendritic type linker. In some embodiments, the dendritic type linker comprises a branching, multifunctional linker moiety. In some embodiments, the dendritic type linker is used to increase the molar ratio of polynucleotide B to the binding moiety A. In some embodiments, the dendritic type linker comprises PAMAM dendrimers.

In some embodiments, the linker is a traceless linker or a linker in which after cleavage does not leave behind a linker moiety (e.g., an atom or a linker group) to a polynucleotide or a targeting molecule. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some embodiments, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., “Traceless solid-phase organic synthesis,” Chem. Rev. 102: 2607-2024 (2002). In some embodiments, a linker is a traceless linker as described in U.S. Pat. No. 6,821,783, incorporated herein by reference in its entirety.

In some embodiments, the linker comprises a functional group that exerts steric hinderance at the site of bonding between the linker and a conjugating moiety (e.g., a polynucleotide or a targeting molecule disclosed herein). In some embodiments, the steric hinderance is a steric hindrance around a disulfide bond. Exemplary linkers that exhibit steric hinderance comprises a heterobifunctional linker, such as a heterobifunctional linker described above. In some embodiments, a linker that exhibits steric hinderance comprises SMCC and SPDB.

In some embodiments, the linker is an acid cleavable linker. In some embodiments, the acid cleavable linker comprises a hydrazone linkage, which is susceptible to hydrolytic cleavage. In some embodiments, the acid cleavable linker comprises a thiomaleamic acid linker. In some embodiments, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some embodiments, the linker is a linker described in U.S. Pat. Nos. 6,884,869; 7,498,298; 8,288,352; 8,609, 105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042, each of which is incorporated herein by reference in its entirety.

In some embodiments, the linker is conjugated to a lysine residue, a cysteine residue, a histidine residue, or a non-natural amino acid residue in the targeting molecule. In some embodiments, the linker is conjugated to a lysine residue in the targeting molecule. In some embodiments, the linker is conjugated to a cysteine residue in the targeting molecule. In some embodiments, the linker is conjugated to a histidine residue in the targeting molecule. In some embodiments, the linker is conjugated to a non-natural amino acid residue in the targeting molecule.

In some embodiments, the linker is conjugated to the targeting molecule by a chemical conjugation or an enzymatic conjugation. In some embodiments, the linker is conjugated to the targeting molecule by a chemical conjugation. The chemical conjugation may comprise acylation and click chemistry. In some embodiments, the linker is conjugated to the targeting molecule by an enzymatic conjugation. The enzymatic conjugation may be via a sortase or a transferase enzyme.

In some embodiments, the polynucleotide is conjugated to the targeting molecule by a chemical ligation process. In some embodiments, the polynucleotide is conjugated to the targeting molecule by a native ligation. In some embodiments, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some embodiments, the conjugation is as described in U.S. Pat. No. 8,936,910. In some embodiments, the polynucleotide is conjugated to the targeting molecule either site-specifically or non-specifically via native ligation chemistry.

In some embodiments, the polynucleotide is conjugated to the targeting molecule by a site-directed method utilizing a “traceless” coupling technology (Philochem). In some embodiments, the “traceless” coupling technology utilizes an N-terminal 1,2-aminothiol group on the targeting molecule which is then conjugate with a polynucleotide containing an aldehyde group. (see Casi et al., “Site-specific traceless coupling of potent cytotoxic drugs to recombinant antibodies for pharmacodelivery,” JACS 134(13): 5887-5892 (2012)).

In some embodiments, the polynucleotide is conjugated to the targeting molecule by a site-directed method utilizing an unnatural amino acid incorporated into the targeting molecule. In some embodiments, the unnatural amino acid comprises p-acetylphenylalanine (pAcPhe). In some embodiments, the keto group of pAcPhe is selectively coupled to an alkoxy-amine derivatized conjugating moiety to form an oxime bond. (see Axup et al., “Synthesis of site-specific antibody-drug conjugates using unnatural amino acids,” PNAS 109(40): 16101-16106 (2012)).

In some embodiments, the polynucleotide is conjugated to the targeting molecule by a site-directed method utilizing an enzyme-catalyzed process. In some embodiments, the site-directed method utilizes SMARTag™ technology (Redwood). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleotide via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some embodiments, the enzyme-catalyzed process comprises microbial transglutaminase (mTG). In some embodiments, the polynucleotide is conjugated to the targeting molecule utilizing a microbial transglutaminase catalyzed process. In some embodiments, mTG catalyzes the formation of a covalent bond between the amide side chain of a glutamine within the recognition sequence and a primary amine of a functionalized polynucleotide. In some embodiments, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some embodiments, the polynucleotide is conjugated to the targeting molecule by a method as described in PCT Publication No. WO2014/140317 (incorporated herein by reference in its entirety), which utilizes a sequence-specific transpeptidase. In some embodiments, the polynucleotide is conjugated to the targeting molecule by a method as described in U.S. Patent Publication Nos. 2015/0105539 and 2015/0105540, each of which is incorporated herein by reference in its entirety.

In some embodiments, each targeting molecule is conjugated to between one and eight polynucleotide molecules (i.e. a Drug:Antibody Ratio (DAR) between 1 and 8). In some embodiments, each targeting molecule is conjugated to one polynucleotide molecule (DAR of 1). In some embodiments, each targeting molecule is conjugated to two polynucleotide molecules (DAR of 2). In some embodiments, each targeting molecule is conjugated to three polynucleotide molecules (DAR of 3). In some embodiments, each targeting molecule is conjugated to four polynucleotide molecules (DAR of 4). In some embodiments, each targeting molecule is conjugated to five polynucleotide molecules (DAR of 5). In some embodiments, each targeting molecule is conjugated to six polynucleotide molecules (DAR of 6). In some embodiments, each targeting molecule is conjugated to seven polynucleotide molecules (DAR of 7). In some embodiments, each targeting molecule is conjugated to eight polynucleotide molecules (DAR of 8).

In some embodiments, the polynucleotide-conjugated targeting molecule has a molecular weight greater than about 30 kDa. In some embodiments, the polynucleotide-conjugated targeting molecule has a molecular weight greater than about 40 kDa. The polynucleotide-conjugated targeting molecule may have a molecular weight greater than about 50 kDa. In some embodiments, the polynucleotide-conjugated targeting molecule has a molecular weight greater than about 60 kDa. In some embodiments, the polynucleotide-conjugated targeting molecule has a molecular weight no greater than about 7,500 kDa.

In some embodiments, the polynucleotide-conjugated targeting molecule has a molecular weight greater than 30 kDa. In some embodiments, the polynucleotide-conjugated targeting molecule has a molecular weight greater than 40 kDa. The polynucleotide-conjugated targeting molecule may have a molecular weight greater than 50 kDa. In some embodiments, the polynucleotide-conjugated targeting molecule has a molecular weight greater than 60 kDa. In some embodiments, the polynucleotide-conjugated targeting molecule has a molecular weight no greater than 7,500 kDa.

In some embodiments, the polynucleotide conjugate is selected from the group consisting of Cetuximab-DBCO-C9-M30m3 (DAR of 3); Cetuximab-DBCO-C4/P5-M30m3 (DAR of 3); Cetuximab-DBCO-PEG9-M30m3 (DAR of 3); Cetuximab-DBCO-PEG9-M30m3 (DAR of 2); Cetuximab-DBCO-PEG9-M30m3 (DAR of 4); Cetuximab-DBCO-PEG9-M30m3 (DAR of 6); Cetuximab-Linear-PEG13-M30m3 (DAR of 4); 3tf12-DBCO-PEG8-NCD5 (DAR of 1); 3tf12-DBCO-PEG8-M30m3 (DAR of 1); Fv55-SMCC-M30m3 (DAR of 1); Fv55-PEG8-DBCO-M30m3(DAR of 1) and Fv55-PEG8-DBCO-M30m3(DAR of 2). In some embodiments, the polynucleotide conjugate is Cetuximab-DBCO-C9-M30m3 (DAR of 3). In some embodiments, the polynucleotide conjugate is Cetuximab-DBCO-C4/P5-M30m3 (DAR of 3). In some embodiments, the polynucleotide conjugate is Cetuximab-DBCO-PEG9-M30m3 (DAR of 3). In some embodiments, the polynucleotide conjugate is Cetuximab-DBCO-PEG9-M30m3 (DAR of 2). In some embodiments, the polynucleotide conjugate is Cetuximab-DBCO-PEG9-M30m3 (DAR of 4). In some embodiments, the polynucleotide conjugate is Cetuximab-DBCO-PEG9-M30m3 (DAR of 6). In some embodiments, the polynucleotide conjugate is Cetuximab-Linear-PEG13-M30m3 (DAR of 4). In some embodiments, the polynucleotide conjugate is 3tf12-DBCO-PEG8-NCD5 (DAR of 1). In some embodiments, the polynucleotide conjugate is 3tf12-DBCO-PEG8-M30m3 (DAR of 1). In some embodiments, the polynucleotide conjugate is Fv55-SMCC-M30m3 (DAR of 1). In some embodiments, the polynucleotide conjugate is Fv55-PEG8-DBCO-M30m3(DAR of 1). In some embodiments, the polynucleotide conjugate is Fv55-PEG8-DBCO-M30m3(DAR of 2). In some embodiments, the polynucleotide conjugate is selected from the antibody-polynucleotide conjugates listed in Table 5, supra. In some embodiments, the polynucleotide conjugate is selected from the antibody-polynucleotide conjugates listed in Table 6, supra. Each of the APCs disclosed in Table 5 or Table 6 is considered a separate embodiment.

### Methods of Treating Genetic Diseases

A third aspect of this disclosure provides methods for treating genetic diseases in a subject in need thereof. In some embodiments, the method comprises administering to the subject a therapeutically effective amount of any of the compositions for delivering polynucleotides disclosed herein. In some embodiments, the method comprises administering to the subject a therapeutically effective amount of any of the polynucleotide conjugates disclosed herein.

A genetic disease, as disclosed herein, may be a cancer, a neurological disorder, a fibrosis disease, a scarring disease, an autoimmune disease, or an inherited genetic disorder.

In some embodiments, the genetic disease is a neurological disorder. In some embodiments, the neurological disorder is selected from the group consisting of Acquired Epileptiform Aphasia, Acute Disseminated Encephalomyelitis, Adrenoleukodystrophy, Agenesis of the corpus callosum, Agnosia, Aicardi syndrome, Alexander disease, Alpers' disease, Alternating hemiplegia, Alzheimer's disease, Amyotrophic lateral sclerosis (see Motor Neuron Disease), Anencephaly, Angelman syndrome, Angiomatosis, Anoxia, Aphasia, Apraxia, Arachnoid cysts, Arachnoiditis, Arnold-Chiari malformation, Arteriovenous malformation, Asperger's syndrome, Ataxia Telangiectasia, Attention Deficit Hyperactivity Disorder, Autism, Auditory processing disorder, Autonomic Dysfunction, Back Pain, Batten disease, Bechet's disease, Bell's palsy, Benign Essential Blepharospasm, Benign Focal Amyotrophy, Benign Intracranial Hypertension, Bilateral frontoparietal polymicrogyria, Binswanger's disease, Blepharo-spasm, Bloch-Sulzberger syndrome, Brachial plexus injury, Brain abscess, Brain damage, Brain in-jury, Brain tumor, Brown-Sequard syndrome, Canavan disease, Carpal tunnel syndrome (CTS), Causalgia, Central pain syndrome, Central pontine myelinolysis, Centronuclear myopathy, Cephalic disorder, Cerebral aneurysm, Cerebral arteriosclerosis, Cerebral atrophy, Cerebral gigantism, Cerebral palsy, Charcot-Marie-Tooth disease, Chiari malformation, Chorea, Chronic inflammatory de-myelinating polyneuropathy (CIDP), Chronic pain, Chronic regional pain syndrome, Coffin Lowry syndrome, Coma, including Persistent Vegetative State, Congenital facial diplegia, Corticobasal degeneration, Cranial arteritis, Craniosynostosis, Creutzfeldt-Jakob disease, Cumulative trauma disorders, Cushing's syndrome, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Neurological Dyspraxia, Diabetic neuropathy, Diffuse sclerosis, Dysautonomia, Dyscalculia, Dysgraphia, Dyslexia, Dystonia, Early infantile epileptic encephalopathy, Empty sella syndrome, Encephalitis, Encephalocele, Encephalotrigeminal angiomatosis, Encopresis, Epilepsy, Erb's palsy, Erythromelalgia, Essential tremor, Fabry's disease, Fahr's syndrome, Fainting, Familial spastic paralysis, Febrile seizures, Fisher syndrome, Friedreich's ataxia, FART Syndrome, Gaucher's disease, Gerstmann's syndrome, Giant cell arteritis, Giant cell inclusion disease, Globoid cell Leukodystrophy, Gray matter heterotopia, Guillain-Barre syndrome, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Holoprosencephaly, Huntington's disease, Hydranencephaly, Hydrocephalus, Hypercortisolism, hypertrophic cardiomyopathy, Hypoxia, Immune-Mediated encephalomyelitis, Inclusion body myositis, Incontinentia pigmenti, Infantile phytanic acid storage disease, Infantile Refsum disease, Infantile spasms, Inflammatory myopathy, Intracranial cyst, Intracranial hypertension, Joubert syndrome, Kearns-Sayre syndrome, Kennedy disease, Kinsbourne syndrome, Klippel Feil syndrome, Krabbe disease, Kugelberg-Welander disease, Kuru, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Lewy body dementia, Lissencephaly, Locked-In syndrome, Lou Gehrig's disease, Lumbar disc disease, Lyme disease—Neurological Sequelae, Macha-do-Joseph disease (Spinocerebellar ataxia type 3), Macrencephaly, Maple Syrup Urine Disease, Marfan syndrome, Megalencephaly, Melkersson-Rosenthal syndrome, Menieres disease, Meningitis, Menkes disease, Metachromatic leukodystrophy, Microcephaly, Migraine, Miller Fisher syndrome, Mini-Strokes, Mitochondrial Myopathies, Mobius syndrome, Monomelic amyotrophy, Motor Neuron Disease, Motor skills disorder, Moyamoya disease, Mucopolysaccharidoses, Multi-Infarct Dementia, Multi-focal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neuro-fibromatosis, Neuroleptic malignant syndrome, Neurological manifestations of AIDS, Neurological sequelae of lupus, Neuromyotonia, Neuronal ceroid lipofuscinosis, Neuronal migration disorders, Niemann-Pick disease, Non 24-hour sleep-wake syndrome, Nonverbal learning disorder, O'Sulli-van-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Overuse syndrome, Palinopsia, Paresthesia, Parkinson's disease, Paramyotonia Con-genita, Paraneoplastic diseases, Paroxysmal attacks, Parry-Romberg syndrome, Rombergs Syndrome, Pelizaeus-Merzbacher disease, Periodic Paralyses, Peripheral neuropathy, Persistent Vegetative State, Pervasive neurological disorders, Photic sneeze reflex, Phytanic Acid Storage disease, Pick's disease, Pinched Nerve, Pituitary Tumors, PMG, Polio, Polymicrogyria, Polymyositis, Porencephaly, Post-Polio syndrome, Postherpetic Neuralgia (PHN), Postinfectious Encephalomyelitis, Postural Hypotension, Prader-Willi syndrome, Primary Lateral Sclerosis, Prion diseases, Progressive Hemifacial Atrophy also known as Rombergs Syndrome, Progressive multifocal leukoencephalopathy, Progressive Sclerosing Poliodystrophy, Progressive Supranuclear Palsy, Pseudotumor cerebri, Ramsay-Hunt syndrome (Type I and Type II), Rasmussen's encephalitis, Reflex sympathetic dystrophy syndrome, Refsum disease, Repetitive motion disorders, Repetitive stress injury, Restless legs syndrome, Retrovirus-associated myelopathy, Rett syndrome, Reye's syndrome, Rombergs Syndrome, Rabies, Saint Vitus dance, Sandhoff disease, Schytsophrenia, Schilder's disease, Schizencephaly, Sensory Integration Dysfunction, Septooptic dysplasia, Shaken baby syndrome, Shingles, Shy-Drager syndrome, Sjogren's syndrome, Sleep apnea, Sleeping sickness, Snatiation, Sotos syndrome, Spasticity, Spina bifida, Spinal cord injury, Spinal cord tumors, Spinal muscular atrophy, Spinal stenosis, Steele-Richardson-Olszewski syndrome, see Progressive Supranuclear Palsy, Spinocerebellar ataxia, Stiff-person syndrome, Stroke, Sturge-Weber syndrome, Subacute sclerosing panencephalitis, Subcortical arteriosclerotic encephalopathy, Superficial siderosis, Sydenham's chorea, Syncope, Synesthesia, Syringomyelia, Tardive dyskinesia, Tay-Sachs disease, Temporal arteritis, Tethered spinal cord syndrome, Thomsen disease, Thoracic outlet syndrome, Tic Douloureux, Todd's paralysis, Tourette syndrome, Transient ischemic attack, Transmissible spongiform encephalopathies, Transverse myelitis, Traumatic brain injury, Tremor, Trigeminal neuralgia, Tropical spastic paraparesis, Trypanosomiasis, Tuberous sclerosis, Vasculitis including temporal arteritis, Von Hippel-Lindau disease (VHL), Viliuisk Encephalomyelitis (VE), Wallenberg's syn-drome, Werdnig-Hoffman disease, West syndrome, Whiplash, Williams syndrome, Wilson's dis-ease, X-Linked Spinal and Bulbar Muscular Atrophy, and Zellweger syndrome. In some embodiments, the neurological disorder is a movement disorder, for example multiple system atrophy (MSA).

In some embodiments, the genetic disease is an autoimmune disease. In some embodiments, the autoimmune disease is selected from the group consisting of acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Bechet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophillic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syn-drome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosis, ligneous conjunctivitis, linear IgA disease (LAD), Lupus (SLE), Lyme dis-ease, Meniere's disease, microscopic polyangitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syn-drome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syn-drome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some embodiment, the genetic disease may be selected from the group consisting of AIDS, anthrax, botulism, brucellosis, chancroid, chlamydial infection, cholera, coccidioidomycosis, cryptosporidiosis, cyclosporiasis, dipheheria, ehrlichiosis, arboviral encephalitis, enterohemorrhagic Escherichia coli, giardiasis, gonorrhea, dengue fever, haemophilus influenza, Hansen's disease (Leprosy), hantavirus pulmonary syn-drome, hemolytic uremic syndrome, hepatitis A, hepatitis B, hepatitis C, human immunodeficiency virus, legionellosis, listeriosis, Lyme disease, malaria, measles. Meningococcal disease, mumps, pertussis (whooping cough), plague, paralytic poliomyelitis, psittacosis, Q fever, rabies, rocky mountain spotted fever, rubella, congenital rubella syndrome, shigellosis, smallpox, streptococcal disease (invasive group A), streptococcal toxic shock syndrome, Streptococcus pneumonia, syphilis, tetanus, toxic shock syndrome, trichinosis, tuberculosis, tularemia, typhoid fever, vancomycin intermediate resistant staphylocossus aureus, varicella, yellow fever, variant Creutzfeldt-Jakob dis-ease (vCJD), Ebola hemorrhagic fever, Echinococcosis, Hendra virus infection, human monkey-pox, influenza A, influenza B, H5N1, lassa fever, Margurg hemorrhagic fever, Nipah virus, O'nyong fever, Rift valley fever, Herpes, HIV, HCV genotype 1, HCV genotype 2, HCV genotype 3, HCV genotype 4, HCV genotype 5, HCV genotype 6, SARS-CoV-2 (COVID-19), SARS-CoV (SARS), MERS-CoV (MERS), 229E coronavirus, NL63 coronavirus, OC43 coronavirus, CoV-HKU1(HKU1), alpha coronavirus, beta coronavirus, Venezuelan equine encephalitis and West Nile virus.

In some embodiments, the genetic disease is a fibrosis disease, a scarring disease or both. In some embodiments, the fibrosis disease or the scarring disease is selected from the group consisting of pulmonary fibrosis, cystic fibrosis, idiopathic pulmonary fibrosis, radiation induced fibrosis, myocardial fibrosis, bridging fibrosis, cirrhosis, gliosis, arterial stiffness, arthrofibrosis, Chron's disease, Dupuytren's contracture, keloid, mediastinal fibrosis, myelofibrosis, Peyronie's disease, nephrogenic systemic fibrosis, progressive massive fibrosis, retroperitoneal fibrosis, scleroderma/systemic sclerosis, and adhesive capsulitis.

In some embodiments, the genetic disease is a cancer. In some embodiments, the cancer is selected from the group consisting of thyroid cancer, adrenal cortical cancer, anal cancer, aplastic anemia, bile duct cancer, bladder cancer, bone cancer, bone metastasis, central nervous system (CNS) cancers, peripheral nervous system (PNS) cancers, breast cancer, Castleman's disease, cervical cancer, childhood Non-Hodgkin's lymphoma, lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, non-melanoma skin cancer, stomach cancer, testicular cancer, thymus cancer, uterine cancer (e.g. uterine sarcoma), vaginal cancer, vulvar cancer, or Waldenstrom's macroglobulinemia. In some cases, a cancer may be selected from a list maintained by the National Cancer Institute (https://www.cancer.gov/types).

A condition or a disease, as disclosed herein, can include hyperproliferative disorders. Malignant hyperproliferative disorders can be stratified into risk groups, such as a low risk group and a medium-to-high risk group. Hyperproliferative disorders can include but may not be limited to cancers, hyperplasia, or neoplasia. In some cases, the hyperproliferative cancer can be breast cancer such as a ductal carcinoma in duct tissue of a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarcinoma that has migrated to the bone; pancreatic cancer such as epithelioid carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which may be divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which may be a skin condition that sometimes develops into squamous cell carcinoma; eye retinoblastoma; cutaneous or intraocular (eye) melanoma; primary liver cancer (cancer that begins in the liver); kidney cancer; autoimmune deficiency syndrome (AIDS)-related lympho-ma such as diffuse large B-cell lymphoma, B-cell immunoblastic lymphoma and small non-cleaved cell lymphoma; Kaposi's Sarcoma; viral-induced cancers including hepatitis B virus (HBV), hepatitis C virus (HCV),and hepatocellular carcinoma; human lymphotropic virus-type 1 (HTLV-1) and adult T-cell leukemia/lymphoma; and human papilloma virus (HPV) and cervical cancer; central nervous system (CNS) cancers such as primary brain tumor, which includes gliomas (astrocytoma, anaplastic astrocytoma, or glioblastoma multiforme), oligodendrogliomas, ependymomas, meningiomas, lymphomas, schwannomas, and medulloblastomas; peripheral nervous system (PNS) cancers such as acoustic neuromas and malignant peripheral nerve sheath tumors (MPNST) including neurofibromas and schwannomas, malignant fibrous cytomas, malignant fibrous histiocytomas, malignant meningiomas, malignant mesotheliomas, and malignant mixed Mullerian tumors; oral cavity and oropharyngeal cancer such as hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer. In some cases, the diseases stratified, classified, characterized, or diagnosed by the methods of the present disclosure include but may not be limited to thyroid disorders such as for example benign thyroid disorders including but not limited to follicular adenomas, Hurthle cell adenomas, lymphocytic thyroiditis, and thyroid hyperplasia. In some cases, the diseases stratified, classified, characterized, or diagnosed by the methods of the present disclosure include but may not be limited to malignant thyroid disorders such as for example follicular carcinomas, follicular variant of papillary thyroid carcinomas, medullary carcinomas, and papillary carcinomas.

In some embodiments, the genetic disease is an inherited genetic disorder caused by abnormalities in genes or chromosomes. Inherited genetic disorders can be grouped into two categories: single gene disorders and multifactorial and polygenic (complex) dis-orders. A single gene disorder may be the result of a single mutated gene. Inheriting a single gene disorder can include but not be limited to autosomal dominant, autosomal recessive, X-linked dominant, X-linked recessive, Y-linked and mitochondrial inheritance. In some embodiments, one mutated copy of the gene is necessary for a person to be affected by an autosomal dominant disorder. Examples of autosomal dominant type of disorder can include but are not limited to Huntington's disease, Neurofibromatosis 1, Marfan Syndrome, Hereditary nonpolyposis colorectal cancer, or Hereditary multiple exostoses. In autosomal recessive disorders, two copies of the gene must be mutated for a subject to be affected by the autosomal recessive disorder. Examples of this type of disorder can include but may not be limited to cystic fibrosis, sickle-cell disease (also partial sickle-cell disease), Tay-Sachs disease, Niemann-Pick disease, or spinal muscular atrophy. X-linked dominant disorders are caused by mutations in genes on the X chromosome such as X-linked hypophosphatemic rickets. Some X-linked dominant conditions such as Rett syndrome, Incontinentia Pigmenti type 2 and Aicardi Syndrome can be fatal. X-linked recessive disorders are also caused by mutations in genes on the X chromosome. Examples of this type of disorder can include but are not limited to Hemophilia A, Duchenne muscular dystrophy, red-green color blindness, muscular dystrophy and Androgenetic alopecia. Y-linked disorders are caused by mutations on the Y chromo-some. Examples can include but are not limited to Male Infertility and hypertrichosis pinnae. The genetic disorder of mitochondrial inheritance, also known as maternal inheritance, can apply to genes in mitochondrial DNA such as in Leber's Hereditary Optic Neuropathy.

Inherited genetic disorders may also be complex, multifactorial or polygenic. Polygenic inherited genetic disorders may be associated with the effects of multiple genes in combination with lifestyle and environmental factors. Although complex genetic disorders can cluster in families, they do not have a clear-cut pattern of inheritance. Multifactorial or polygenic disorders include, but are not limited to, heart disease, diabetes, asthma, autism, autoimmune diseases such as multiple sclerosis, cancers, ciliopathies, cleft palate, hypertension, inflammatory bowel disease, mental retardation or obesity.

Other exemplary inherited genetic disorders include but may not be limited to 1p36 deletion syndrome, 21-hydroxylase deficiency, 22q11.2 deletion syndrome, aceruloplasminemia, achondrogenesis, type II, achondroplasia, acute intermittent porphyria, adenylosuccinate lyase deficiency, Adrenoleu-kodystrophy, Alexander disease, alkaptonuria, alpha-1 antitrypsin deficiency, Alstrom syndrome, Alzheimer's disease (type 1, 2, 3, and 4), Amelogenesis Imperfecta, amyotrophic lateral sclerosis, Amyotrophic lateral sclerosis type 2, Amyotrophic lateral sclerosis type 4, amyotrophic lateral sclerosis type 4, androgen insensitivity syndrome, Anemia, Angelman syndrome, Apert syndrome, ataxia-telangiectasia, Beare-Stevenson cutis gyrata syndrome, Benjamin syndrome, beta thalassemia, biotimidase deficiency, Birt-Hogg-Dube syndrome, bladder cancer, Bloom syndrome, Bone diseases, breast cancer, Camptomelic dysplasia, Canavan disease, Cancer, Celiac Disease, Chronic Granulomatous Disorder (CGD), Charcot-Marie-Tooth disease, Charcot-Marie-Tooth disease Type 1, Charcot-Marie-Tooth disease Type 4, Charcot-Marie-Tooth disease Type 2, Charcot-Marie-Tooth disease Type 4, Cockayne syndrome, Coffin-Lowry syndrome, collagenopathy types II and XI, Colorectal Cancer, Congenital absence of the vas deferens, congenital bilateral absence of vas deferens, congenital diabetes, congenital erythropoietic porphyria, Congenital heart disease, congenital hypothyroidism, Connective tissue disease, Cowden syndrome, Cri du chat syndrome, Crohn's dis-ease, fibrostenosing, Crouzon syndrome, Crouzonodermoskeletal syndrome, cystic fibrosis, De Grouchy Syndrome, Degenerative nerve diseases, Dent's disease, developmental disabilities, Di-George syndrome, Distal spinal muscular atrophy type V, Down syndrome, Dwarfism, Ehlers-Danlos syndrome, Ehlers-Danlos syndrome arthrochalasia type, Ehlers-Danlos syndrome classical type, Ehlers-Danlos syndrome dermatosparaxis type, Ehlers-Danlos syndrome kyphoscoliosis type, vascular type, erythropoietic protoporphyria, Fabry's disease, Facial injuries and disorders, factor V Leiden thrombophilia, familial adenomatous polyposis, familial dysautonomia, fanconi anemia, FG syndrome, fragile X syndrome, Friedreich ataxia, Friedreich's ataxia, G6PD deficiency, galactosemia, Gaucher's disease (type 1, 2, and 3), Genetic brain disorders, Glycine encephalopathy, Haemochromatosis type 2, Haemochromatosis type 4, Harlequin Ichthyosis, Head and brain malformations, Hearing disorders and deafness, Hearing problems in children, hemochromatosis (neonatal, type 2 and type 3), hemophilia, hepatoerythropoietic porphyria, hereditary coproporphyria, Hereditary Multiple Exostoses, hereditary neuropathy with liability to pressure palsies, hereditary non-polyposis colorectal cancer, homocystinuria, Huntington's disease, Hutchinson Gilford Progeria Syndrome, hyperoxaluria, primary, hyperphenylalaninemia, hypochondrogenesis, hypochondroplasia, idic15, incontinentia pigmenti, Infantile Gaucher disease, infantile-onset ascending hereditary spastic paralysis, Infertility, Jackson-Weiss syndrome, Joubert syndrome, Juvenile Primary Lateral Sclerosis, Kennedy disease, Klinefelter syndrome, Kniest dysplasia, Krabbe disease, Learning disability, Lesch-Nyhan syndrome, Leukodystrophies, Li-Fraumeni syndrome, lipoprotein lipase deficiency, familial, Male genital disorders, Marfan syndrome, McCune-Albright syndrome, McLeod syndrome, Mediterranean fever, familial, Menkes disease, Menkes syndrome, Metabolic disorders, methemoglobinemia beta-globin type, Methemoglobinemia congenital methaemoglobinaemia, methylmalonic acidemia, Micro syndrome, Microcephaly, Movement disorders, Mowat-Wilson syndrome, Mucopolysaccharidosis (MPS I), Muenke syndrome, Muscular dystrophy, Muscular dystrophy, Duchenne and Becker type, muscular dystrophy, Duchenne and Becker types, myotonic dystrophy, Myotonic dystrophy type 1 and type 2, limb girdle muscular dystrophy, Pompe disease, Neonatal hemochromatosis, neurofibromatosis, neurofibromatosis 1, neurofibromatosis 2, Neurofibromatosis type I, neurofibromatosis type II, Neurologic diseases, Neuromuscular disorders, Niemann-Pick disease, Nonketotic hyperglycinemia, nonsyndromic deafness, Nonsyndromic deafness autosomal recessive, Noonan syn-drome, osteogenesis imperfecta (type I and type III), otospondylomegaepiphyseal dysplasia, pantothenate kinase-associated neurodegeneration, Patau Syndrome (Trisomy 13), Pendred syndrome, Peutz-Jeghers syndrome, Pfeiffer syndrome, phenylketonuria, porphyria, porphyria cutanea tarda, Prader-Willi syndrome, primary pulmonary hypertension, prion disease, Progeria, propionic acidemia, protein C deficiency, protein S deficiency, pseudo-Gaucher disease, pseudoxanthoma elasticum, Retinal disorders, retinoblastoma, retinoblastoma FA-Friedreich ataxia, Rett syndrome, Rubinstein-Taybi syndrome, Sandhoff disease, sensory and autonomic neuropathy type III, sickle cell anemia, skeletal muscle regeneration, Skin pigmentation disorders, Smith Lemli Opitz Syn-drome, Speech and communication disorders, spinal muscular atrophy, spinal-bulbar muscular atrophy, spinocerebellar ataxia, spondyloepimetaphyseal dysplasia, Strudwick type, spondyloepiphyseal dysplasia congenita, Stickler syndrome, Stickler syndrome COL2A1, Tay-Sachs disease, tetrahydrobiopterin deficiency, thanatophoric dysplasia, thiamine-responsive megaloblastic anemia with diabetes mellitus and sensorineural deafness, Thyroid disease, Tourette's Syndrome, Treacher Collins syndrome, triple X syndrome, tuberous sclerosis, Turner syndrome, Usher syndrome, variegate porphyria, von Hippel-Lindau disease, Waardenburg syndrome, Weissenbacher-Zweymüller syndrome, Wilson disease, Wolf-Hirschhorn syndrome, Xeroderma Pigmentosum, X-linked severe combined immunodeficiency, X-linked sideroblastic anemia, or X-linked spinal-bulbar muscle atrophy.

In some embodiments, the genetic disease is a viral infection. The viral infection may be by a virus selected from the group consisting of an adenovirus, an anellovirus, an arenavirus, an astrovirus, a bunyavirus, a calicivirurs, a coronavirus, a filovirus, a flavivirus, a hepadnavirus, a herpesvirus, an orthomyxovirus, a papillomavirus, a paramyxovirus, a parvovirus, a picornavirus, a pneumovirus, a polyomavirus, a poxvirus, a reovirus, a retrovirus, a rhabdovirus, and a togavirus. In some embodiments, the virus is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments, the polynucleotide comprises a siRNA, a miRNA, a miRNA mimic, an ASO, or a guide RNA that targets a viral gene. In some embodiments, the polynucleotide comprises a siRNA that targets a viral gene. In some embodiments, the polynucleotide comprises a miRNA that targets a viral gene. In some embodiments, the polynucleotide comprises a miRNA mimic that targets a viral gene. In some embodiments, the polynucleotide comprises an ASO that targets a viral gene. In some embodiments, the polynucleotide comprises a guide RNA that targets a viral gene. The polynucleotide may be conjugated to a targeting molecule that specifically binds to a viral protein or a protein on the surface of a host cell for the virus. In some embodiments, the polynucleotide and the targeting molecule synergize in the treatment of the viral infection.

In some embodiments, the genetic disease is cancer. In some embodiments, the cancer is characterized by overexpression of an oncogene. In some embodiments, the polynucleotide comprises a siRNA, a miRNA, a miRNA mimic, an ASO, or a guide RNA that targets the oncogene. In some embodiments, the polynucleotide comprises a siRNA that targets the oncogene. In some embodiments, the polynucleotide comprises a miRNA that targets the oncogene. In some embodiments, the polynucleotide comprises a miRNA mimic that targets the oncogene. In some embodiments, the polynucleotide comprises an ASO that targets the oncogene. In some embodiments, the polynucleotide comprises a guide RNA that targets the oncogene.

In some embodiments, the cancer is characterized by reduced expression of a tumor suppressor gene. The polynucleotide may comprise a mRNA molecule encoding the tumor suppressor gene. In some embodiments, the polynucleotide comprises a guide RNA that that restores expression of the tumor suppressor gene.

In some embodiments, the polynucleotide is conjugated to a targeting molecule that specifically binds a tumor cell of the cancer. In some embodiments, the targeting molecule specifically binds epidermal growth factor receptor; and wherein the polynucleotide is a miR-30 miRNA or a mimic thereof. In some embodiments, the targeting molecule specifically binds ACVR1; and wherein the polynucleotide is a miR-30 miRNA or a mimic thereof. In some embodiments, the targeting molecule specifically binds ACVR1; and wherein the polynucleotide is a miR-26 miRNA or a mimic thereof. In some embodiments, the targeting molecule specifically binds TFR. In some embodiments, the targeting molecule that specifically binds TFR is selected from the group consisting of FV55 scFv, Fv55 diabody, and 3TF12. In some embodiments, the targeting molecule that specifically binds TFR is FV55 scFv. In some embodiments, the targeting molecule that specifically binds TFR is Fv55 diabody. In some embodiments, the targeting molecule that specifically binds TFR is 3TF12. The polynucleotide and the targeting molecule may synergize in the treatment of the cancer.

In some embodiments, the genetic disease is a neuromuscular disorder. The neuromuscular disorder may be a muscular dystrophy. In some embodiments, the muscular dystrophy is facioscapulohumeral muscular dystrophy (FSHD). In some embodiments, the polynucleotide comprises a siRNA, a miRNA, a miRNA mimic, an ASO, or a guide RNA that targets DUX4, DMPK or CAPN3. In some embodiments, the polynucleotide comprises a siRNA that targets DUX4. In some embodiments, the polynucleotide comprises a miRNA that targets DUX4. In some embodiments, the polynucleotide comprises a miRNA mimic that targets DUX4. In some embodiments, the polynucleotide comprises an ASO that targets DUX4. In some embodiments, the polynucleotide comprises a guide RNA that targets DUX4. In some embodiments, the ASO that targets DUX is selected from the group consisting of the DUX4-targeted ASOs disclosed in Table 4. In some embodiments, the DUX4-targeted ASO is selected from the group consisting of ASDX2, ASDX4, ASDX23, ASDX26, and ASDX32. In some embodiments, the DUX4-targeted ASO is ASDX2. In some embodiments, the DUX4-targeted ASO is ASDX4. In some embodiments, the DUX4-targeted ASO is ASDX23. In some embodiments, the DUX4-targeted ASO is ASDX26. In some embodiments, the DUX4-targeted ASO is ASDX32. In some embodiments, the polynucleotide comprises a siRNA that targets DMPK. In some embodiments, the polynucleotide comprises a miRNA that targets DMPK. In some embodiments, the polynucleotide comprises a miRNA mimic that targets DMPK. In some embodiments, the polynucleotide comprises an ASO that targets DMPK. In some embodiments, the polynucleotide comprises a siRNA that targets CAPN3. In some embodiments, the polynucleotide comprises a miRNA that targets CAPN3. In some embodiments, the polynucleotide comprises a miRNA mimic that targets CAPN3. In some embodiments, the polynucleotide comprises an ASO that targets CAPN3.

In some embodiments, the muscular dystrophy is Duchenne muscular dystrophy. In some embodiments, the polynucleotide is a mRNA encoding dystrophin or utrophin. In some embodiments, the polynucleotide is a guide RNA that restores the expression of dystrophin or utrophin.

In some embodiments, the polynucleotide is conjugated to a targeting molecule that specifically binds a marker on the surface of a skeletal muscle cell of the subject. The targeting molecule may specifically bind ACVR1. In some embodiments, the targeting molecule specifically binds ACVR1; and wherein the polynucleotide is a DUX4-targeted ASO.

In some embodiments, the polynucleotide and the targeting molecule synergize in the treatment of the muscular dystrophy.

### Anti-Transferrin Receptor Antibodies

A fourth aspect of this disclosure provides antibodies or antigen-binding fragments thereof that specifically bind human transferrin receptor (TfR1). In some embodiments, the antibody or antigen-binding fragment thereof comprises a heavy chain variable region (VH) comprising the amino acid sequence (CDRs underlined):

(SEQ ID NO: 1)
QVQVQDSGGELVQPGGSLRVSCKASGFNIKDSYMHWVRQAPG
KGLEWVAFIDPETGNTYYADSVKGRFTISRDNSKNTLYLQMN
SLRAEDTAVYYCARGSIYWYFDVWGKGTTVTVSS;

and a light chain variable region (VL) comprising the amino acid sequence (CDRs underlined):

(SEQ ID NO: 2)
DIQMTQSPSSLSASVGQRVTITCRASQSLLNSSNQKNSLGWY
QQKPGKAPKLLIYFASTRQSGVPSRFSGSGSGTDFTLTISSL
QPEDFATYYCQQHYSTPLTFGQGTKVDIKRC.

In some embodiments, the antibody or antigen-binding fragment thereof is selected from the group consisting of a full-length antibody, a Fab, a Fab-Fc, a Fv, a single chain Fv (scFv), a diabody, a minibody, and an immunoglobulin single variable domain (ISV) such as an Nanobody® molecule. In some embodiments, the antibody or antigen-binding fragment thereof is a scFv. In some embodiments, the antibody or antigen-binding fragment thereof is a diabody.

In some embodiments, the VH and VL are connected a linker. In some embodiments, the linker comprises the amino acid sequence GGGGS (SEQ ID NO: 3). In some embodiments, the linker comprises the amino acid sequence (GGGGS)_{N} (SEQ ID NO: 3), wherein N is 1-3. In some embodiments, the antibody or antigen-binding fragment thereof is a scFv and the VH and VL are connected by a linker, wherein the linker comprises the amino acid sequence (GGGGS)₃ (SEQ ID NO: 3). In some embodiments, the antibody or antigen-binding fragment thereof is a diabody and the VH and VL are connected by a linker, wherein the linker comprises the amino acid sequence (GGGGS)_{N} (SEQ ID NO: 3), wherein N is 1 or 2.

### Exemplary Embodiments

Particular embodiments of the disclosure are set forth in the following numbered paragraphs:

1. A composition comprising a hybrid polymer and a polynucleotide, wherein the hybrid polymer comprises a cationic portion and a neutral portion.

2. The composition according to embodiment 1, wherein the cationic portion of the hybrid polymer interacts non-covalently with the polynucleotide, e.g. via an ionic interaction.

3. The composition according to embodiment 1 or 2, wherein the cationic portion of the hybrid polymer is a cationic polypeptide.

4. The composition according to embodiment 3, wherein the cationic polypeptide is a poly-arginine polypeptide.

5. The composition according to embodiment 3, wherein the cationic polypeptide is a poly-lysine polypeptide.

6. The composition according to embodiment 3, wherein the cationic polypeptide comprises arginine and lysine residues.

7. The composition according to embodiment 3, wherein the cationic polypeptide comprises protamine.

8. The composition according to any one of embodiments 3-7, wherein the cationic polypeptide comprises L-amino acid residues.

9. The composition according to any one of embodiments 3-7, wherein the cationic polypeptide comprises D-amino acid residues.

10. The composition according to any one of embodiments 3-7, wherein the cationic polypeptide comprises L-amino acid residues and D-amino acid residues.

11. The composition according to any one of embodiments 3-10, wherein the cationic polypeptide comprises between 9 and 18 amino acid residues.

12. The composition according to embodiment 11, wherein the cationic polypeptide comprises 12 amino acid residues.

13. The composition according to embodiment 3, wherein the cationic polypeptide is selected from the group of cationic polypeptides disclosed in Table 1.

14. The composition according to embodiment 1 or 2, wherein the cationic portion of the hybrid polymer comprises a cationic polymer between about 600 and about 2000 Daltons in size.

15. The composition according to embodiment 14, wherein the cationic polymer is a linear polymer.

16. The composition according to embodiment 14, wherein the cationic polymer is a branched polymer.

17. The composition according to any one of embodiments 14-16, wherein the cationic polymer is selected from the group consisting of gelatin, glucosamine, N-acetylglucosamine, chitosan, cationic dextran, cationic cyclodextrin, cationic cellulose, polyethylenimine (PEI), polyamidoamine (PAA), poly(amino-co-ester)s (PAEs), poly[2-(N,N-dimethylamino)ethyl methacrylate](PDMAEMA), or cationic lipids, such as DOTAP (N-(1-(2,3-dioleoyloxy) propyl)-N,N,N trimethylammonium) chloride, poly[N,N-Diethylaminoethyl Methacrylate](PDEAEMA), a cationic mucic acid polymer (cMAP) and DOPE (dioleoyl phosphatidylethanolamine).

18. The composition according to any one of embodiments 1-17, wherein the neutral portion of the hybrid polymer comprises a polymer between about 100 and about 1000 Daltons in size.

19. The composition according to embodiment 18, wherein the neutral portion of the hybrid polymer comprises poly(ethylene glycol)(PEG).

20. The composition according to any one of embodiments 1-13 and 18-19, wherein the hybrid polymer is a PEGylated cationic polypeptide.

21. The composition according to embodiment 19 or 20, wherein the hybrid polymer comprises a PEG12 to PEG24 polymer.

22. The composition according to any one of embodiments 1-21, wherein the hybrid polymer and the polynucleotide do not form aggregates or nanoparticles.

23. The composition according to any one of embodiments 1-22, wherein the charge ratio of the cationic polypeptide to the polynucleotide is between 0.25:1 and 5:1.

24. The composition according to embodiment 23, wherein the charge ratio of the cationic polypeptide to the polynucleotide is between 0.5:1 and 5:1.

25. The composition according to embodiment 23, wherein the charge ratio of the cationic polypeptide to the polynucleotide is between 1:1 and 4:1.

26. The composition according to embodiment 23, wherein the charge ratio of the cationic polypeptide to the polynucleotide is between 1:1 and 2:1.

27. The composition according to embodiment 23, wherein the charge ratio of the cationic polypeptide to the polynucleotide is 1:1 or 2:1.

28. The composition according to embodiment 1 or 2, wherein the hybrid polymer is selected from the group consisting of PEG12PolyArg12{d}, PEG12PolyArg6, PEG12PolyArg6C, PEG24PolyArg12C, PEG24PolyArg12, PEG24PolyArg9, PolyArg12C-PEG2000 Da, PolyArg12C-PEG5000 Da, PolyArg12C-Dextran5000 Da, PEG12PolyArg12, PEG12PolyArg9d, PEG1000DaPolyArg12, PEG2000DaPolyArg12, PEG5000DaPolyArg12, PolyArg12Cbp1.5 kDa, PolyArg12Cbp3.9 kDa, PolyArg12Cbp16 kDa, CPolyArg12Cbp1.5 kDa, PolyArg12Cbp2 kDa, PolyArg12bp2 kDa, Amide Dextran, Lysine Dextran, PEG PEI 15kda, BPEI-G-PEG 550, and BPEI-G-PEG 5000.

29. The composition according to any one of embodiments 1-28, wherein the polynucleotide is conjugated to a targeting molecule.

30. The composition according to embodiment 29, wherein the targeting molecule is an antibody or an antigen-binding fragment thereof, or a binding protein.

31. The composition according to embodiment 30, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, and an immunoglobulin single variable domain (ISV) such as an Nanobody® molecule.

32. The composition according to embodiment 31, wherein the bispecific antibody is a bispecific T-cell engager (BiTE) or a dual-affinity retargeting antibody (DART). 33. The composition according to embodiment 31, wherein the Nanobody® is a Nanobody-HSA®.

34. The composition according to any one of embodiments 30-33, wherein the antibody or antigen-binding fragment thereof is an IgG molecule or is derived from an IgG molecule.

35. The composition according to embodiment 34, wherein the IgG molecule is an IgG1 or an IgG4 molecule.

36. The composition according to embodiment 30, wherein the binding protein is a soluble receptor or a soluble ligand.

37. The composition according to embodiment 36, wherein the soluble receptor comprises the extracellular domain of a receptor.

38. The composition according to embodiment 36 or 37, wherein the soluble receptor is a Fc fusion protein.

39. The composition according to any one of embodiments 29-38, wherein the targeting molecule is a therapeutically active molecule or a biologically active molecule.

40. The composition according to any one of embodiments 1-39, wherein the polynucleotide is selected from the group consisting of a siRNA, an ncRNA mimic, a short-harpin RNA (shRNA), a dicer-dependent siRNA (di-siRNA), an antisense oligonucleotide (ASO), a gapmer, a mixmer, a double-stranded RNA (dsRNA), a single stranded RNAi, (ssRNAi), a DNA-directed RNA interference (ddRNAi), an RNA activating oligonucleotide (RNAa), an aptamer, an exon skipping oligonucleotide, a miRNA, a miRNA mimic, an mRNA, and a guide RNA.

41. The composition according to embodiment 40, wherein the polynucleotide is a miRNA mimic.

42. The composition according to embodiment 41, wherein the miRNA mimic mimics miR-30.

43. The composition according to embodiment 42, wherein the polynucleotide is miRNA mimic is selected from the group consisting of M30 ml, M30m2, M30m3, and M30m4.

44. The composition according to embodiment 43, wherein the polynucleotide is M30m3.

45. The composition according to embodiment 40, where in the polynucleotide is an ASO.

46. The composition according to embodiment 45, wherein the ASO is a DUX4-targeted ASO.

47. The composition according to embodiment 46, wherein the DUX4-targeted ASO is selected from the group consisting of the DUX4-targeted ASOs disclosed in Table 4.

48. The composition according to embodiment 47, wherein the DUX4-targeted ASO is selected from the group consisting of ASDX2, ASDX4, ASDX23, ASDX26 and ASDX32.

49. The composition according to any one of embodiments 40-48, wherein the targeting molecule and the polynucleotide result in a synergistic therapeutic or biological effect.

50. The composition according to any one of embodiments 29-49, wherein the polynucleotide is conjugated directly to the targeting molecule.

51. The composition according to any one of embodiments 29-49, wherein the polynucleotide is conjugated to the targeting molecule via a linker.

52. The composition according to embodiment 51, wherein the linker is a hydrophobic linker.

53. The composition according to embodiment 51, wherein the linker is a peptide linker.

54. The composition according to embodiment 51, wherein the linker is a chemical linker.

55. The composition according to embodiment 54, wherein the chemical linker is a polymeric linker.

56. The composition according to embodiment 54 or 55, wherein the chemical linker is linear.

57. The composition according to embodiment 54 or 55, wherein the chemical linker is cyclic.

58. The composition according to embodiment 55, wherein the polymeric linker comprises PEG, a sugar, a fatty acid, a phosphate, a pyrophosphate or a polysarcosine.

59. The composition according to embodiment 58, wherein the linker is a high molecular weight PEG linker.

60. The composition according to embodiment 58, wherein the linker is a low molecular weight PEG linker.

61. The composition according to any one of embodiments 51-60, wherein the linker is non-cleavable.

62. The composition according to any one of embodiment 51-60, wherein the linker is cleavable.

63. The composition according to embodiment 62, wherein the linker is cleavable in vivo.

64. The composition according to embodiment 62 or 63, wherein the cleavable linker is selected from the group consisting of a disulfide linker, a self-immolative peptide polymer hybrid, and a sulfatase-promoted arylsulfate linker.

65. The composition according to embodiment 64, wherein the self-immolative peptide polymer hybrid comprises glucuronic acid, para-amino-benzoyloxy (PAB), 7-amino-3-hydroxyethyl-coumarin (7-AHC), or Fe(II)-reactive 1,2,4-trioxolane scaffold (TRX).

66. The composition according to any one of embodiments 62-65, wherein the cleavable linker may be cleaved through reduction, hydrolysis, proteolysis, photo cleavage, chemical cleavage, enzymatic cleavage, and bio-orthogonal-cleavage.

67. The composition according to embodiment 66, wherein the chemical cleavage is by Fe II mediated R elimination of TRX.

68. The composition according to embodiment 66, wherein the enzymatic cleavage is by non-proteolytic sulfatase, β-galactosidase/glucuronidase or pyrophosphatase.

69. The composition according to embodiment 66, wherein the bio-orthogonal cleavage is by Cu I-BTTAA or free copper ion mediated cleavage.

70. The composition according to any one of embodiments 51-69, wherein the linker is conjugated to a lysine residue, a cysteine residue, histidine residue, or a non-natural amino acid residue in the targeting molecule.

71. The composition according to any one of embodiments 51-70, wherein the linker is conjugated to the targeting molecule by a chemical conjugation or an enzymatic conjugation.

72. The composition according to embodiment 71, wherein the chemical conjugation comprises acylation and click chemistry.

73. The composition according to embodiment 71, wherein the enzymatic conjugation is via a sortase or a transferase enzyme.

74. The composition according to any one of embodiments 29-73, wherein each targeting molecule is conjugated to between one and eight polynucleotide molecules (DAR of between 1 and 8).

75. The composition according to embodiment 74, wherein each targeting molecule is conjugated to one polynucleotide molecule (DAR 1).

76. The composition according to embodiment 74, wherein each targeting molecule is conjugated to two polynucleotide molecules (DAR 2).

77. The composition according to embodiment 74, wherein each targeting molecule is conjugated to three polynucleotide molecules (DAR 3).

78. The composition according to embodiment 74, wherein each targeting molecule is conjugated to four polynucleotide molecules (DAR 4).

79. The composition according to embodiment 74, wherein each targeting molecule is conjugated to five polynucleotide molecules (DAR 5).

80. The composition according to embodiment 74, wherein each targeting molecule is conjugated to six polynucleotide molecules (DAR 6).

81. The composition according to embodiment 74, wherein each targeting molecule is conjugated to seven polynucleotide molecules (DAR 7).

82. The composition according to embodiment 74, wherein each targeting molecule is conjugated to eight polynucleotide molecules (DAR 8).

83. The composition according to any one of embodiments 29-82, wherein the polynucleotide-conjugated targeting molecule has a molecular weight greater than 30 kDa.

84. The composition according to embodiment 83, wherein the polynucleotide-conjugated targeting molecule has a molecular weight greater than 40 kDa.

85. The composition according to embodiment 84, wherein the polynucleotide-conjugated targeting molecule has a molecular weight greater than 50 kDa.

86. The composition according to embodiment 85, wherein the polynucleotide-conjugated targeting molecule has a molecular weight greater than 60 kDa.

87. The composition according to any one of embodiments 29-86, wherein the polynucleotide-conjugated targeting molecule has a molecular weight no greater than 7,500 kDa.

88. The composition according to embodiment 29, wherein the polynucleotide conjugate is selected from the group consisting of Cetuximab-DBCO-C9-M30m3 (DAR3); Cetuximab-DBCO-C4/P5-M30m3 (DAR3); Cetuximab-DBCO-PEG9-M30m3 (DAR3); Cetuximab-DBCO-PEG9-M30m3 (DAR2); Cetuximab-DBCO-PEG9-M30m3 (DAR4); Cetuximab-DBCO-PEG9-M30m3 (DAR6); Cetuximab-Linear-PEG13-M30m3 (DAR4); Cetuximab-PEG4-azide-DBCO-PEG5-M30m3 (DAR1), Cetuximab-PEG4-azide-DBCO-PEG5-M30m3 (DAR2), Cetuximab-PEG4-azide-DBCO-PEG5-M30m3 (DAR4), Cetuximab-PEG4-azide-DBCO-PEG5-M30m3 (DAR2.5), Cetuximab-PEG4-azide-DBCO-PEG5-M30m3 (DAR4.5), Cetuximab-PEG4-azide-DBCO-PEG5-M30m3 (DAR6.5), Cetuximab-SMCC-M30m3 (DAR4) (SMCC), Cetuximab-MCVCPABcPNP-M30m3 (DAR4) (MCVCPABcPNP), Cetuximab-MCPEG4VCPABcPNP-M30m3 (DAR4) (MCPEG4VCPABcPNP), Cetuximab-C4-Azide-DBCO-C5-M30m3, Cetuximab-PEG4-azide-DBCO-PEG4-m30m3 (DAR4), Cetuximab-MC-PEG4-ValCit-PABc-M30m3 (DAR1), Cetuximab-MC-PEG4-ValCit-PABc-M30m3 (DAR2), Cetuximab-MC-PEG4-ValCit-PABc-M30m3 (DAR3), Cetuximab-MC-PEG4-ValCit-PABc-M30m3 (DAR4), 3tf12-DBCO-PEG8-NCD5 (DAR1); 3tf12-DBCO-PEG8-M30m3 (DAR1); Fv55-SMCC-M30m3 (DAR1); Fv55-PEG8-DBCO-M30m3 (DAR1), Fv55-PEG8-DBCO-M30m3 (DAR2), Fv55-linker-M30m3 (DAR2), Fv55-DBCO-PEG8-M30 ml(DAR1), Fv55-DBCO-PEG8-M30 ml(DAR2), and ASO-carbon4-DBCO-Carbon5-3tf12 (DAR1).

89. The composition according to embodiment 29, wherein the polynucleotide conjugate is selected from the group consisting for the antibody-polynucleotide conjugates disclosed in Table 5 or Table 6.

90. The composition according to embodiment 1, wherein the composition comprises:
(a) Cetuximab-DBCO-C9-M30m3 (DAR3) and PEG12-Poly-(D-Arg)12;
(b) Cetuximab-DBCO-C4/P5-M30m3 (DAR3) and PEG12-Poly-(D-Arg)12;
(c) Cetuximab-DBCO-PEG9-M30m3 (DAR3) and PEG12-Poly-(D-Arg)12;
(d) Cetuximab-DBCO-PEG9-M30m3 (DAR2) and PEG12-Poly-(D-Arg)12;
(e) Cetuximab-DBCO-PEG9-M30m3 (DAR4) and PEG12-Poly-(D-Arg)12;
(f) Cetuximab-DBCO-PEG9-M30m3 (DAR6) and PEG12-Poly-(D-Arg)12;
(g) Cetuximab-Linear-PEG13-M30m3 (DAR4) and PEG12-Poly-(D-Arg)12;
(h) 3tf12-DBCO-PEG8-NCD5 and Poly(L-Arg)9;
(i) 3tf12-DBCO-PEG8-M30m3 and Poly(L-Arg)9;
(j) Fv55-SMCC-M30m3 and PEG12-Poly(L-Arg)12;
(k) Fv55-PEG30-M30m3 and PEG12-Poly(L-Arg)12;
(l) Cetuximab-PEG4-azide-DBCO-PEG5-M30m3 (DAR2) and PEG12PolyArg12{d};
(m) Cetuximab-PEG4-azide-DBCO-PEG5-M30m3 (DAR2) and PolyArg12Cbp3.9 kDa;
(n) Cetuximab-PEG4-azide-DBCO-PEG5-M30m3 (DAR4) and PEG12PolyArg12{d};
(o) Cetuximab-PEG4-azide-DBCO-PEG5-M30m3 (DAR4) and PolyArg12Cbp3.9 kDa;
(p) Cetuximab-PEG4-azide-DBCO-PEG5-M30m3 (DAR4) and PolyArg12C-PEG2000 Da;
(q) Cetuximab-PEG4-azide-DBCO-PEG5-M30m3 (DAR4) and PolyArg12C-PEG5000 Da;
(r) Cetuximab-PEG4-azide-DBCO-PEG5-M30m3 (DAR4) and PolyArg12C-Dextran5000 Da;
(s) Cetuximab-SMCC-M30m3 (DAR4) and PEG12PolyArg12{d};
(t) Cetuximab-MCVCPABcPNP-M30m3 (DAR4) and PEG12PolyArg12{d};
(u) Cetuximab-MCPEG4VCPABcPNP-M30m3 (DAR4) and PEG12PolyArg12{d};
(v) Cetuximab-PEG4-azide-DBCO-PEG5-M30m3 (DAR2.5) and PEG12PolyArg12{d};
(w) Cetuximab-PEG4-azide-DBCO-PEG5-M30m3 (DAR4.5) and PEG12PolyArg12{d};
(x) Cetuximab-PEG4-azide-DBCO-PEG5-M30m3 (DAR6.5) and PEG12PolyArg12{d};
(y) Cetuximab-C4(Azide-DBCO)C5-M30m3 and PEG12PolyArg12; or
(z) any of the antibody-polynucleotide conjugate and hybrid polymer combinations disclosed in Table 5.

91. A polynucleotide conjugate comprising a polynucleotide conjugated to a targeting molecule.

92. The polynucleotide conjugate according to embodiment 91, wherein the targeting molecule is an antibody or an antigen-binding fragment thereof, or a binding protein.

93. The polynucleotide conjugate according to embodiment 92, wherein the antibody or antigen-binding fragment thereof is selected from the group consisting of a monoclonal antibody, a bispecific antibody, a Fab, a Fab-Fc, a Fv, a single chain Fv (scFv), a diabody, a minibody, a vNAR, a Centyrin and an immunoglobulin single variable domain (ISV) such as an Nanobody® molecule.

94. The polynucleotide conjugate according to embodiment 93, wherein the bispecific antibody is a bispecific T-cell engager (BiTE) or a dual-affinity retargeting antibody (DART).

95. The polynucleotide conjugate according to embodiment 93, wherein the Nanobody® is a Nanobody-HSA®.

96. The polynucleotide conjugate according to any one of embodiments 92-95, wherein the antibody or antigen-binding fragment thereof is an IgG molecule or is derived from an IgG molecule.

97. The polynucleotide conjugate according to embodiment 96, wherein the IgG molecule is an IgG1 or an IgG4 molecule.

98. The polynucleotide conjugate according to embodiment 92, wherein the binding protein is a soluble receptor or a soluble ligand.

99. The polynucleotide conjugate according to embodiment 98, wherein the soluble receptor comprises the extracellular domain of a receptor.

100. The polynucleotide conjugate according to embodiment 98 or 99, wherein the soluble receptor is a Fc fusion protein.

101. The polynucleotide conjugate according to any one of embodiments 91-100, wherein the targeting molecule is a therapeutically active molecule or a biologically active molecule.

102. The polynucleotide conjugate according to any one of embodiments 91-101, wherein the polynucleotide is selected from the group consisting of a siRNA, an ncRNA mimic, a short-harpin RNA (shRNA), a dicer-dependent siRNA (di-siRNA), an antisense oligonucleotide (ASO), a gapmer, a mixmer, a double-stranded RNA (dsRNA), a single stranded RNAi, (ssRNAi), a DNA-directed RNA interference (ddRNAi), an RNA activating oligonucleotide (RNAa), an aptamer, an exon skipping oligonucleotide, a miRNA, a miRNA mimic, an mRNA, and a guide RNA.

103. The polynucleotide conjugate according to embodiment 102, wherein the polynucleotide is a miRNA mimic.

104. The polynucleotide conjugate according to embodiment 103, wherein the miRNA mimic mimics miR-30.

105. The polynucleotide conjugate according to embodiment 104, wherein the polynucleotide is miRNA mimic is selected from the group consisting of M30 ml, M30m2, M30m3, and M30m4.

106. The polynucleotide conjugate according to embodiment 105, wherein the polynucleotide is M30m3.

107. The polynucleotide conjugate according to embodiment 102, where in the polynucleotide is an ASO.

108. The polynucleotide conjugate according to embodiment 107, wherein the ASO is a DUX4-targeted ASO.

109. The polynucleotide conjugate according to embodiment 108, wherein the DUX4-targeted ASO is selected from the group consisting of the DUX4-targeted ASOs disclosed in Table 4.

110. The polynucleotide conjugate according to embodiment 109, wherein the DUX4-targeted ASO is selected from the group consisting of ASDX2, ASDX4, ASDX23, ASDX26 and ASDX32.

111. The polynucleotide conjugate according to any one of embodiments 101-110, wherein the targeting molecule and the polynucleotide result in a synergistic therapeutic or biological effect.

112. The polynucleotide conjugate according to any one of embodiments 91-111, wherein the polynucleotide is conjugated directly to the targeting molecule.

113. The polynucleotide conjugate according to any one of embodiments 91-111, wherein the polynucleotide is conjugated to the targeting molecule via a linker.

114. The polynucleotide conjugate according to embodiment 113, wherein the linker is a hydrophobic linker.

115. The polynucleotide conjugate according to embodiment 113, wherein the linker is a peptide linker.

116. The polynucleotide conjugate according to embodiment 113, wherein the linker is a chemical linker.

117. The polynucleotide conjugate according to embodiment 116, wherein the chemical linker is a polymeric linker.

118. The polynucleotide conjugate according to embodiment 116 or 117, wherein the chemical linker is linear.

119. The polynucleotide conjugate according to embodiment 116 or 117, wherein the chemical linker is cyclic.

120. The polynucleotide conjugate according to embodiment 117, wherein the polymeric linker comprises PEG, a sugar, a fatty acid, a phosphate, a pyrophosphate or a polysarcosine.

121. The polynucleotide conjugate according to embodiment 120, wherein the linker is a high molecular weight PEG linker.

122. The polynucleotide conjugate according to embodiment 120, wherein the linker is a low molecular weight PEG linker.

123. The polynucleotide conjugate according to any one of embodiments 113-122, wherein the linker is non-cleavable.

124. The polynucleotide conjugate according to any one of embodiment 113-122, wherein the linker is cleavable.

125. The polynucleotide conjugate according to embodiment 124, wherein the linker is cleavable in vivo.

126. The polynucleotide conjugate according to embodiment 124 or 125, wherein the cleavable linker is selected from the group consisting of a disulfide linker, a self-immolative peptide polymer hybrid, and a sulfatase-promoted arylsulfate linker.

127. The polynucleotide conjugate according to embodiment 126, wherein the self-immolative peptide polymer hybrid comprises glucuronic acid, para-amino-benzoyloxy (PAB), 7-amino-3-hydroxyethyl-coumarin (7-AHC), or Fe(II)-reactive 1,2,4-trioxolane scaffold (TRX).

128. The polynucleotide conjugate according to any one of embodiments 124-127, wherein the cleavable linker may be cleaved through reduction, hydrolysis, proteolysis, photo cleavage, chemical cleavage, enzymatic cleavage, and bio-orthogonal-cleavage.

129. The polynucleotide conjugate according to embodiment 128, wherein the chemical cleavage is by Fe II mediated R elimination of TRX.

130. The polynucleotide conjugate according to embodiment 128, wherein the enzymatic cleavage is by non-proteolytic sulfatase, β-galactosidase/glucuronidase or pyrophosphatase.

131. The polynucleotide conjugate according to embodiment 128, wherein the bio-orthogonal cleavage is by Cu I-BTTAA or free copper ion mediated cleavage.

132. The polynucleotide conjugate according to any one of embodiments 113-131, wherein the linker is conjugated to a lysine residue, a cysteine residue, histidine residue, or a non-natural amino acid residue in the targeting molecule.

133. The polynucleotide conjugate according to any one of embodiments 113-132, wherein the linker is conjugated to the targeting molecule by a chemical conjugation or an enzymatic conjugation.

134. The polynucleotide conjugate according to embodiment 133, wherein the chemical conjugation comprises acylation and click chemistry.

135. The polynucleotide conjugate according to embodiment 133, wherein the enzymatic conjugation is via a sortase or a transferase enzyme.

136. The polynucleotide conjugate according to any one of embodiments 91-135, wherein each targeting molecule is conjugated to between one and eight polynucleotide molecules (DAR 1-8).

137. The polynucleotide conjugate according to embodiment 136, wherein each targeting molecule is conjugated to one polynucleotide molecule (DAR 1).

138. The polynucleotide conjugate according to embodiment 136, wherein each targeting molecule is conjugated to two polynucleotide molecules (DAR 2).

139. The polynucleotide conjugate according to embodiment 136, wherein each targeting molecule is conjugated to three polynucleotide molecules (DAR 3).

140. The polynucleotide conjugate according to embodiment 136, wherein each targeting molecule is conjugated to four polynucleotide molecules (DAR 4).

141. The polynucleotide conjugate according to embodiment 136, wherein each targeting molecule is conjugated to five polynucleotide molecules (DAR 5).

142. The polynucleotide conjugate according to embodiment 136, wherein each targeting molecule is conjugated to six polynucleotide molecules (DAR 6).

143. The polynucleotide conjugate according to embodiment 136, wherein each targeting molecule is conjugated to seven polynucleotide molecules (DAR 7).

144. The polynucleotide conjugate according to embodiment 136, wherein each targeting molecule is conjugated to eight polynucleotide molecules (DAR 8).

145. The polynucleotide conjugate according to any one of embodiments 91-144, wherein the polynucleotide-conjugated targeting molecule has a molecular weight greater than 30 kDa.

146. The polynucleotide conjugate according to embodiment 145, wherein the polynucleotide-conjugated targeting molecule has a molecular weight greater than 40 kDa.

147. The polynucleotide conjugate according to embodiment 146, wherein the polynucleotide-conjugated targeting molecule has a molecular weight greater than 50 kDa.

148. The polynucleotide conjugate according to embodiment 147, wherein the polynucleotide-conjugated targeting molecule has a molecular weight greater than 60 kDa.

149. The composition according to any one of embodiments 91-148, wherein the polynucleotide-conjugated targeting molecule has a molecular weight no greater than 7,500 kDa.

150. The polynucleotide conjugate according to embodiment 91, wherein the polynucleotide conjugate is selected from the group consisting of Cetuximab-DBCO-C9-M30m3 (DAR3); Cetuximab-DBCO-C4/P5-M30m3 (DAR3); Cetuximab-DBCO-PEG9-M30m3 (DAR3); Cetuximab-DBCO-PEG9-M30m3 (DAR2); Cetuximab-DBCO-PEG9-M30m3 (DAR4); Cetuximab-DBCO-PEG9-M30m3 (DAR6); Cetuximab-Linear-PEG13-M30m3 (DAR4); Cetuximab-PEG4-azide-DBCO-PEG5-M30m3 (DAR1), Cetuximab-PEG4-azide-DBCO-PEG5-M30m3 (DAR2), Cetuximab-PEG4-azide-DBCO-PEG5-M30m3 (DAR4), Cetuximab-PEG4-azide-DBCO-PEG5-M30m3 (DAR2.5), Cetuximab-PEG4-azide-DBCO-PEG5-M30m3 (DAR4.5), Cetuximab-PEG4-azide-DBCO-PEG5-M30m3 (DAR6.5), Cetuximab-SMCC-M30m3 (DAR4) (SMCC), Cetuximab-MCVCPABcPNP-M30m3 (DAR4) (MCVCPABcPNP), Cetuximab-MCPEG4VCPABcPNP-M30m3 (DAR4) (MCPEG4VCPABcPNP), Cetuximab-C4-Azide-DBCO-C5-M30m3, Cetuximab-PEG4-azide-DBCO-PEG4-m30m3 (DAR4), Cetuximab-MC-PEG4-ValCit-PABc-M30m3 (DAR1), Cetuximab-MC-PEG4-ValCit-PABc-M30m3 (DAR2), Cetuximab-MC-PEG4-ValCit-PABc-M30m3 (DAR3), Cetuximab-MC-PEG4-ValCit-PABc-M30m3 (DAR4), 3tf12-DBCO-PEG8-NCD5 (DAR1); 3tf12-DBCO-PEG8-M30m3 (DAR1); Fv55-SMCC-M30m3 (DAR1); Fv55-PEG8-DBCO-M30m3 (DAR1), Fv55-PEG8-DBCO-M30m3 (DAR2), Fv55-linker-M30m3 (DAR2), Fv55-DBCO-PEG8-M30 ml(DAR1), Fv55-DBCO-PEG8-M30 ml(DAR2), and ASO-carbon4-DBCO-Carbon5-3tf12 (DAR1).

151. The polynucleotide conjugate according to embodiment 91, wherein the polynucleotide conjugate is selected from the group consisting for the antibody-polynucleotide conjugates disclosed in Table 5 or Table 6.

152. A method of treating a genetic disease in a subject in need thereof, the method comprising administering to the subject a therapeutically effective amount of the composition according to any one of embodiments 1-90 or the polynucleotide conjugate according to any one of embodiments 91-151.

153. The method according to embodiment 152, wherein the genetic disease is a viral infection.

154. The method according to embodiment 153, where in the viral infection is by a virus selected from the group consisting of an adenovirus, an anellovirus, an arenavirus, an astrovirus, a bunyavirus, a calicivirurs, a coronavirus, a filovirus, a flavivirus, a hepadnavirus, a herpesvirus, an orthomyxovirus, a papillomavirus, a paramyxovirus, a parvovirus, a picornavirus, a pneumovirus, a polyomavirus, a poxvirus, a reovirus, a retrovirus, a rhabdovirus, and a togavirus.

155. The method according to embodiment 154, wherein the virus is selected from the group consisting of Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

156. The method according to any one of embodiments 153-155, wherein the polynucleotide comprises a siRNA, a miRNA, a miRNA mimic, an ASO, or a guide RNA that targets a viral gene.

157. The method according to any one of embodiments 153-156, wherein the polynucleotide is conjugated to a targeting molecule that specifically binds to a viral protein or a protein on the surface of a host cell for the virus.

158. The method according to embodiment 157, wherein the polynucleotide and the targeting molecule synergize in the treatment of the viral infection.

159. The method according to embodiment 152, wherein the genetic disease is cancer.

160. The method according to embodiment 159, wherein the cancer is characterized by overexpression of an oncogene.

161. The method according to any embodiment 160, wherein the polynucleotide comprises a siRNA, a miRNA, a miRNA mimic, an ASO, or a guide RNA that targets the oncogene.

164. The method according to embodiment 159, wherein the cancer is characterized by reduced expression of a tumor suppressor gene.

163. The method according to embodiment 162, wherein the polynucleotide comprises a mRNA molecule encoding the tumor suppressor gene.

164. The method according to embodiment 162, wherein the polynucleotide comprises a guide RNA that that restores expression of the tumor suppressor gene.

165. The method according to any one of embodiments 159-166, wherein the polynucleotide is conjugated to a targeting molecule that specifically binds a tumor cell of the cancer.

166. The method according to embodiment 165, wherein the targeting molecule specifically binds epidermal growth factor receptor; and wherein the polynucleotide is a miR-30 miRNA or a mimic thereof.

167. The method according to embodiment 165, wherein the targeting molecule specifically binds TFR.

168. The method according to embodiment 167, wherein the targeting molecule is selected from the group consisting of FV55 scFv, Fv55 diabody, and 3TF12.

169. The method according to embodiment 165, wherein the targeting molecule specifically binds ACVR1; and wherein the polynucleotide is a miR-30 miRNA or a mimic thereof.

170. The method according to embodiment 165, wherein the targeting molecule specifically binds ACVR1; and wherein the polynucleotide is a miR-26 miRNA or a mimic thereof.

171. The method according to any one of embodiments 163-170, wherein the polynucleotide and the targeting molecule synergize in the treatment of the cancer.

172. The method according to embodiment 152, wherein the genetic disease is a neuromuscular disorder.

173. The method according to embodiment 172, wherein the neuromuscular disorder is a muscular dystrophy.

174. The method according to embodiment 173, wherein the muscular dystrophy is facioscapulohumeral muscular dystrophy (FSHD).

175. The method according to embodiment 174, wherein the polynucleotide comprises a siRNA, a miRNA, a miRNA mimic, an ASO, or a guide RNA that targets DUX4, DMPK or CAPN3.

176. The method according to embodiment 175, wherein the polynucleotide is an ASO that targets DUX4.

177. The method according to embodiment 176, wherein the DUX4-targeted ASO is selected from the group consisting of the DUX4-targeted ASOs disclosed in Table 4.

178. The composition according to embodiment 177, wherein the DUX4-targeted ASO is selected from the group consisting of ASDX2, ASDX4, ASDX23, ASDX26 and ASDX32.

179. The method according to embodiment 173, wherein the muscular dystrophy is Duchenne muscular dystrophy.

180. The method according to embodiment 179, wherein the polynucleotide is a mRNA, a cDNA, or a vector encoding dystrophin or utrophin.

181. The method according to embodiment 178, wherein the polynucleotide is a guide RNA that restores the expression of dystrophin or utrophin.

182. The method according to any one of embodiments 173-181, wherein the polynucleotide is conjugated to a targeting molecule that specifically binds a marker on the surface of a skeletal muscle cell of the subject.

183. The method according to embodiment 182, wherein the targeting molecule specifically binds ACVR1.

184. The method according to embodiment 183, wherein the targeting molecule specifically binds ACVR1; and wherein the polynucleotide is a DUX4-targeted ASO.

185. The method of embodiment 184, wherein the polynucleotide and the targeting molecule synergize in the treatment of the muscular dystrophy.

186. An antibody or an antigen-binding fragment thereof that specifically binds human transferrin receptor (TfR1), wherein the antibody or antigen-binding fragment thereof comprises a heavy chain variable region (VH) comprising the amino acid sequence QVQVQDSGGELVQPGGSLRVSCKASGFNIKDSYMHWVRQAPGKGLEWVAFIDPET GNTYYADSVKGRFTISRDNSKNTLYLQMNSLRAEDTAVYYCARGSIYWYFDVWGK GTTVTVSS (SEQ ID NO: 1) and a light chain variable region (VL) comprising the amino acid sequence DIQMTQSPSSLSASVGQRVTITCRASQSLLNSSNQKNSLGWYQQKPGKAPKLLIYFAS TRQSGVPSRFSGSGSGTDFTLTISSLQPEDFATYYCQQHYSTPLTFGQGTKVDIKRC (SEQ ID NO: 2).

187. The antibody or antigen-binding fragment thereof according to embodiment 186, wherein the antibody or antigen-binding fragment thereof is selected from the group consisting of a full-length antibody, a Fab, a Fab-Fc, a Fv, a single chain Fv (scFv), a diabody, a minibody, and an immunoglobulin single variable domain (ISV) such as an Nanobody® molecule.

188. The antibody or antigen-binding fragment thereof according to embodiment 187, wherein the antibody or antigen-binding fragment thereof is a scFv.

189. The antibody or antigen-binding fragment thereof according to embodiment 187, wherein the antibody or antigen-binding fragment thereof is a diabody.

190. The antibody or antigen-binding fragment thereof according to any one of embodiments 186-189, wherein the VH and VL are connected a linker.

191. The antibody or antigen-binding fragment thereof according to embodiment 190, wherein the linker comprises the amino acid sequence GGGGS (SEQ ID NO: 3).

192. The antibody or antigen-binding fragment thereof according to embodiment 190, wherein the linker comprises the amino acid sequence (GGGGS)_{N} (SEQ ID NO: 3), wherein N is 1-3.

193. The antibody or antigen-binding fragment thereof according to embodiment 188, wherein the VH and VL are connected by a linker, wherein the linker comprises the amino acid sequence (GGGGS)₃ (SEQ ID NO: 3).

194. The antibody or antigen-binding fragment thereof according to embodiment 189, wherein the VH and VL are connected by a linker, wherein the linker comprises the amino acid sequence (GGGGS)_{N} (SEQ ID NO: 3), wherein N is 1 or 2.

### EXAMPLES

The following Examples are provided merely for purposes of illustrating certain aspects and embodiments and are not intended to limit the invention in any way.

### Example 1

### A. Structures of the Hybrid Polymers and Hybrid Polymer Coated Antibody/Ligand-Polynucleotide Conjugates

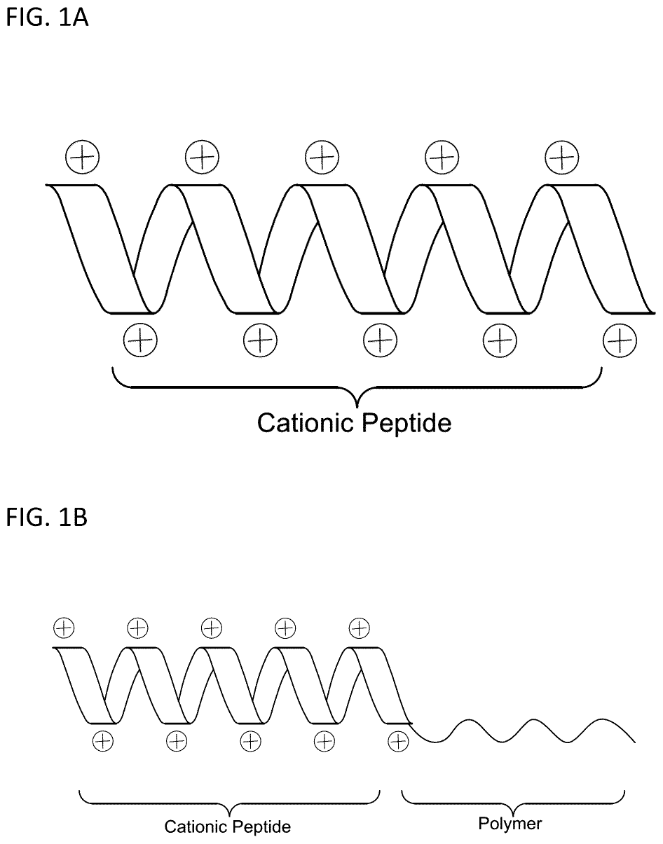

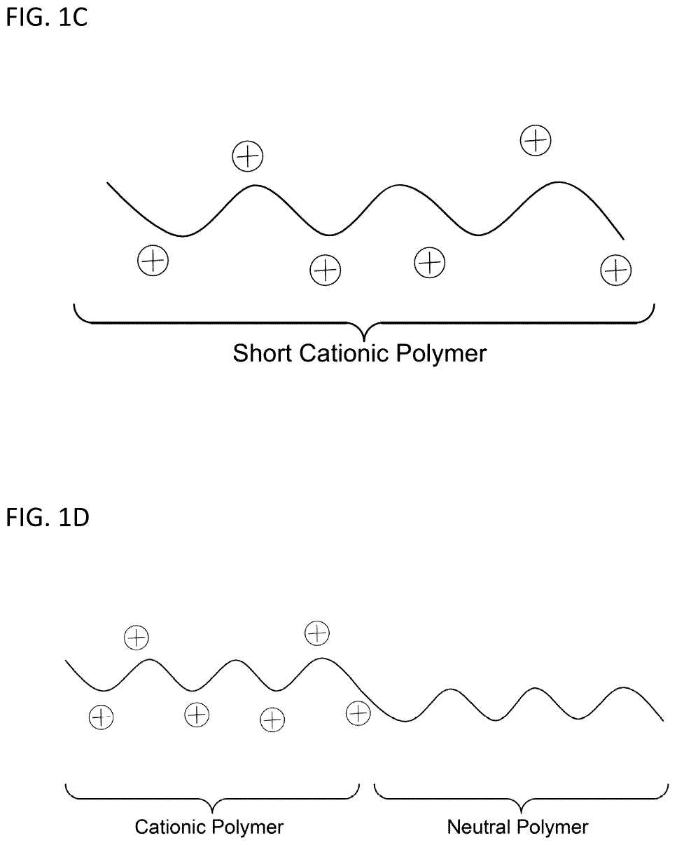

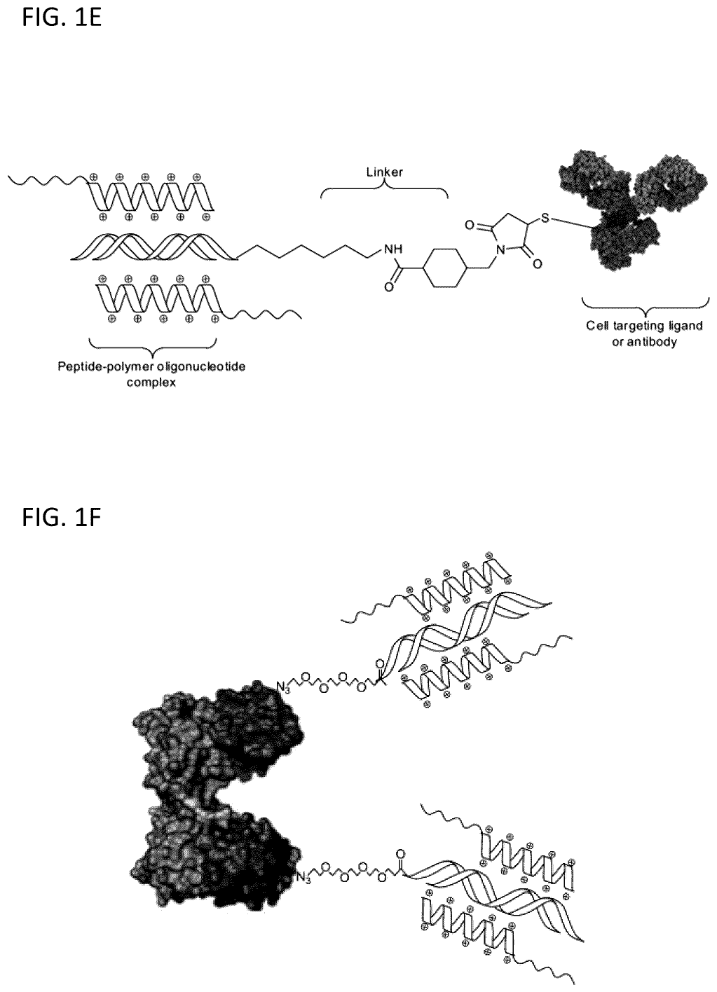

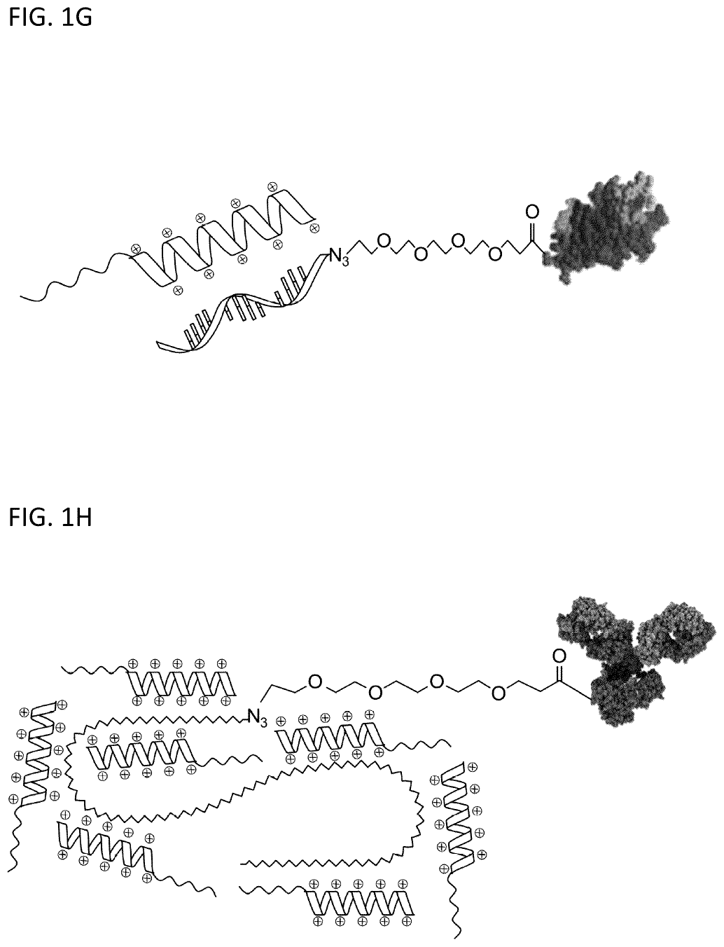

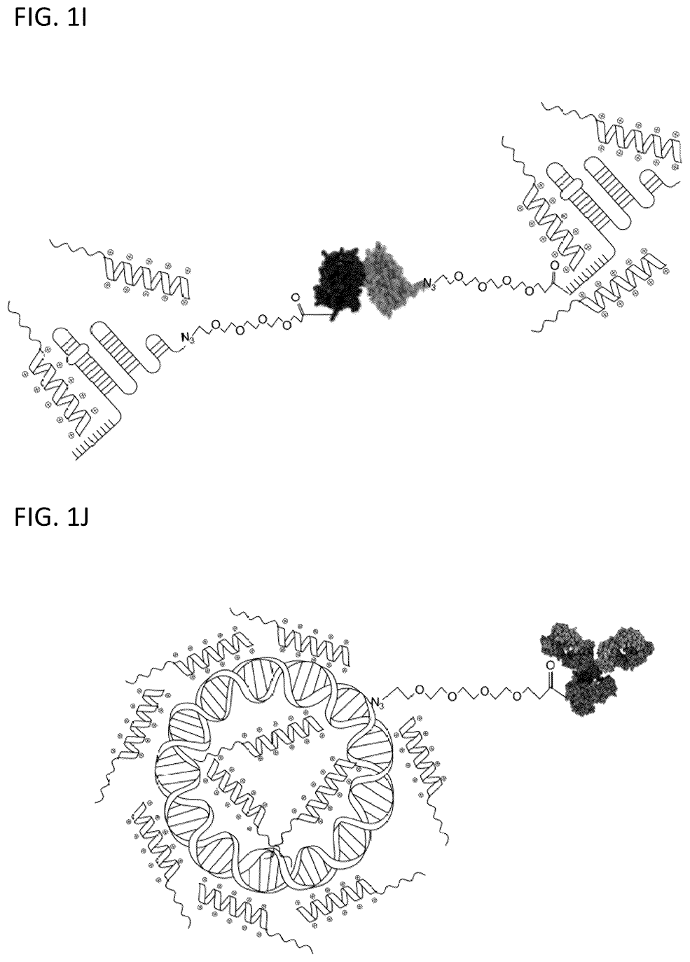

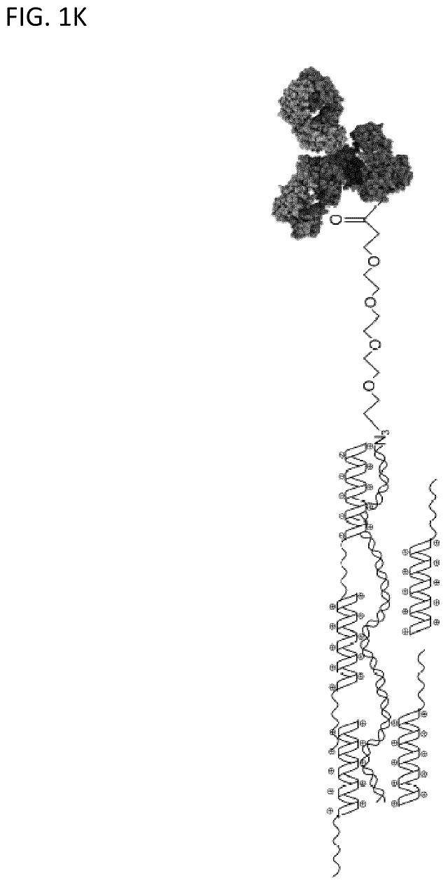

Various exemplary structures of hybrid polymers, which are capable of coating and stabilizing polynucleotides in antibody/ligand-polynucleotides conjugates at low cation/anion ratios without forming nanoparticles or generating aggregation and may be prepared as described herein are set forth in FIG. 1 (Panels A-D). They include (1) a cationic polypeptide (FIG. 1, Panel A); (2) a hybrid molecule comprising a cationic polypeptide and a neutral polymer (FIG. 1, Panel B); (3) a cationic polymer (FIG. 1, Panel C); and (4) a hybrid molecule comprising a cationic polymer and a neutral polymer (FIG. 1, Panel D).

Exemplary structures of antibody/ligand-polynucleotide conjugates which may be coated with hybrid polymers and used as targeted polynucleotide therapy complexes and may be prepared as described herein are set forth in FIG. 1 (Panels E-I). They include (1) antibody-oligonucleotide conjugate (FIG. 1, Panel E); (2) diabody-siRNA conjugate (FIG. 1, Panel F); (3) nanobody-antisense oligonucleotide (ASO) conjugate (FIG. 1, Panel G); (4) antibody-mRNA conjugate (FIG. 1, Panel H); and (5) cytokine-gRNA conjugate (FIG. 1, Panel I).

### B. Targeting Molecules Used in the Exemplified Conjugates

1. FV55: a monospecific scFv that that binds to human transferrin receptor (TfR1). The CDRs are identical to HB21 (Haynes B F et al., Characterization of a monoclonal antibody (5E9) that defines a human cell surface antigen of cell activation. J Immunol. 1981; 127:347-351). The heavy chain variable region (VH) of FV55 comprises the amino acid sequence (CDRs underlined):

(SEQ ID NO: 1)
QVQVQDSGGELVQPGGSLRVSCKASGFNIKDSYMHWVRQAPG
KGLEWVAFIDPETGNTYYADSVKGRFTISRDNSKNTLYLQMN
SLRAEDTAVYYCARGSIYWYFDVWGKGTTVTVSS.

The light chain variable region (VL) of FV55 comprises the amino acid sequence (CDRs underlined):

(SEQ ID NO: 2)
DIQMTQSPSSLSASVGQRVTITCRASQSLLNSSNQKNSLGWY
QQKPGKAPKLLIYFASTRQSGVPSRFSGSGSGTDFTLTISSL
QPEDFATYYCQQHYSTPLTFGQGTKVDIKRC.

The FV55 scFv is oriented VH-VL connected by (G4S)₃ (SEQ ID NO: 3) linker and has a c-terminal cysteine for conjugation. The Molecular weight of FV55 is about 26.5 kDa.

2. FV55 Diabody comprises the same sequence as the monospecific scFv FV55, but the linker is (G4S)_{N} (SEQ ID NO: 3), where N is 1 or 2. The molecular weight of FV55 Diabody is about 53 kDa.

3. 3TF12 is a monospecific scFv that binds to human transferrin receptor (TfR1). It is oriented VH-VL connected by (G4S)_{N}. When N is 3 (SEQ ID NO: 3), 3TF12 is a monomeric scFv. When N is 1 (SEQ ID NO: 3), 3TF12 dimerizes to form a diabody. (Ronan Crepin et al., Development of Human Single-Chain Antibodies to the Transferrin Receptor that Effectively Antagonize the Growth of Leukemias and Lymphomas; Cancer Research, Jun. 8, 2010).

4. Cetuximab is a chimeric (mouse/human) monoclonal antibody and an epidermal growth factor receptor (EGFR) inhibitor medication used for the treatment of metastatic colorectal cancer and head and neck cancer. It has a molecular weight of 145,781.92 g/mol.

### Example 2. Synthetic Scheme of Antibody-Polynucleotide Conjugate Via Click Chemistry

The general synthetic chemistry for preparing an antibody-polynucleotide conjugate with a heterocyclic PEG linker using click chemistry is set forth in FIG. 2A. Briefly, the method includes (a) conjugating the NHS ester of the PEG-azide linker to the antibody via Lysine acylation; (b) conjugating the NHS ester of the PEG DBCO linker to the polynucleotide (e.g., miRNA) duplex via amine acylation; and (c) coupling of the conjugated antibody and polynucleotide (e.g., miRNA) duplex via the click chemistry to produce the antibody-polynucleotide conjugate.

### Example 3. Synthetic Scheme of Antibody-Polynucleotide Conjugate Via Acylation Chemistry

The general synthetic chemistry for preparing an antibody-polynucleotide conjugate with a linear PEG linker via acylation chemistry is set forth in FIG. 2B. Briefly, the method includes (a) conjugating the PFP ester of the bis-functional linear PEG linker to the polynucleotide (e.g., miRNA) duplex via selective amine acylation; and (b) conjugating the remaining PFP ester of the intermediate polynucleotide (e.g., miRNA)-PEG linker to the antibody via Lysine acylation to produce the antibody-polynucleotide conjugate.

### Example 4. Synthetic Scheme of Antibody-Polynucleotide Conjugate Via Site-Specific Cysteine Conjugation with a Cleavable Linker

The general synthetic chemistry for preparing an antibody-polynucleotide conjugate with a cleavable linker via site-specific Cysteine conjugation is set forth in FIG. 2C. Briefly, the method includes (a) conjugating the MC-Val-Cit-PAB-PNP linker to a polynucleotide (e.g., miRNA) duplex via nucleophilic amine displacement; (b) selective reduction of the interchain Cysteine disulfide bonds of the antibody; and (s) site-specific conjugation of the Cysteine thiol group to the maleimide group of the polynucleotide (e.g., miRNA)-Val-Cit-PAB linker to produce the antibody-polynucleotide conjugate.

### Example 5. Manufacturing Process of a Representative Hybrid Polymer Coated Polynucleotide-Antibody Conjugate Complex

The general process for manufacturing hybrid polymer coated antibody polynucleotide conjugate complexes is set forth in FIG. 2D.I-III. Briefly, the method includes (a) conjugating the NHS ester of the PEG-azide linker to the antibody via Lysine acylation followed by purification; (b) conjugating the NHSS ester of the PEG-DBCO linker to the polynucleotide (e.g., miRNA) duplex via amine acylation followed by purification; (c) coating the DBCO linked polynucleotide (e.g., miRNA) duplex with the hybrid polymers via complexation; (d) coupling the conjugated antibody and polynucleotide (e.g., miRNA) duplex via click chemistry followed by purification to produce the hybrid polymer coated antibody-oligonucleotide conjugate.

### Example 6. Purification of Polynucleotide-ScFv Conjugates

FV55 diabody was expressed in E. coli and purified by affinity chromatography (protein L-agarose) followed by SEC (Superdex 75) (See FIG. 3A). Click reactions described above were performed using an excess of miRNA to form FV55-DBCO-PEG8-miRNA conjugate. The final conjugate was purified from unreacted miRNA by SEC using PBS as a mobile phase (see FIG. 3B).

### Example 7. Purification of mAb-Oligo Conjugates

Cetuximab-PEG4(DBCO-azide)PEG5-M30m3 (Avg. DAR 3) and Cetuximab-Carbon4(DBCO-azide)Carbon5-M30m3 (Avg. DAR 3) were synthesized as described above and then purified using SEC Superdex 75 (See FIG. 4A). In brief, excess linker was used to conjugate an azide containing linker to the antibody and an excess of linker was used to conjugate a DBCO containing linker to the oligo. Reactions were purified via dialysis and then clicked together for 72 hours at 4 degrees using an excess of oligo-DBCO vs Antibody-azide. Separation of excess oligo was achieved above using PBS as mobile phase. See FIG. 4B.

### Example 8. Purification of Multiple DAR mAb-miRNA Conjugates

Cetuximab-PEG5(azide-DBCO)Carbon4-M30 ml (Avg. DAR 3) were synthesized as described above and purified using AEX QA (Hi Trap). See FIG. 5. In brief, excess linker was used to conjugate an azide containing linker to the antibody and an excess of linker was used to conjugate a DBCO containing linker to the oligo. Reactions were purified via dialysis and then clicked together for 72 hours at 4 degrees using an excess of oligo-DBCO vs Antibody-azide. Separation of conjugate based on DAR was achieved by stepwise increase in salt concentrations after loading of conjugate onto Q AEX column. The final fraction contains a mixture of high DAR conjugate and unbound oligo.

### Example 9. Effect of polyArg12 and PEG-polyArg12 on TfR-targeting conjugates

Fv55-DBCO-PEG8 miRNA-AF488 conjugates were mixed with a molar excess of polyArg12 for 10 minutes. FIG. 6A displays a sample observation of aggregation of conjugate peptide complexes observed by microscopy at 100× magnification. FIG. 6B provides observations of non-aggregated and aggregated complexes. There was a significant increase in precipitation of the conjugate with increased polyArg12. This was not observed when complexed with PEG-polyArg12. Arg12-coated Fv55-DBCO-PEG8 miRNA-AF488 conjugates aggregated at N⁺:O⁻ ratio 1:1 and 2:1 (FIG. 6A and FIG. 6B). In contrast, PEG12Arg12-coated Fv55-DBCO-PEG8 miRNA-AF488 conjugates did not aggregate or precipitate at N⁺:O⁻ ratio 1:1 and 2:1 (FIG. 6B).

### Example 10. Effect of Hybrid Polymers on Cetux-PEG4-Azide-DBCO-PEG5-M30m3 Conjugates

Cetux-PEG4-Azide-DCBO-PEG5-M30m3 (DAR2) was mixed in water with the cationic peptides or hybrid polymers in Table 7, infra, at either a 1:1 or 1:2−charge/+charge ratio. Samples were then spun at 15,000g for 15 min and the presence of precipitate was noted by visual inspection and microscopy at 100× or 200× magnification was used to detect smaller aggregates, an example of which is displaying in FIG. 6A.

Cetux-PEG4-Azide-DCBO-PEG5-M30m3 (DAR2) conjugates coated with polyR9-cys, polyR12-cys, and polyR18-cys at a charge ratio (N/P) of 1 and Cetuximab-PEG9-DBCO-M30m3 conjugates coated with polyR9, polyR9-cys, polyR12, polyR12-cys, and polyR18-cys at a charge ratio (N/P) of 2 precipitated, whereas Cetuximab-PEG9-DBCO-M30m3 conjugates coated with PEG-polyR did not, indicating that adding a neutral polymer to the polyarginine helps to avoid aggregation of the coated conjugates. No differences were observed at various charge ratios (i.e., if a particular complex did not precipitate at a 1:1 charge ratio, then it did not precipitate at a 2:1 charge ratio and if a particular complex precipitated, then it did so at both charge ratios). See Table 7. (Y=Precipitated or Aggregated, N=Did not precipitate or aggregate, *=Was not tested)

TABLE 7
Precipitation with Peptide Library

| | | Molecular | | Precipitated |
|---|---|---|---|---|
| | | Weight | | or |
| # | Peptide Name | (g/mol) | Sequence | Aggregated |
| 1 | PEG12PolyArg12{d} | 2491 | [PEG12]RdRdRdRdRdRdRdRdRdRdRd | N |
| | | | Rd (SEQ ID NO: 21) | |
| 2 | PEG12PolyArg12PEG | 3090 | [PEG12]RRRRRRRRRRRR[PEG12] | * |
| | 12 | | (SEQ ID NO: 115) | |
| 3 | PolyArg12Ginsert | 2234.6 | RGRRGRRGRRGRRGRRGR (SEQ ID | Y |
| | | | NO: 116) | |
| 4 | PolyArg12Qinsert | 2661.0512 | RQRRQRRQRRQRRQRRQR (SEQ ID | Y |
| | | | NO: 117) | |
| 5 | PolyArg12Kinsert | 2661.3116 | RKRRKRRKRRKRRKRRKR (SEQ ID | Y |
| | | | NO: 118) | |
| 6 | PolyArg12Sinsert | 2414.7356 | RSRRSRRSRRSRRSRRSR (SEQ ID | Y |
| | | | NO: 119) | |
| 7 | PolyArg12Tinsert | 2498.897 | RTRRTRRTRRTRRTRRTR (SEQ ID | Y |
| | | | NO: 120) | |
| 8 | PolyArg12Winsert | 3009.5462 | RWRRWRRWRRWRRWRRWR (SEQ | Y |
| | | | ID NO: 121) | |
| 9 | S3PolyArg12 | 2153.5009 | SSSRRRRRRRRRRRR (SEQ ID NO: | Y |
| | | | 122) | |
| 10 | S6PolyArg12 | 2414.7356 | SSSSSSRRRRRRRRRRRR (SEQ ID | Y |
| | | | NO: 123) | |
| 11 | S9PolyArg12 | 2675.9702 | SSSSSSSSSRRRRRRRRRRRR (SEQ | Y |
| | | | ID NO: 124) | |
| 12 | S3PolyArg12S3 | 2414.7355 | SSSRRRRRRRRRRRRSSS (SEQ ID | Y |
| | | | NO: 125) | |
| 13 | T6PolyArg12 | 2498.897 | TTTTTTRRRRRRRRRRRR (SEQ ID | Y |
| | | | NO: 126) | |
| 14 | PolyArg6 | 955.1407 | RRRRRR (SEQ ID NO: 127) | N |
| 15 | PEG12PolyArg6 | 1554 | [PEG12]RRRRRR (SEQ ID NO: 128) | N |
| 16 | PEG12PolyArg6C | 1657.3 | [PEG12]RRRRRRC (SEQ ID NO: 129) | N |
| 17 | PolyArg12C | 1995.4111 | RRRRRRRRRRRRC (SEQ ID NO: | Y |
| | | | 130) | |
| 18 | PolyArg11 | 1736.0787 | RRRRRRRRRRR (SEQ ID NO: 131) | Y |
| 19 | PEG24PolyArg12C | 3194.84 | [PEG12][PEG12]RRRRRRRRRRRRC | N |
| | | | (SEQ ID NO: 30) | |
| 20 | PEP24PolyArg12 | 3091.69 | [PEG12][PEG12]RRRRRRRRRRRR | N |
| | | | (SEQ ID NO: 27) | |
| 21 | PEG24PolyArg9 | 2623.13 | [PEG12][PEG12]RRRRRRRRR (SEQ | N |
| | | | ID NO: 28) | |
| 22 | PolyArg12C- | 3995.4 | RRRRRRRRRRRRC-[Maleimide- | N |
| | PEG2000Da | | PEG2000] (SEQ ID NO: 132) | |
| 23 | PolyArg12C- | 6995.4 | RRRRRRRRRRRRC-[Maleimide- | N |
| | PEG5000Da | | PEG5000] (SEQ ID NO: 133) | |
| 24 | PolyArg12C- | 6995.4 | RRRRRRRRRRRRC- | * |
| | QuadPeg5000Da | | [QuadPEG5000da] (SEQ ID NO: 134) | |
| 25 | PolyArg12C- | 6995.4 | RRRRRRRRRRRRC-[Maleimide- | N |
| | Dextran5000Da | | Dextran5k] (SEQ ID NO: 135) | |
| 26 | PolyArg15 | 2360.8292 | RRRRRRRRRRRRRRR (SEQ ID NO: | Y |
| | | | 29) | |
| 27 | PEG12PolyArg12 | 2491 | [PEG12]RRRRRRRRRRRR (SEQ ID | N |
| | | | NO: 22) | |
| 28 | PEG12PolyArg18 | 3428 | [PEG12]RRRRRRRRRRRRRRRRRR | Y |
| | | | (SEQ ID NO: 31) | |
| 29 | PEG12PolyArg15 | 2959 | [PEG12]RRRRRRRRRRRRRRR (SEQ | Y |
| | | | ID NO: 32) | |
| 30 | PolyArg9 | 1423.7035 | RRRRRRRRR (SEQ ID NO: 136) | Y |
| 31 | PolyArg9C | 1526.8483 | RRRRRRRRRC (SEQ ID NO: 137) | Y |
| 32 | PEG12PolyArg9d | 2022 | [PEG12]RdRdRdRdRdRdRdRdRd | N |
| | | | (SEQ ID NO: 23) | |
| 33 | PolyArg18C | 2932.5368 | RRRRRRRRRRRRRRRRRRC (SEQ ID | Y |
| | | | NO: 26) | |
| 34 | PolyArg12 | 1893 | RRRRRRRRRRRR (SEQ ID NO: 25) | Y |
| 35 | PEG12PolyArg12C | 2594 | [PEG12]RRRRRRRRRRRRC (SEQ ID | Y |
| | | | NO: 33) | |
| 36 | PEG1000DaPolyArg12 | 2893 | [PEG1000]-RRRRRRRRRRRR (SEQ | N |
| | | | ID NO: 138) | |
| 37 | PEG2000DaPolyArg12 | 3893 | [PEG2000]-RRRRRRRRRRRR (SEQ | N |
| | | | ID NO: 139) | |
| 38 | PEG5000DaPolyArg12 | 6893 | [PEG5000]-RRRRRRRRRRRR (SEQ | N |
| | | | ID NO: 140) | |
| 39 | CPolyArg12C | 2097 | CRRRRRRRRRRRRC (SEQ ID NO: | Y |
| | | | 141) | |
| 40 | PolyArg12Cbp1.5kDa | 3393 | RRRRRRRRRRRRC-[Maleimide- | N |
| | | | BranchedPEG1.5kda] (SEQ ID NO: | |
| | | | 142) | |
| 41 | PolyArg12Cbp3.9kDa | 5793 | RRRRRRRRRRRRC-[Maleimide- | N |
| | | | BranchedPEG3.9kda] (SEQ ID NO: | |
| | | | 143) | |
| 42 | PolyArg12Cbp16kDa | 17793 | RRRRRRRRRRRRC-[Maleimide- | N |
| | | | BranchedPEG16kda] (SEQ ID NO: 144) | |
| 43 | CPolyArg12Cbp1.5kDa | 5097 | [Maleimide-BranchedPEG1.5kda]- | N |
| | | | CRRRRRRRRRRRRC-[Maleimide- | |
| | | | BranchedPEG1.5kda] (SEQ ID NO: | |
| | | | 145) | |
| 44 | PolyArg12Cbp2kDa | 4355 | RRRRRRRRRRRRC-[Maleimide- | N |
| | | | BranchedPEG2kda] (SEQ ID NO: 146) | |
| 45 | PolyArg12bp2kDa | 4198 | RRRRRRRRRRRR-[NHS Ester- | N |
| | | | BranchedPEG2kda] (SEQ ID NO: 147) | |

Table 8 provides precipitation results, CAS numbers and suppliers of some cationic polymers used herein. Cetux-PEG4-Azide-DCBO-PEG5-M30m3 (DAR2) was mixed in water with the cationic polymers at either a 1:1 or 1:2−charge/+ charge ratio. Samples were then spun at 15,000 g for 15 min and the presence of precipitate or aggregate was noted. See Table 8. (Y=Precipitated, N=Did not precipitate, *=Was not tested) No differences were observed at various charge ratios (i.e., if a particular complex did not precipitate at a 1:1 charge ratio, then it did not precipitate at a 2:1 charge ratio and if a particular complex precipitated, then it did so at both charge ratios).

TABLE 8
Precipitation with polymers

| | | | Supplier, | Precipitated/ |
|---|---|---|---|---|
| # | Sample ID | CAS# | Catalog Number | Aggregated |
| 1 | No Polymer | | | N |
| 2 | PEG12PolyArg12 | | miRecule | Y |
| 3 | PEI 2 kda | 9002-98-6 | Sigma, 408700 | Y |
| 4 | Branched PEI | 25987-06-8 | Sigma, 408719 | Y |
| 5 | Acelated | | Sigma, 913235 | Y |
| | Branched PEI | | | |
| 6 | Amide Dextran | | Nanocs, DX5-AM-1 | N |
| 7 | Lysine Dextran | | Nanocs, DX5-LS-1 | N |
| 8 | Spermine | 71-44-3 | Sigma, S4264 | N |
| 9 | Agmatine* | 2482-00-0 | Sigma, A7127-1G | N |
| 10 | PEG PEI 15 kda | | Sigma, 910791 | N |
| 11 | BPEI-G-PEG 550 | | Sigma, 900926 | N |
| 12 | BPEI-G-PEG | | Sigma, 900743 | N |
| | 5000 | | | |
| 13 | Arginine* | 74-79-3 | Sigma, A1270000 | N |

*Agmatine and Arginine were included as negative controls for precipitation.

### Example 11. Hybrid Polymer Coated scFV-Polynucleotide Conjugates Retain Antigen-Binding Function

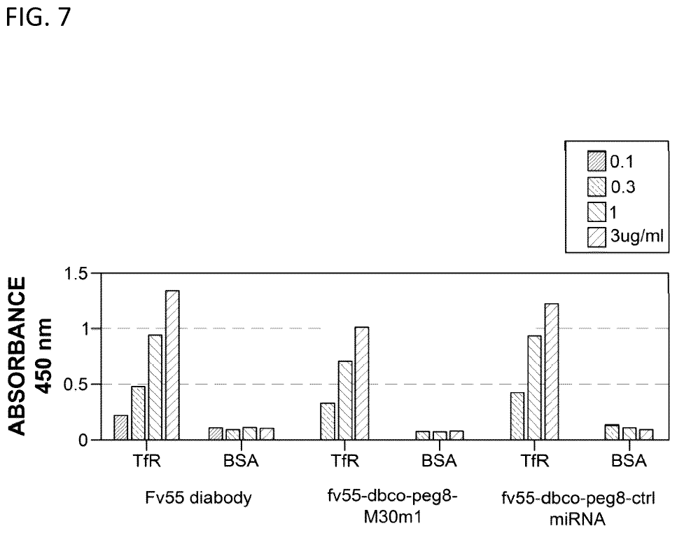

The FV55 diabody was conjugated to either control miRNA or M30 ml via click chemistry as described above to form FV55-DBCO-PEG8-miRNA conjugates. ELISA assay was performed to confirm that FV55-DBCO-PEG8-miRNA conjugates retained TfR-binding function. Briefly, wells coated with either 1 g TfR or BSA were incubated with FV55 or FV55-DBCO-PEG8-miRNA conjugate at 0.1, 0.3, 1, and 3 g/mL. Samples were incubated for 3 hours at room temperature, washed with PBST, and incubated for an additional hour with proteinL-HRP, which binds FV55. Wells were washed with PBST, incubated with TMB substrate and quenched with HCl. The absorbance at 450 nm was measured, and the conjugates showed the same dose-dependent signal specific for TfR as the parental Fv55 diabody. See FIG. 7.

### Example 12. Hybrid Polymer Coated mAb-miRNA Conjugates Retain Antigen-Binding Function

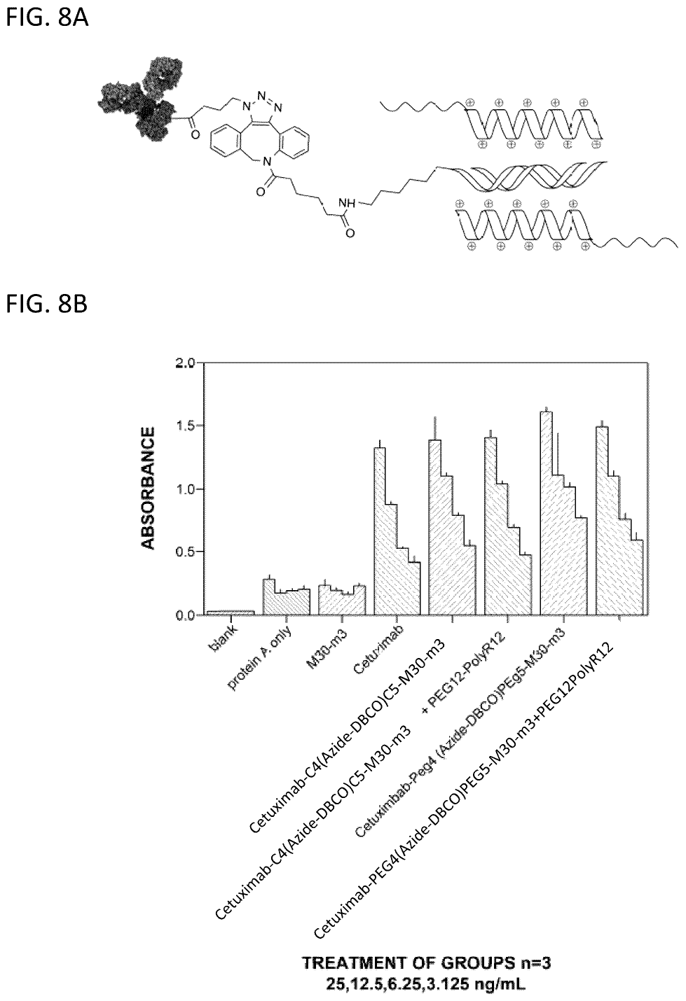

Cetuximab-Linker-M30m3 Conjugates (FIG. 8A) were assayed for activity via ELISA with and without peptide-polymer addition. In brief, 0.1 g/well of EGFR or BSA (Control) was coated onto nunc maxisorb plates for 24 hours at 4 degrees. Plates were blocked with 5% milk in PBS 0.1% tween for 2 hours at RT. Plates were washed then treated with unmodified cetuximab (Positive control), protein A HRP alone (negative control) or Cetuximab with M30m3 conjugated using two different linkers with or without PEG12-PolyR12 polymer at 25, 12.5, 6.25 and 3.125 ng/mL (n=3). After 1 hour Protein-A-HRP was added to the wells at 10 ug/mL. After 1 hour plate was washed 3× with PBS 0.1% tween. TMB reagent was added and reaction was stopped with HCL after 10m. Data as reported by +/−SEM for 3 replicates. Data suggests all conjugates (in both the presence of and absence of a hybrid polymer) retain full binding activity of unmodified cetuximab. See FIG. 8B.

### Example 13. Serum Stability of miRNA Mimic+Peptide

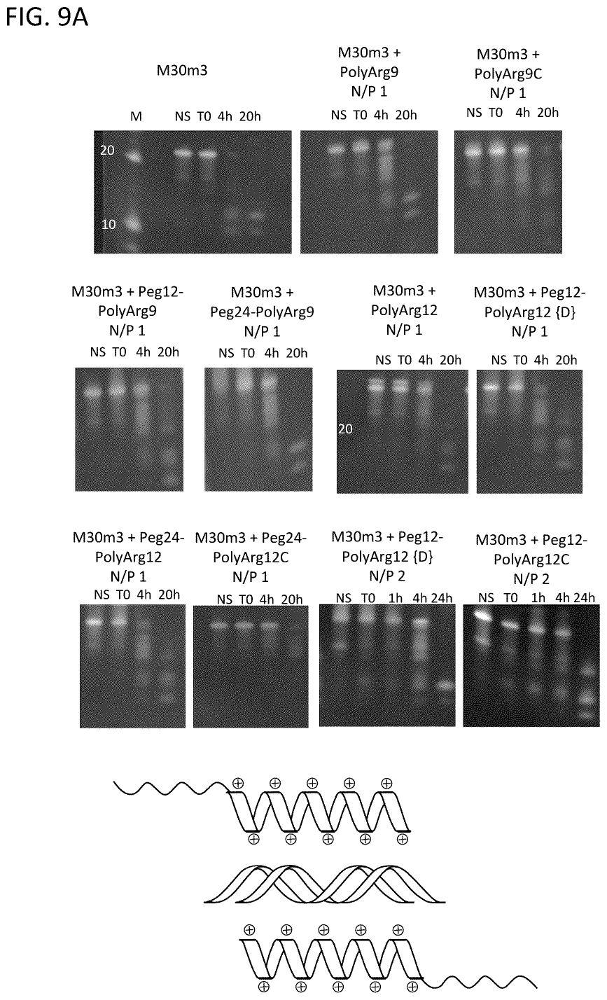

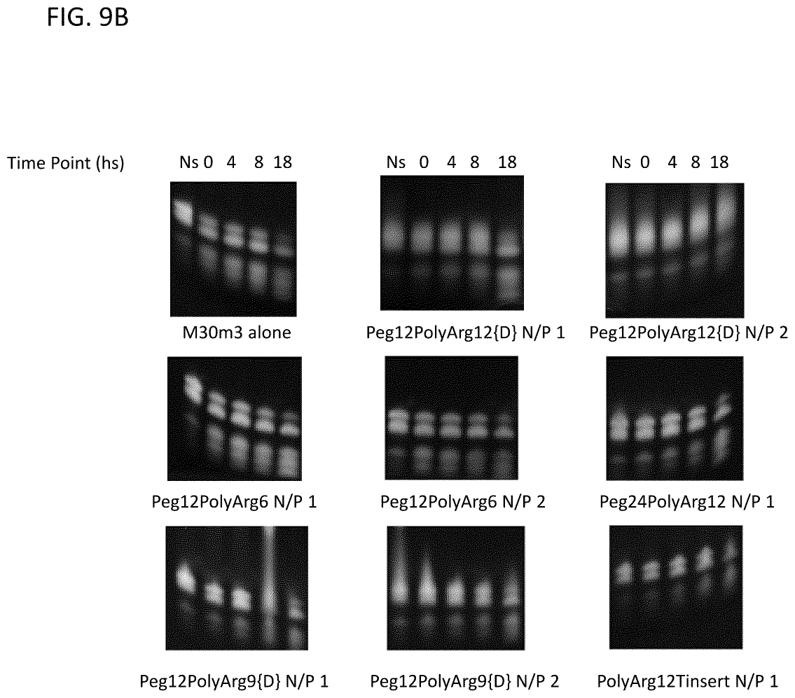

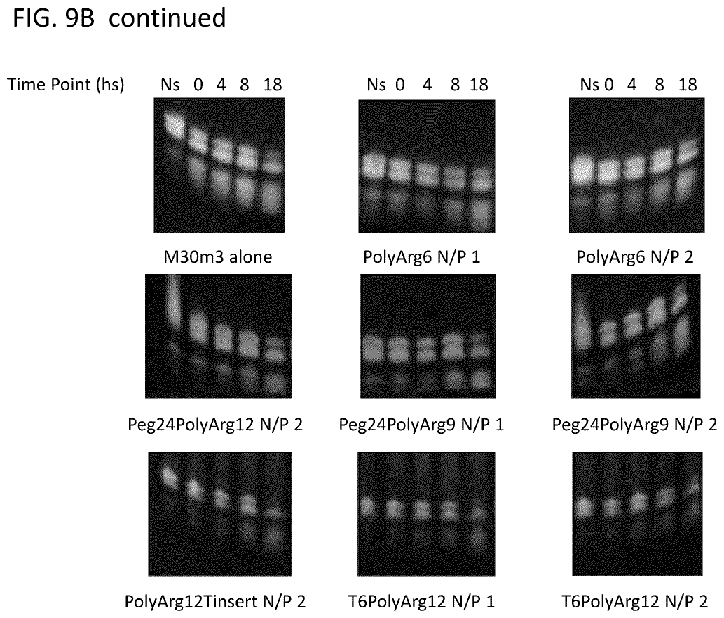

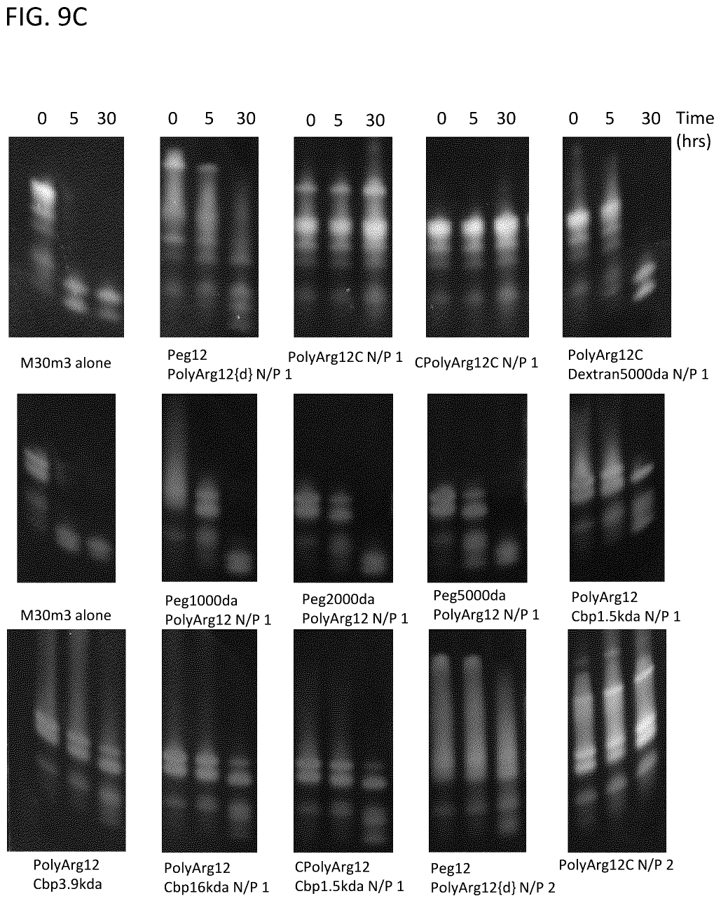

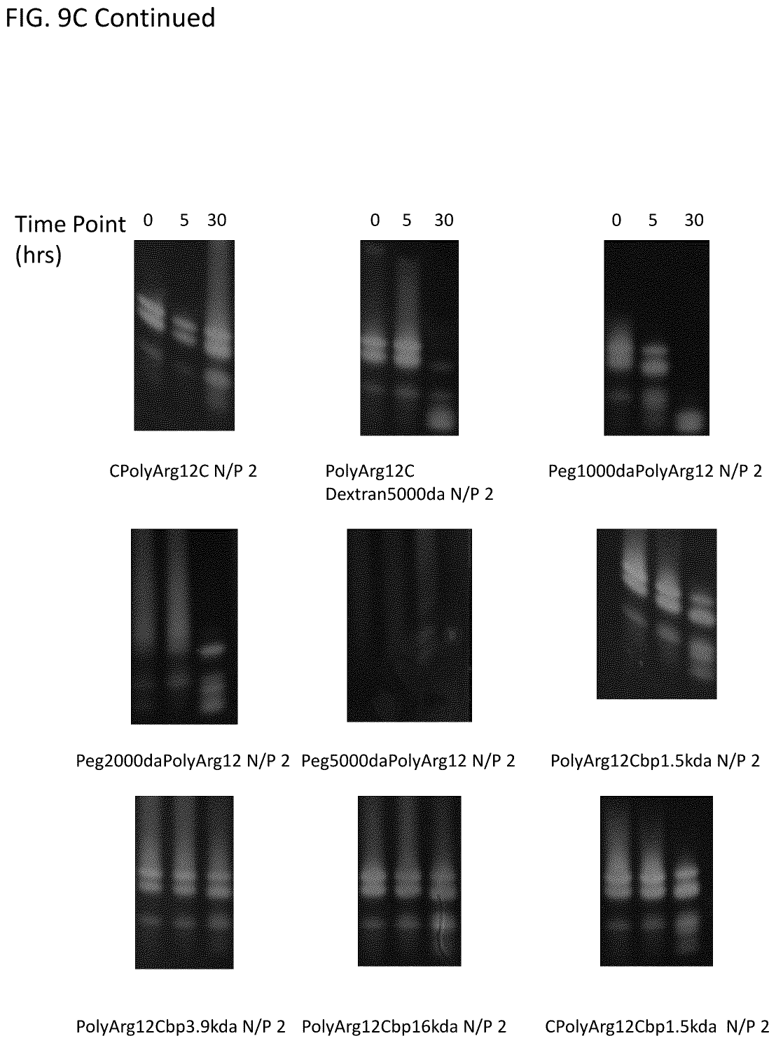

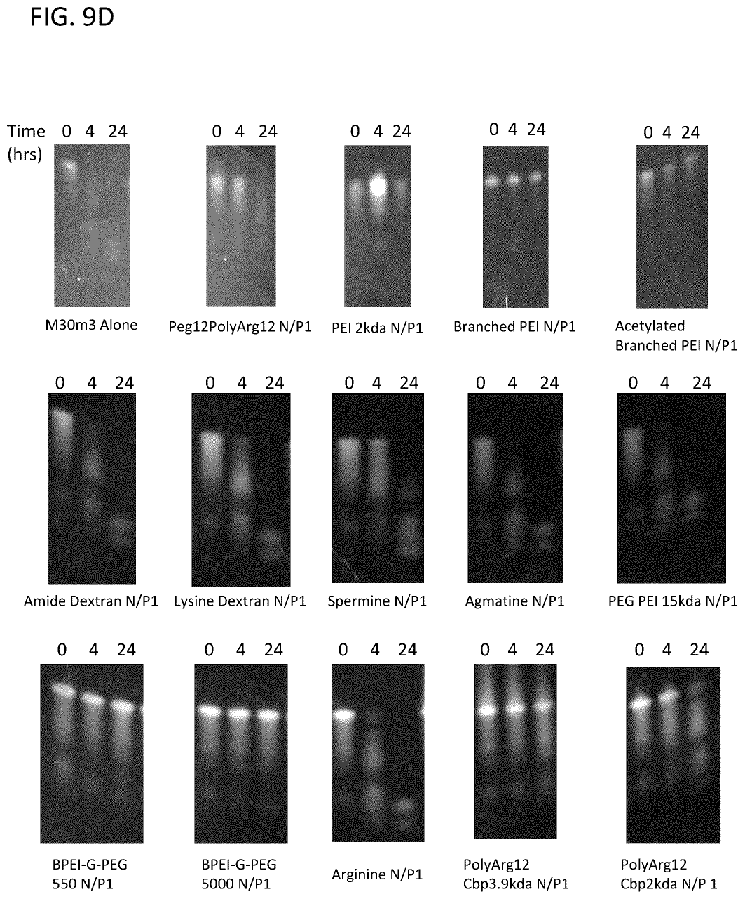

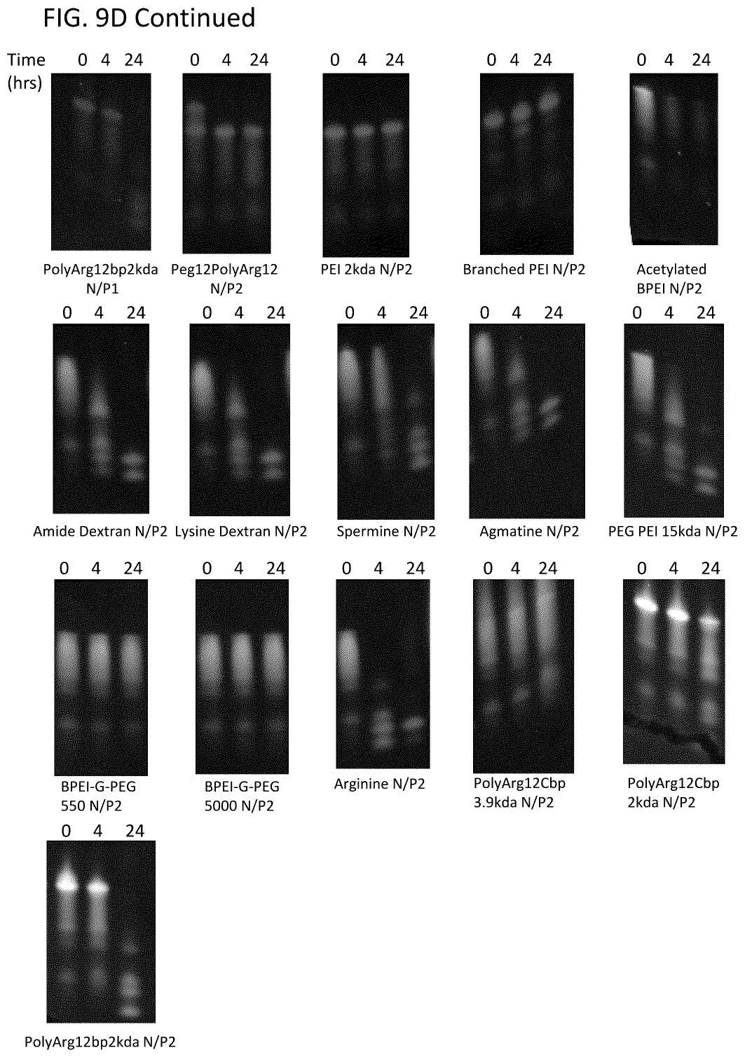

miRNA mimic M30m3 alone or with a cationic peptide or a hybrid polymer at N/P charge ratios of 1 or 2 were incubated with 5% human serum at 37° C. and samples were collected at t=0, 1 hr, 4 hrs, 20 hrs and 24 hrs (1 day). Upon collection, samples were mixed with sample loading buffer containing urea, bromophenol blue, xylene cylanol and nuclease inhibitor. Samples were then reduced, heated to 95° C. for 30 minutes, run on a TBE-urea gel and then stained for oligo using sybergold. The cationic peptide and hybrid polymer increased the serum stability of the M30m3 molecule. See FIG. 9A.

miRNA mimic M30m3 alone or with a cationic peptide or a hybrid polymer at N/P charge ratios of 1 or 2 were incubated with 5% human serum at 37° C. and samples were collected t=0 hr, 1 hr, 4 hrs, 8 hrs and 18 hrs (ns=no serum). Upon collection, samples were mixed with sample loading buffer containing urea, bromophenol blue, xylene cylanol and nuclease inhibitor. Samples were then reduced, heated to 95° C. for 30 minutes, run on a TBE-urea gel and then stained for oligo using sybergold. The cationic peptide and hybrid polymer increased the serum stability of the M30m3 molecule. See FIG. 9B.

miRNA mimic M30m3 alone or with a cationic peptide or a hybrid polymer at N/P charge ratios of 1 or 2 were incubated with 5% human serum at 37° C. and samples were collected at t=0 hr, 5 hrs, and 30 hrs (ns=no serum). Upon collection, samples were mixed with sample loading buffer containing urea, bromophenol blue, xylene cylanol and nuclease inhibitor. Samples were then reduced, heated to 95° C. for 30 minutes, run on a TBE-urea gel and then stained for oligo using sybergold. The cationic peptide and hybrid polymer increased the serum stability of the M30m3 molecule. See FIG. 9C.

miRNA mimic M30m3 alone or with a cationic peptide or a hybrid polymer at N/P charge ratios of 1 or 2 were incubated with 5% human serum at 37° C. and samples were collected at t=0 hr, 4 hrs, and 24 hrs. Upon collection, samples were mixed with sample loading buffer containing urea, bromophenol blue, xylene cylanol and nuclease inhibitor. Samples were then reduced, heated to 95° C. for 30 minutes, run on a TBE-urea gel and then stained for oligo using sybergold. The cationic peptide and hybrid polymer increased the serum stability of the M30m3 molecule. See FIG. 9D.

Table 9 summarized the serum stability of M30m3, alone or with a cationic peptide or a hybrid polymer, of samples from FIG. 9A-9D, calculated based on first order decay comparing two time points.

TABLE 9
M30m3 serum stability with polymers

| | | Serum Stability | Serum Stability |
|---|---|---|---|
| # | Sample Name | at N/P1 (hrs) | at N/P2 (hrs) |
| 1 | M048 Alone | 21.56914013 | |
| 2 | Peg12PolyArg12{d} | 57.62844639 | 90.34914189 |
| 3 | PolyArg12Tinsert | 19.20694059 | 16.89841929 |
| 4 | T6PolyArg12 | 25.59887279 | 18.06179363 |
| 5 | PolyArg6 | 26.73811134 | 22.22810851 |
| 6 | Peg12PolyArg6 | 19.74449857 | 27.95785993 |
| 7 | PolyArg12C | 25.98828465 | 31.12260161 |
| 8 | P24PolyArg12 | 21.21404616 | 39.40654616 |
| 9 | P24PolyArg9 | 20.19250481 | 50.60495789 |
| 10 | PolyArg12C-PEG2000da | 43.58015169 | 40.71236215 |
| 11 | PolyArg12C-PEG5000da | 21.42560355 | 719.1696743 |
| 12 | PolyArg12C-Dextran5000da | 20.03273829 | 27.78771899 |
| 13 | Peg12PolyArg9{d} | 24.70082807 | 29.03370344 |
| 14 | Peg1000daPolyArg12 | 33.58397349 | 20.29098313 |
| 15 | Peg2000daPolyArg12 | 14.99554801 | 46.66592651 |
| 16 | Peg5000daPolyArg12 | 14.07505464 | 20.90840649 |
| 17 | CPolyArg12C | 34.91221728 | 18.10332412 |
| 18 | PolyArg12Cbp1.5kda | 28.48587483 | 63.04573534 |
| 19 | PolyArg12Cbp3.9kda | 141.8998757 | 167.202566 |
| 20 | PolyArg12Cbp16kda | 30.10744999 | 72.18188516 |
| 21 | CPolyArg12Cbp1.5kda | 19.91318544 | 77.38462876 |
| 22 | PolyArg12Cbp2kda | 30.77283238 | 83.15863248 |
| 23 | PolyArg12bp2kda | 19.95913875 | 19.199647348 |
| 24 | PEI 2 kda | 16.31215249 | 253.4014674 |
| 25 | Branched PEI | 36.24957005 | 1020.653909 |
| 26 | Acelated Branched PEI | 23.85946172 | 5.092442432 |
| 27 | Amide Dextran | 15.78135352 | 19.13098763 |
| 28 | Lysine Dextran | 26.78906667 | 14.23050222 |
| 29 | Spermine | 18.94643183 | 12.98356883 |
| 30 | agmatine | 20.78000775 | 17.3322552 |
| 31 | PEG PEI 15 kda | 17.97578478 | 18.5606294 |
| 32 | BPEI-G-PEG 550 | 44.44239755 | 97.92981175 |
| 33 | BPEI-G-PEG 5000 | 80.36702265 | 350.7895093 |
| 34 | Arginine | 7.179135119 | 12.25949462 |

### Example 14. Serum Stability of Cetuximab-miRNA Conjugate+Peptide

Cetuximab-PEG4(azide-DBCO)PEG5-M30m3 conjugates with or without a cationic peptide or a hybrid polymer at N/P charge ratios of 1 or 2 were incubated with 5% human serum at 37° C. and samples collected at t=0, 1 hr, 4 hrs, 20 hrs and 24 hrs(1 day). Upon collection, samples were mixed with sample loading buffer containing urea, bromophenol blue, xylene cylanol and nuclease inhibitor. Samples were then reduced, mixed with proteinase K, heated to 95° C. for 30 minutes, run on a TBE-urea gel and then stained for oligo using sybergold. The cationic peptide and hybrid polymer increased the serum stability of the Cetuximab-PEG4(azide-DBCO)PEG5-M30m3 conjugate. See FIG. 10.

### Example 15. Serum Stability of Diabody ScFv-miRNA Conjugate+Peptide

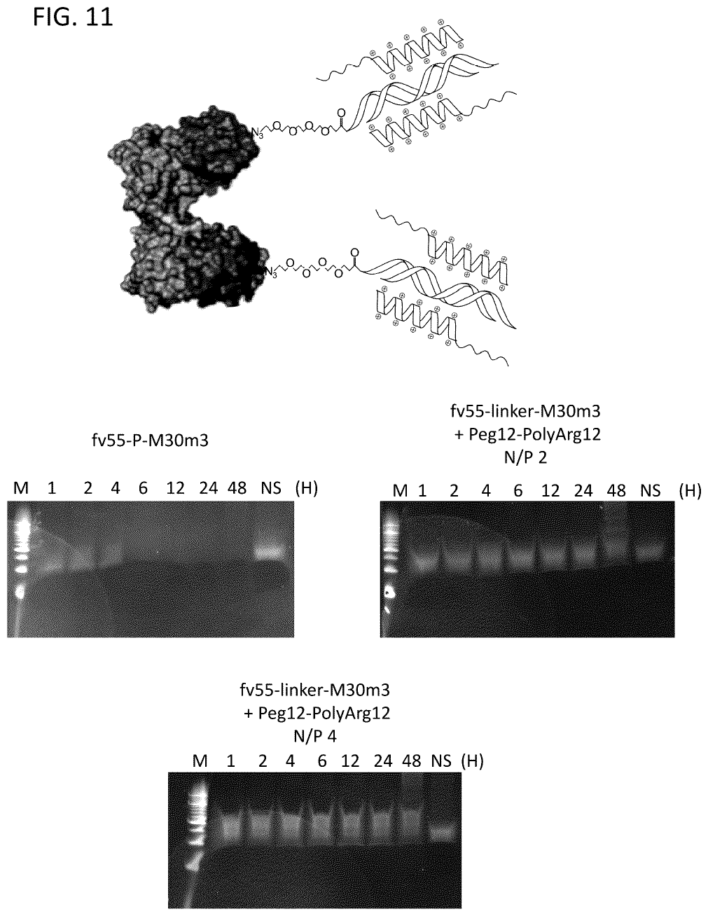

Fv55 diabody was reduced with 10× molar excess of TCEP for 30 minutes, desalted, and reacted with 10× molar excess of azido-PEG3-maleimide. M30m3 was reacted with 6× molar excess DBCO-PEG5-NHS ester. Reactions were quenched and desalted to remove unreacted linker. Fv55-PEG8-DBCO-M30m3 (DAR 2) was formed via click chemistry as described above and purified by SEC. 10 M conjugate (alone or in the presence of PEG12polyArg12 (N:P 2 or 4)) was incubated with 5% human serum at 37° C. for 48 hours. Samples were removed at the indicated time points (1, 2, 4, 6, 12, 24 and 48 hours) and flash frozen to stop further degradation. Samples were resolved by gel electrophoresis on 15% TBE-urea and stained with sybergold to visualize miRNA. The hybrid polymer increased the serum stability of the Fv55 conjugate. See FIG. 11.

### Example 16. Serum Stability of ScFv-Duplex Conjugate

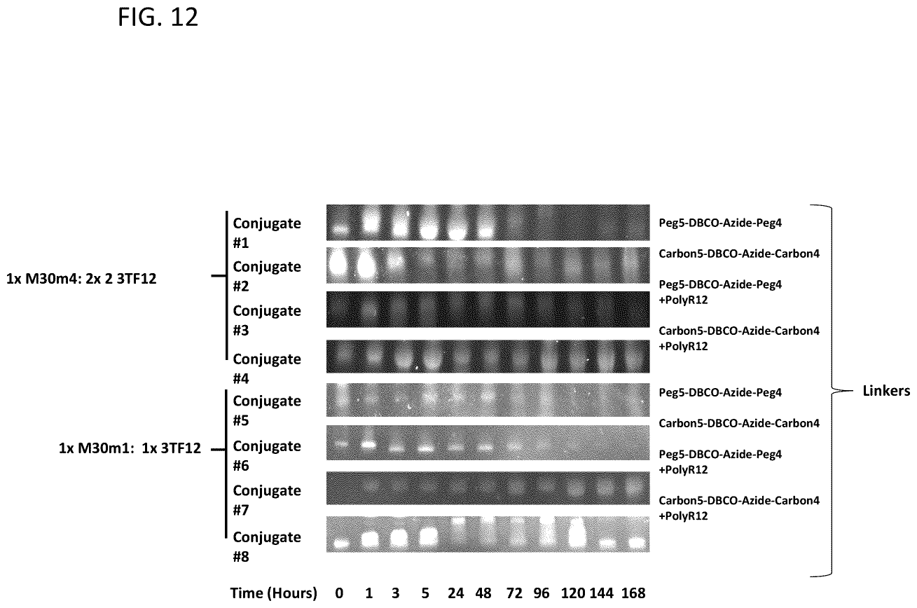

Conjugates were incubated with 5% human serum at 37° C. and samples were collected at the indicated time points (1, 3, 5, 24, 48, 72, 96, 120, 144 and 168 hours (7 days)). Samples were then reduced, heated to 95° C. for 30 minutes, run on a TBE-urea gel and then stained for oligo using sybergold. Conjugate #1-4 contain 2 ScFvs conjugated to one oligo while conjugate #5-8 contain 1 ScFv conjugated per oligo. Conjugates 1 and 5 contain a hydrophilic linker, Linker PEG9. Conjugates 2 and 6 contain a hydrophobic linker, C9 Linker. Conjugates 3 and 7 contain a hydrophilic linker with nuclease protective properties PEG9+PolyR12. Conjugates 4 and 8 contain a hydrophobic linker with nuclease protective properties, C9+PolyR. See FIG. 12.

### Example 17: In Vitro Efficacy of Cetuximab-miRNA Conjugate+/−Peptide

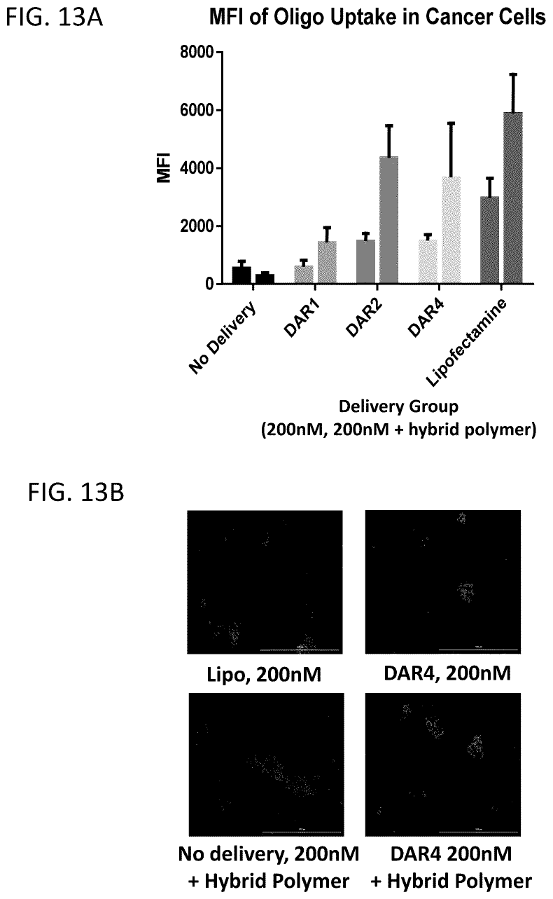

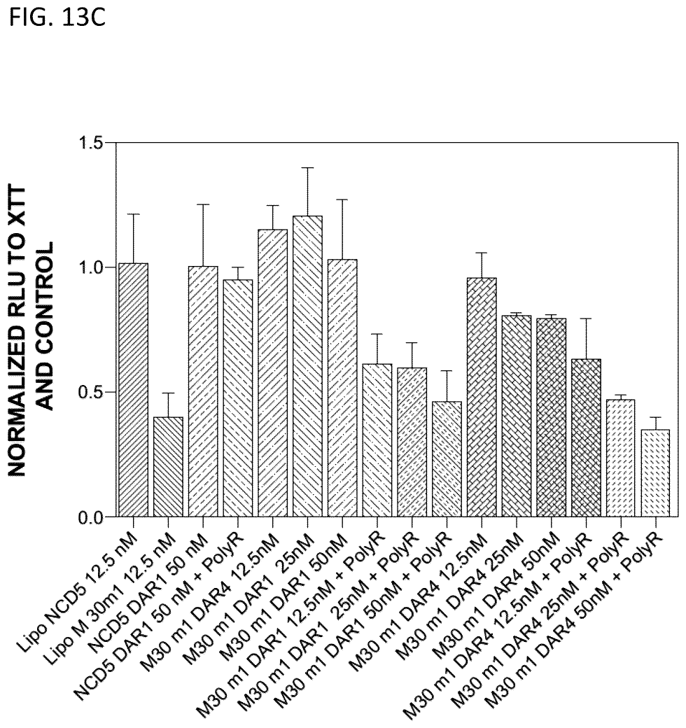

AZDye-647 fluorophore was attached to M30m3 and then conjugated to Cetuximab using a PEG4-azide-DBCO-peg5 linker. Cells were treated with Cetuximab-PEG4-azide-DBCO-PEG5-M30m3-AZdye647 APCs with 3 different DARs at 200 nM with (right bar) and without (left bar) the hybrid polymer Peg12PolyArg12{d}. After 24 hours, cells were washed with PBS and then imaged for ASD647 (Red) and DAPI (Blue) at 4× magnification (n=2 wells, approx. 200 cells/field). Mean fluorescence intensity was averaged based on ASD647 signal in each cell. See FIG. 13A and FIG. 13B. * denotes p<0.005 by students t test as compared to no delivery. Error bars represent standard deviation. These results demonstrate that the conjugates coated with hybrid peptides improve the uptake of the conjugate by target cells in vitro.

UM-SCC-1^{luc} is a cancer cell line that is genetically engineered to overexpress a luciferase reporter containing miR-30 target sites within its 3′ untranslated region (UTR) of the luciferase transcript. This reporter was knocked down by the test miR-30 microRNA mimics such as M30 ml. More specifically, UM-SCC-1^{luc} cells were plated in 96-well plates at 1,500 cells/well. Wells were treated with Oligo without delivery, Lipofectamine Delivery or antibody conjugated delivery at a drug to antibody ratio (DAR) of either 1 or 4 and with or without the addition of PolyR12. Cells were incubated at doses ranging from 50-12.5 nM (oligo) in 96 well format for 5 days. After 5 days XTT assay was performed to determine relative cell number. Luminescence was then determined using firefly luciferase assay kit (Pierce). RLU was normalized to XTT and then to Control Oligo conjugated to antibody. M30-40 conjugates but not Control oligo (NCD5) successfully knockdown reporter activity. Data are reported as average+/−SEM for 3 replicates. Amount of M30 ml added was kept constant between DAR groups. FIG. 13C demonstrates that coating of Cetuximab-miRNA Conjugate improves knockdown of a luciferase reporter that is sensitive to our miR-30 miRNA mimic with a full mAB and also with different DAR ratios for the conjugate.

UM-SCC-1^{luc} cells were plated in 96-well plates at 2,500 cells/well. Wells were treated with Oligo-Lipofectamine Delivery or antibody conjugated delivery at a drug to antibody ratio (DAR) of either 1 or 4 and with or without the addition of two different hybrid polymers, Peg12PolyArg12{d}(FIG. 14A) or PolyArg12Cbp3.9kda (FIG. 14B) (Cetuximab-PEG4-azide-DBCO-PEG5-M30m3 DAR2=APC1, Cetuximab-PEG4-azide-DBCO-PEG5-M30m3 DAR4=APC2). Cells were incubated at doses ranging from 200-25 nM (oligo) in 96 well format for 2 days. Luminescence was then determined using firefly luciferase assay kit (Pierce). RLU was normalized to blank treatment. Data are reported as average+/−SEM for 3 replicates. Amount of M30m3 added was kept constant between DAR groups. See FIG. 14C. These results demonstrate that M30m3 conjugates successfully knocked down reporter activity, with increased knockdown seen in the presence of two different hybrid polymers.

UM-SCC-1^{luc} cells were plated in 96-well plates at 2,500 cells/well. Wells were treated with Oligo-Lipofectamine Delivery or antibody conjugated delivery at a drug to antibody ratio (DAR) of 4 with or without the addition of four different hybrid polymers: Peg12PolyArg12{d}, PolyArg12C-PEG2000da, PolyArg12C-PEG5000da, or PolyArg12C-Dextran5000da (Cetuximab-PEG4-azide-DBCO-PEG5-M30m3 DAR4=APC2). Cells were incubated at doses ranging from 250-31.25 nM (oligo) in 96 well format for 2 days. The amount of M30m3 added was kept constant between treatment groups. Luminescence was then determined using firefly luciferase assay kit (Pierce). RLU was normalized to APC2 treatment. M30m3 conjugate successfully knocked down reporter activity, with increased knockdown seen in the presence of different hybrid polymers. See FIG. 14D. Data are reported as average+/−SEM for 3 replicates.

UM-SCC-1^{luc} cells were plated in 96-well plates at 2,500 cells/well. Wells were treated with Oligo-Lipofectamine Delivery or antibody conjugated delivery at a drug to antibody ratio (DAR) 4 with or without the addition of Peg12PolyArg12{d}(Cetuximab-SMCC-M30m3 DAR4=SMCC Conjugate, Cetuximab-MCVCPABcPNP-M30m3 DAR4=MCVCPABcPNP Conjugate, and Cetuximab-MCPEG4VCPABcPNP-M30m3 DAR4=MCPEG4VCPABcPNP Conjugate). Cells were incubated at doses ranging from 500-62.5 nM (oligo) in 96 well format for 2 days. The amount of M30m3 added was kept constant between DAR groups. Luminescence was then determined using firefly luciferase assay kit (Pierce). RLU was normalized to blank treatment. M30m3 conjugates successfully knocked down reporter activity, with increased knockdown seen in the presence of hybrid polymers. See FIG. 14E.Data are reported as average+/−SEM for 3 replicates.

UM-SCC-109 cells are an immortalized head and neck cancer cell line. UM-SCC-109 cells were plated in 12-well plates at 250,000cells/well. Wells were treated with a mixture of unconjugated Cetuximab+Peg12PolyArg12{d}+M30m3 as a control, or antibody conjugated delivery at a drug to antibody ratio (DAR) 4 with or without the addition of Peg12PolyArg12{d}(Cetuximab-SMCC-M30m3 DAR4=SMCC Conjugate, Cetuximab-MCVCPABcPNP-M30m3 DAR4=MCVCPABcPNP Conjugate, Cetuximab-MCPEG4VCPABcPNP-M30m3 DAR4=MCPEG4VCPABcPNP Conjugate). Cells were incubated at 200 nM (oligo) for 2 days. The amount of M30m3 added was kept constant between groups. Cells were then harvested for RNA and Taqman qPCR was performed on EGFR and Serpine1 (two target genes of miR30) to demonstrate efficacy of conjugate and entry of oligo into cells. RLU was normalized to mixture control treatment. M30m3 conjugates successfully knocked down EGFR and Serpine1, with increased knockdown seen in the presence of hybrid polymers. See FIG. 14F. Data are reported as average+/−SEM for 3 replicates.

UM-SCC-1^{luc} cells were plated in 96-well plates at 2,500 cells/well. Wells were treated with Cetuximab alone or antibody conjugated delivery at a drug to antibody ratio DAR4 with or without the addition of Peg12PolyArg12{d}), BPEI-G-550, or BPEI-PEG5000 in a 1/1 charge for charge at the indicated concentrations (APC1=Cetuximab-PEG4-azide-DBCO-PEG5-M30m3 DAR4 Conjugate). Cells were incubated at doses ranging from 200-25 nM (oligo) in 96 well format for 2 days. Luminescence was then determined using firefly luciferase assay kit (Pierce). RLU was normalized to blank treatment. M30m3 conjugates successfully knocked down reporter activity, with increased knockdown seen in the presence of the hybrid polymer. See FIG. 14G. Data are reported as average+/−SEM for 3 replicates.

In summary, FIG. 14C-G demonstrate that coating Cetuximab-miRNA Conjugate with different hybrid polymers results in knockdown of a luciferase reporter that is sensitive to the miR-30 miRNA mimic. The knockdown was demonstrated using conjugates with different DAR ratios, as well as different linkers.

### Example 18: In Vitro Activity of 3tf12-M30m3 XTT and Lum

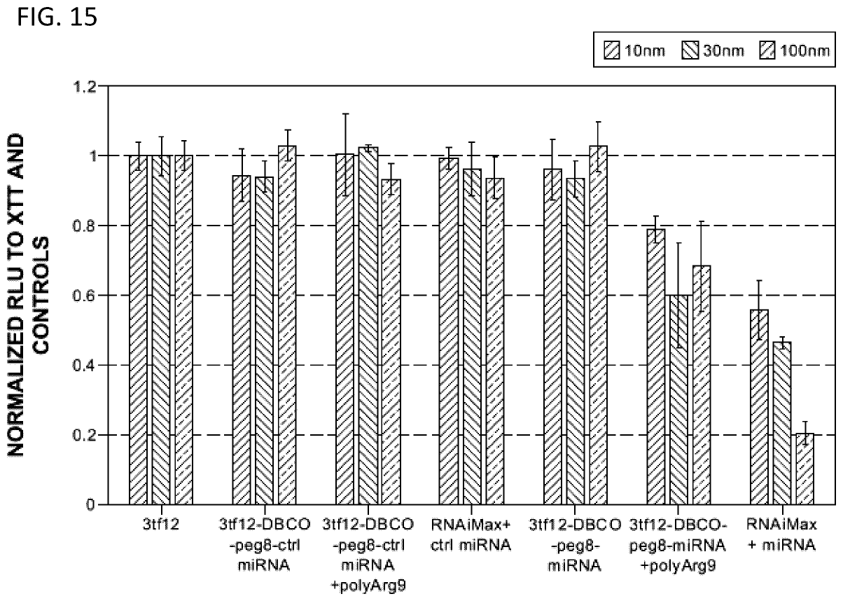

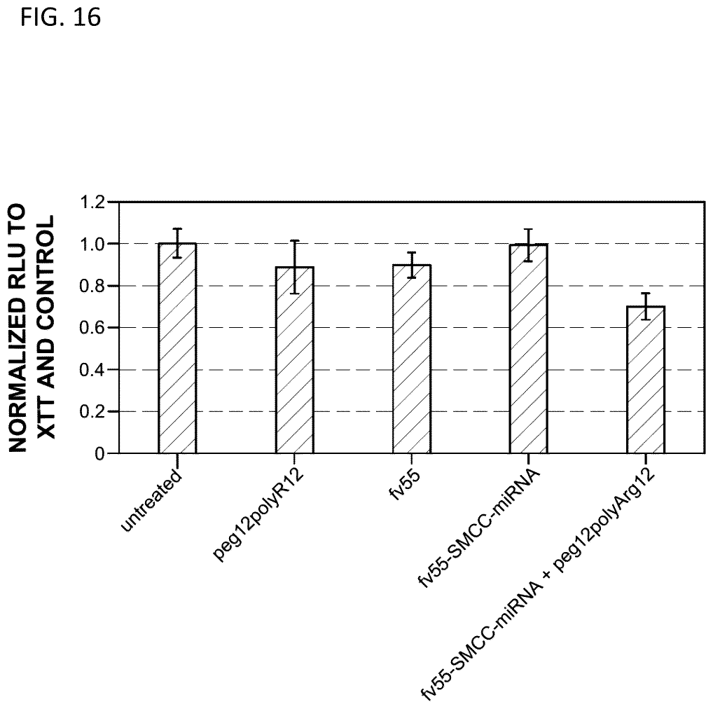

3tf12 diabody was reacted with sulfoDBCO-PEG4-maleimide while control miRNA and miRNA (M30m3) were separately reacted with azido-PEG4-NHS ester. 3tf12-DBCO-PEG8-miRNA conjugates were generated via click chemistry as described above. 1500 UM-SCC-1^{luc} (a luciferase reporter cell line for HNSCC) cells were plated and treated with TfR-targeting conjugates in the presence and absence of polyArg9 peptide (N:O 1:1). Cells were assayed for viability and luminescence four days post-treatment. Cells were transfected with RNAiMAX to deliver miRNAs for comparison. The results demonstrate that the 3tf12-M30m3 conjugate was able to knockdown expression of the reporter gene when administered in the presence of the polyArg9 peptide. See FIG. 15. Data are reported as average+/−SEM for three replicates.

### Example 19: In Vitro Activity of Fv55-SMCC-M30m3 with Hybrid Polymer Peptide

Fv55-SMCC-miRNA was created by first reacting SMCC with miRNA via NHS ester for 2 hours at room temperature. Unreacted SMCC was removed by desalting and SMCC-miRNA was incubated with reduced Fv55. Fv55-SMCC-miRNA was purified by SEC and determined to contain 1 miRNA per diabody. 1000 UM-SCC-1luc cells were plated and treated with 300 nM conjugate. PEG12polyArg12 was used at a 7.2 molar excess over miRNA for an N:O charge ratio of 2:1. Cells were assayed for viability and luminescence 8 days post-treatment. The results demonstrate that the Fv55-SMCC-miRNA conjugate was able to knockdown expression of the reporter gene when administered in the presence of the PEG12polyArg12 hybrid polymer. See FIG. 16. Data are reported as average+/−SEM for five replicates.

### Example 20: In Vitro Efficacy of Cetuximab-miRNA Conjugate+/−Cationic Peptide

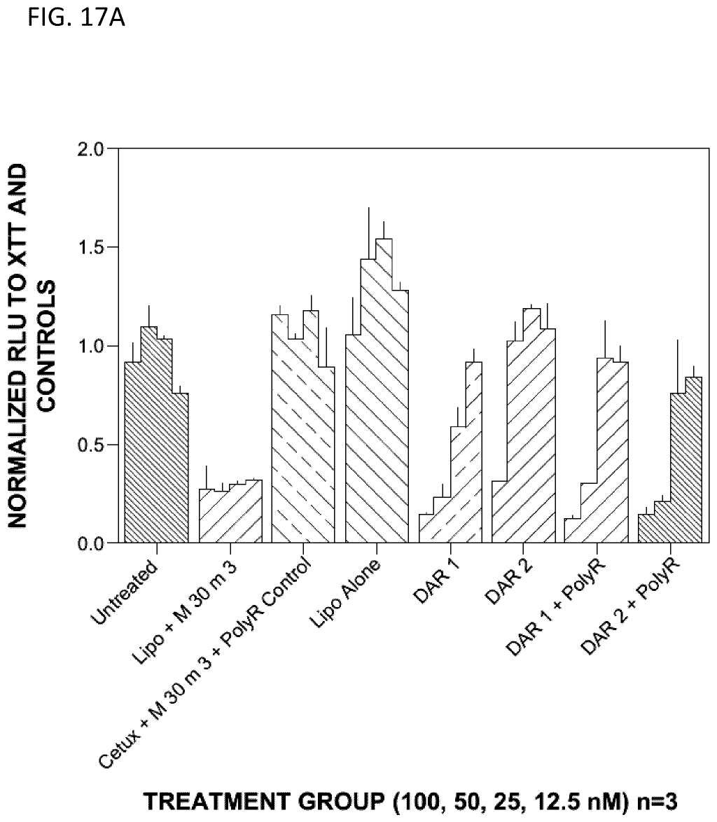

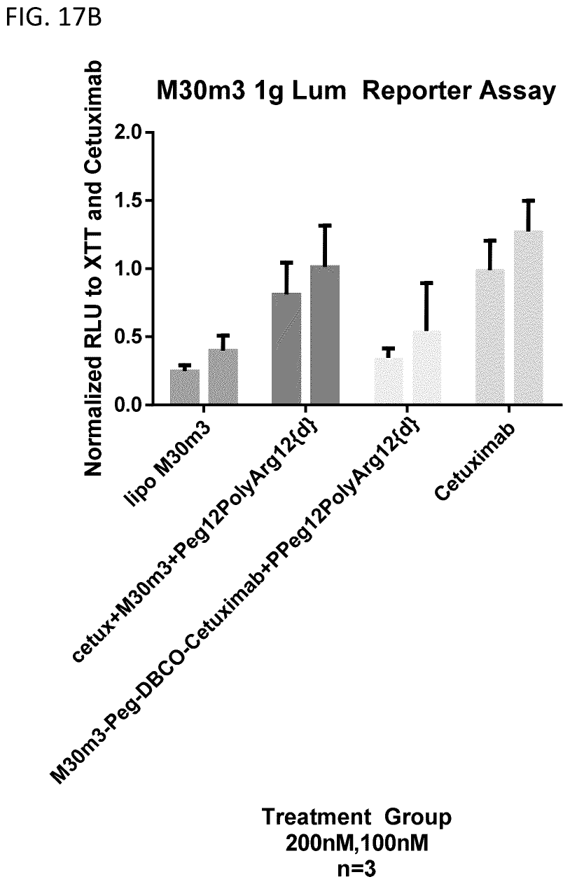

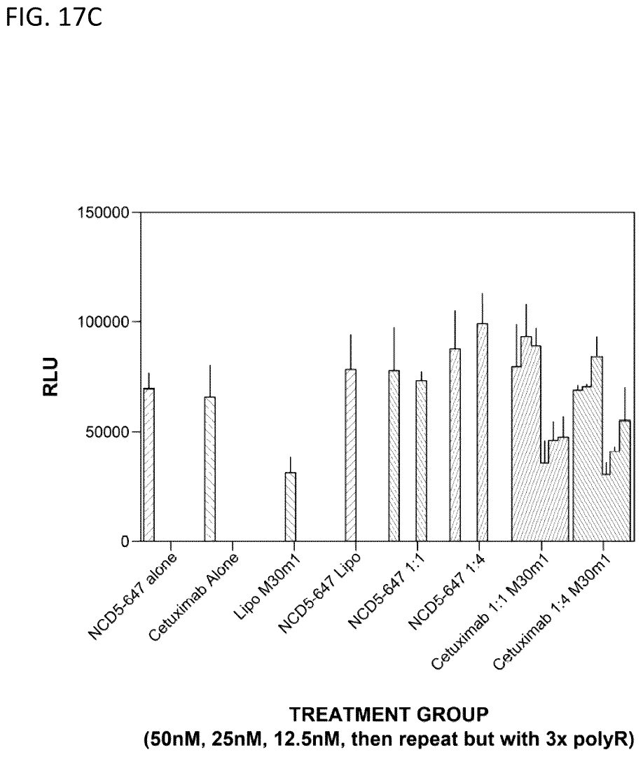

DAR purified M30m3-PEG5-(DBCO-Azide)-PEG4-Cetuximab was added with and without polyR12 (1:1 N/P charge ratio) to UM-SCC-1^{luc} at various doses after plating of 2,500 cells/well. The amount of M30m3 added was kept constant between groups. Cells were assayed for viability and luminescence 3 days post-treatment. Luminescence was normalized to XTT and then to average of Cetuximab+M30m3+polyR control mixture. The Cetusixmab-M30m3 conjugates at DAR1 and DAR2 were able to knockdown expression of the reporter gene both in the presence and absence of the cationic peptide. See FIG. 17A. Data are reported as average+/−SEM for 3 replicates.

M30m3-PEG5-(DBCO-Azide)-PEG4-Cetuximab was added with PEG12PolyR12{d}polymer at a 1:1 N/P charge ratio at 200 nM and 100 nM to UM-SCC-1^{luc} after plating of 2,500 cells/well. The amount of Cetuximab and M30m3 added was kept constant between groups. Treatment concentration as reported for oligo concentration. Cells were assayed for viability and luminescence 3 days post-treatment. Luminescence was normalized to XTT and then to average of Cetuximab alone control treatment. The coated conjugate was able to knockdown the expression of the reporter gene. See FIG. 17B. Data are reported as average+/−SEM for 3 replicates.

M30 ml-PEG5-(DBCO-Azide)-PEG4-Cetuximab was added with and without polyR12 (1:1 N/P charge ratio and 1:4 charge ratio) at 12.5, 25 and 50 nM to UM-SCC-1^{luc} after plating of 2,500 cells/well. The amount of M30 ml added was kept constant between groups. Concentration as reported for oligo dose. Cells were assayed for luminescence 3 days post-treatment. The Cetuximab-M30 ml conjugates were able to knockdown expression of the reporter gene at both charge ratios. See FIG. 17C. Data are reported as average+/−SEM for 3 replicates.

### Example 21: In Vitro Efficacy of ScFv-ASO Conjugate

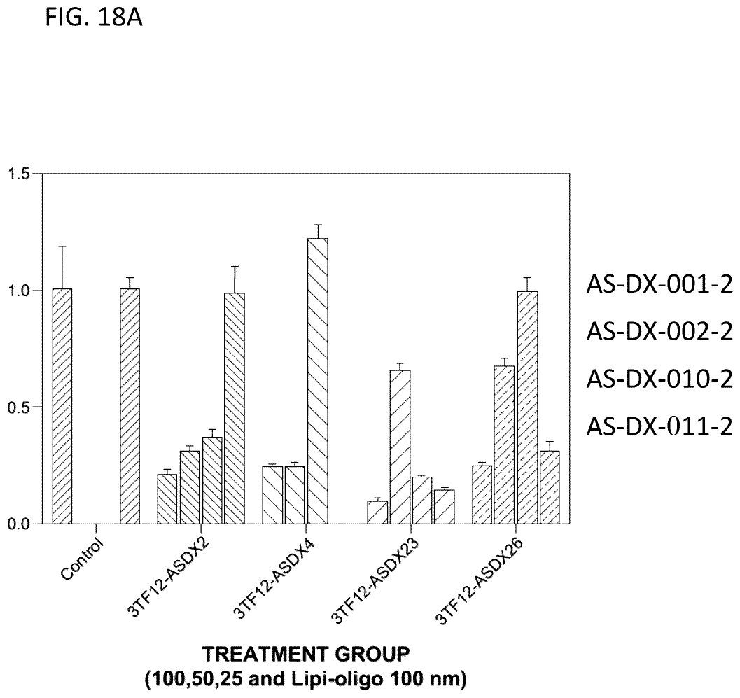

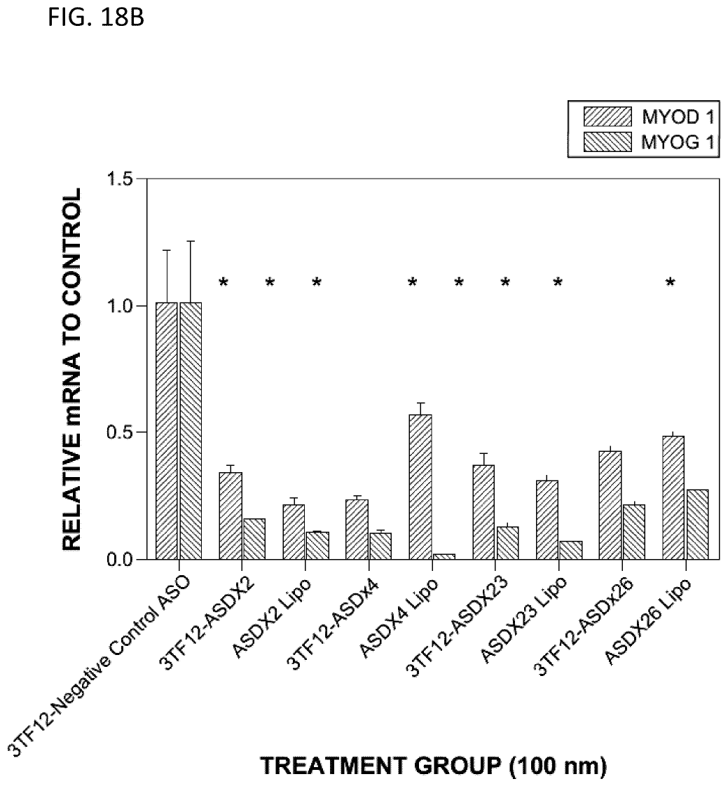

FSHD myoblasts were differentiated for 48 hours and then treated with 3TF12 conjugated to different antisense oligonucleotides (ASOs) at 100 nM, 50 nM and 25 nM (oligo concentration). Lipofectamine+oligo was used as a positive control for each ASO-ScFv conjugate. Negative Control ASO conjugated to 3TF12 was used to normalize results. After 72 additional hours, total RNA was collected, and qPCR was performed using DUX4 primers and GAPDH as internal control. Each ASO was able to knockdown expression of the DUX4 gene. See FIG. 18A. Values represent the mean of two experiments with three technical repeats each, and error bars represent+/−SEM.

FSHD myoblasts were differentiated for 48 hours and then treated with 3TF12 conjugated to different ASOs at 100 nM. Lipofectamine-ASO complex was used as a positive control for each ASO-antibody conjugate coated with PEG12polyArg12 hybrid polymer. Negative Control ASO conjugated to 3TF12 or mixed with lipofectamine was used as a negative control. The scFv conjugated ASOs were able to knockdown expression of the downstream DUX4 target genes MYOD1 and MYOG1 in muscle cells from an FSHD patient. See FIG. 18B. Values represent the mean of two experiments with three technical repeats each, and error bars represent SEM **p<0.005 by Student's t-test for both doses.

### Example 22: Cetuximab-miRNA Conjugate+Cationic Peptide or Hybrid Polymer Coating Improves Delivery to Tumor

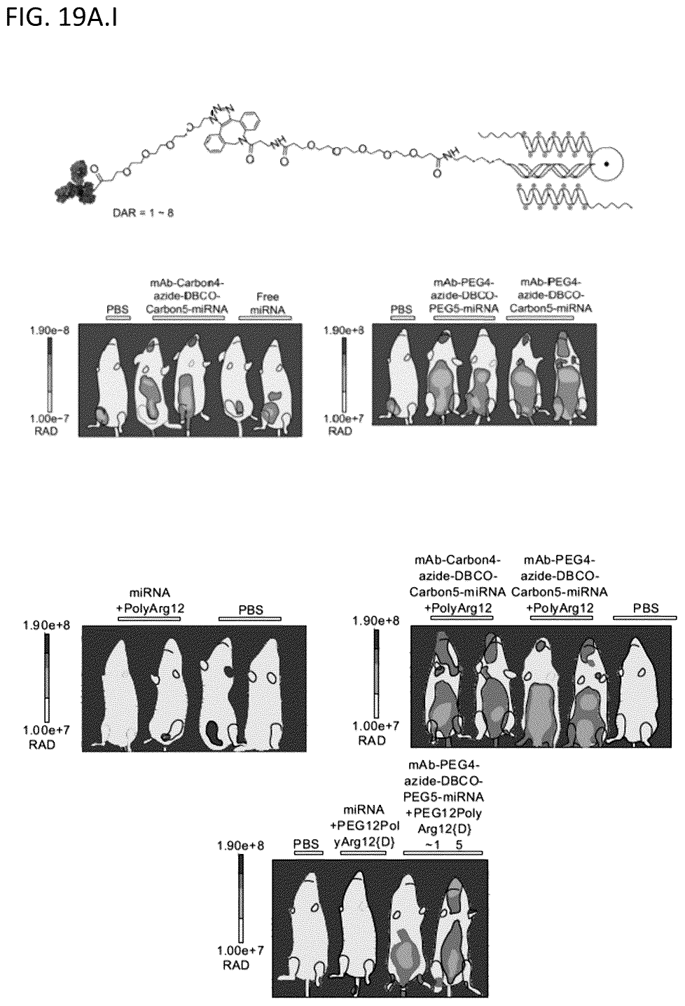

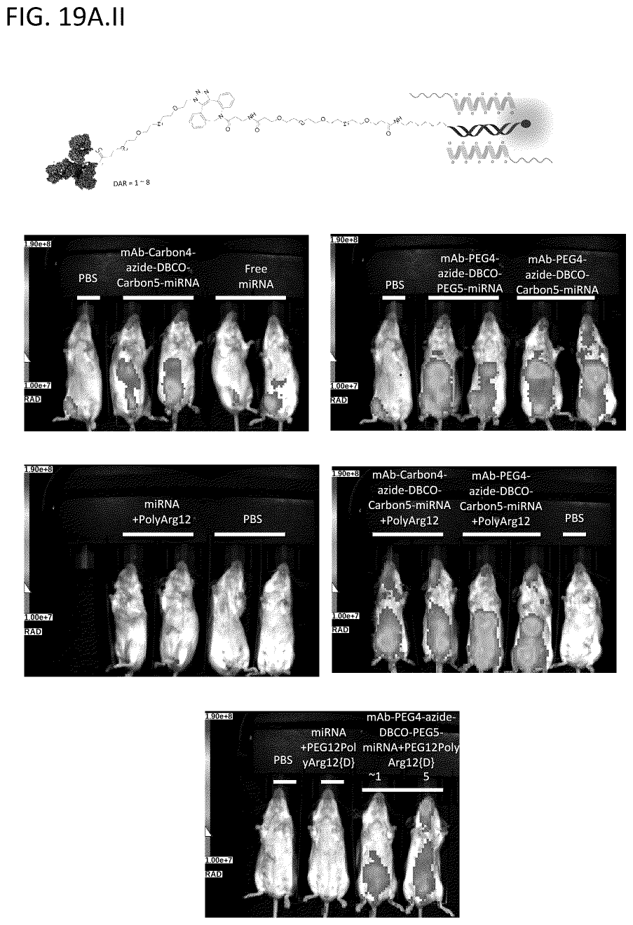

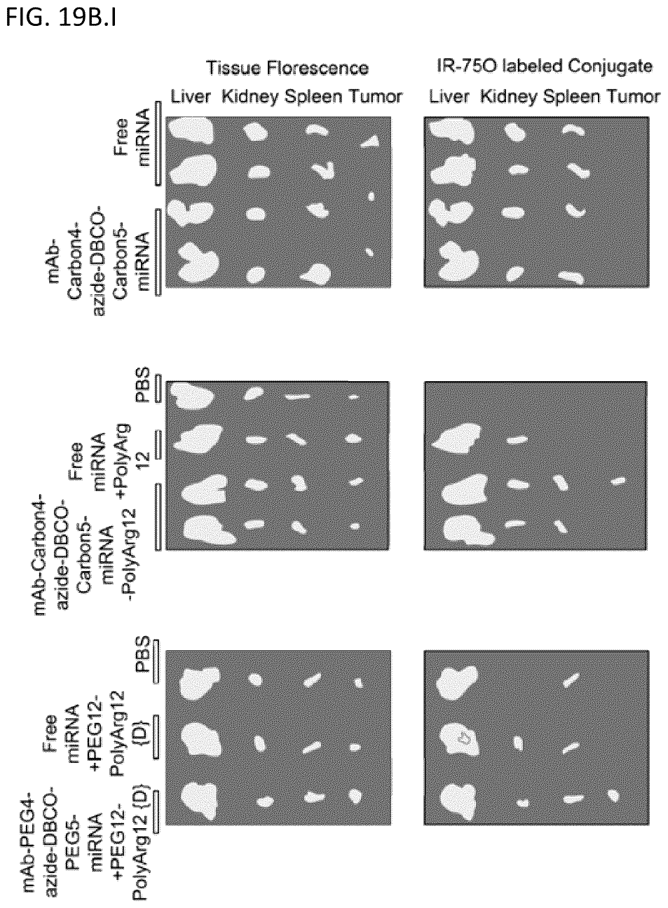

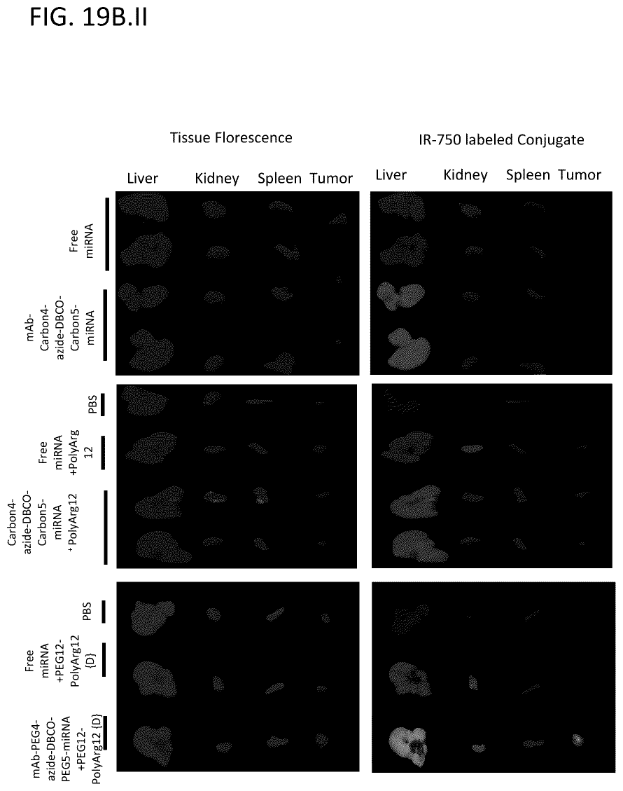

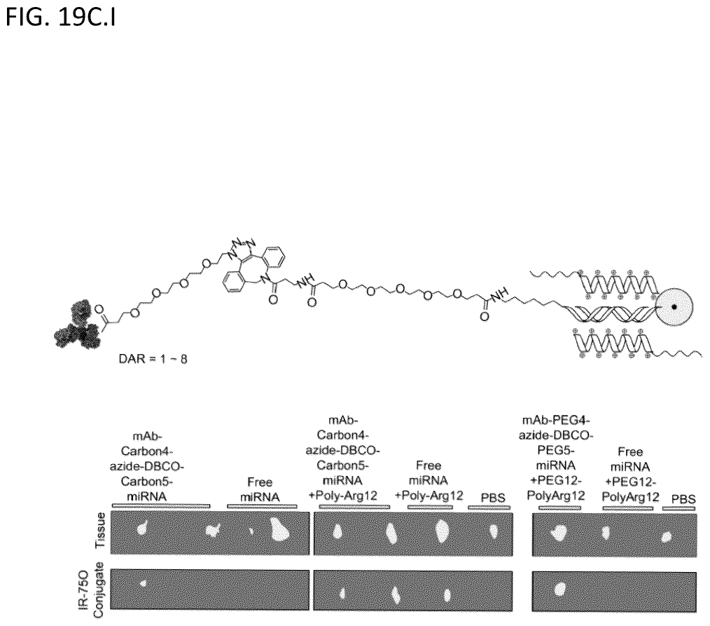

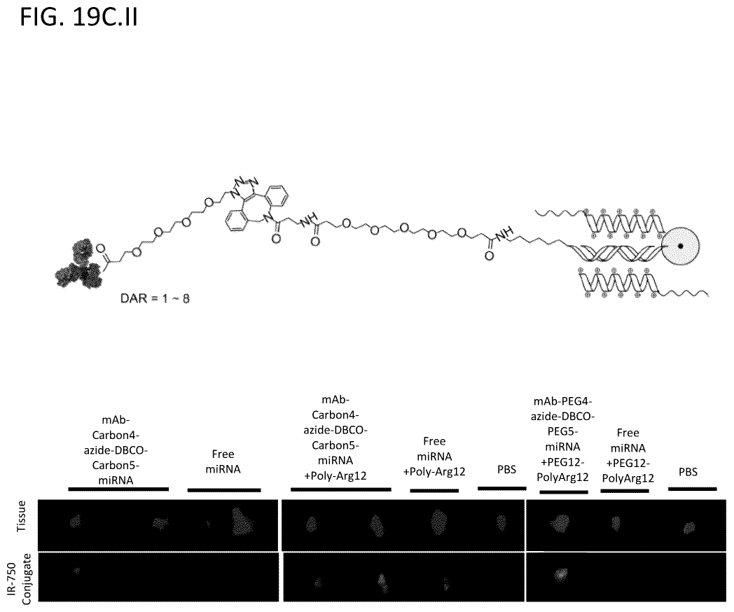

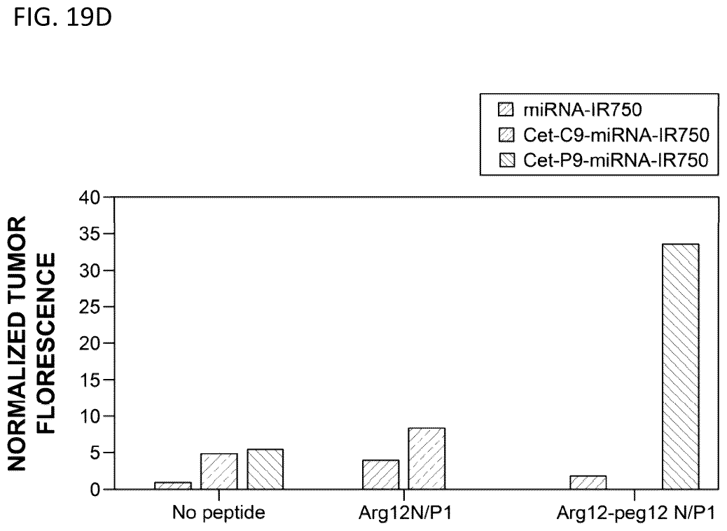

mAb conjugates at DAR1 with variable linker chemistry were prepared and an infrared fluorophore was added on the terminal end of a NCD5 control miRNA mimic. The conjugates were used naked or complexed with various cationic peptides or hybrid polymers. Briefly, SCID/NCR mice were injected orthotopically with 1×10⁶ UM-SCC-1^{luc} cells and tumor growth was tracked by luminescence from the cells. Once signal reached 1×10⁶ Photons/s/cm²/sr, mice were injected with 200 μL of 5 mg/kg IR750-NCD5 that is either naked, formulated with peptide or conjugated to cetuximab with either a C4(Azide-DBCO)C5, PEG4(Azide-DBCO)C5, or a PEG4(Azide-DBCO)C5 linker+/−cationic peptide or hybrid polymer at charge ratio (N/P) of 1. After 24 hours mice were imaged for IR-750 to track conjugate biodistribution. See FIG. 19A. In addition, after 48 hours, mice were sacrificed, and tissues collected and washed in PBS. Tissues were imaged on an Odyssey CLx imager. Background tissue auto-florescence was displayed from the 700 channel (See FIG. 19B, left panel), and IR-750 fluorophore was detected in the 800 channel (See FIG. 19B, right panel).

Cetuximab-miRNA Conjugate+peptide Tissue Biodistribution was determined by imaging the tumor tissue on an Odyssey CLx imager. Background auto-florescence was displayed from the 700 channel (See FIG. 19C, top panel), and IR-750 fluorophore was detected in the 800 channel (See FIG. 19C, bottom panel). IR-750 florescence intensity was quantified in (ImageQuant v X.x). See FIG. 19D, which displays mean florescence intensity over the tumor area for each treatment.

These results demonstrate that complexation of PEG12-Arg12 at an N/P of 1 markedly improves delivery to the tumor and reduces liver and kidney delivery. (FIGS. 19A and 19B). FIG. 19B displays uptake of the conjugates in dissected tumors from the same animals. FIGS. 19C and 19D displays quantitation of tumor delivery.

### Example 23: IR750-FV55 Diabody-miRNA Conjugate+Hybrid Polymer Coating Improves Delivery to Tumor

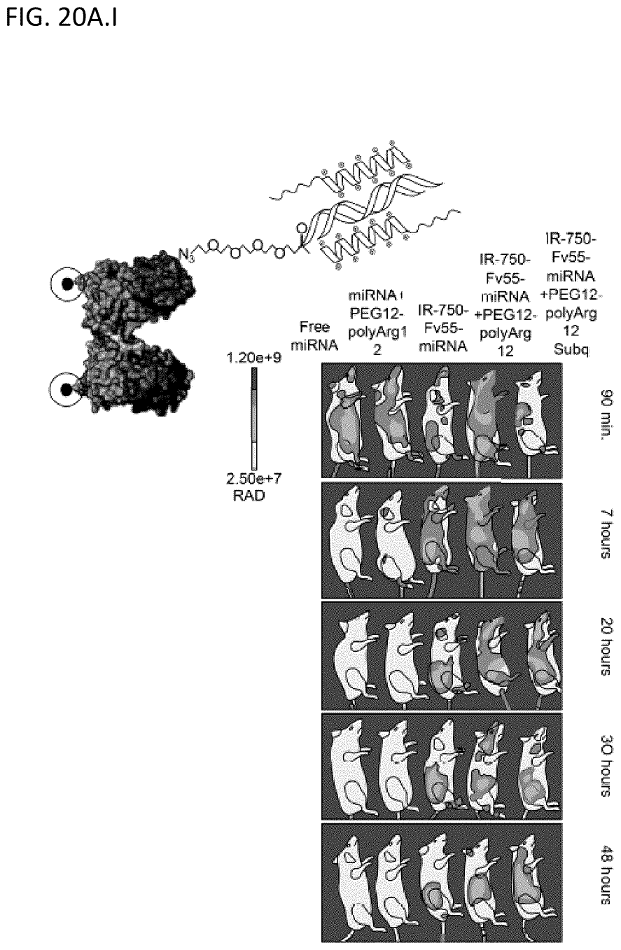

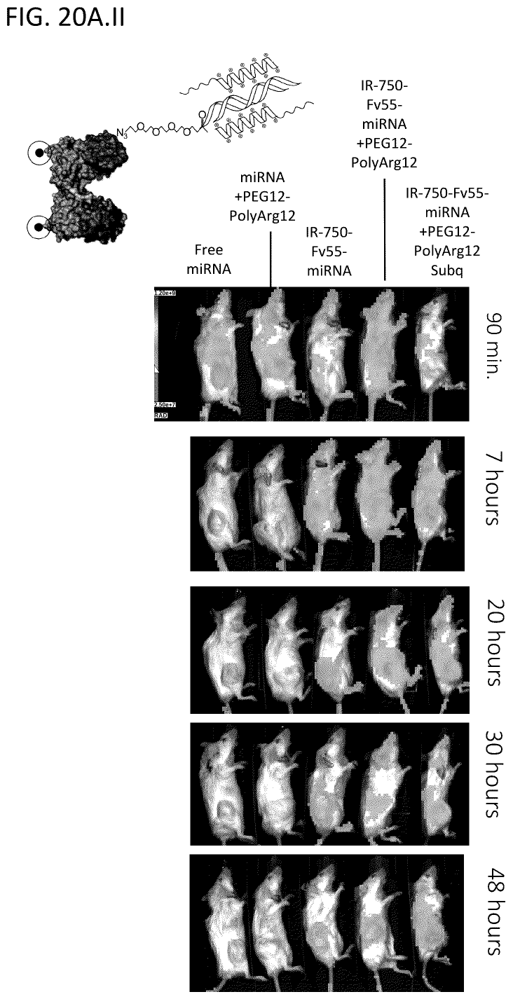

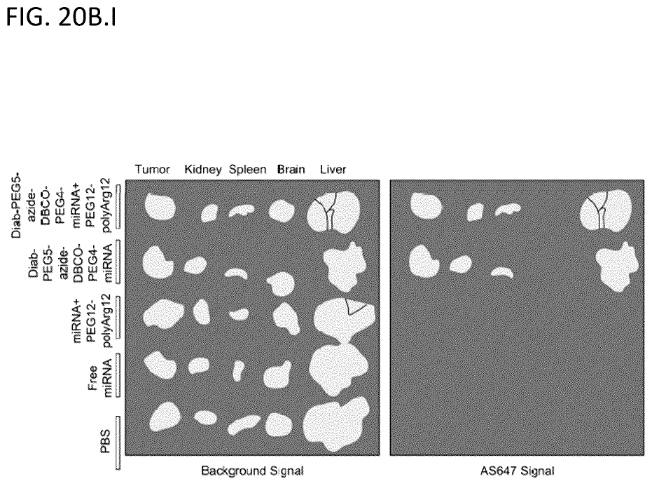

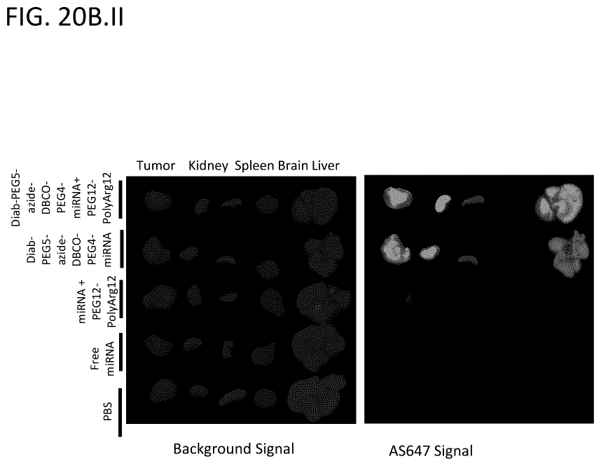

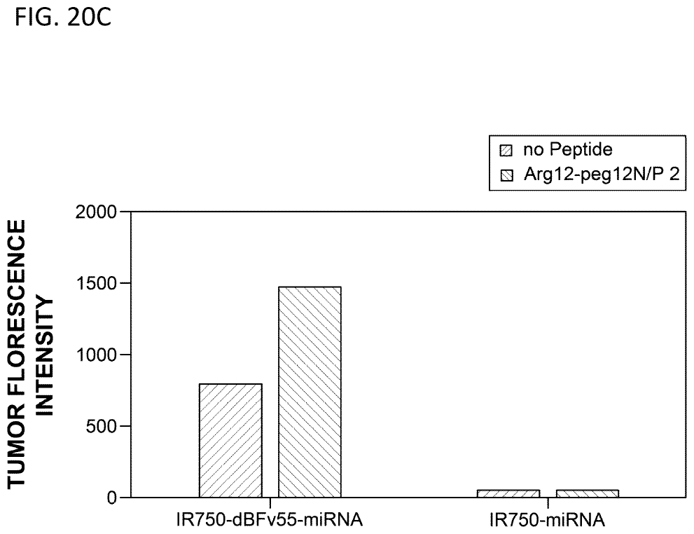

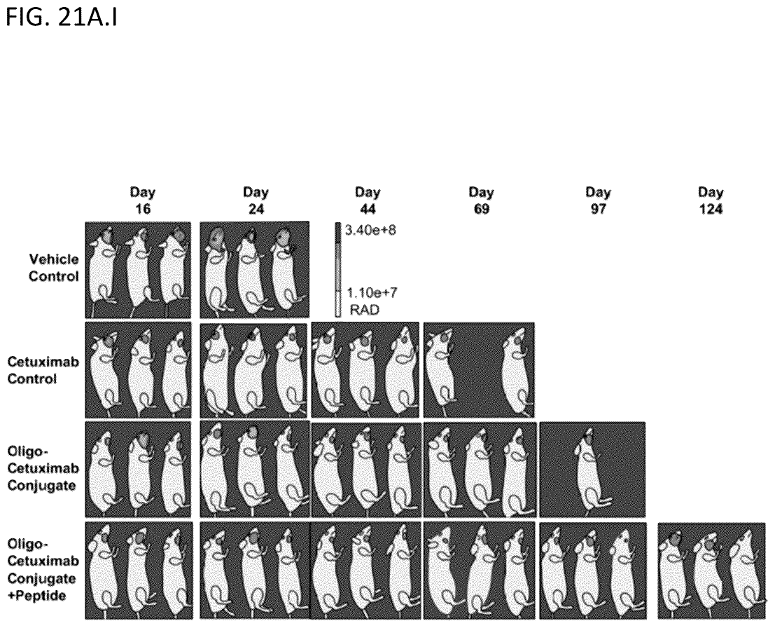

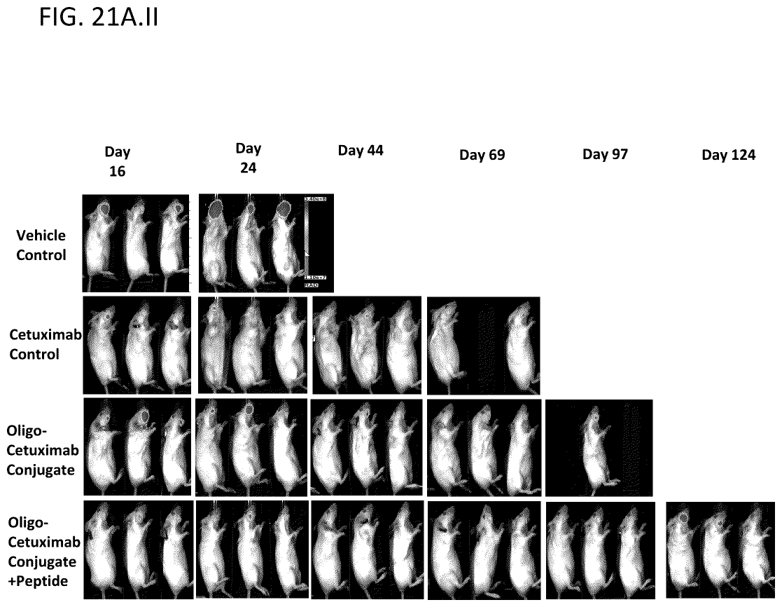

Fv55 diabody conjugates at DAR1 with an infrared fluorophore on the diabody were constructed. Fv55 diabody was reduced with 10x molar excess of TCEP for 30 minutes, desalted, and reacted with 10x molar excess of azido-PEG3-maleimide. M30m3 was reacted with 6× molar excess DBCO-PEG5-NHS ester. Reactions were quenched and desalted to remove unreacted linker. Fv55-PEG8-DBCO-M30m3 was formed via click chemistry and purified by SEC. Finally, the conjugate was labeled with IR750 and dialyzed against PBS to remove free fluorophore. Animals with UMSCC-109 tumors on the right flank were dosed iv with control miRNA-IR750, control miRNA-IR750+PEG12polyArg12, fv55-PEG8-DBCO-miRNA-IR750, or Fv55-PEG8-DBCO-miRNA-IR750+PEG12polyArg12 at 5 mg/kg miRNA. PEG12polyArg12 was used at a concentration to achieve N:P 2:1. Four animals were IV-injected while the fifth animal received a subcutaneous injection. Animals were imaged on an IVIS imager at the indicated time points, sacrificed after 48 hours, and harvested for organs. After 48 hours, mice were sacrificed, and tissues collected and washed in PBS. Tissues were imaged on an Odyssey CLx imager. FIG. 20A demonstrates that the hybrid polymer increased the circulation time of the Fv55-miRNA conjugate. FIG. 20B and FIG. 20C demonstrate delivery to target dissected tissue and higher delivery to the tumor for the conjugates coated with the hybrid polymers than the naked conjugates. Background tissue auto-florescence was displayed from the 700 channel (see FIG. 20B, left panel), and IR-750 fluorophore was detected in the 800 channel (see FIG. 20B, right panel). IR-750 florescence intensity was quantified in (ImageQuant software) for tumor tissue. See FIG. 20C, which displays mean florescence intensity over the tumor area for each treatment.

### Example 24: Biocoating of Cetuximab-C9-M30m3 Conjugate with Hybrid Polymer Improves Anti-Tumor Efficacy

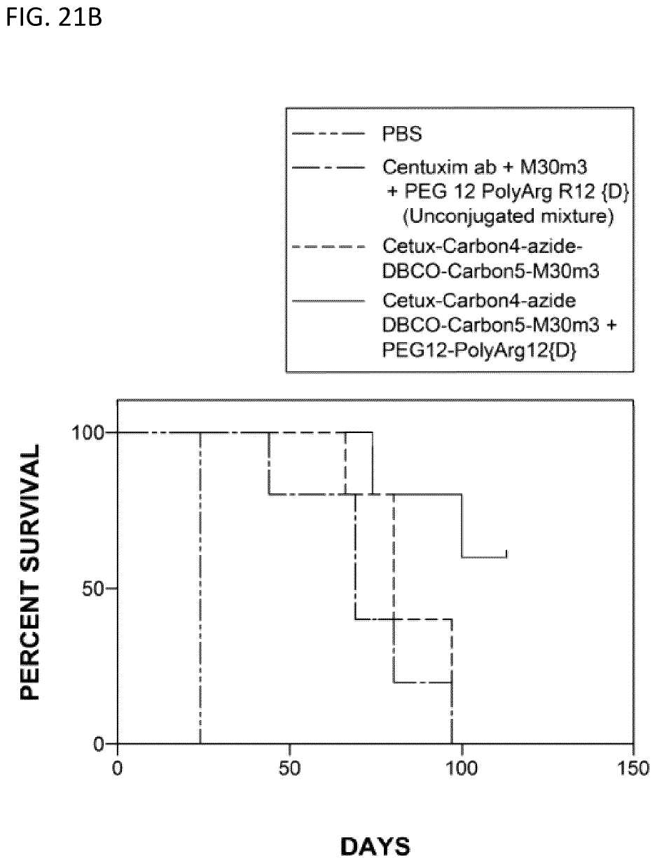

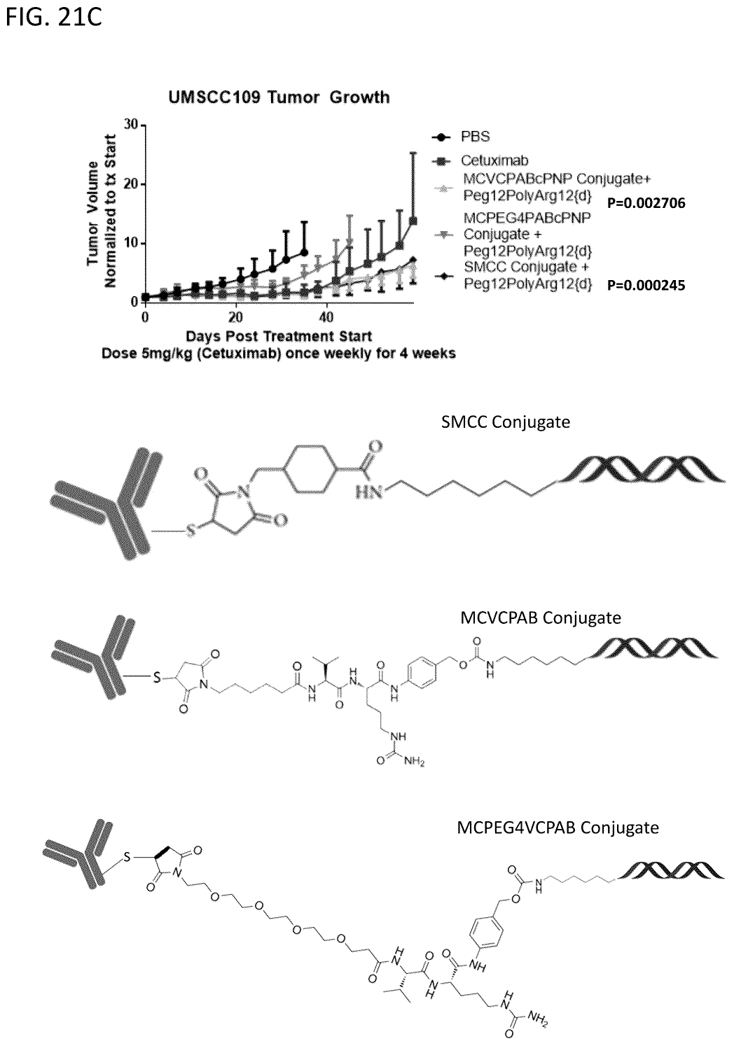

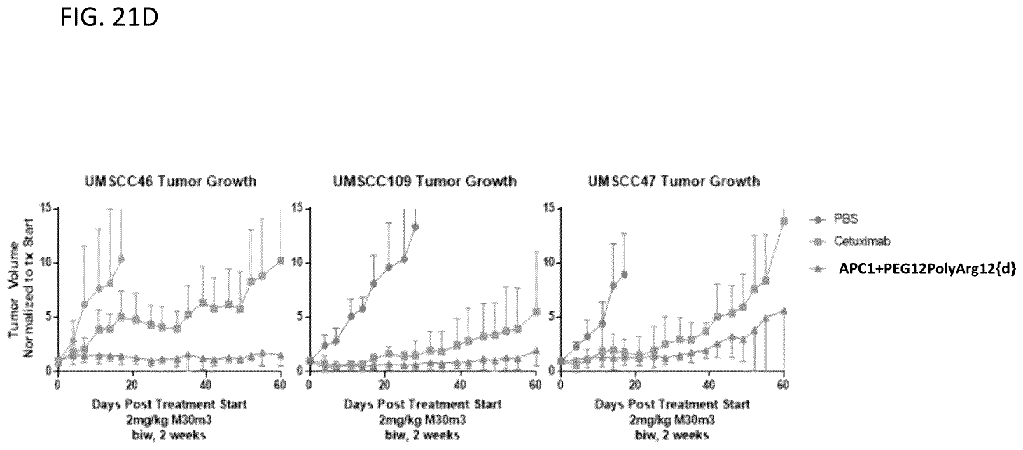

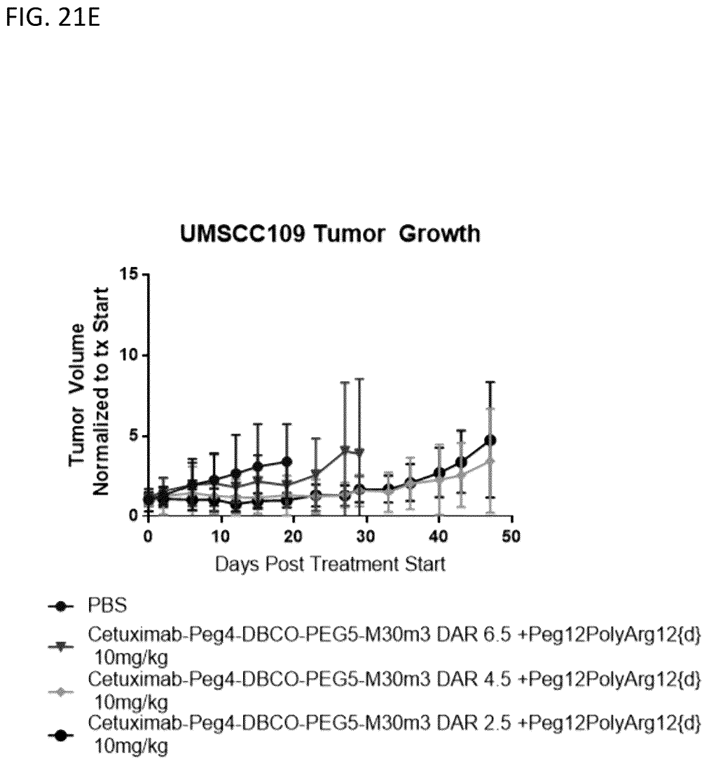

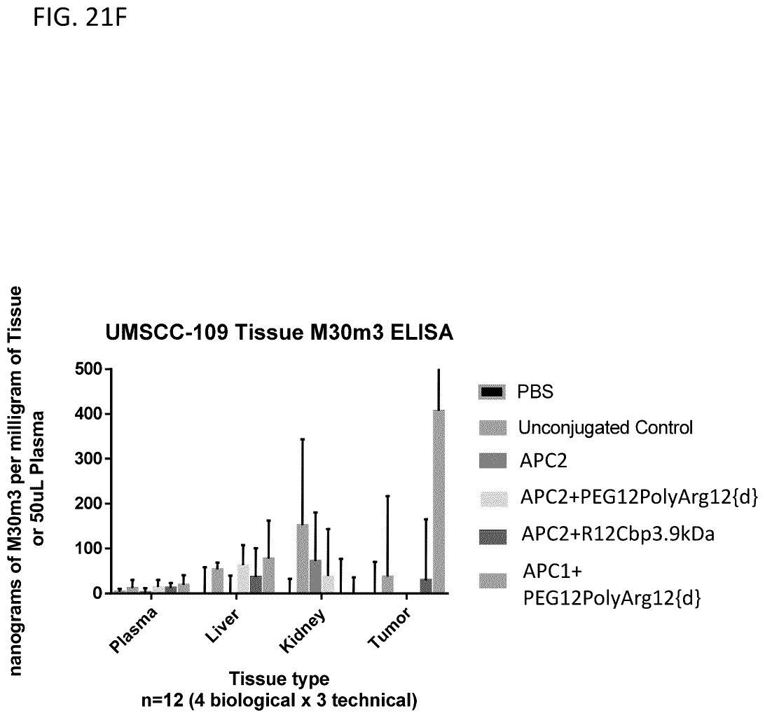

13-week-old female SCID/NCR mice were injected orthotopically with 1×10⁶ UM-SCC-1^{luc} cells and tumor growth was tracked by luminescence from the cells. Once signal reached 1×10⁷ Photons/s/cm²/sr, mice were injected with 200 μL of 5 mg/kg M30m3 that is either mixed with cetuximab at same ratio as conjugates (DAR3, aprox. 20 mg/kg cetuximab and 5 mg/kg of M30-40) and hybrid polymer (cetuximab control), conjugated to cetuximab using a C4(Azide-DBCO)C5 linker without hybrid polymer or with the PEG12-Arg12 hybrid polymer at a charge-to-charge ratio of 1:1. Mice were injected biweekly for 2 weeks (4 total doses). Imaging on an IVIS live animal imager was performed biweekly for luminescence from cells as well as mouse weight measurements. A total of 5 mice per group was used and tracked from day 14 (first injection) to day 124 (end of study). FIG. 21A shows selected images from three representative animals from each group this study. Once mouse tumors exceed 1×10⁹ Photons/s/cm²/sr, or mouse weight dropped by >20% from study start or mouse health deteriorated, mice were sacrificed, and survival plotted. See FIG. 21B. By day 124, 3 mice treated with conjugate with the hybrid polymer remained while all others had to be euthanized. The hybrid polymers, thus, improved tumor control and survival.

Female SCID/NCR mice were injected with 2.5×10⁶ UM-SCC-109 HNSCC cells on the right flank and tumor growth was tracked by measuring tumor dimensions with a caliper and applying the formula of L*W²/2=Volume. Once tumors reached 100 mm³, mice were randomized and injected with 200 μL of 5 mg/kg conjugate (based on cetuximab dose) that is mixed with the PEG12PolyArg12 hybrid polymer at a charge-to-charge ratio of 1:1. Mice were injected biweekly for 2 weeks (4 total doses) (Cetuximab-SMCC-M30m3 DAR4=SMCC Conjugate, Cetuximab-MCVCPABcPNP-M30m3 DAR4=MCVCPABcPNP Conjugate, and Cetuximab-MCPEG4VCPABcPNP-M30m3 DAR4=MCPEG4VCPABcPNP Conjugate). Vehicle (PBS) control and Cetuximab control was also done. A total of 5 mice per group was used. Mouse tumor growth was tracked biweekly over time and weight weekly. If tumors reached 2 cm³ or mouse weight dropped by >20% from study start or mouse health deteriorated, the mice were euthanized. MCVCPABcPNP and SMCC conjugates with the PEG12PolyArg12 hybrid polymer demonstrated an improvement in tumor growth inhibition compared to cetuximab control. See FIG. 21C.

Three HNSCC cell lines were implanted into SCID mice and treated with either PBS control, Cetuximab or APC1 (Cetuximab-PEG4-azide-DBCO-PEG5-M30m3 DAR2=APC1), utilizing MC-30, NAVIgGator™ and the PEG12PolyArg12 hybrid polymer. Tumor volume was measured twice weekly, and weight was tracked once weekly. Once tumors reached 100 mm³, mice were randomized into equal groups. Four mice per group were treated twice weekly for two weeks at 2 mg/kg M30m3 (comparative dose for Cetuximab). The hybrid polymer coated conjugates demonstrated superior tumor inhibition for all three tumor types. See FIG. 21D. * denotes p<0.0005 based on linear regression analysis. Error bars are standard deviation.

UM-SCC-109 HNSCC cells were implanted into SCID mice and treated with either PBS control or Cetuximab-PEG4-azide-DBCO-PEG5-M30m3 at DAR 2.5, 4.5 and 6.5 combined with the Peg12PolyArg12{d}hybrid polymer at a charge:charge ratio of 1:1. Tumor volume was measured twice weekly, and weight was tracked once weekly. Once tumors reached 100 mm³, mice were randomized into equal groups. Five mice per group were treated twice weekly for two weeks at 10 mg/kg cetuximab. DAR 4.5 and 2.5 were found to be superior to DAR 6.5 for inhibiting tumor growth, despite having lower overall M30m3 dose. See FIG. 21E. Error bars are standard deviation.

UM-SCC-109 HNSCC cell line was implanted into SCID mice and treated with either PBS control, unconjugated control mixture (Cetuximab, M30m3, and Peg12PolyArg12{d}), AOC2=Cetuximab-PEG4-azide-DBCO-PEG5-M30m3 at DAR 2 with and without either Peg12PolyArg12{d} or R12Cbp3.9kda at a charge:charge ratio of 0.5:1, or AOC1=Cetuximab-PEG4-azide-DBCO-PEG5-M30m3 at DAR 4 mixed with Peg12PolyArg12{d} at a charge ratio of 0.5:1. After tumors reached 200mm3, mice were randomized into even groups with 4 mice per group and then injected at 10 mg/kg cetuximab conjugate (2 mg/kg of RNA for DAR2 and 4 mg/kg of RNA for DAR4) biweekly for 3 doses. 72 hours after the last dose, plasma, liver, kidney and tumor were collected, homogenized with a Qiagen tissuelyzer at 1 mg/50 uL of Quantigene homogenization buffer and then run through Hybridization ELISA protocol. In brief, homogenized tissue was treated with proteinase K for 1 hour at 37 degrees. A probe specific for M30m3 that has biotin on one end and digoxigenin on the other was then mixed with the tissue sample and the samples were then boiled for 5 min and cooled to anneal the probe to the M30m3 guide strand. The sample was then added to streptavidin plate, incubated for 1 hour, washed and then incubated for 1 hour with S1 nuclease to degrade unbound probe. Wells were then washed and treated with an antibody specific for digoxigenin that is also conjugated to alkaline phosphatase for 1 hour. Excess antibody was washed off and then each well was treated with AttoPhos fluorescent alkaline phosphatase reagent. After 18 hours, wells were read on a plate reader and compared to a spike in standard curve to determine ng of M30m3 per mL. Values were then converted to ng/mg of tissue input. See FIG. 21F. Error bars are standard deviation. Each bar represents 4 biological repeats each with 2 technical repeats each. *represents a p-value<0.05 by student's t-test compared to unconjugated control.

1-52. (canceled)

53. A composition, comprising: hybrid polymer-polynucleotide complexes, each complex, comprising: a single polynucleotide molecule and at least one hybrid polymer, wherein:
(a) the hybrid polymer, comprises: a cationic portion and a neutral portion;
(b) the cationic portion of the hybrid polymer is a polypeptide, comprising:
a. 9 to 18 amino acid residues; and,
b. at least 6 of the amino acid residues are arginine residues; and,
(c) the neutral portion of the hybrid polymer comprises poly(ethylene glycol)(PEG).

54. The composition of claim 53, wherein the cationic polypeptide comprises 12 amino acid residues.

55. The composition of claim 53, wherein the cationic portion of the polypeptide, further comprises: lysine and/or histidine residues.

56. The composition of claim 53, wherein the cationic portion of the polypeptide is a poly-arginine polypeptide.

57. The composition of claim 56, wherein the poly-arginine polypeptide comprises L-amino acid residues.

58. The composition of claim 56, wherein the poly-arginine polypeptide comprises D-amino acid residues.

59. The composition of claim 53, wherein the charge ratio of the cationic polypeptide to the polynucleotide is between 0.25:1 and 5:1.

60. The composition of claim 53, wherein the charge ratio of the cationic polypeptide to the polynucleotide is between 1:1 and 2:1.

61. The composition of claim 53, wherein the PEG comprises: a PEG12 to PEG24 polymer.

62. The composition of claim 53, wherein: the hybrid polymer is selected from the group consisting of
a) PEG12PolyArg12,
b) PolyArg12Cbp3.9 kDa,
c) PEG12PolyArg12{d},
d) PEG24PolyArg12C,
e) PEG24PolyArg12,
f) PolyArg12C-PEG2000 Da,
g) PolyArg12C-PEG5000 Da,
h) PEG1000DaPolyArg12,
i) PEG2000DaPolyArg12,
j) PEG5000DaPolyArg12,
k) PolyArg12Cbp1.5 kDa,
l) PolyArg12Cbp16 kDa,
m) CPolyArg12Cbp1.5 kDa,
n) PolyArg12Cbp2 kDa,
o) PolyArg12bp2 kDa.

63. A method of protecting a polynucleotide from in vivo biological degradation, comprising: contacting the polynucleotide with a hybrid polymer prior to administration of the polynucleotide to form a hybrid polymer-polynucleotide complex, comprising: a single polynucleotide molecule and at least one hybrid polymer, wherein:
(a) the hybrid polymer, comprises: a cationic portion and a neutral portion;
(b) the cationic portion of the hybrid polymer is a polypeptide, comprising:
a. 9 to 18 amino acid residues; and,
b. at least 6 of the amino acid residues are arginine residues; and,
(c) the neutral portion of the hybrid polymer comprises poly(ethylene glycol)(PEG).

64. The method of claim 63, wherein the cationic polypeptide comprises 12 amino acid residues.

65. The method of claim 63, wherein the cationic portion of the polypeptide, further comprises: lysine and/or histidine residues.

66. The method of claim 63, wherein the cationic portion of the polypeptide is a poly-arginine polypeptide.

67. The method of claim 66, wherein the poly-arginine polypeptide comprises L-amino acid residues.

68. The method of claim 66, wherein the poly-arginine polypeptide comprises D-amino acid residues.

69. The method of claim 63, wherein the charge ratio of the cationic polypeptide to the polynucleotide is between 0.25:1 and 5:1.

70. The method of claim 63, wherein the charge ratio of the cationic polypeptide to the polynucleotide is between 1:1 and 2:1.

71. The method of claim 63, wherein the PEG comprises: a PEG12 to PEG24 polymer.

72. The method of claim 63, wherein: the hybrid polymer is selected from the group consisting of
a) PEG12PolyArg12,
b) PolyArg12Cbp3.9 kDa,
c) PEG12PolyArg12{d},
d) PEG24PolyArg12C,
e) PEG24PolyArg12,
f) PolyArg12C-PEG2000 Da,
g) PolyArg12C-PEG5000 Da,
h) PEG1000DaPolyArg12,
i) PEG2000DaPolyArg12,
j) PEG5000DaPolyArg12,
k) PolyArg12Cbp1.5 kDa,
l) PolyArg12Cbp16 kDa,
m) CPolyArg12Cbp1.5 kDa,
n) PolyArg12Cbp2 kDa,
o) PolyArg12bp2 kDa.
